(19) United States
(12) Patent Application Publication
(10) Pub. No.: US 2024/0288442 A1
(43) Pub. Date: Aug. 29, 2024

(54) METHODS OF ASSAYING BIOMOLECULES WITHIN A MICROFLUIDIC DEVICE

(71) Applicant: BERKELEY LIGHTS, INC., Emeryville, CA (US)

(72) Inventors: Phillip J. M. ELMS, Emeryville, CA (US); Preston Lock NG, Emeryville, CA (US); Jonathan CHENG, Emeryville, CA (US); Nathan J. VER HEUL, Emeryville, CA (US); Jason King Mckenzie YEE, Emeryville, CA (US); Hariharasudhan CHIRRA DINAKAR, Emeryville, CA (US); Renee Lynn TOBIAS, Emeryville, CA (US); Eric K. SACKMANN, Emeryville, CA (US); Wesley Arthur ZINK, Emeryville, CA (US); Hector D. NEIRA-QUINTERO, Emeryville, CA (US)

(73) Assignee: BERKELEY LIGHTS, INC., Emeryville, CA (US)

(21) Appl. No.: 18/476,798

(22) Filed: Sep. 28, 2023

Related U.S. Application Data
(63) Continuation of application No. PCT/US2022/071426, filed on Mar. 29, 2022.
(60) Provisional application No. 63/167,335, filed on Mar. 29, 2021.
(60) Provisional application No. 63/225,364, filed on Jul. 23, 2021.
(60) Provisional application No. 63/230,547, filed on Aug. 6, 2021.

(51) Int. Cl. G01N 33/68

(52) U.S. Cl. CPC G01N 33/6854; G01N 2458/00

(57) ABSTRACT

Methods for identifying a cell population secreting a biomolecule with desirable attributes are disclosed. The desirable attributes can include, for example, quantity and quality (e.g., minimal aggregation and/or desired configuration). Cell populations identified by the disclosed methods are more likely to successfully scale during production. The methods can include assessing multiple domains/binding sites of a complex biomolecule and/or the formation of aggregates by the biomolecules. Methods of assessing a secretion level for biomolecules having a wide range of molecular weights and method for enhancing loading of cells into chambers of a microfluidic device. The labelling of viable cells and/or cells actively expressing a biomolecule of interest, for example, can permit selection and subsequent analysis of the cells most likely to successfully expand and express the biomolecule of interest, thereby reducing effort, risk, and cost associated with the screening of cells and increase the probability of identifying cell lines that are optimal producers of biomolecules of interest.

### CROSS-REFERENCE TO RELATED APPLICATIONS

This application is a continuation of International Patent Application No. PCT/2022/071426, filed Mar. 29, 2022, which claims the benefit of priority to U.S. Provisional Application No. 63/167,335, filed on Mar. 29, 2021, U.S. Provisional Application No. 63/225,364, filed on Jul. 23, 2021, and U.S. Provisional Application No. 63/230,547, filed on Aug. 6, 2021, each of which disclosures are herein incorporated by reference in their entirety.

### BACKGROUND TO THE DISCLOSURE

In the bioproduction industry, the expense, time, and difficulty involved in identifying cell lines that produce product high quality product in large quantities has been a major obstacle to greater progress. The embodiments disclosed herein are generally directed towards, systems, apparatuses and methods for optically measuring a quantity or quality parameter associated with a micro-object confined within a defined space. More specifically, there is a need for imaging systems and methods that can accurately determine the quantity and/or quality of an analyte produced by a micro-object confined in a chamber (e.g., sequestration pen) within a microfluidic device.

### SUMMARY OF THE DISCLOSURE

In a first aspect, methods for characterizing a biological micro-object producing an analyte of interest are provided. In certain embodiments, the analyte of interest can include at least a first portion and a second portion different from the first portion, and the methods can include: introducing the biological micro-object into a chamber of a microfluidic device, wherein the microfluidic device includes an enclosure having a flow region, and wherein the chamber is fluidically connected to the flow region; allowing the biological micro-object (or a clonal population of biological micro-objects generated therefrom) to secret the analyte of interest within the chamber; introducing a plurality of first reporter molecules into the flow region and allowing a portion of the plurality of first reporter molecules to diffuse into the chamber, wherein each of the plurality of first reporter molecules is configured to emit a detectable signal (e.g., intrinsically or via a first detectable label) and includes a first binding component configured to bind the first portion of the secreted analyte of interest and thereby form a first reporter molecule: secreted analyte complex (first RMSA complex); introducing a plurality of second reporter molecules into the flow region and allowing a portion of the plurality of second reporter molecules to diffuse into the chamber, wherein each of the plurality of second reporter molecules is configured to emit a detectable signal (e.g., intrinsically or via a second detectable label) and includes a second binding component configured to bind the second portion of the secreted analyte of interest and thereby form a second reporter molecule: secreted analyte complex (second RMSA complex); detecting a first signal associated with the first reporter molecules (e.g., associated with the first detectable label) within a first area of interest within the microfluidic device; detecting a second signal associated with the second reporter molecules (e.g., associated with the second detectable label) within a second area of interest within the microfluidic device; and determining whether a ratio of the detected first signal to the detected second signal falls within a pre-selected range. In certain embodiments, the first area of interest and the second area of interest are proximal to one another, substantially overlapping, or substantially the same. In certain embodiments, detecting the first signal includes determining a first absolute quantitation value of the detected first signal; detecting the second signal includes determining a second absolute quantitation value of the detected second signal; and determining whether the ratio of the detected first signal to the detected second signal falls within a pre-selected range includes determining whether a ratio of the first absolute quantitation value to the second absolute quantitation value falls within the pre-selected range. In certain embodiments, allowing the portion of the plurality of first reporter molecules (and/or the plurality of second reporter molecules) to diffuse into the chamber includes allowing the plurality of first reporter molecules (and/or the plurality of second reporter molecules) to attain a steady state equilibrium between the flow region and the chamber. In certain embodiments, the microfluidic device comprises a plurality of chambers, each fluidically connect and opening to the flow region, and the methods further comprise: introducing each of a plurality of biological micro-object into a respective chamber of the microfluidic device and performing the remaining steps of the methods upon the plurality of biological micro-objects in parallel.

In another aspect, methods of assessing a secretion level of a biological micro-object secreting an analyte of interest are provided. Such methods can include: introducing a biological micro-object into a chamber of a microfluidic device, wherein the microfluidic device includes an enclosure having a flow region and the chamber, and wherein the chamber is fluidically connected to the flow region; allowing the biological micro-object (or a population of biological micro-objects generated therefrom) to secret an analyte mixture within the chamber, wherein the analyte mixture includes a plurality of analytes each having a molecule weight from about 1 kDa to about 600 kDa; introducing a first fluidic medium including a plurality of reporter molecules into the flow region, wherein each reporter molecule of the plurality of reporter molecules includes a detectable label and a binding component configured to bind the analyte of interest, and further wherein a concentration of the plurality of reporter molecules in the first fluidic medium is about 1 to 10 times (e.g., about 1 to about 5 times, or about 1 to about 3 times) a dissociation constant (K_{D}) between the reporter molecule and the analyte of interest, and a molecular weight of the reporter molecule is equal to or less than about 150 kDa (e.g., about 2 kDa to about 150 kDa, about 2 kDa to about 100 kDa, about 2 kDa to about 75 kDa, about 2 kDa to about 50 kDa, about 2 kDa to about 25 kDa, or about 2 kDa to about 10 kDa); allowing the plurality of reporter molecules to diffuse into the chamber for a selected period of time, wherein the selected period of time is sufficient for establishing a steady state equilibrium of the reporter molecules between the flow region and the chamber and is within 3 hours (e.g., within 2.5 hours, within 2 hours, from about 2 hours to about 3 hours, or from about 2 hours to about 2.5 hours); and detecting a signal associated with the detectable label of reporter molecule located within an area of interest within the microfluidic device. In certain embodiments, the methods further including: after the first fluidic medium is introduced into the flow region, introducing a second fluidic medium; wherein the second fluidic medium does not include the reporter molecule. In certain embodiments, the second fluidic medium is introduced into the flow region after detecting the signal associated with the detectable label of reporter molecules located within the area of interest. In certain embodiments, the second fluidic medium is introduced into the flow region before detecting the signal associated with the detectable label of reporter molecules located within the area of interest.

In another aspect, methods for selecting a biological micro-object producing an analyte of interest are provided. The methods can include: introducing a biological micro-object into a chamber of a microfluidic device, wherein the microfluidic device includes an enclosure having a flow region, and wherein the chamber is fluidically connected to the flow region; allowing the biological micro-object (or a clonal population of biological micro-objects generated therefrom) to secrete the analyte of interest within the chamber; introducing a plurality of reporter molecules into the flow region, wherein each reporter molecule of the plurality reporter molecules is configured to emit a detectable signal and includes a binding component configured to bind the analyte of interest; allowing a portion of the plurality of reporter molecules to diffuse into the chamber and bind to the secreted analyte of interest therein, thereby producing a plurality of reporter molecule:secreted analyte (RMSA) complexes; identifying one or more (e.g., plurality of) punctate regions emitting the detectable signal in an area of interest within the microfluidic device; and quantifying the one or more (e.g., plurality of) punctate regions in the area of interest. In certain embodiments, the one or more (e.g., plurality of) punctate regions include aggregated analytes of interest produced by the biological micro-object(s). In certain embodiments, the area of interest includes a region within the chamber that does not contain the biological micro-object (or the clonal population of biological micro-objects generated therefrom). In some embodiments, the area of interest lies along an axis of diffusion between the chamber and the flow region (e.g., along an axis of diffusion between an isolation region of the chamber and a microfluidic channel of the flow region, such as along an axis of diffusion defined by a connection region of the chamber). In other embodiments, the area of interest does not lie along an axis of diffusion between the chamber and the flow region (e.g., not along an axis of diffusion between an isolation region of the chamber and a microfluidic channel of the flow region, or not along an axis of diffusion defined by a connection region of the chamber).

In another aspect, methods for enhanced loading of biological micro-objects secreting a molecule of interest (e.g., an analyte of interest) into chambers of a microfluidic device are provided. In certain embodiments, the method include (in any order): identifying a first subset of a plurality of biological micro-objects having a viable phenotype (e.g., by labeling the plurality of biological micro-objects with a first label); and/or identifying a second subset of the plurality of the biological micro-objects having an expressor cell phenotype (e.g., by labeling the plurality of biological micro-objects with a second label), and wherein the method further includes: introducing the plurality of biological micro-objects into a flow region of the microfluidic device; and selectively disposing biological micro-objects that are members of both the first subset and the second subset of the plurality of biological micro-objects into respective chambers of the plurality of chambers. In certain embodiments, identifying biological micro-objects having a viable phenotype includes labeling the plurality of biological micro-objects with a first label configured to positively or negatively label a live cell and/or determining a physical characteristic (e.g., size and/or shape) of each biological micro-object of the plurality of the biological micro-objects. In certain embodiments, identifying biological micro-objects having an expressor cell phenotype includes labeling the plurality of biological micro-objects with a second label configured to label a molecule of interest (e.g., the analyte of interest) at either a cell surface of a secreting biological micro-object or in a region proximal to (e.g., surrounding) the secreting biological micro-object. In certain embodiments, selectively disposing biological micro-objects that are members of both the first subset and the second subset of the plurality of biological micro-objects further includes: differentiating the biological micro-objects into at least two tiers based on the extent of the expressor cell phenotype (e.g., labeling of the second label); and prioritizing the disposing of biological micro-objects exhibiting a superior expressor cell phenotype (e.g., greater labeling of the second label).

In another aspect, methods for determining relative stability for a plurality of clonal cell lines are provided. In certain embodiments, the method include: receiving imaging data of a microfluidic device that includes a flow region and a first plurality of chambers that are fluidically connected and open to the flow region, wherein the imaging data includes a first analyte assay image taken of a plurality of subclones of a first cell line, wherein each subclone of the first cell line is disposed in an individual chamber of the first plurality of chambers; defining an area of interest for each chamber of the first plurality of chambers, wherein the area of interest includes an image area within the chamber that is suitable for assessing the level of secretion of an analyte of interest by the subclone disposed in the chamber (e.g., the area of interest may be a region that is most sensitive for measuring analyte concentration fluctuations, is least sensitive to the position of biological micro-objects in the chamber when analyte fluctuations are measured, and/or extends along an axis of diffusion between the chamber and the flow region); and generating a prediction of cell line stability based on a signal obtained from the area of interest, wherein the signal is an indicator of cell line stability. In certain embodiments, the signal obtained from the area of interest represents a level of secretion of an analyte of interest, wherein the obtained signal is generated by assaying an intrinsic diffusion gradient (e.g., which may be an equilibration assay and/or a flush assay).

In another aspect, non-transitory computer-readable medium including a program for causing a computer to perform any of the methods disclosed herein are provided. Such methods include any of the foregoing methods, including the methods for characterizing a biological micro-object producing an analyte of interest, methods of assessing a secretion level of a biological micro-object secreting an analyte of interest, methods for selecting a biological micro-object producing an analyte of interest, methods for enhanced loading of biological micro-objects secreting a molecule of interest into chambers of a microfluidic device, and methods for determining relative stability for a plurality of clonal cell lines.

In yet another aspect, kits for performing any of the methods disclosed herein are provided. Such methods include any of the foregoing methods, including the methods for characterizing a biological micro-object producing an analyte of interest, methods of assessing a secretion level of a biological micro-object secreting an analyte of interest, methods for selecting a biological micro-object producing an analyte of interest, methods for enhanced loading of biological micro-objects secreting a molecule of interest into chambers of a microfluidic device, and methods for determining relative stability for a plurality of clonal cell lines.

Additional aspects and advantages of the disclosed methods and compositions will be evident from the following detailed description and related drawings, the examples and partial listing of embodiments, and the claims appended hereto.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A illustrates a microfluidic device and a system with associated control equipment according to some embodiments of the disclosure.

FIG. 1B illustrates a microfluidic device with sequestration pens according to an embodiment of the disclosure.

FIGS. 2A to 2B illustrate a microfluidic device having sequestration pens according to some embodiments of the disclosure.

FIG. 2C illustrates a sequestration pen of a microfluidic device according to some embodiments of the disclosure.

FIG. 3 illustrates a sequestration pen of a microfluidic device according to some embodiments of the disclosure.

FIGS. 4A to 4B illustrate electrokinetic features of a microfluidic device according to some embodiments of the disclosure.

FIG. 5A illustrates a system for use with a microfluidic device and associated control equipment according to some embodiments of the disclosure.

FIG. 5B illustrates an imaging device according to some embodiments of the disclosure.

FIGS. 6A to 6C are graphical representations of diffusion gradient assays according to some embodiments of the disclosure.

FIG. 7 is a graphical representation of a cross-section of a chamber of a micro-fluidic device showing the region of interest and subregions thereof of a diffusion gradient assay according to some embodiments of the disclosure.

FIG. 8A and FIG. 8B are graphical representation of the intensities of a reporter molecule detected within an area of interest (FIG. 8A) and a background image (FIG. 8B).

FIG. 9 is a graphical representation of the intensities of a reporter molecule detected within an area of interest within Pen #1148. The intensities are flat fielded to remove noise and background.

FIG. 10 is a graphical representation of fraction bound as a function of the concentration of reporter molecules.

FIG. 11 is a graphical representation of the signal-to-noise of the target molecule (A) (previously referred to as [M]0) as a function of the reporter molecules (L). The dotted line (arrow) shows the optimal reporter molecule concentration as a function of target molecule concentration.

FIG. 12 is a graphical representation of the diffusion rate as a function of molecular weight of the reporter molecules.

FIG. 13 is a graphical representation showing that the secretion level of cells cultured is one factor affecting the time required to reach a steady state equilibrium. Two cell lines, which were pre-determined as a high secreting cell line and a low secreting cell line respectively are shown. The pen of the high secreting cell line requires a longer time to reach a steady state equilibrium.

FIG. 14 is a graphical representation showing that the secretion level of the cells cultured is a factor affecting the time required to reach a steady state equilibrium. Nine pens having cells of various secretion level or no cells were shown.

FIG. 15 is a graphical representation of assay values for pens based on SpotLight Kappa score (y axis) and SpotLight Fc score (x axis).

FIG. 16 is a graphical representation of assay values for pens based on their Fab score (y axis) and SpotLight Fc score (x axis). The upper figure shows the data from using 50 nM Fab and the bottom figures shows the data from using 250 nM Fab. The pens of the X region and the Y region respectively were selected arbitrarily in the figure to show that the pens of these two regions had similar level of Fab score and distinct level of SpotLight Fc score and the Fab score didn't truthfully represent the secretion of those pens.

FIG. 17 illustrates a method for cell line development according to some embodiments of the disclosure.

FIGS. 18A to 18B are exemplary brightfield and fluorescent photographic representations of cells secreting an analyte according to some embodiments of the disclosure.

FIG. 19A to 19E are exemplary brightfield and fluorescent photographic representations of various chambers containing cells that may or may not secret an analyte according to some embodiments of the disclosure.

FIGS. 20A to 20D illustrate conducting a diffusion assay and an aggregation assay sequentially according to some embodiments of the disclosure.

FIGS. 21A to 21B are brightfield images of a same chamber after a diffusion assay is performed and before (FIG. 21A) and after (FIG. 21B) additional media flow containing no reporter molecules is performed according to some embodiments of the disclosure.

FIGS. 22A to 22B show exemplary fluorescent images of three representative chambers having cells within that secrete an analyte and demonstrate punctate regions of fluorescence (red) according to some embodiments of the disclosure and quantification (FIG. 22B) thereof indicating chamber 819 has 19 punctate regions; chamber 374 has 27 punctate regions, and chamber 835 has 37 punctate regions.

FIG. 23 illustrates plotting the results of both a diffusion assay (along x axis) and an aggregation assay (along y axis) according to some embodiments of the disclosure and selecting pens having cells which both secrete greater quantities of product molecule while producing relatively less aggregation product (boxed region) based on the plot.

FIGS. 24A to 24D are images of a chamber taken during an aggregation assay according to some embodiments of the disclosure. FIG. 24A shows a brightfield image of a chamber containing cells that express an analyte. FIG. 24B shows a functional punctate detection region of the chamber, where the black regions are excluded from the functional detection region while the remainder of the chamber is within the functional detection region.

FIG. 24C is the base fluorescent image of the same chamber showing fluorescence associated with the cells as well as fluorescence associated with punctate fluorescent regions. FIG. 24D is the processed fluorescent image of the same chamber showing the punctate regions identified as two red colored regions 2410, 2420, according to some embodiments of the disclosure.

FIGS. 25A and 25B illustrate a result of an aggregation assay according to some embodiments of the disclosure. FIG. 25A is a ranking of chambers, e.g, pens, of the associated aggregation score obtained from quantifying the punctate regions, according to some embodiments of the disclosure. FIG. 25B is a graphic relating productivity vs growth rate, and inverse of aggregation scores against growth rate, according to some embodiments of the disclosure.

FIG. 26 is a graphical representation of distribution of productive cells possible in wellplate format vs microfluidic format, and further showing a region within the distribution for microfluidic screening that identifies highly productive clones.

FIGS. 27A to 27C are graphical and photographic representations of diffusion characteristics within a chamber of a microfluidic device and an area of interest for assessing levels of secretion of a product from a biological micro-object, according to some embodiments of the disclosure.

FIG. 28 is a photographic and a graphical representation of a course of a culturing and assay sequence according to some embodiments of the disclosure.

FIG. 29A is a graphical representation of assay values for pens based on based on their intensity scores of two types of reporter molecules: Antigen G (y axis) and Antigen I (x axis). Four cell lines were tested for assessing the assembly of the bispecific antibodies they produced. The pens were circled into three subpopulations based on the distinct distribution on the plot.

FIG. 29B is a graphical representation of assay values for pens based on their intensity scores of two types of reporter molecules: Antigen G (y axis) and SpotLight Kappa (x axis). Four cell lines were tested for assessing the assembly of the bispecific antibodies they produced.

FIG. 30 shows separately the graphical representation of assay values of the four cell lines in FIG. 29A and circles subpopulations among the pens of each cell lines.

FIG. 31 shows a graphical representation of absolute quantitation values for pens based on based on their intensity scores of two types of reporter molecules: Antigen G (y axis) and Antigen I (x axis). The absolute quantitation values were calculated from the assay values as shown in FIG. 29A. Only Cell Lines B and C were shown in this figure.

FIG. 32 shows the images of Pen #1166, #510, and #1711 taken in Example 2 as an example of conducting a diffusion assay and an aggregation assay sequentially according to some embodiments of the disclosure.

FIGS. 33A to 33C are photographic representations showing the co-localization of two independent fluorescent antibody binding reagents to aggregate spots in the chambers. FIG. 33A: SpotLight Human Fc staining; FIG. 33B: anti-human Fc FAb, Jackson ImmunoResearch #109-546-170; FIG. 33C: Overlay image of FIG. 33A and FIG. 33B where yellow color indicates where both molecules have bound. The white dots indicate CHO cells cultured at bottom of the pens.

FIGS. 34A to 34D are photographic representations showing the specificity of SpotLight Human Fc to the aggregate spots in the pens. FIG. 34A: SpotLight Human Fc staining; FIG. 34B: anti-human Fc FAb, Jackson ImmunoResearch #109-546-170; FIG. 34C: Non-binding antibody (FITC); FIG. 34D: Cell membrane stain ANS (10 ug/mL).

FIGS. 35A and 35B show the fluorescent punctate pattern can be reproduced with aggregated purified human IgG. FIG. 35A: Heat-stressed IgG forms a spotting pattern in the chip fluidic channel when stained with SpotLight Human Fc reagent. FIG. 35B. A dilution series of aggregated IgG samples shows the number of spots counted in the channel increases with concentration of IgG aggregates loaded, confirming the spots represent the aggregated protein.

FIGS. 36A and 36B are boxplots showing that the on-chip aggregation scores correlate with lower growth and titer. Comparison of distribution of FIG. 36A. viable cell density and FIG. 36B. titer of clones with high versus low aggregation scores. High aggregation score is defined as >/=10.

FIG. 37A shows that the on-chip aggregation scores are predictive of purification success. The x axis presents twelve experiments arranged by their aggregation scores (from low to high). The histogram (blue) represents the levels of high molecular weight aggregates (Arca %, y axis at the left) produced by clones of various aggregation scores. The yellow circles show the yield from protein A purification (ug/mL, y axis at the right), and the dotted line in yellow gives the trend of the yield along the clones.

FIG. 37B is a boxplot showing the distribution of the percent of high molecular weight species detected by the SE-UPLC for clones with on-chip aggregation score. The percent of high molecular weight species at y axis were normalized by Protein A yield. The high punctate count group and low punctate count group at x axis were determined by a threshold of 10.

FIG. 37C is a boxplot showing the total aggregates for clones with on-chip aggregation score. The total aggregates at y axis were normalized by density. The high punctate count group and low punctate count group at x axis were determined by a threshold of 10.

FIG. 37D is a histogram showing the distribution of the scores of pens. The x axis lists out the aggregation score from 0 to 37 and the y axis shows the number of the pens with the corresponding scores.

FIG. 37E shows the correlation between the percent of aggregated subclones from secondary screen and the percent of high molecular weight species values from the SE-UPLC. Eleven clones of different percent of high molecular weight species values were selected and scaled up. About 300 cells from each clone were then loaded onto a new chip for secondary screen (based on the percent punctate count above a threshold of 6). The correlation coefficient (r value) was 0.90.

FIGS. 38A to 38C show the aggregation scores correlate with aggregation observed in shake flask culture. FIG. 38A and FIG. 38B: qLD histogram distributions of particle concentrations binned by size. When viewed by high (FIG. 38B) versus low (FIG. 38A) on-chip aggregation score, large aggregate particles are primarily observed in high scoring clones. FIG. 38C: Correlation of total aggregate concentration determined with qLD (integrated value of qLD particle concentration curves, Q) with on-chip aggregation scores normalized to productivity (Au) scores from Opto CLD secretion assay. Outlier sample in red showed top % high molecular weight species in SE-UPLC and is not included in calculation of R2.

FIG. 39 is an exemplary workflow for verifying a method for expressor enhanced penning according to some embodiments of the disclosure.

FIG. 40 illustrates the doubling time of cells cultured at 4° C., 25° C., and 37° C. for 1 hour or 5 hours respectively, evaluating the effect of pre-loading preparation on cell viability.

FIG. 41A is a photographic representation of FITC fluorescent pre-stained cells (for example, see cells that are pointed by the arrows) within a microfluidic channel according to some embodiments of the disclosure.

FIG. 41B is a graphical representation of the separation between pre-stained cells and non-stained cells according to some embodiments of the disclosure.

FIG. 42A is a photographic representation of cells distributed within a portion of the microfluidic channel.

FIG. 42B is a photographic CY5 fluorescent image of the same region of the microfluidic channel according to some embodiments of the disclosure.

FIG. 43A is a photographic representation of cells showing the same view as that of FIG. 42A.

FIG. 43B is a photographic TxRed fluorescent representation of the same region of the microfluidic channel according to some embodiments of the disclosure.

FIG. 44 is a graphical representation of the distribution of cells within the channel based on FITC or TxRed fluorescent properties of the cells.

FIG. 45A is a graphical representation of the distribution of cells that were selected for penning by the method according to some embodiments of the disclosure.

FIG. 45B shows detail photographs of individual cells in brightfield and in TxRed fluorescent image for selection for penning according to some embodiments of the disclosure.

FIGS. 46A to 46D show various images of individual cell identification and penning according to some embodiments of the disclosure.

FIG. 47A shows a graphical presentation of the identification of desired cell type and final ration of desired secreting cells: undesired non-secreting cells using the dual stained assisted screening method according to some embodiments of the disclosure.

FIG. 47B shows a graphical presentation of the identification of desired cell type and final ration of desired secreting cells: undesired non-secreting cells using a standard single cell penning method.

FIGS. 48A to 48D are photographic representations of cells disposed within the same row of chambers under various imaging conditions.

FIGS. 49A and 49B are photographic representations of cells disposed within the same row of chambers in brightfield illumination and during a diffusion-based assay for secretion according to some embodiments of the disclosure.

FIG. 50 is a graphical representation of the correlation between AuScore and the intensity of SpotLight Kappa. The AuScore was obtained after the cells were penned and cultured on chip for 4 days.

FIG. 51 is a boxplot showing that the mean intensities of CHOsecH, CHOsecL, and CHOnon cells tested in Example 6 were distinguishable from each other.

FIG. 52A is a boxplot comparing the effects of various kinds of enhancers at various dilution ratio.

FIG. 52B is a heatmap table showing the Cohen's d value of the mean intensities shown in FIG. 52A.

FIG. 53 shows the AuScore (left) and rQp (right) of three chips at which the cells were loaded using the basic method or the expressor enhanced penning according to the Example 7. CLD 1.0: the basic penning method; TPS: the expressor enhanced penning method.

FIG. 54 is a photographic representation of the images of a diffusion assay showing the enrichment achieved by the expressor enhanced penning compared with the basic method according to the Example 7.

FIG. 55 shows the enrichment effect that the expressor enhanced penning method of the present disclosure can achieve. Comparing the basic method and the expressor enhanced penning method according to the Example 7, FIG. 55(A) shows the load throughput; FIG. 55(B) shows on-chip viability; FIG. 55(C) shows overall higher AuScore (y axis) of chip penned using expressor enhanced penning method and also shows the distribution of pens having non-secretors (red) and secretors (green) in the plot; the number to the left indicates the number of pens having secretors; FIG. 55(D) shows the overall AuScore (y axis) of the top 48 clones on the chips of the basic method and the expressor enhanced penning respectively; FIG. 55(E) shows the top 96 clones among all the pens on the chips of the basic method and the expressor enhanced penning. CLD 1.0: the basic penning method; TPS: the expressor enhanced penning method.

FIGS. 56A to 56C shows the enrichment effect that the expressor enhanced penning method of the present disclosure can achieve. FIG. 56A shows overall higher AuScore (y axis) of chip penned using expressor enhanced penning method and also shows the distribution of pens having non-secretors (red) and secretors (green) in the plot; FIG. 56B shows the overall AuScore (y axis) of the top 48 clones on the chips of the basic method and the expressor enhanced penning respectively; FIG. 56C shows the top 96 clones among all the pens on the chips of the basic method and the expressor enhanced penning. CLD 1.0: the basic penning method; TPS: the expressor enhanced penning method.

FIG. 57A shows histogram plot for cells binned by the brightness of Annexin V before (left) and after (right) penning.

FIG. 57B shows histogram plot for cells binned by the brightness of SpotLight Kappa before (left) and after (right) penning.

FIG. 57C is a histogram showing the on-chip cell expansion (OCCE) of cells penned by using OEP only (with staining or labeling described in the present disclosure) or expressor enhanced penning methods (using CellTracker or Annexin V, respectively as sorting criteria). Each penning method was performed in triplicate.

FIG. 58A is a graphical representation of the distribution of relative productivity across a plurality of chambers for each of five secreting cell lines that have known levels of secretion.

FIG. 58B is a graphical representation of the relationship of the average relative productivity rQp compared to the observed macroscale productivity Qp for each of the five cell lines of FIG. 58A.

FIGS. 59A to 59C are graphical representations of the relationship of median relative productivity rQp with rank order of the stability of each of five cells lines of FIG. 58A.

FIGS. 60A and 60B illustrate exemplary data showing improvement in signal from averaged images taken for an equilibration assay.

FIGS. 61A and 61B depict photographic images of a microfluidic device before and after normalization according to some embodiments of the disclosure.

FIGS. 62A to 62C are graphical and photographic representations of assay images within a microfluidic device and assay data for an area of interest thereof, according to some embodiments of the disclosure.

FIG. 63 is a graphical representation of an overlay of median intensity values for a plurality of chambers within a microfluidic device, according to some embodiments of the disclosure.

FIGS. 64A and 64B are graphical representations of analyte secretion by biological micro-objects disposed within a microfluidic device, according to some embodiments of the disclosure.

### DETAILED DESCRIPTION

This specification describes exemplary embodiments and applications of the disclosure. The disclosure, however, is not limited to these exemplary embodiments and applications or to the manner in which the exemplary embodiments and applications operate or are described herein. Moreover, the figures may show simplified or partial views, and the dimensions of elements in the figures may be exaggerated or otherwise not in proportion. In addition, as the terms “on,” “attached to,” “connected to,” “coupled to,” or similar words are used herein, one element (e.g., a material, a layer, a substrate, etc.) can be “on,” “attached to,” “connected to,” or “coupled to” another element regardless of whether the one element is directly on, attached to, connected to, or coupled to the other element or there are one or more intervening elements between the one element and the other element. Also, unless the context dictates otherwise, directions (e.g., above, below, top, bottom, side, up, down, under, over, upper, lower, horizontal, vertical, “x,” “y,” “z,” etc.), if provided, are relative and provided solely by way of example and for ease of illustration and discussion and not by way of limitation. In addition, where reference is made to a list of elements (e.g., elements a, b, c), such reference is intended to include any one of the listed elements by itself, any combination of less than all of the listed elements, and/or a combination of all of the listed elements. Section divisions in the specification are for ease of review only and do not limit any combination of elements discussed.

Where dimensions of microfluidic features are described as having a width or an area, the dimension typically is described relative to an x-axial and/or y-axial dimension, both of which lie within a plane that is parallel to the substrate and/or cover of the microfluidic device. The height of a microfluidic feature may be described relative to a z-axial direction, which is perpendicular to a plane that is parallel to the substrate and/or cover of the microfluidic device. In some instances, a cross sectional area of a microfluidic feature, such as a channel or a passageway, may be in reference to a x-axial/z-axial, a y-axial/z-axial, or an x-axial/y-axial area.

As used herein, “substantially” means sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance. When used with respect to numerical values or parameters or characteristics that can be expressed as numerical values, “substantially” means within ten percent.

The term “ones” means more than one. As used herein, the term “plurality” can be 2, 3, 4, 5, 6, 7, 8, 9, 10, or more.

As used herein: μm means micrometer, μm³ means cubic micrometer, pL means picoliter, nL means nanoliter, and μL (or uL) means microliter.

As used herein, “air” refers to the composition of gases predominating in the atmosphere of the earth. The four most plentiful gases are nitrogen (typically present at a concentration of about 78% by volume, e.g., in a range from about 70-80%), oxygen (typically present at about 20.95% by volume at sea level, e.g. in a range from about 10% to about 25%), argon (typically present at about 1.0% by volume, e.g. in a range from about 0.1% to about 3%), and carbon dioxide (typically present at about 0.04%, e.g., in a range from about 0.01% to about 0.07%). Air may have other trace gases such as methane, nitrous oxide or ozone, trace pollutants and organic materials such as pollen, diesel particulates and the like. Air may include water vapor (typically present at about 0.25% or may be present in a range from about 10 ppm to about 5% by volume). Air may be provided for use in culturing experiments as a filtered, controlled composition and may be conditioned as described herein.

As used herein, the term “disposed” encompasses within its meaning “located.”

As used herein, a “microfluidic device” or “microfluidic apparatus” is a device that includes one or more discrete microfluidic circuits configured to hold a fluid, each microfluidic circuit comprised of fluidically interconnected circuit elements, including but not limited to region(s), flow path(s), channel(s), chamber(s), and/or pen(s), and at least one port configured to allow the fluid (and, optionally, micro-objects suspended in the fluid) to flow into and/or out of the microfluidic device. Typically, a microfluidic circuit of a microfluidic device will include a flow region, which may include a microfluidic channel, and at least one chamber, and will hold a volume of fluid of less than about 1 mL, e.g., less than about 750, 500, 250, 200, 150, 100, 75, 50, 25, 20, 15, 10, 9, 8, 7, 6, 5, 4, 3, or 2 μL. In certain embodiments, the microfluidic circuit holds about 1-2, 1-3, 1-4, 1-5, 2-5, 2-8, 2-10, 2-12, 2-15, 2-20, 5-20, 5-30, 5-40, 5-50, 10-50, 10-75, 10-100, 20-100, 20-150, 20-200, 50-200, 50-250, or 50-300 μL. The microfluidic circuit may be configured to have a first end fluidically connected with a first port (e.g., an inlet) in the microfluidic device and a second end fluidically connected with a second port (e.g., an outlet) in the microfluidic device.

As used herein, a “nanofluidic device” or “nanofluidic apparatus” is a type of microfluidic device having a microfluidic circuit that contains at least one circuit element configured to hold a volume of fluid of less than about 1 μL, e.g., less than about 750, 500, 250, 200, 150, 100, 75, 50, 25, 20, 15, 10, 9, 8, 7, 6, 5, 4, 3, 2, 1 nL or less. A nanofluidic device may comprise a plurality of circuit elements (e.g., at least 2, 3, 4, 5, 6, 7, 8, 9, 10, 15, 20, 25, 50, 75, 100, 150, 200, 250, 300, 400, 500, 600, 700, 800, 900, 1000, 1500, 2000, 2500, 3000, 3500, 4000, 4500, 5000, 6000, 7000, 8000, 9000, 10,000, or more). In certain embodiments, one or more (e.g., all) of the at least one circuit elements is configured to hold a volume of fluid of about 100 pL to 1 nL, 100 pL to 2 nL, 100 pL to 5 nL, 250 pL to 2 nL, 250 pL to 5 nL. 250 pL to 10 nL, 500 pL to 5 nL, 500 pL to 10 nL, 500 pL to 15 nL, 750 pL to 10 nL, 750 pL to 15 nL, 750 pL to 20 nL, 1 to 10 nL, 1 to 15 nL, 1 to 20 nL, 1 to 25 nL, or 1 to 50 nL. In other embodiments, one or more (e.g., all) of the at least one circuit elements are configured to hold a volume of fluid of about 20 nL to 200 nL, 100 to 200 nL, 100 to 300 nL, 100 to 400 nL, 100 to 500 nL, 200 to 300 nL, 200 to 400 nL, 200 to 500 nL, 200 to 600 nL, 200 to 700 nL, 250 to 400 nL, 250 to 500 nL, 250 to 600 nL, or 250 to 750 nL.

A microfluidic device or a nanofluidic device may be referred to herein as a “microfluidic chip” or a “chip”; or “nanofluidic chip” or “chip”.

A “microfluidic channel” or “flow channel” as used herein refers to flow region of a microfluidic device having a length that is significantly longer than both the horizontal and vertical dimensions. For example, the flow channel can be at least 5 times the length of either the horizontal or vertical dimension, e.g., at least 10 times the length, at least 25 times the length, at least 100 times the length, at least 200 times the length, at least 500 times the length, at least 1,000 times the length, at least 5,000 times the length, or longer. In some embodiments, the length of a flow channel is about 100,000 microns to about 500,000 microns, including any value therebetween. In some embodiments, the horizontal dimension is about 100 microns to about 1000 microns (e.g., about 150 to about 500 microns) and the vertical dimension is about 25 microns to about 200 microns, (e.g., from about 40 to about 150 microns). It is noted that a flow channel may have a variety of different spatial configurations in a microfluidic device, and thus is not restricted to a perfectly linear element. For example, a flow channel may be, or include one or more sections having, the following configurations: curve, bend, spiral, incline, decline, fork (e.g., multiple different flow paths), and any combination thereof. In addition, a flow channel may have different cross-sectional areas along its path, widening and constricting to provide a desired fluid flow therein. The flow channel may include valves, and the valves may be of any type known in the art of microfluidics. Examples of microfluidic channels that include valves are disclosed in U.S. Pat. Nos. 6,408,878 and 9,227,200, each of which is herein incorporated by reference in its entirety.

As used herein, the term “transparent” refers to a material which allows visible light to pass through without substantially altering the light as is passes through.

As used herein, “brightfield” illumination and/or image refers to white light illumination of the microfluidic field of view from a broad-spectrum light source, where contrast is formed by absorbance of light by objects in the field of view.

As used herein, “structured light” is projected light that is modulated to provide one or more illumination effects. A first illumination effect may be projected light illuminating a portion of a surface of a device without illuminating (or at least minimizing illumination of) an adjacent portion of the surface, e.g., a projected light pattern, as described more fully below, used to activate DEP forces within a DEP substrate. When using structured light patterns to activate DEP forces, the intensity, e.g., variation in duty cycle of a structured light modulator such as a DMD, may be used to change the optical power applied to the light activated DEP actuators, and thus change DEP force without changing the nominal voltage or frequency. Another illumination effect that may be produced by structured light includes projected light that may be corrected for surface irregularities and for irregularities associated with the light projection itself, e.g., fall-off at the edge of an illuminated field. Structured light is typically generated by a structured light modulator, such as a digital mirror device (DMD), a microshutter array system (MSA), a liquid crystal display (LCD), or the like. Illumination of a small area of the surface, e.g., a selected area of interest, with structured light improves the signal-to-noise-ratio (SNR), as illumination of only the selected area of interest reduces stray/scattered light, thereby lowering the dark level of the image. An important aspect of structured light is that it may be changed quickly over time. A light pattern from the structured light modulator, e.g., DMD, may be used to autofocus on difficult targets such as clean mirrors or surfaces that are far out of focus. Using a clean mirror, a number of self-test features may be replicated such as measurement of modulation transfer function and field curvature/tilt, without requiring a more expensive Shack-Hartmann sensor. In another use of structured light patterns, spatial power distribution may be measured at the sample surface with a simple power meter, in place of a camera. Structured light patterns may also be used as a reference feature for optical module/system component alignment as well used as a manual readout for manual focus. Another illumination effect made possible by use of structured light patterns is selective curing, e.g., solidification of hydrogels within the microfluidic device.

As used herein, the term “micro-object” refers generally to any microscopic object that may be isolated and/or manipulated in accordance with the present disclosure. Non-limiting examples of micro-objects include: inanimate micro-objects such as microparticles; microbeads (e.g., polystyrene beads, glass beads, amorphous solid substrates, Luminex™ beads, or the like); magnetic beads; microrods; microwires; quantum dots, and the like; biological micro-objects such as cells; biological organelles; vesicles, or complexes; synthetic vesicles; liposomes (e.g., synthetic or derived from membrane preparations); lipid nanorafts, and the like; or a combination of inanimate micro-objects and biological micro-objects (e.g., microbeads attached to cells, liposome-coated micro-beads, liposome-coated magnetic beads, or the like). Beads may include moieties/molecules covalently or non-covalently attached, such as fluorescent labels, proteins (including receptor molecules), carbohydrates, antigens, small molecule signaling moieties, or other chemical/biological species capable of use in an assay. In some variations, beads/solid substrates including moieties/molecules may be capture beads, e.g., configured to bind molecules including small molecules, peptides, proteins or nucleic acids present in proximity either selectively or nonselectively. In one nonlimiting example, a capture bead may include a nucleic acid sequence configured to bind nucleic acids having a specific nucleic acid sequence or the nucleic acid sequence of the capture bead may be configured to bind a set of nucleic acids having related nucleic acid sequences. Either type of binding may be understood to be selective. Capture beads containing moieties/molecules may bind nonselectively when binding of structurally different but physico-chemically similar molecules is performed, for example, size exclusion beads or zeolites configured to capture molecules of selected size or charge. Lipid nanorafts have been described, for example, in Ritchie et al. (2009) “Reconstitution of Membrane Proteins in Phospholipid Bilayer Nanodiscs,” Methods Enzymol., 464:211-231.

As used herein, the term “cell” is used interchangeably with the term “biological cell.” Non-limiting examples of biological cells include eukaryotic cells, plant cells, animal cells, such as mammalian cells, reptilian cells, avian cells, fish cells, or the like, prokaryotic cells, bacterial cells, fungal cells, protozoan cells, or the like, cells dissociated from a tissue, such as muscle, cartilage, fat, skin, liver, lung, neural tissue, and the like, immunological cells, such as T cells, B cells, natural killer cells, macrophages, and the like, embryos (e.g., zygotes), oocytes, ova, sperm cells, hybridomas, cultured cells, cells from a cell line, cancer cells, infected cells, transfected and/or transformed cells, reporter cells, and the like. A mammalian cell can be, for example, from a human, a mouse, a rat, a horse, a goat, a sheep, a cow, a primate, or the like.

A colony of biological cells is “clonal” if all of the living cells in the colony that are capable of reproducing are daughter cells derived from a single parent cell. In certain embodiments, all the daughter cells in a clonal colony are derived from the single parent cell by no more than 10 divisions. In other embodiments, all the daughter cells in a clonal colony are derived from the single parent cell by no more than 14 divisions. In other embodiments, all the daughter cells in a clonal colony are derived from the single parent cell by no more than 17 divisions. In other embodiments, all the daughter cells in a clonal colony are derived from the single parent cell by no more than 20 divisions. The term “clonal cells” refers to cells of the same clonal colony.

As used herein, a “colony” of biological cells refers to 2 or more cells (e.g., about 2 to about 20, about 4 to about 40, about 6 to about 60, about 8 to about 80, about 10 to about 100, about 20 to about 200, about 40 to about 400, about 60 to about 600, about 80 to about 800, about 100 to about 1000, or greater than 1000 cells).

As used herein, the term “maintaining (a) cell(s)” refers to providing an environment comprising both fluidic and gaseous components and, optionally a surface, that provides the conditions necessary to keep the cells viable and/or expanding.

As used herein, the term “expanding” when referring to cells, refers to increasing in cell number.

As referred to herein, “gas permeable” means that the material or structure is permeable to at least one of oxygen, carbon dioxide, or nitrogen. In some embodiments, the gas permeable material or structure is permeable to more than one of oxygen, carbon dioxide and nitrogen and may further be permeable to all three of these gases.

A “component” of a fluidic medium is any chemical or biochemical molecule present in the medium, including solvent molecules, ions, small molecules, antibiotics, nucleotides and nucleosides, nucleic acids, amino acids, peptides, proteins, sugars, carbohydrates, lipids, fatty acids, cholesterol, metabolites, or the like.

As used herein in reference to a fluidic medium, “diffuse” and “diffusion” refer to thermodynamic movement of a component of the fluidic medium down a concentration gradient.

The phrase “flow of a medium” means bulk movement of a fluidic medium primarily due to any mechanism other than diffusion, and may encompass perfusion. For example, flow of a medium can involve movement of the fluidic medium from one point to another point due to a pressure differential between the points. Such flow can include a continuous, pulsed, periodic, random, intermittent, or reciprocating flow of the liquid, or any combination thereof. When one fluidic medium flows into another fluidic medium, turbulence and mixing of the media can result. Flowing can comprise pulling solution through and out of the microfluidic channel (e.g., aspirating) or pushing fluid into and through a microfluidic channel (e.g., perfusing).

The phrase “substantially no flow” refers to a rate of flow of a fluidic medium that, when averaged over time, is less than the rate of diffusion of components of a material (e.g., an analyte of interest) into or within the fluidic medium. The ratio of a rate of flow of a component in a fluidic medium (i.e., advection) divided by the rate of diffusion of such component can be expressed by a dimensionless Peclet number. Thus, a region within a microfluidic device that experiences substantially no flow in one in which the Peclet number is less than 1. The Peclet number associated with a particular region within the microfluidic device can vary with the component or components of the fluidic medium being considered (e.g., the analyte of interest), as the rate of diffusion of a component or components in a fluidic medium can depend on, for example, temperature, the size, mass, and/or shape of the component(s), and the strength of interactions between the component(s) and the fluidic medium. In certain embodiments, the Peclet number associated with a particular region of the microfluidic device and a component located therein can be 0.95 or less, 0.9 or less, 0.85 or less, 0.8 or less, 0.75 or less, 0.7 or less, 0.65 or less, 0.6 or less, 0.55 or less, 0.5 or less, 0.4 or less, 0.3 or less, 0.2 or less, 0.1 or less, 0.05 or less, 0.01 or less, 0.005 or less, or 0.001 or less.

As used herein in reference to different regions within a microfluidic device, the phrase “fluidically connected” means that, when the different regions are substantially filled with fluid, such as fluidic media, the fluid in each of the regions is connected so as to form a single body of fluid. This does not mean that the fluids (or fluidic media) in the different regions are necessarily identical in composition. Rather, the fluids in different fluidically connected regions of a microfluidic device can have different compositions (e.g., different concentrations of solutes, such as proteins, carbohydrates, ions, or other molecules) which are in flux as solutes move down their respective concentration gradients and/or fluids flow through the device.

As used herein, a “flow path” refers to one or more fluidically connected circuit elements (e.g., channel(s), region(s), chamber(s) and the like) that define, and are subject to, the trajectory of a flow of medium. A flow path is thus an example of a swept region of a microfluidic device. Other circuit elements (e.g., unswept regions) may be fluidically connected with the circuit elements that comprise the flow path without being subject to the flow of medium in the flow path.

As used herein, “isolating a micro-object” confines a micro-object to a defined area within the microfluidic device.

As used herein, “pen” or “penning” refers to disposing micro-objects within a chamber (e.g., a sequestration pen) within the microfluidic device. Forces used to pen a micro-object may be any suitable force as described herein such as dielectrophoresis (DEP), e.g., an optically actuated dielectrophoretic force (OEP); gravity; magnetic forces; or tilting. In some embodiments, penning a plurality of micro-objects may reposition substantially all the micro-objects. In some other embodiments, a selected number of the plurality of micro-objects may be penned, and the remainder of the plurality may not be penned. In some embodiments, when selected micro-objects are penned, a DEP force, e.g., an optically actuated DEP force or a magnetic force may be used to reposition the selected micro-objects. Typically, micro-objects may be introduced to a flow region, e.g., a microfluidic channel, of the microfluidic device and introduced into a chamber by penning.

As used herein, “unpen” or “unpenning” refers to repositioning micro-objects from within a chamber, e.g., a sequestration pen, to a new location within a flow region, e.g., a microfluidic channel, of the microfluidic device. Forces used to unpen a micro-object may be any suitable force as described herein such as dielectrophoresis, e.g., an optically actuated dielectrophoretic force; gravity; magnetic forces; or tilting. In some embodiments, unpenning a plurality of micro-objects may reposition substantially all the micro-objects. In some other embodiments, a selected number of the plurality of micro-objects may be unpenned, and the remainder of the plurality may not be unpenned. In some embodiments, when selected micro-objects are unpenned, a DEP force, e.g., an optically actuated DEP force or a magnetic force may be used to reposition the selected micro-objects.

As used herein, “export” or “exporting” refers to repositioning micro-objects from a location within a flow region, e.g., a microfluidic channel, of a microfluidic device to a location outside of the microfluidic device, such as a 96 well plate or other receiving vessel. The orientation of the chamber(s) having an opening to the microfluidic channel permits easy export of micro-objects that have been positioned or repositioned (e.g., unpenned from a chamber) to be disposed within the microfluidic channel. Micro-objects within the microfluidic channel may be exported without requiring disassembly (e.g., removal of the cover of the device) or insertion of a tool into the chamber(s) or microfluidic channel to remove micro-objects for further processing.

A microfluidic (or nanofluidic) device can comprise “swept” regions and “unswept” regions. As used herein, a “swept” region is comprised of one or more fluidically interconnected circuit elements of a microfluidic circuit, each of which experiences a flow of medium when fluid is flowing through the microfluidic circuit. The circuit elements of a swept region can include, for example, regions, channels, and all or parts of chambers. As used herein, an “unswept” region is comprised of one or more fluidically interconnected circuit element of a microfluidic circuit, each of which experiences substantially no flux of fluid when fluid is flowing through the microfluidic circuit. An unswept region can be fluidically connected to a swept region, provided the fluidic connections are structured to enable diffusion but substantially no flow of media between the swept region and the unswept region. The microfluidic device can thus be structured to substantially isolate an unswept region from a flow of medium in a swept region, while enabling substantially only diffusive fluidic communication between the swept region and the unswept region. For example, a flow channel of a micro-fluidic device is an example of a swept region while an isolation region (described in further detail below) of a microfluidic device is an example of an unswept region.

As used herein, a “non-sweeping” rate of fluidic medium flow means a rate of flow sufficient to permit components of a second fluidic medium in an isolation region of the sequestration pen to diffuse into the first fluidic medium in the flow region and/or components of the first fluidic medium to diffuse into the second fluidic medium in the isolation region; and further wherein the first medium does not substantially flow into the isolation region.

As used herein, the term “equilibrium” refers to a state of a system in which the average quantity of one or more species of interest (e.g., reporter, analyte, and/or reporter-analyte (or RMSA) complex) does not change as a function of time. In some instances, the system is a closed system that attains equilibrium from non-equilibrium initial conditions. In other instances, the system is an open system that attains equilibrium when the rate of generation and/or addition of the species of interest to the system is equal to the rate of destruction and/or removal of the species of interest from the system. As used herein, the term “steady state” refers to an equilibrium condition in an open system in which the net change of a species of interest over time is zero. As used herein, the term “non-equilibrium” refers to a state of a system in which the average quantity of one or more species of interest (e.g., reporter, analyte, and/reporter-analyte (or RMSA) complex) changes as a function of time.

As used herein, the term “intrinsic diffusion gradient” as defined herein refers to a difference in concentration of a species of interest (e.g., reporter, analyte, and/or reporter-analyte (or RMSA) complex) between a first region and a second region within a system in which the species of interest is capable of diffusing between the first region and the second region. For example, the system can have a first region in which the species of interest has a first concentration and a second region in which the species of interest has a second concentration that is less than the first concentration. In some instances, the intrinsic diffusion gradient can arise from the generation of a soluble analyte in a first region of a system, where the system includes a second region in which there is no generation of the soluble analyte (or less generation than in the first region). In some instances, the intrinsic diffusion gradient can arise from continuous generation of a soluble analyte in a first region of the system, diffusion of the soluble analyte from the first region to a second region of the system, and continuous removal of the soluble analyte from the second region of the system. Thus, the first region of the system can contain a “source” of the soluble analyte, and the second region of the system can contain a “sink” for the soluble analyte. If the rate of generation of the soluble analyte by the source remains substantially constant over time, the rate of removal of the soluble analyte by the sink remains substantially constant over time, and the rate of generation is substantially the same as the rate of removal, then a “stable concentration gradient” can form. Alternatively, if the rate of generation of the soluble analyte by the source remains substantially constant over time, the rate of removal of the soluble analyte by the sink remains substantially constant over time, but the rate of generation differs from the rate of removal, then a “transient concentration gradient” can form. In some instances, the intrinsic diffusion gradient is a stable concentration gradient. In other instances, the intrinsic diffusion gradient is a transient concentration gradient.

In certain embodiments, a system useful for measuring an intrinsic diffusion gradient can include: a chamber (e.g., a chamber of a microfluidic device) having an opening (e.g., to a larger chamber or to a flow channel of the microfluidic device); and a source located within the chamber, the source comprising one or more biological micro-objects secreting a soluble analyte of interest, where the opening of the chamber provides a sink for removal of species of interest from the chamber. For the purpose of the methods disclosed herein, a biological micro-object can comprise any micro-object configured or capable of secreting, producing, or otherwise generating a secreted analyte of interest. An intrinsic diffusion gradient can be formed in such a system and, as described further herein, measured in such a system.

As used herein, the terms “region of interest” (or “ROI”) and “area of interest” (or “AOI”) are used interchangeably and, when used in reference to the measurement of an intrinsic diffusion gradient, refer to a region where an intrinsic diffusion gradient or a portion of the intrinsic diffusion gradient can be measured. As used herein, the term “axis of diffusion” refers to an axis within a system which is parallel to the predominant direction of flow of a species of interest as it moves down its intrinsic diffusion gradient. In some instances, the region of interest can include one or more regions that lie along an axis of diffusion within the system. In other instances, the region of interest can include one or more regions that lie off of an axis of diffusion within the system.

### I. DIFFUSION GRADIENT ASSAYS (GENERALLY)

The present disclosure provides methods, systems, and devices for assaying an intrinsic diffusion gradient. Such methods can include detecting soluble molecules (or analytes, reporter molecules, or reporter-analyte complexes) in a microfluidic device. In some instances, the methods include capturing images of a reporter component (e.g., a molecule capable of being detected by an image through emission or absorption of electromagnetic energy typically in the form of photons). In some instances, the intrinsic diffusion gradient can be detected in one or more images as signals which can be correlated with the soluble molecules that form the intrinsic diffusion gradient. Such signals, for example, can have spatial and/or temporal distributions that can be correlated with one or more properties of the intrinsic diffusion gradient. In some instances, the intrinsic diffusion gradient is generated by secretion of an analyte of interest by one or more biological micro-objects, and measuring a secretion profile of the biological micro-object(s) can comprise assaying the intrinsic diffusion gradient.

The present disclosure further provides methods, systems, and devices for quantifying a level of secretion of a biological molecule by a biological micro-object(s). The biological micro-object(s) can be disposed in one or more chambers of a microfluidic device disclosed herein. In some embodiments, the biological molecule is an analyte of interest secreted by the biological micro-object(s) (e.g., a biological cell or population of cells, which may be clonal in nature).

As described herein, the amount of a secreted analyte of a biological micro-object may be quantified using a reporter molecule that binds to the secreted analyte. The reporter molecule includes a binding component that binds the secreted analyte to be quantified and a signal component that is used to detect a quantity of the reporter molecule.

Secreted analyte of interest. An analyte of interest (e.g., target protein) secreted by the biological micro-object may include a protein, a saccharide, a nucleic acid, an organic molecule other than a protein, saccharide, or nucleic acid, or a complex formed by any one or more of the foregoing. In other embodiments, the analyte of interest comprises a supramolecular structure, such as a vesicle or a virus. In some embodiments, the analyte secreted by the biological micro-object may be an antibody. In some embodiments, the analyte secreted by the biological micro-object may be a protein other than an antibody. Whether an antibody or a protein other than an antibody, the secreted analyte of interest may be glycosylated.

A secreted analyte of interest may be a naturally expressed analyte (e.g., natively expressed) or may be a bioengineered analyte (e.g., a product resulting from gene insertion, deletion, modification and the like). A secreted analyte of interest that is a nucleic acid may be a ribonucleic or a deoxynucleic acid, and may include natural or unnatural nucleotides. A secreted analyte of interest that is a virus may be a viral particle, a vector or a phage. A secreted analyte that is a saccharide may be a mono-, di- or polysaccharide. Non-limiting examples of saccharides may include glucose, trehalose, mannose, arabinose, fructose, ribose, xanthan or chitosan. A secreted small, organic molecule may include but is not limited to biofuels, oils, polymers, or pharmaceutics such as macrolide antibiotics. A secreted analyte of interest that is a protein can be an antibody or fragment thereof; a blood protein, such as an albumin, a globulin (e.g., alpha2-macroglobulin, gamma globulin, beta-2 microglobulin, haptoglobulin), a complement protein (e.g., component 3 or 4), transferrin, prothrombin, alpha 1 antitrypsin, and the like; a hormone, such as insulin, glucagon, somatostatin, growth hormone, growth factors (e.g., FGF, HGF, NGF, EGF, PDGF, TGF, Erythropoietin, IGF, TNF), follicle stimulating hormone, luteinizing hormone, leptin, and the like; a fibrous protein, such as a silk or an extracellular matrix protein (e.g., a fibronectin, laminin, collagen, elastin, vitronectin, tenascin, versican, bone sialoprotein); an enzyme, such as a metalloprotease (e.g., matrix metalloproteinase (MMP)) or other type of protease (e.g., serine protease, cysteine protease, threonine protease, aspartic protease, glutamic protease, asparagine peptide lyase), an amylase, a cellulase, a catalase, a pectinase, and the like; a bacterial, yeast, or protozoan protein; a plant protein; or a viral protein, such as a capsid or envelope protein. The secreted analyte may be an antibody-drug conjugate. A non-limiting example of a secreted analyte that may have a combination of a protein, a saccharide, a nucleic acid, an organic molecule having a molecular weight of less than 3 Kd, and/or a virus, can include a proteoglycan or glycoprotein. Secreted analyte can be comprise a engineered binding site commonly used for purification, said purification tags can include but are not limited to be a structured or unstructured binding domain configured to associate with a reporter molecule. This list is not limiting and any protein that is naturally expressed or may be engineered to be secreted may be evaluated by the disclosed methods of assaying an intrinsic diffusion gradient and/or quantifying a level of secretion of a biological molecule by a biological micro-object(s).

A secreted analyte of interest (e.g., analyte) can comprise a broad range of molecular weights while retaining the ability to diffuse through appropriate media. The secreted analyte of interest can comprise a molecular weight, wherein said molecular weight is proportional to a diffusion rate and therefore correlated with how much (e.g., the concentration) of the secreted analyte that accumulates in the pen under a steady state equilibrium.

Reporter molecules. Methods of assaying an intrinsic diffusion gradient and/or quantifying a level of secretion of a biological molecule by a biological micro-object(s) disclosed herein can comprise the use of one or more reporter molecules (e.g., detection reagents). In certain embodiments, such reporter molecules can be configured to: covalently or non-covalently bind to a secreted analyte of interest; and generate a signal that can be detected (e.g., using imaging). The signal (raw or processed using one or more methods disclosed here in) can provide a direct or indirect measure of diffusion related properties, such as concentration(s) and/or diffusion rate constant(s), which are proportional to the molecular weight of the reporter molecule and/or reporter molecule-secreted analyte (RMSA) complex. Sec, e.g., International Publication No. WO 2021/183458, published on 16 Sep. 2021, the entire contents of which is incorporated herein by reference. In some embodiments, the signal is proportional to one or more of the amount of accumulated reporter molecule/RMSA complex resulting from one or more of: the secretion rate of a biological micro-object, the number of biological micro-objects, and/or the fraction bound of the analyte.

A reporter molecule typically includes a binding component configured to bind the secreted analyte of interest. Thus, the binding component may be any suitable binding partner capable of specifically binding to the secreted analyte of interest (e.g., with a binding constant less than 10 micromolar). As used herein, specific binding refers to a preference for binding the secreted analyte of interest over one or more other components of the system (e.g., one or more components on or within the microfluidic device). The binding component may comprise a protein, a peptide, a nucleic acid, a small organic molecule, or any combination thereof.

In some embodiments, the reporter molecule may be multi-valent, comprising more than one binding component such that the reporter molecule is able to bind more than one copy of the secreted analyte of interest or to bind more than one member of a group of secreted analytes. The stoichiometry of the reporter molecule-secreted analyte (RMSA) complex can therefore vary. One or more reporter molecules may bind to one or more secreted analytes, and additionally or alternatively one or more secreted analytes may bind to one or more reporter molecules. Thus, for example, a reporter molecule that binds a single copy of the secreted analyte may form an RMSA complex with a 1:1 stoichiometry. Alternatively, the RMSA complex may have a stoichiometric ratio of 2:1, 3:1, 4:1, 1:2, 1:3, 1:4, 2:2, 4:2, 2:4, etc. of reporter molecule: secreted analyte.

The reporter molecule may have any suitable molecular weight, provided that the reporter molecule is soluble and capable of diffusing in media disposed within the microfluidic device. For example, the reporter molecule may have a molecular weight that is about 10%, 20%, 30%, 40%, 50%, 60%, 70%, 80%, 90%, or about the same as the molecular weight of the secreted analyte of interest. Alternatively, the reporter molecule may have a molecular weight that is greater than the molecule weight on the secreted analyte of interest.

In some embodiments, the analyte of interest may be an antibody (or a fragment thereof) and the reporter molecule may comprise a binding component suitable for binding to antibodies (or fragments thereof). In some embodiments, the binding component of the reporter molecule may bind to an antibody Fc region (e.g., the Fc region of an IgG antibody) or an antibody light chain region (e.g., a lambda light chain region or a kappa light chain region). Thus, for example, the binding component of the reporter molecule may include a peptide, protein, aptamer, etc. configured to bind one or more portions/regions of an antibody (e.g., an IgG antibody or fragment thereof). Molecules capable of specifically binding antibodies are well-known in the art. Sec, e.g., International Publication Nos. WO 2017/181135 and WO 2021/183458.

In some embodiments, the binding component of the reporter molecule may intrinsically possess the ability to generate a detectable signal, such as a visible, luminescent, phosphorescent, or fluorescent signal. In other embodiments, the reporter molecule may comprise a detectable label, which may be covalently attached directly or indirectly to the binding component of the reporter molecule. In other embodiments, the reporter molecule may comprise a detectable label that binds non-covalently to the binding component of the reporter molecule. The detectable label may be a visible, luminescent, phosphorescent, or fluorescent detectable label. In some embodiments, the detectable label may be a fluorescent label. Any suitable fluorescent label may be used, including but not limited to fluorescein, rhodamine, cyanine, phenanthrene or any other class of fluorescent dye.

In some embodiments, the binding component of the reporter molecule may comprise a capture oligonucleotide and the detectable label may be an intercalating dye. For example, the reporter molecule may comprise a capture oligonucleotide and either an intrinsic or extrinsic fluorescent dye may be the detectable label. In some embodiments, the detectable label of the reporter molecule may not be detectable until the capture oligonucleotide binds the analyte of interest, as might be the case when the detectable label is an intercalating dye. More generally, in some embodiments, a detectable label of a reporter molecule may not be detectable until after the RMSA complex has formed, as formation of the RMSA complex may shift the detectable signal to a new wavelength not present prior to binding.

Media. Media suitable for use in methods of assaying an intrinsic diffusion gradient and/or quantifying a level of secretion of a biological molecule by a biological micro-object(s) can be a liquid or a gas, and may comprise reagents (e.g., reporter molecules) or other diffusible components. In various embodiments, the methods may include flowing such a medium (e.g., by stopped flow, continuous flow, pulsed flow, etc., as needed) into a flow region of a microfluidic device. Such flowing (or perfusing) can occur before and/or after introducing biological micro-object(s) into one or more chambers of the microfluidic device.

In certain embodiments, the medium can comprise standard tissue culture components. Exemplary tissue culture components can include: a buffer (e.g., for providing a defined pH and/or ionic strength), dissolved oxygen, one or more soluble stimulatory components, one or more soluble feeder cell components, and/or an exhausted growth medium component. In some embodiments, the amount of dissolved oxygen in the medium may be measured and altered or adjusted as desired, which may be facilitated within the microfluidic environments described herein, as compared to such adjustment in macro-scale culture wellplates, shake flasks, and the like. In some embodiments, the pH of the culture medium within the microfluidic environment may be monitored and altered or adjusted, again which may be facilitated within the microfluidic environments described herein, as compared to plasticware standardly used. In some embodiments, soluble stimulatory components such as cytokines, growth factors, antibodies which activate cell-surface signaling proteins, and the like, any of which may stimulate the cells within the microfluidic environment to reproduce more rapidly or to produce different analytes than prior to introduction of the stimulatory components. In some embodiments, viability of the biological micro-object(s) being cultured (e.g., within the microfluidic device) may be improved by including a portion of the supernatant culture medium of feeder cells that provide auxiliary biomolecules that stimulate or otherwise support the cells. The feeder cells themselves may not be present within the microfluidic device but may be cultured in standard reaction vessels. Accordingly, portions of the culture medium conditioned by the feeder cells may be harvested and delivered to the microfluidic device free of the feeder cells. In some embodiments, one or more compounds and/or reagents configured to prevent biological micro-objects from adhering to each other and the chambers may be added to the culture medium. In some embodiments, exhausted growth medium may be added to the microfluidic environment, which can act as a selection mechanism for analyzing which clones within the microfluidic environment are still able to produce the secreted analyte (or even more readily) and/or may be used to approximate the scaleup environment of various types of reaction vessels, which may include wellplates, shaker flasks and bioreactors. In some embodiments, one or more of these additions to the culture medium may confer a selective pressure on one or more of the cells within the chambers.

In certain embodiments, the medium can further comprise one or more reporter molecules and/or analytes. In other embodiments, the medium can lack a reporter molecule and/or analyte. For example, in methods involving more than one reporter molecule or analyte, the medium can comprise all or less than all of the reporter molecules and/or analytes.

Methods. The disclosed methods for assaying an intrinsic diffusion gradient and/or assessing (e.g., quantifying) a level of secretion of an analyte of interest from a biological micro-object or a population of biological micro-objects (e.g., a clonal population) can include assessing a concentration of a particular diffusible species (e.g., free reporter, reporter bound to secreted analyte, etc.) and/or properties of the species that can change spatially and/or temporally. The disclosed methods can comprise one or more operations (or steps), which can include but are not limited to: disposing one or more biological micro-objects (e.g., a biological cell or cells) in each of one or more chambers of a microfluidic device; flowing one or more medium (e.g., as described above or elsewhere herein) into a flow region of the microfluidic device (e.g., by continuous flow, pulsed flow, stopped flow, etc.) for a period of time (e.g., a first period of time, a second period of time, a third period of time, etc.), where each of the one or more chambers are fluidically connected and open to the flow region and where each medium may comprise one or more reporter molecules (e.g., a first reporter molecule, a second reporter molecule, a third reporter molecule, etc.); and assessing signal in a region of interest (e.g., a first area of interest, one or more areas along an axis of diffusion, or any location that is free of biological micro-objects) for the purpose of detecting a concentration of each of the one or more reporter molecules (e.g., the concentration of free reporter molecule and/or reporter molecule bound to a secreted analyte of interest, etc.). Each operation can be executed with a particular intent, outcome, or aim.

In some embodiments, the methods for assaying an intrinsic diffusion gradient and/or assessing (e.g., quantifying) a level of secretion of an analyte of interest from a biological micro-object or a population of biological micro-objects (e.g., a clonal population) can comprise steps for generating background data. Such steps can be performed to measure fluorescence (e.g., autofluorescence) within a system, including in the microfluidic device and/or the media. Background data can comprise background images taken using methods comprising steps to generate said background data. Background data can be used for correction and/or subtraction of fluorescence images taken under non-background conditions. The methods can comprise steps of capturing one or more images using a filter cube relevant to the background and non-background conditions, and taken at a defined exposure time and/or at defined periods of time relative to non-background conditions. In some embodiments, one or more background images may be taken under conditions where medium is flowing into and through the flow region of the microfluidic device. Alternatively, or in addition, one or more background images can be taken under non-flow conditions (e.g., under conditions where medium is present in, but not being actively flowed through, the flow region of the microfluidic device).

In some embodiments, the methods for assaying an intrinsic diffusion gradient and/or assessing (e.g., quantifying) a level of secretion of an analyte of interest from a biological micro-object or a population of biological micro-objects (e.g., a clonal population) can comprise steps for obtaining data under conditions leading to or at an equilibrium state (e.g., a steady-state equilibrium). In some instances, such data can be generated using steps that produce an equilibrium state or steady-state equilibrium conditions within a microfluidic device.

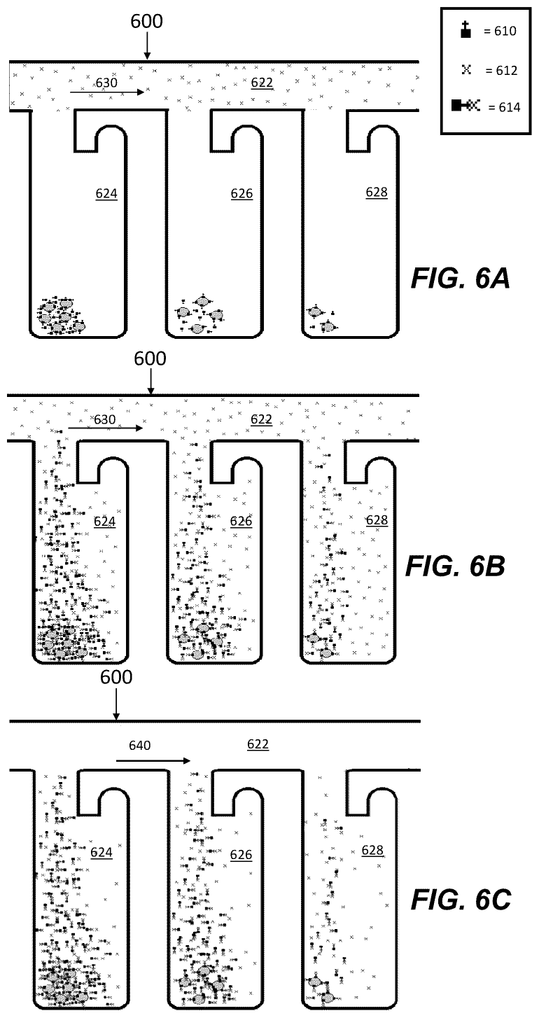

Methods comprising steps for obtaining data under steady-state equilibrium conditions can comprise performing an equilibration assay. Sec, e.g., FIGS. 6A-6B. In some embodiments, methods that include performing an equilibration assay can comprise disposing a biological micro-object into a chamber 624 (and/or 626 and/or 628) of a microfluidic device, where the microfluidic device includes an enclosure having a flow region 622 and the chamber 624 is fluidically connected to the flow region 622, and where the chamber 624 contains a first fluidic medium; allowing the biological micro-object, or a population of biological micro-objects generated therefrom, to secrete an analyte of interest 610 into the first fluidic medium within the chamber 624; introducing a second fluidic medium 630 into the flow region, where the second fluidic medium contains a plurality of reporter molecules 612; allowing a portion of the plurality of reporter molecules 612 to diffuse into the chamber and bind to the analyte of interest 612 secreted therein, thereby producing a plurality of reporter molecule: secreted analyte (RMSA) complexes 614; and detecting reporter molecules 612 located within a region of interest within the microfluidic device 600, wherein the region of interest includes at least a portion of the chamber 624. In some embodiments, the first fluidic medium differs from the second fluidic medium. For example, the first fluidic medium may lack reporter molecules 612, while otherwise sharing the same buffer, pH, level of dissolved oxygen, and/or stimulating factors, etc. In some embodiments, allowing the portion of the plurality of reporter molecules 612 to diffuse into the chamber 624 and bind to the analyte of interest 610 secreted therein is performed for a period of time (e.g., a first period of time) sufficient for unbound reporter molecules to reach a state of equilibrium between the flow region and the chamber. In some embodiments, detecting reporter molecules 612 in the region of interest comprises detecting unbound reporter molecules 612 as well as detecting reporter molecules 612 that are part of RMSA complexes 614. Reporter molecules 612 (and RMSA complexes 614) can be detected within the region of interest via one or more images taken of the associated chamber(s) 624 (and/or 626 and/or 628) and flow region(s) 622, with the image(s) being analyzed to determine or calculate a score correlated with the level of secreted analyte of interest 610 in the chamber(s) 624 (and/or 626 and/or 628), as discussed further below and/or elsewhere herein. In various embodiments, methods that include performing an equilibration assay can comprise flowing one or more additional fluidic media (e.g., a third fluidic medium, a fourth fluidic medium, etc.) through the flow region of the microfluidic device, either before detecting the reporter molecules (e.g., in connection with generating background data) or after detecting the reporter molecules (e.g., using a different reporter molecule to further analyze the analyte of interest or to analyze a second analyte of interest).

In some embodiments, the methods for assaying an intrinsic diffusion gradient and/or assessing (e.g., quantifying) a level of secretion of an analyte of interest from a biological micro-object or a population of biological micro-objects (e.g., a clonal population) can comprise (or further comprise) steps for performing a flush assay. Sec, e.g., FIGS. 6A-6C. Such methods may further include: introducing a third fluidic medium 640 into the flow region 622, where the third fluidic medium 640 does not include reporter molecules 612; and, in some embodiments, allowing at least a portion of unbound reporter molecules 612 to diffuse out of the chamber 624 for a period of time (e.g., a second period of time). In some embodiments, the second period of time may be selected based on modelling of diffusion profiles for unbound reporter molecules and RMSA complexes. In some embodiments, introducing the third fluidic medium 640 into the flow region is performed after allowing a portion of the plurality of reporter molecules 612 to diffuse into the chamber and bind to the analyte of interest 610 secreted therein; and, optionally, after allowing unbound reporter molecules 612 to reach a state of equilibrium between the flow region 622 and the chamber 624 (see, e.g., FIG. 6B). In some embodiments, introducing the third fluidic medium 640 into the flow region 622 is performed after detecting reporter molecules 612 located within a region of interest within the microfluidic device 600. Thus, the flush assay can be performed with a single detection step (i.e., after introducing the third fluidic medium 640 into the flow region but before detecting the reporter molecules 612 located within the region of interest) or with at least two detecting steps (e.g., with the detection of reporter molecules 612 in the region of interest occurring both before and after introducing the third fluidic medium 640 into the flow region 622, thereby allowing for a combined equilibration and flush assay).

In some embodiments, the methods for assaying an intrinsic diffusion gradient and/or assessing (e.g., quantifying) a level of secretion of an analyte of interest from a biological micro-object or a population of biological micro-objects (e.g., a clonal population) can comprise steps for obtaining kinetic data. The kinetic data can include one or more rates of change. In certain embodiments, the kinetic data can be obtained under conditions of differential diffusion, for example, using a flush assay. In a flush assay, the fluorescence of unbound reporter molecules, RMSA complexes, and/or some combination thereof is detected while flushing the flow region of the microfluidic device with a medium that substantially lacks reporter molecules, secreted analyte of interest, and RMSA complexes. During the flush, molecules at higher concentrations within the chamber (e.g., reporter molecules, secreted analyte of interest, RMSA complexes) diffuse down their concentration gradients, moving from a region of relatively high concentration (e.g., an unswept region of the chamber) to a region of relatively low concentration (i.e., the flow region). As a result of differences in the size and mass of the reporter molecules as compared to the RMSA complexes, differential diffusion can take place, with the lower mass reporter molecules diffusing out of the chamber and into the flow region at a faster rate than the higher mass RMSA complexes. By detecting the reporter molecules and RMSA complexes at a plurality of time points after the start of the flush, the relative contributions of the reporter molecules and the RMSA complexes to the detected signal can be determined. In some embodiments, determining the relative contributions of the reporter molecules and the RMSA complexes comprises comparing the detected signal to signal detected in another chamber of the microfluidic device, where the other chamber lacks a biological micro-object (and thus a source of analyte of interest).

In some embodiments, detecting the reporter molecules and/or RMSA complexes in the methods for assaying an intrinsic diffusion gradient and/or assessing (e.g., quantifying) a level of secretion of an analyte of interest from a biological micro-object or a population of biological micro-objects (e.g., a clonal population) can comprise imaging a region of interest within the microfluidic device. The image(s) can be taken of the associated chamber(s) and flow region(s) in the microfluidic device. The image can then be analyzed, e.g., to calculate a score correlated with the level of secreted analyte of interest in the chamber(s). In some embodiments, one or more (e.g., a plurality of) images is taken, e.g., over a period of time, or across one or more fields of view, regions of interest, fluorescent channels, etc. In some embodiments, an image or plurality of images may be taken at a first period of time, and subsequently an image or plurality of images can be taken at a second period of time. In some instances, images are taken as the system is approaching the equilibrium state and/or after the system reaches steady state. The period of time required to reach equilibration (e.g., steady state equilibrium) can be up to 3 hours or more (e.g., greater than about 10 minutes, greater than about 30 minutes, greater than about 1 hour, greater than about 1.5 hours, greater than about 2 hours, greater than about 2.5 hours, greater than about 3 hours, greater than about 3.5 hours, greater than about 4 hours, greater than about 4.5 hours, or longer). The images can be taken, for example, during continuous flow, during pulsed flow, or during stopped flow.

In some embodiments, the methods for assaying an intrinsic diffusion gradient and/or assessing (e.g., quantifying) a level of secretion of an analyte of interest from a biological micro-object or a population of biological micro-objects (e.g., a clonal population) can further comprise expanding the biological micro-object within the chamber into a clonal population (e.g., derived from a single cell) of biological micro-objects. Expanding the biological micro-object within the chamber can include flowing a culture medium through the flow region for a period of time.

Optical Calibration. Prior to any imaging, the optical system and microfluidic device may be configured using one or more methods of optical calibration. In certain embodiments, optical calibration can any method of optical alignment commonly known in the art. Optical alignment can comprise determining alignment of a filter cube, dichroic, or other optical component relative to a desired output or output range (e.g., power density at a particular location-including but not limited to the microfluidic device, a CCD camera, or another detector capable of calculating power density or a variable equivalent to or correlated with power density or another desirable output indicative of alignment of light moving through the optical train of the system, where the optical train is configured for operating and/or imaging the microfluidic device (e.g., microfluidic chip). In some instances, optical alignment can comprise aligning the focal plane and or objective of the optical train; such methods can include but are not limited to collecting one or more z dimension images and assessing the focus according to one or more features of the system configured for operating and/or imaging the microfluidic device.

In some embodiments, optical calibration can comprise applying methods for performing one or more image processing operations (flat fielding, normalization, masking, image subtraction, etc. on an image or set of images obtained from a microfluidic device. In any number of instances, a combination of the image processing operations (flat fielding, normalization, image subtraction, masking etc.) can be stacked together to yield a useful corrected image or corrected image set that can be used for further analysis. Methods for subtraction can comprise image subtraction and or pixel subtraction, whereby the digital numeric value of one pixel or a whole image is subtracted from another image. Methods of normalization can comprise taking a value of a given image (e.g., intensity value) and dividing it by an aggregated value (e.g., a global average intensity value). Methods for masking can comprise eliminating one or more sections of an image (e.g., sections where no signal should be present and/or where excessive background may interfere with, for example, calculation of a score correlated with the level of secreted analyte of interest in the chamber(s)).

In certain embodiments, the methods disclosed herein can comprise flat fielding. The term “flat fielding” as used herein refers to methods known it the art for improving the quality of the image relative to a result by eliminating artifacts (e.g., variations in pixel-to-pixel sensitivity of a detector, distortions of an optical path, etc.) by applying a flat field to compensate for variations in gains and dark currents across a detector such that a uniform signal detected by the detector can generate a uniform output. In some embodiments, the optical system and microfluidic device may be aligned across one or more axes (x, y, z), and, optionally, additional flat fielding may be applied. Such flat fielding can comprise, for example, applying a quadratic correction derived from the measurement of a uniform optical target. Flat fielding can be used in conjunction with any other image processing operations known in the art or combinations thereof.

Flat fielding and any other image processing operations or combination of image processing operations can be performed using one or more reference images. Examples of reference images include a Dark Reference image of the microfluidic device and a signal reference image. A dark reference image can be taken in the absence of one or more of: cells, medium, reporter molecule, etc., with the aim of providing dark reference values that can be used to correct for autofluorescence errors and other system errors at each pixel of the corrected image. A signal reference image, which can be taken of the microfluidic device with, for example, a measurable signal associated with a known quantity of signal producing component (e.g., fluorescent dye of a known concentration flowing through the microfluidic device) can be utilized with the aim of correcting for optical roll off, photobleaching errors, camera errors, etc. which have effects on the signal being measured.

In some embodiments, detecting the reporter molecules located within the area of interest further may include determining a background-subtracted signal intensity by subtracting an intensity of a background signal from the measured intensity of the detectable signal. The background signal may not be measured every time reporter molecules are detected. In some embodiments, the background signal may be pre-determined based on known/standard conditions (e.g., chip type, location of chamber in the chip, type of detectable label, components of first fluidic medium).

Methods disclosed herein may further include measuring an intensity of a background signal within the area of interest, at a time prior to introducing the biological micro-object into the chamber. In various embodiments, the measured intensity of the detectable signal or the background-subtracted signal intensity may be normalized for a number of cells observed within the chamber. The micro-objects may be measured using brightfield imaging, and counted using a cell counting method such as that disclosed International Publication No. WO 2018/102748.

In one or more embodiments, optical calibration can comprise calibration of the location of key features of a microfluidic device relative to the field of view or fields of view in images of the microfluidic device. In some instances, chip can include but is not limited to obtaining an image of a chip comprising one or more pattern(s) or feature(s) (e.g., a pattern or design etched, embedded or otherwise disposed) located at know locations on the microfluidic chip. Optical calibration of the microfluidic chip can comprise taking images of the pattern(s) or feature(s) and determining location of the microfluidic chip.

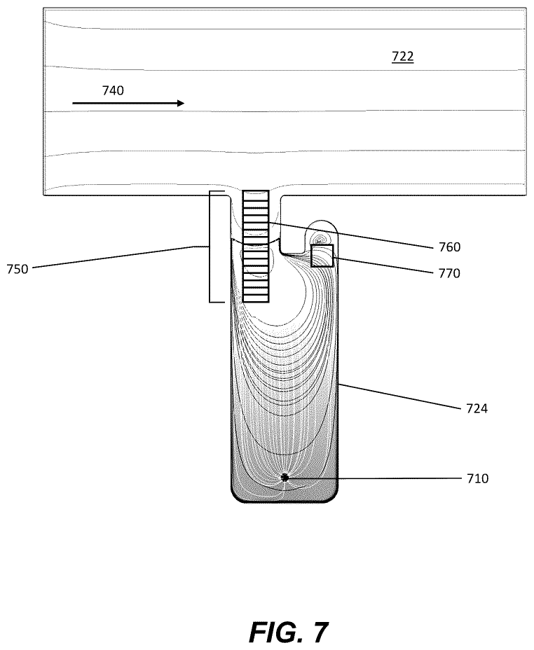

Region of Interest. In accordance with various embodiments, the region of interest may comprise at least a portion of the chamber (e.g., an upswept region of the chamber), as depicted in FIG. 7. In certain embodiments, the chamber 724 may have an isolation region and a connection region fluidically connecting the isolation region to the flow region, wherein the isolation region and the connection region are configured such that components of a fluidic medium in the isolation region are exchanged with components of a fluidic medium 740 in the flow region substantially only by diffusion. In such embodiments, the region of interest 760, 770 may comprise a portion of the isolation region, a portion of the connection region, a portion of the flow region 722 immediately adjacent to the connection region, or any combination thereof.

In some embodiments, a region of interest 760, 770 can comprise a region located between the location of biological micro-object(s) 710 within a chamber 724 and the opening of the chamber to a flow region 722 of the microfluidic device. In certain embodiments, the region of interest 760 may include at least a portion of the chamber 724 aligned along an axis of diffusion (e.g., 750) from within the chamber 724 to out into the flow region 722. Thus, the region of interest 760 can include one or more regions that lie along an axis of diffusion (e.g., 750) within the system. In certain embodiments, an axis of diffusion (e.g., 750) can comprise a portion of or the entirety of a connection region of the chamber, and, optionally, the axis of diffusion (e.g., 750) can further comprise a portion of an isolation region and/or a flow region of a microfluidic device. In other instances, the region of interest 770 can include one or more regions that lie off of an axis of diffusion (e.g., 750) within the system. For example, the region of interest 770 can include a portion or extension of the chamber 724 (or an isolation region thereof), that extends away from the region where biological micro-object(s) 710 would typically accumulate. Such a portion or extension can be a blind/dead-end extension, such as a hook region.

In certain embodiments, the region of interest may be located in a region of the chamber that lacks biological micro-objects and/or is less sensitive to the location of the biological micro-object(s) within the chamber. In certain related embodiments, the region of interest may be located at least 1 micron, 2 microns, 3 microns, 4 microns, 5 microns, 6 microns, 7 microns, 8 microns, 9 microns or 10 microns from the biological micro-object (e.g., cell) within the chamber. In certain embodiments, the region of interest can be located in a region of the chamber that is less sensitive to artificial background signal generated by, e.g., the edges of the chamber. Thus, in certain embodiments, the region of interest will be located at least 1 micron, 2 microns, 3 microns, 4 microns, 5 microns, 6 microns, 7 microns, 8 microns, 9 microns or 10 microns away from the edge or wall the chamber. In certain embodiments, the region of interest (or a sub-region thereof) can include a dimension (e.g., a width or a length) of at least about 10 microns (e.g., at least about 15 microns, at least about 20 microns, about least about 25 microns, or more). In certain embodiments, the region of interest (or a region thereof) can include an area of at least about 100 square microns (e.g., at least about 150, 200, 250, 300, 350, 400, 450, 500, 550, 600, 650, or more square microns). More generally, the size of the region of interest may be as small as a single pixel (e.g., the smallest unit of resolution of the detection device), but there is a trade-off with reducing the size of the region of interest because the signal:noise ratio generally goes down as the size of the region of interest is reduced. Moreover, as the size of the region of interest increases, the likelihood that the region of interest encompasses regions of the microfluidic device that may contain signaling artifacts (e.g., regions that include biological micro-objects) or signal arising from adjacent chambers also increases. Accordingly, regions of interest having an intermediate size (e.g., about 500 square microns to about 6000 square microns, or about 500 square microns to about 3000 square microns) can be advantageous for various embodiments of the methods of assaying an intrinsic diffusion gradient and/or methods of assessing or quantifying a level of secretion of an analyte of interest disclosed herein.

In certain embodiments, the region of interest may be divided into sub-regions. Sec, e.g., FIG. 7A & element 760. A region of interest (e.g., 760) may have subregions of differing sizes (e.g., pixel size) or each subregion may have the same size (e.g., pixel size). In some embodiments, a region of interest (e.g., 760) can comprise a plurality of subregions, wherein said plurality is from about 2 to about 50 (e.g., about 3 to about 35, about 4 to about 25, about 5 to about 15, or any range defined by two of the foregoing endpoints). In some embodiments, a most distal subregion of a region of interest (e.g., 760) is located furthest away from the flow region (or channel) 722 but is selected such that it does not overlap with the biological micro-object(s) 710, and the most proximal subregion of the set of subregions of the region of interest (e.g., 760) is located closest to or within the flow region (or channel) 722. The plurality of sub-regions from the most proximal subregion to the most distal subregion may be located at a region from which the detected signal is used to assess the relative or absolute amount of a secreted analyte of a biological micro-object within a chamber (e.g., 724). In certain embodiments, a region of interest that includes a plurality of sub-regions can include an aggregate area of at least about 500 square microns (e.g., at least about 750, 1000, 1500, 2000, 2500, 3000, 3500, 4000, 4500, 5000, 5500, 6000, 6500, or more square microns).

In certain embodiments, the region of interest, or subregions thereof, can be used to quantify imaging data, onto which a variety of mathematical operations may be performed to extract information about the relative or absolute amount of the secreted analyte. Such operations can include calculation of median, mean, and, when using a set of subregions, one or more values such as a slope indicative of an average intensity change across two or more subregion from the region of interest or integration of total intensity across two or more subregions.

Imaging data. Imaging data (e.g., an image, series of images, etc.) can comprise one of or a combination of background image(s), signal reference (e.g., fluorescence reference) image(s), diffusion reference image(s), and assay image(s).

A background image can be taken by an imaging device prior to any foreign matter (such as, for example, micro-objects, reporter molecules, or other reagents) being introduced into the microfluidic device. In so doing, the background image captures any background noise in the device, particularly in regions of interest. Background noise can be due to, for example, artifacts, or instrument setup and imaging parameters, including but not limited to light from the excitation source, camera noise, and ambient light. Background noise can also be due to background signal (e.g., fluorescence) imparted by, for example, auto-fluorescence of samples, vessels, imaging media, or the fluorescence resulting from fluorophores not bound to specific targets. The image area included in the background image may depend on how the image is implemented on the system going forward. For example, as will be described in detail below, depending on the calibration methods used, a different background image area may be desired.

A signal reference image can be taken by an imaging device after a reporter molecule is introduced into the flow region of the microfluidic device and the reporter molecule concentration equilibrates between the flow region and chambers of the microfluidic device, including in any regions of interest. In so doing, the signal reference image captures image acquisition distortions in the device and system. Such distortions can stem from, for example, microfluidic device and/or imaging element design. Image distortion types can include, for example, image edge effects, perspective distortion, barrel distortion, pincushion distortion, mustache distortion, and chromatic aberration. The signal reference image area can include an image of the region of interest, the flow region proximate the chamber and associated region of interest, or both. The image area included in the signal reference image can depend upon how that image is implemented by the system going forward. For example, as provided in further detail below, depending on the calibration methods implemented by the system, a different signal reference image area may be utilized.

In some instances, a diffusion reference image can be taken following introduction of a fluidic medium free of reporter molecules, where the fluidic medium free of the binding agent is perfused after the fluidic medium comprising the binding agent is introduced into the microfluidic device. In such instances, the signal reference image may be referred to as a “diffusion reference image.” The diffusion reference image may comprise a series of images taken over time of one or more fields of view. Analysis of the diffusion reference image can comprise measuring a change in signal as a function of time, determining a correction value or a slope from which correction values can be derived, and applying the correction value comparison of images to a single reference timepoint.

Normalization of the Assay image. Before the Assay Image can be processed to assess relative or absolute amounts of a secreted analyte, the raw Assay Image may be normalized. In some embodiment, the raw Assay Image may be normalized by subtracting both a Dark Reference image and a Signal Reference image correction from each pixel in the raw Assay Image as in the following equation:

$$Normalized Assay value = \frac{Assay Intensity value - Dark Reference value}{Signal Intensity value - Dark Reference value}$$

The Dark Reference image may be obtained by imaging the microfluidic device before introducing the biological micro-object. Autofluorescence errors and other system errors can be corrected by subtracting the Dark Reference value at each pixel. The Signal Reference Image may correct for roll off, vignetting, and other optical artifacts. The Signal Reference Image can be obtained by flowing reporter molecule (e.g., just the reporter molecule label) throughout the microfluidic device to reach an equilibrated concentration of the reporter molecule or label. Each pixel in the raw Assay Image may be corrected in this manner, before extracting the signal data for quantitation purposes. In some embodiments, a smoothing algorithm may be further applied to reduce noise.

Quantification of the assay signal. In some embodiments, the diffusion profile of the RMSA may be used to quantify the amount of the RMSA complex present in the chamber. The diffusion profile provides a series of values (e.g., signal intensity values) that represent the concentration of the RMSA complex as it diffuses from its source to the channel. After identification of the region of interest, other transformations may be applied. For example, the pixels in each line may be processing by discarding outlier and/or aberrant pixels, other forms of global/local normalization, space conversion, and transforming the space of the pixel (e.g. from a multi-dimensional space to a two-dimensional space or vice-versa).

Depending on the embodiment, the signal intensity values may be used in different ways to calculate values that are proportional to concentration values. In some embodiments, the AOI may be sampled at fixed points to generate a set of concentration values corresponding to the signal intensity values at the fixed points. In some embodiments, the region of interest may be segmented into a series of subregion and the median or mean intensity of each subregion may be calculated. Based on the embodiment and the degree of resolution required, the number of concentration values calculated can be as low as 1 and as high as the number of pixels representing the diffusion trajectory.

Depending on the embodiment, the concentration values may be combined in different ways in order to quantify the amount of signal from the bound reporter molecules (and therefore an amount of secreted analyte of interest) present. In some embodiments, the concentration values may be plotted to assess whether concentration values exhibit characteristics consistent with a diffusion profile. Depending on the embodiment, a number of algorithms may be used to fit a line to the concentration values and calculate characteristics of the line such as the slope and error associated with the line. Suitable line-fitting algorithms include: least-squares, polynomial fit, curve-fitting, and erfc fitting. Other suitable algorithms are known to those skilled in the art.

### II. ASSAYS FOR MULTICONFIGURATION MULTIDOMAIN BIOMOLECULE PRODUCTION

Assays for multiconfiguration multidomain biomolecule production can benefit from the diffusing gradient assays disclosed herein, such as the assays of an intrinsic diffusion gradient and/or the assays for assessing or quantifying a level of secretion of an analyte of interest. Accordingly, the foregoing description of such assays should be understood to be fully applicable to the discussion of assays for multiconfiguration multidomain biomolecule production presented below.

Multiconfiguration multidomain biomolecules. A multiconfiguration multidomain biomolecule, as described herein, refers to a biomolecule having two or more components that can be differentially assembled and/or linked. Such biomolecules can form a group of biomolecules that all share at least one component in common (e.g., a primary component) but differ from other members of the group with regard to the presence or absence of other components (e.g., one or more secondary components) in the fully-assembled form of the biomolecule. For example, a multiconfiguration multidomain biomolecule may include a single primary component, A, and a single secondary component selected from B1 and B2, resulting in a group of multiconfiguration multidomain biomolecules having two different configurations, AB1 and AB2. As another example, a multiconfiguration multidomain biomolecule may include two primary components, each A, and two secondary components each selected from B1 and B2, resulting in a group of multiconfiguration multidomain biomolecules having three different configurations: A2B12, A2B1B2, and A2B22. As another example, a multiconfiguration multidomain biomolecule may include two primary components, each selected from A1 and A2, and two secondary components, each selected from B1 and B2, resulting in a group of multiconfiguration multidomain biomolecules having nine different configurations: A12B12, A12B1B2, A12B22, A1A2B12, A1A2B1B2, A1A2B22, A22B12, A22B1B2, and A22B22. As yet another example, a multiconfiguration multidomain biomolecule may include two primary components, each selected from A1 and A2, two secondary components, each selected from B1 and B2, two tertiary components (e.g., a modification of B1 or B2), each of which may be independently present, M, or absent, thereby resulting in a group of multiconfiguration multidomain biomolecules having thirty-six different configurations: A12(B1-M)2, A12(B1-M)(B2-M), A12(B2-M)2, A1A2(B1-M)2, A1A2(B1-M)(B2-M), A1A2(B2-M)2, A22(B1-M)2, A22(B1-M)(B2-M), A22(B2-M)2, A12(B1-M)2, A12(B1-M)B2, A12B22, A1A2(B1-M)2, A1A2(B1-M)B2, A1A2B22, A22(B1-M)2, A22(B1-M)B2, A22B22, A12B12, A12B1(B2-M), A12(B2-M)2, A1A2B12, A1A2B1(B2-M), A1A2(B2-M)2, A22B12, A22B1(B2-M), A22(B2-M)2, A12B12, A12B1B2, A12B22, A1A2B12, A1A2B1B2, A1A2B22, A22B12, A22B1B2, and A22B22. As is readily apparent, groups of multiconfiguration multidomain biomolecules can be complex and extensive, growing exponentially as the number of possible components and variants of each component increase.

A multiconfiguration multidomain biomolecule can be a single molecular entity (e.g. a single polypeptide chain or a single strand nucleic acid), which may or may not be modified (e.g., glycosylated, phosphorylated, methylated, etc.). Alternatively, a multiconfiguration multidomain biomolecule can be a complex of two or more molecular entities (e.g., a protein complex, a nucleic acid complex, of combinations thereof, such as a riboprotein), which likewise may or may not be modified (e.g., glycosylated, phosphorylated, methylated, etc.). Regardless, the multiconfiguration multidomain biomolecule will include at least a first portion and a second portion, where the first and second portions are different. Accordingly, in some embodiments, the multiconfiguration multidomain biomolecule may be a single polypeptide chain that is alternatively spliced, and the first portion may be a first amino acid chain segment of the protein (e.g., an amino acid chain segment always present in the mature protein) and the second portion may be an alternative amino acid chain segment (e.g., an amino acid chain segment that is present sometimes, but not always, in the mature protein). In other embodiments, the multiconfiguration multidomain biomolecule may be a glycosylated protein, and the first portion of the protein may be an amino acid chain segment and the second portion may be a modification of the amino acid chain segments, such as a glycosylation. In other embodiments, the multiconfiguration multidomain biomolecule may be a protein-nucleic acid complex, and the first portion of the complex may be an amino acid chain segment and the second portion may be a nucleic acid chain. In still other embodiments, the multiconfiguration multidomain biomolecule may be a protein complex, and the first portion of the protein may be a first amino acid chain segment and the second portion may be a second amino acid chain segments. In particular embodiments, the protein complex may be an antibody (or fragment or single-chain version thereof), and the first portion may be a first light chain amino acid sequence and the second portion may be a second light chain amino acid sequence. In cases where the multiconfiguration multidomain biomolecule is an antibody such as a bi-specific (or tri-specific, etc.) antibody, the multiconfiguration multidomain biomolecule can be termed a “multispecific protein.” As will be readily apparent, many other examples of multiconfiguration multidomain biomolecule exist, all of which may be suitably analyzed using the assays for multiconfiguration multidomain biomolecule production disclosed herein.

In the production of multiconfiguration multidomain biomolecules (e.g., multispecific proteins), a portion of the product might not fully or correctly assemble. Taking the production of bispecific antibodies as an example, the bispecific antibody to be produced is expected to have two types of Fv regions (or Fab regions) so that the produced bispecific antibodies can bind to two types of epitopes. When the two types of Fv regions are produced, there is a chance that two of the same type of Fv regions assemble together resulting in a product that has only one kind of Fv region and therefore does not form the bispecific structure expected. Therefore, it will be particularly beneficial if the likelihood of whether the assembly of the multispecific antibody is correct can be assessed on-chip so that a clonal population of biological micro-objects producing the multispecific antibody can be identified. The assessment of the likelihood of whether the assembly of the multispecific protein is correct can be performed by a diffusion gradient assay of the present disclosure.

In some embodiments, given that the assembly of the multispecific protein to be produced may not occur properly, the proteins secreted by the biological micro-objects might contain various assemblies (including the correct assembly as well as other incorrectly assembled proteins, which are of no interest) having various molecular weights. This situation poses a difficulty in determining a suitable time required for the diffusion gradient assay to reach a steady state equilibrium. Thus, a generalizable diffusion assay that can work on analytes of a wide range of molecular weights will be favorable, as is described in more detail herein.

In some embodiments, the multispecific protein can be a multispecific antibody, a bispecific antibody, or a trispecific antibody, having multiple binding regions each bind to a selected epitope. The binding region can be the Fv region or Fab region of the antibody. Reporter molecules useful to detect the binding regions of an antibody are described throughout this disclosure.

In some embodiments, the multispecific protein can be a protein other than an antibody, and wherein the binding region can be a motif including but not limited to a proteolytic motif, calcium binding motifs, and/or glycosylation motifs. Motif, as used herein, can be a protein, a nucleic acid, or a glycan sequence of a biomolecule. Reporter molecules for proteins other than an antibody may include a binding component configured to specifically recognize the motif of the multispecific protein. In one non-limiting example, a glycan motif of a multispecific protein may be detected by a reporter molecule having a binding component that is a lectin configured to bind a specific glycan such as mannose, fucose, or sialic acid.

Methods. The method of assaying for multiconfiguration multidomain biomolecule production can include introducing a biological micro-object into a chamber of a microfluidic device, wherein the microfluidic device comprises an enclosure having a flow region and the chamber fluidically connected to the flow region; allowing the biological micro-object to secret an analyte within the chamber; introducing a plurality of first reporter molecules into the flow region, wherein each of the plurality of first reporter molecules comprises a first detectable label and a first binding component configured to bind a first binding regions of the more than one type of binding region and form a first reporter molecule: secreted analyte complex (first RMSA complex); and introducing a plurality of second reporter molecules into the flow region, wherein each of the plurality of second reporter molecules comprises a second detectable label and a second binding component configured to bind a second binding regions of the more than one type of binding region and form a second reporter molecule: secreted analyte complex (second RMSA complex); detecting a first signal associated with the first detectable label and detecting a second signal associated with the second detectable label within an area of interest within the microfluidic device; determining a first absolute quantitation value of the first signal, and a second absolute quantitation value of the second signal; analyzing a ratio of the first absolute quantitation value and the second absolute quantitation value thereby identifying the biological micro-object having a pre-selected ratio of the first binding regions and the second binding regions.

Suitable pre-selected ratios can depend upon the composition (e.g., stoichiometry) of the multiconfiguration multidomain biomolecule. For example, for a multiconfiguration multidomain biomolecule having one copy of the first component and one copy of the second component, a 1:1 ratio (e.g., about 0.7:1 or higher, about 0.8:1 or higher, about 0.9:1 or higher, or about 1:1 or higher) may be appropriate. Alternatively, for a multiconfiguration multidomain biomolecule having one copy of the first component and two copies of the second component, a 1:2 ratio may be appropriate (e.g., about 0.7:2 or higher, about 0.8:2 or higher, about 0.9:2 or higher, or about 1:2 or higher). In view of the teachings provided herein, persons skilled in the art can readily identify suitable pre-selected ratios based on expected biomolecule composition and experimentation.

In some embodiments, introducing a plurality of first reporter molecules into the flow region comprises allowing at least a portion of the first reporter molecules to diffuse into the chamber to attain a steady state equilibrium of the first reporter molecule across the flow region and the chamber. In certain embodiments, the steady state equilibrium of the first reporter molecule is attained within 3 hours, within 2.5 hours, within 2 hours, from 2 to 3 hours, or from 2 to 2.5 hours, from introducing first reporter molecule. In some embodiments, introducing a plurality of first reporter molecules into the flow region comprises perfusing a fluidic medium comprising the first reporter molecule into the flow region, and allowing at least a portion of the first reporter molecules to diffuse into the chamber comprises continuing the perfusion.

In some embodiments, introducing a plurality of second reporter molecules into the flow region comprises allowing at least a portion of the second reporter molecules to diffuse into the chamber to attain a steady state equilibrium of the second reporter molecule across the flow region and the chamber. In certain embodiments, the steady state equilibrium of the second reporter molecule is attained within 3 hours, within 2.5 hours, within 2 hours, from 2 to 3 hours, or from 2 to 2.5 hours, from introducing the second reporter molecule. In some embodiments, introducing a plurality of second reporter molecules into the flow region comprises perfusing a fluidic medium comprising the second reporter molecule into the flow region, and allowing at least a portion of the second reporter molecules to diffuse into the chamber comprises continuing the perfusion.

In some embodiments, detecting the first signal is conducted after the steady state equilibrium of the first reporter molecule is reached. In some embodiments, detecting the first signal can be also conducted while perfusing another fluidic medium comprising no reporter molecule (e.g. the flush process as described in the diffusion gradient assay).

In some embodiments, detecting the second signal is conducted after the steady state equilibrium of the second reporter molecule is reached. In some embodiments, detecting the first signal can be also conducted while perfusing another fluidic medium comprising no reporter molecule (e.g. the flush process as described in the diffusion gradient assay).

In some embodiments, the first reporter molecule and the second reporter molecule are introduced in a same fluidic medium, so that the first signal associated with the first detectable label and the second signal associated with the second detectable label can be detected while a steady state equilibrium of the two kinds of reporter molecules is reached or while perfusing a fluidic medium that does not comprise the first reporter molecule and the second reporter molecule.

In some embodiments, the method further comprises allowing at least a portion of unbound first reporter molecules and/or unbound second reporter molecules to diffuse out of the chamber. In certain embodiments, the unbound first reporter molecules and/or unbound second reporter molecules is diffusing out of the chamber while a fluidic medium that does not comprise the first reporter molecule and/or the second reporter molecule is perfusing into the flow region.

Analyte of interest. The analyte of interest can be a multiconfiguration multidomain biomolecule, as described above. Thus, the analyte of interest can be a secreted biomolecule, such as a protein, protein complex, glycoprotein, nucleic acid, modified nucleic acid, or any combination thereof produced by biological micro-objects or a population of the biological micro-objects generated therefrom. Often, the secreted biomolecule can be the protein of interest. For example, the secreted analyte can be a multispecific protein. In some embodiments, the analyte of interest can include two or more components, with one or both components being a binding region configured to recognize a motif of a target biomolecule. The motif can comprise an amino acid, a nucleic acid, a glycan, a hapten, or any combination thereof. In certain embodiments, one or both components is/are configured to recognize a region of glycosylation in a target biomolecule. In some embodiments, the protein of interest is a multispecific antibody, a bispecific antibody, or a trispecific antibody of which each of the more than one type of binding regions is configured to recognize an epitope. As described above, the secreted analyte of interest can also be a mis-assembled product. In some embodiments, the secreted analyte may be an analyte mixture comprising a plurality of analytes having a range of molecule weights. In some embodiments, the analytes of the analyte mixture may have a molecule weight from about 1 kDa to 600 kDa. In some embodiments, the analytes has a molecule weight of about 1, 5, 10, 15, 20, 25, 30, 35, 40, 50, 60, 70, 80, 90, 100, 120, 140, 160, 180, 200, 220, 240, 260, 280, 300, 320, 340, 360, 380, 400, 420, 440, 460, 480, 500, 520, 540, 560, 580, 600 kDa, or any range defined by two of the foregoing endpoints.

Reporter molecule(s). Methods disclosed herein can comprise one or more reporter molecules (e.g., detection reagents, reagents, reporter, etc.). Reporter molecules can be configured to covalently or non-covalently bind to a secreted analyte of interest. In methods disclosed herein, the reporter molecule bound to the secreted analyte is configured to generate a signal that can be detected using imaging, such that the signal (raw or processed using one or more methods disclosed here in) provides direct or indirect measure of diffusion related properties such as concentration and diffusion rate constant which are proportional to the molecular weight of the reporter and/or reporter bound with secreted analyte (e.g., RMSA complexes). Signal is proportional to one or more of the amount of accumulated protein/complex resulting from one or more of: the secretion rate of a biological micro-object, the number of biological micro-objects, and/or the fraction bound of the analyte.

A reporter molecule may include a binding component designed to bind the secreted analyte and also may include one or more detectable label(s). The binding component may be any suitable binding partner configured to bind the secreted analyte (e.g., with a binding constant less than 10 micromolar). The binding component may be a protein, a peptide, a nucleic acid or small organic molecule. The binding component specifically binds to the secreted analytes, specific binding comprises a preference for the secreted analyte over one or more other components on or within the microfluidic device. In some embodiments, the reporter molecule may be multi-valent, comprising more than one binding component to bind more than one copy of the secreted analyte or to more than one member of a family of secreted analytes. The stoichiometry of the RMSA complex can therefore vary for example one or more reporter molecules can bind to one or more secreted molecules, and additionally or alternatively one or more secreted molecules can bind to one or more reporter molecules. The reporter molecule or secreted analyte must be soluble and can diffuse is solution or media disposed within the microfluidic device.

The method comprises introducing more than one type (or at least two types) of reporter molecules into the flow region. In some embodiments, introducing a plurality of first reporter molecule and introducing a plurality of second reporter molecule can comprise introducing a first fluidic medium comprising the plurality of first reporter molecule and the plurality of second reporter molecule into the flow region. In some embodiments, the more than one type of reporter molecules can be introduced sequentially in separate flows of medium. For example, introducing a plurality of first reporter molecules and a plurality of second reporter molecules comprises introducing a first fluidic medium comprising the plurality of first reporter molecules and introducing a second fluidic medium comprising the plurality of second reporter molecules into the flow region.

For a given reagent, the concentration of the reporter molecule should be above the dissociation constant K_{D} to drive binding and subsequently defines the background intensity. In some embodiments, 1×K_{D} might be optimal for the concentration of a reporter molecule. In some embodiments, the use of a reporter molecule having poor binding affinity may employ a higher concentration than 1×K_{D}, e.g., about 2×K_{D}, 5×K_{D}), 7×K_{D}, 10×K_{D}, 50×K_{D}, or about 100×K_{D}, to drive binding and result in a higher background signal. Use of a higher concentration of the reporter molecule, however, may be balanced against the increase of noise, reduction of sensitivity, and/or image saturation resulting from such increased concentration.

In the embodiments that the reporter molecule is introduced in a fluidic medium, a concentration of the reporter molecule in the fluidic medium is about 1 to 10 times, about 1 to 5 times, or about 1 to 3 times a K_{D} of the reporter molecules for the analyte secreted. In some embodiments, a molecular weight of the reporter molecule is about 150, 140, 130, 120, 110, 100, 90, 80, 70, 60, 50, 40, 30, 20, 10, 5, 4, 3, 2 kDa, or any range defined by two of the foregoing endpoints.

In some embodiments, each of the more than one type of reporter molecules comprises a detectable label and a binding component. The binding component is configured to bind to a binding site on one of the more than one type of target biomolecule-binding regions. In some embodiments, the binding component comprises an amino acid, a polypeptide, a nucleotide, a nucleic acid, or a combination thereof. In some embodiments, the binding component of the reporter molecule comprises a protein. For instance, in some embodiments, the binding component can be an epitope of an antibody of interest. In some embodiments, the binding site can be on a conserved region of an antibody, for example, a Fc region or a conserved region of the kappa light chain of antibodies. In some embodiments, the binding components of each of the more than one type of reporter molecules do not interfere with each other in their binding with the secreted analyte at the corresponding more than one target biomolecule binding regions. In certain embodiments, the binding component can be a lectin configured to bind a specific glycan such as mannose, fucose, or sialic acid.

The method comprises allowing a portion of the reporter molecules to diffuse into the plurality of chambers and bind to the secreted analyte therein; thereby producing a plurality of reporter molecule:secreted analyte (RMSA) complexes. In some embodiments where two types of reporter molecules (a first reporter molecules and a second reporter molecules) are introduced, a portion of the first reporter molecules is allowed to diffuse into the plurality of chambers and bind to the secreted analyte therein thereby producing a plurality of first reporter molecule: secreted analyte (first RMSA) complexes. Likewise, a portion of the second reporter molecules is allowed to diffuse into the plurality of chambers and bind to the secreted analyte therein thereby producing a plurality of second reporter molecule: secreted analyte (second RMSA) complexes. In some embodiments, the first RMSA and the second RMSA may be part of the same complex provided that the first reporter molecule and the second reporter molecule bind to the secreted analyte without interference from each other.

The method comprises detecting any two types of the more than one type of reporter molecules located within an area of interest within the microfluidic device, which can be the area or interest defined herein. In some embodiments, detection is conducted after one or at least the two types of the more than one type of reporter molecules have reached its steady state equilibrium across the flow region and the chamber. The detections of the two types of the more than one type of reporter molecules can then be calibrated into absolute quantitation values by, for example, the calibration described herein.

In some embodiments, detecting any two types of the more than one type of reporter molecules comprises detecting the respective RMSA thereof. Specifically, when the first RMSA complexes and the second RMSA complexes are formed, detecting the first reporter molecules or the second reporter molecules comprises detecting the first RMSA and the second RMSA respectively.

Detectable label. The reporter molecule may be intrinsically capable of emitting a detectable signal (e.g., in the manner of an auto fluorescing protein such as green fluorescent protein (GFP)). Alternatively, the reporter molecule may include a detectable label, such as a visible, luminescent, phosphorescent, or fluorescent detectable label. In some embodiments, the detectable label may be a fluorescent label. Any suitable fluorescent label may be used, including but not limited to fluorescein, rhodamine, cyanine, phenanthrene or any other class of fluorescent dye label. In some embodiments, the detectable label is covalently attached directly or indirectly to the binding component of the reporter molecule. In some other embodiments, a capture oligonucleotide may be a binding component of a reporter molecule and either an intrinsic or extrinsic fluorescent dye may be the detectable label, such that the detectable label of the reporter molecule may not be detectable until the capture oligonucleotide binds the analyte, for example, an intercalating dye. In some embodiments, a detectable label of a reporter molecule may not be detectable until after the RMSA complex has formed, as the detectable signal is shifted to a new wavelength not present prior to binding. Reporter molecules suitable for binding to antibodies include proteins, peptides and aptamers configured to bind regions of an IgG. Methods disclosed herein may comprise micro objects (e.g., biological micro-objects) on or within the microfluidic device at that time the method is performed. For the purpose of the methods disclosed herein, a biological micro-object can comprise any micro-object configured or capable of secreting, producing, or otherwise generating a secreted analyte of interest.

Absolute Quantitation. The signal (detection of reporter molecule) in the chamber under steady state equilibrium is a function of various factors including, but not limited to: the secretion rate of the cells; the diffusion rate out of the pen of the bound complex (RMSA); the fraction bound of the reporter molecule, which is a function of the K_{D} and the concentration of the reporter molecule, stoichiometry of the bound complex (reporter molecule and secreted analyte, e.g, secreted biomolecule), and the stoichiometry of the detectable label to the reporter molecule (i.e. labeling efficiency).

Diffusion rate out of the pen of the bound complex (RMSA). Since the molecular weight of the secreted analyte is a property of the structure of the secreted analyte, e.g. biomolecule, and thus pre-defined, the diffusion rate of the bound complex can only be adjusted by changing the molecular weight of the reporter molecule. Reporter molecules having a larger molecular weight can slow the diffusion rate out of the pen and increase the amount of bound complex in the chamber (i.e. signal). Nevertheless, the diffusion rate does not always scale linearly with molecular weight. In some embodiments, a suitable molecular weight for a reporter molecule is about ˜150 kDa, which may be, but is not limited to an antibody (e.g., a monoclonal antibody).

Stoichiometry of the bound complex and the number of fluorophores per reporter molecule. In some embodiments, the stoichiometry of the bound complex and the number of fluorophores per reporter molecule may be used to convert the intensity measurement into an absolute concentration. The conjugation efficiency can be measured prior to the diffusion assay. The design and characterization of the reporter molecules and the secreted analyte can be used to determine the stoichiometry.

To convert the measured intensity to a reporter molecule concentration on instrument, a calibration or standard curve may be generated. This calibration or standard curve, in some embodiments, could consist of a two-point standard curve using a background image (zero reagent concentration) to measure the autofluorescence of the media and chip and using the signal intensity of an empty pen or the channel, when reporter molecules are present, where the concentration of the reporter molecule can be, e.g. 1×K_{D}. This calibration may further include taking a standard curve prior to loading cells on the chip and measuring the intensity at varying concentrations of the reporter molecules. For example, a three-point standard curve could consist of a background image (without introducing the reporter molecules), a lower concentration (e.g. 1×K_{D}), and a higher concentration (e.g. 3× K_{D}).

To determine the absolute concentration in a secreting pen, the sum intensity minus the background intensity is equal to the concentration of the bound reporter molecules. Using the KD and the stoichiometry of the interaction, the fraction bound can be determined and the absolute concentration of the secreted biomolecule, e.g., a protein, can be determined. Using the Spotlight FC reporter molecule to detect a monoclonal antibody (mAb) as an example, the binding stichometry is two spotlight FC molecules to one mAb. Using a concentration of 1×KD, half of the binding sites will be bound so that, on average, one Spotlight FC reporter molecule will be bound to each mAb.

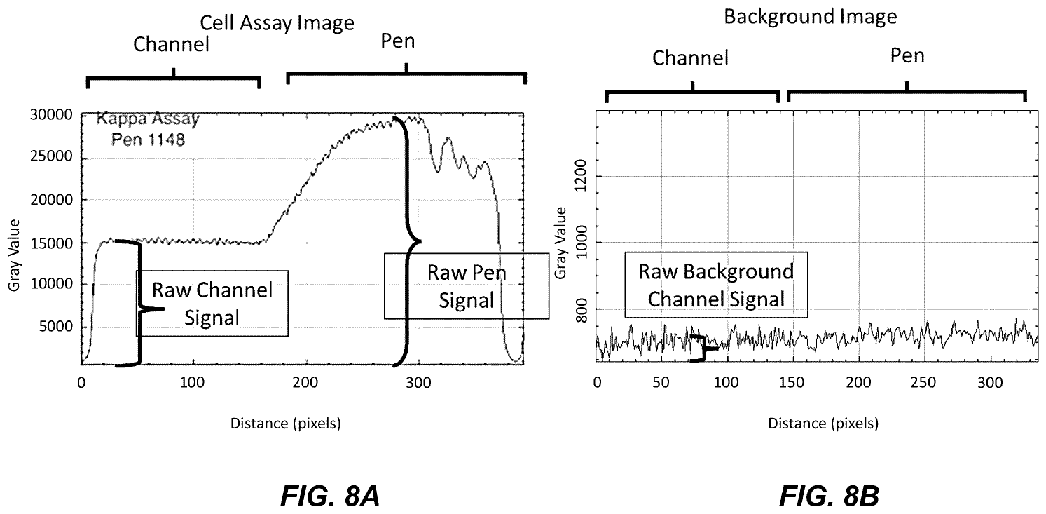

FIG. 8 shows an example of calibration of the present disclosure. A cell assay image and a background image were taken after the steady state equilibrium was reached. The cell assay image can be acquired within any area of interest as described therein; in this example, it was acquired in a region around the opening of the chamber to the flow channel, including a subregion located in the chamber and a subregion located in the channel. The signal (the intensity of the detectable label of the reporter molecule, e.g., SpotLight Kappa reporter molecule, as shown in this experiment) measured in channel is determined as a raw channel signal, representing the basic intensity of the reporter molecules; and the signal measured within a chamber, e.g., in pen. is determined as raw chamber (e.g., pen) signal, representing the secretion of the cells cultured. A raw background channel signal was detected from the background image. In some embodiments, the raw channel signal can be a mean intensity detected at the channel portion of the image, and the raw chamber signal can be a mean intensity detected at the pen portion of the image. Nevertheless, the raw channel signal or the raw chamber signal can also be an intensity measured at a particular position of the channel or the chamber as long as the position is consistent in the experiment. An absolute quantitation value of the chamber can be obtained according to the following equation where the concentration in the formula is the concentration of the reporter molecule.

$$( Raw chamber Signal - Raw Channel Signal ) \times ( \frac{Concentration}{Raw Channel Signal - Raw Background Signal} ) = Absolute Quantitation value$$

In some embodiments, the raw chamber signal may be obtained by detecting an intensity of the detectable label of the reporter molecules within a subregion of the area of interest that is located in the chamber (a chamber subregion of the AOI); the raw channel signal is obtained by detecting an intensity of the detectable label of the reporter molecules within a subregion of the area of interest that is located in the channel (a channel subregion of the AOI). In some embodiments, the raw background signal is obtained by detecting an intensity of the detectable label of the reporter molecules within a cell-free flow region within the microfluidic channel corresponding to the channel subregion of the AOI. In some embodiments, the cell-free flow region is located adjacent to an opening of a chamber where no cells are cultured.

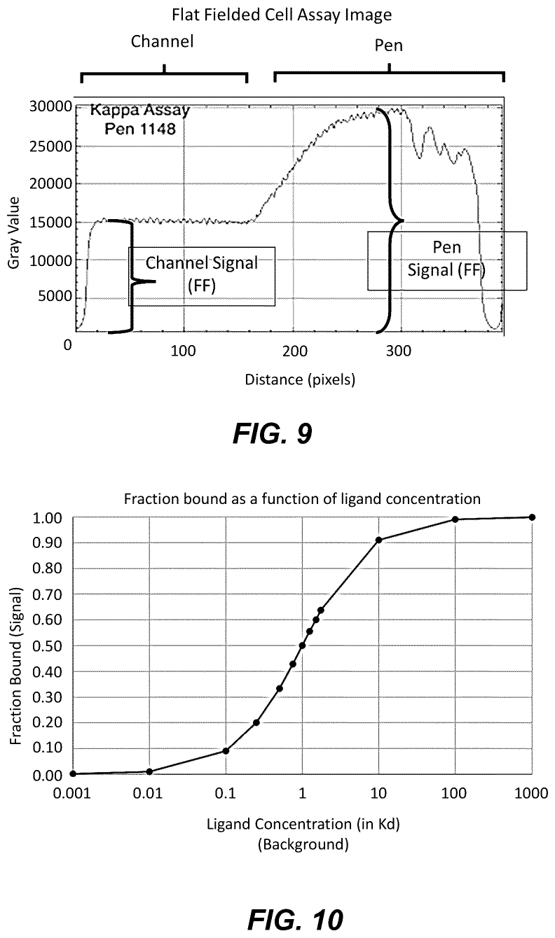

FIG. 9 shows another example using flat field correction in calibration in order to remove noise and improve resolution as shown in the following equation:

$$( Pen {Signal}_{FF} - Channel {Signal}_{FF} ) \times ( \frac{Concentration}{Channel {Signal}_{FF}} ) = Absolute Quantitation Value$$

Absolute value of secreted analyte concentration: titration curve. In some embodiments, a theoretical model of diffusion may be used to generate an absolute value based on the one or more concentration values and/or a known quantity of the secreted analyte of a biological micro-object in one of the chambers. Depending on the embodiment, different theoretical models of diffusion may be used to calculate an absolute value of the analyte based on the one or more concentration values. Depending on the embodiment, the theoretical model may model various phenomena or evaluate different assumptions.

In some embodiments, a titration curve may be used to generate an absolute value of a secreted analyte of a biological micro-object. In these instances, various known amounts of the analyte may be introduced into the microfluidic device and used to generate absolute values representing the known amounts of the analyte. The absolute values representing the known amounts of the analyte may be used to generate a titration curve demonstrating, in part, a linear relationship between the absolute values and the various known amounts of the analyte. In some embodiments, a number of absolute values corresponding to known amounts of the analyte may be generated such that the titration curve contains a “dynamic range” showing the upper and lower bounds of accurate quantification of the analyte given various system parameters (i.e. the highest and lowest amount of the analyte that produces an absolute value having a linear relationship).

Depending on the embodiment, various methods of replicating an anticipated diffusion profile may be used to allow the concentration values for the known concentrations of analyte to be generated in the same manner as the analyte that is generated at a source in the chamber (e.g. by a cell in a chamber). Further details are described in International Publication No. WO 2021/183458, filed on Mar. 8, 2021, which is herein incorporated by reference in its entirety.

Ratiometric Analysis. Variations of the quantitative equilibrium diffusion gradient assay would enable additional applications. In particular, the ratiometric analysis of multiple assays could be used to measure a dissociation constant (Kd) or determine the stoichiometry of an interaction. For affinity measurements, the fraction bound can be determined allowing for the extraction of the Kd. Measuring multiple target biomolecule-binding regions can allow for the determination of the stoichiometry between the target biomolecule-binding regions, allowing the characterization of engineered proteins such as bispecific proteins, including bispecific antibodies capable of binding to two different epitopes.

If the Kd of the reporter molecule is known, multiple binding sites of an analyte can be measured to determine the stoichiometry of the binding sites, such as but not limited to epitopes. This would enable the characterization of engineered proteins such as bispecific proteins. The experimental paradigm would consist of assays measuring epitope A, epitope B, and a conserved epitope present on all molecules (e.g. Fc or Kappa conserved region on a mAb).

The method comprises, analyzing a ratio of the absolute quantitation values from the detection of the two types of the more than one type of reporter molecule by comparing with a pre-selected ratio of the target biomolecule-binding regions on the analyte, e.g., protein of interest. In some embodiments, if the protein of interest is a bispecific antibody designed to bind to two epitopes at equal number, meaning the antibody has two Fv regions and each of them binds to an epitope, the bispecific antibody will have a pre-selected ratio of the Fv regions of 1:1. In this scenario, if the ratio of the first absolute quantitation value and the second absolute quantitation value is close to 1:1, the secreted analyte may be assembled correctly. In some embodiments, for concluding the secreted analyte assembled correctly, the ratio of the first absolute quantitation value and the second absolute quantitation value can be 1±0.5:1±0.5, 1±0.3:1±0.3, or 1±0.1:1±0.1. The clones exhibiting the preferential ratio of absolute quantitation values may be more valuable clones for selection, export and scale-up.

Generalizable diffusion gradient assay. In another aspect, a generalizable diffusion gradient assay for assessing a secretion level, which can work on analytes of a wide range of molecular weights is provided. By applying the generalizable diffusion assay, a trial run to evaluate a suitable concentration of the reporter molecules and/or a time required to establish a steady state equilibrium is not required. As described herein, in some embodiments, by setting a concentration of the reporter molecules at about 1 to 10 times, about 1 to 5 times, or about 1 to 3 times a KD of the reporter molecules for the analyte secreted and a molecular weight of the reporter molecules no more than about 150 kDa or about 2 to 150 kDa, the method can work on analytes of a wide range of molecular weights from about 1 kDa to 600 kDa or 25 kDa to 600 kDa and a steady state equilibrium can be reached within 3 hours, within 2.5 hours, within 2 hours, from 2 to 3 hours, or from 2 to 2.5 hours.

The effect of Kd and detection reagent concentration on the signal-to-background and signal-to-noise. The concentration of the reporter molecule can be a major determining factor in determining the signal-to-background and signal-to-noise. The lower the KD of the reporter molecule, e.g. higher affinity the reporter molecule exhibits towards its target biomolecule-binding region, the better the sensitivity, as a lower concentration of the reporter molecule can be used reducing the background and noise. The signal may be determined by the amount of secreted protein in the pen. All else being equal, larger bound complexes will diffuse out of the pen more slowly than smaller unbound reporter molecule, resulting in a higher concentration in the pen and a higher signal. For a given K_{D}, the fraction of the ligand bound under equilibrium is described by the following equation:

n[L]/K_{d}+[L]=[L]_{bound}/[M]₀

Where n is the number of binding sites, Kd is the equilibrium disassociation constant, [L] is the concentration of the ligand (reporter molecule), and [M]0 is the total concentration of the macromolecule (target secreted analyte). Using the above equation, FIG. 10 shows the fraction bound of the target molecule as a function of the free ligand (i.e. the reporter molecules) concentration as a function of K_{D}. The fraction bound is proportion to the signal above the background while the ligand concentration is proportion to the background. As the concentration of the ligand is increased above the K_{D}, the signal begins to saturate as the background concentration increases. For example, increasing the concentration from 10×K_{D} to 100×K_{D}, the fraction bound increases from 91% to 99%, an 8% increase in the signal. This, however, comes at the cost of a 10-fold increase in the background.

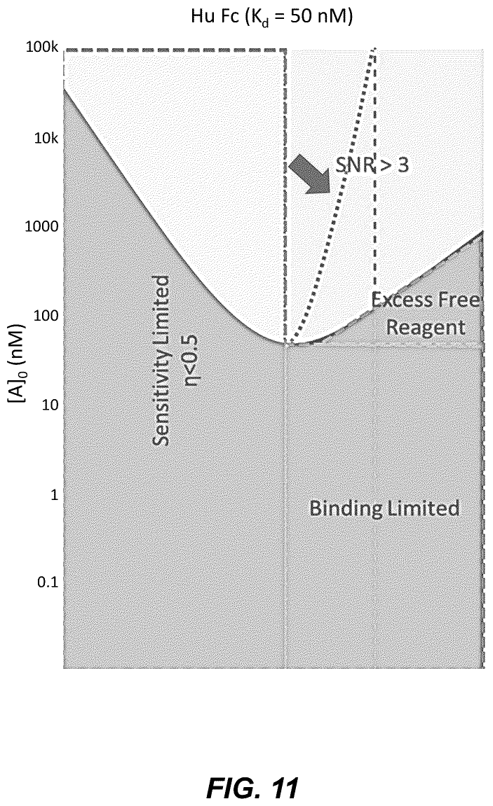

Under equilibrium, the total fluorescent signal from both the free and bound fluorescent detection molecule is a major determinant of the noise and thereby the sensitivity. Assuming the primary contribution is photon noise, the optimal concentration of the detection molecule will be somewhere between 1×−10×K_{D} (FIG. 11).

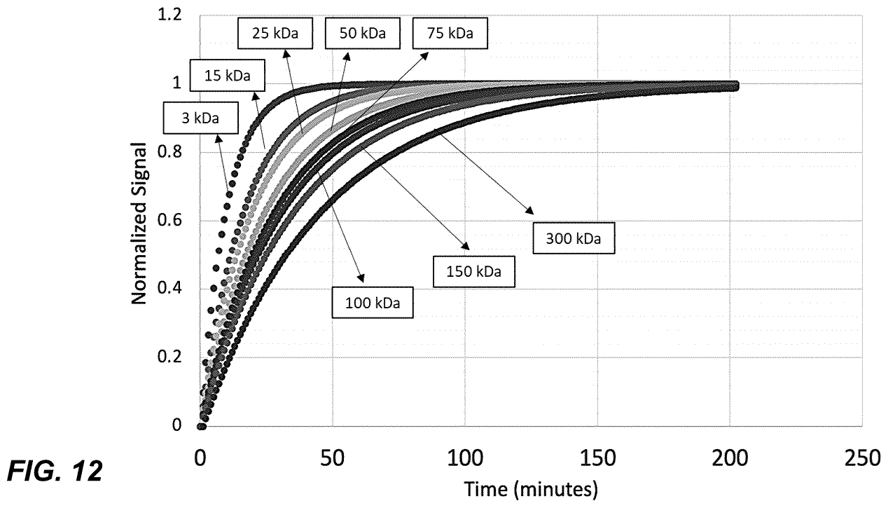

Equilibration Time: Molecular Weight of the Reporter Molecules. FIG. 12 shows a diffusion rate model. In this model, reporter molecules of various molecular weights were introduced and allowed to diffuse into a chamber. The reporter molecules were detected to observe the time required to reach a steady state equilibrium. For the development of new assay reagents, this model can be used to determine the equilibration time for different molecular weight reagents. Alternatively, it can allow for use of a single equilibration time that works for a large range of molecular weights. For example, in the experiment of FIG. 12, using an equilibration time of 2 to 2.5 hours results in 99% equilibration for all reagents less than 150 kDa. Moreover, while a small molecule diffuses out of the chamber quickly and therefore does not accumulate as much in the chamber, using a large detection reagent would result in a larger bound complex slowing the diffusion out of the chamber. As the system equilibrates to a steady state in the presence of the detection reagent, the amount of target protein in the chamber will increase, resulting in a higher signal and improved sensitivity.

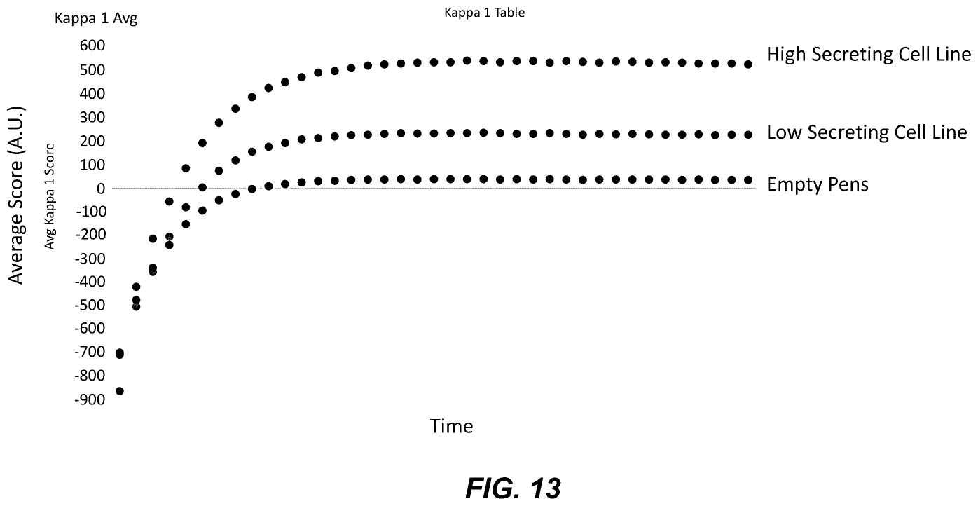

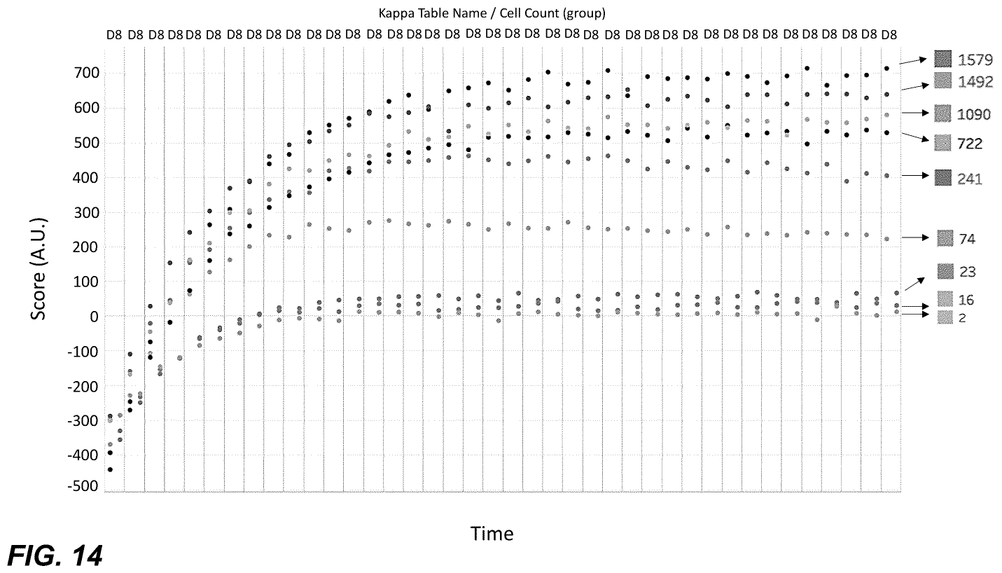

Secretion level of the cells cultured in the pen is also one of the factors affecting the time required to reach a steady state equilibrium. As shown in FIG. 13, reporter molecules (SpotLight Kappa) were introduced and allowed to diffuse into the chambers where a high secreting cell line and a low secreting cell line were cultured respectively. Some of the chambers were empty as a control. The total record time was 180 minutes (3 hours). As shown, the pen of a high secreting cell line requires a longer time to reach a steady state equilibrium compared with the pen of a low secreting cell line. Similarly, in FIG. 14, time lapse for reaching a steady state equilibrium was monitored in nine pens comprising cells of various levels of secretion or without cells. This figure shows different and distinct diffusion profiles reflecting the secretion levels of cells cultured in each chamber. The total record time was also 180 minutes (3 hours).

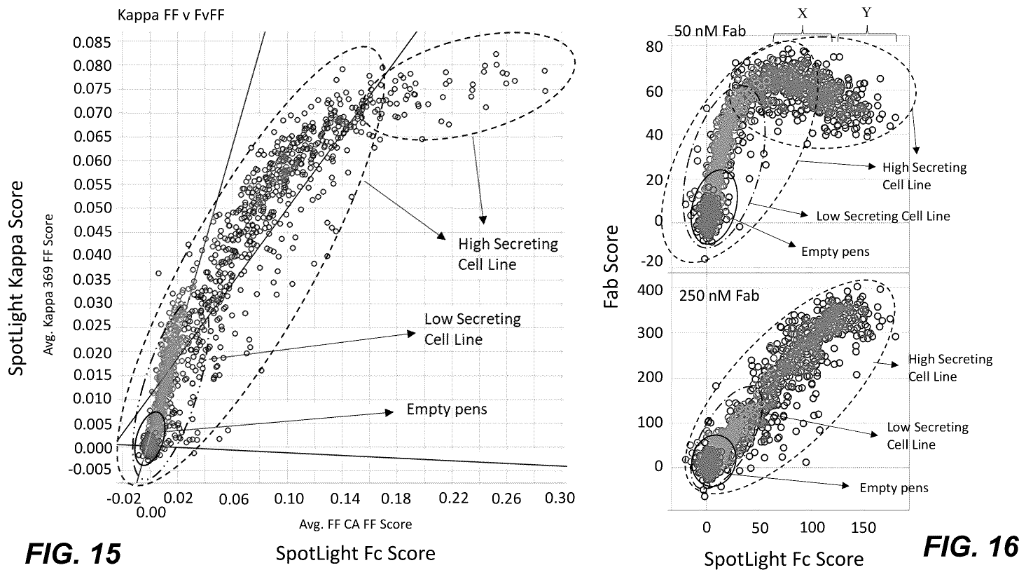

In some embodiments, more than one kind of reporter molecules can be introduced and allowed to diffuse into the chambers to reach a steady state equilibrium as explained above. FIG. 15 shows that SpotLight Kappa (MW: about 13.5 kDa; Concentration: 50 nM) and SpotLight Fc (MW: about 2.5 kDa; Concentration: 50 nM) were sequentially introduced into the chamber and reached a steady state equilibrium respectively. The two reporter molecules were then detected. FIG. 15 shows a linear dynamic range of scores of the two reporter molecules among pens. FIG. 16 shows another example of using two reporter molecules, while in this experiment, the difference of the molecular weight of the two reporter molecules was more significant. SpotLight Fc has a molecular weight of about 2.5 kDa and the concentration thereof used was 50 nM. Fab, the other reporter molecule (designed to bind to Fab regions) used in this experiment, has a molecular weight of about 50 kDa (20 times of the MW of SpotLight Fc). In this experiment, Fab was used at concentration of 50 nM (upper) and 250 nM (bottom). The result shows that reporter molecules of larger molecular weight require higher concentration to increase the diffusion rate so that they can reach the steady state equilibrium faster. This is particularly critical when more than one kind of reporter molecules are used because it is important to have the time required for the reporter molecules to reach steady state equilibrium aligned. Otherwise, as shown in FIG. 16 (upper), the X region pens seem having similar or even higher Fab score than Y region pens, but it was actually because the concentration of Fab reporter molecules was not sufficient, and many of the secreted analytes in the pens had not yet bound with the reporter molecules. Compared with FIG. 16 (bottom), when higher concentration of Fab reporter molecules was used, a linear dynamic range similar with the one observed in FIG. 15 can be obtained. Furthermore, the upper limit of the linear dynamic range can be determined by the secretion rate of the target protein.

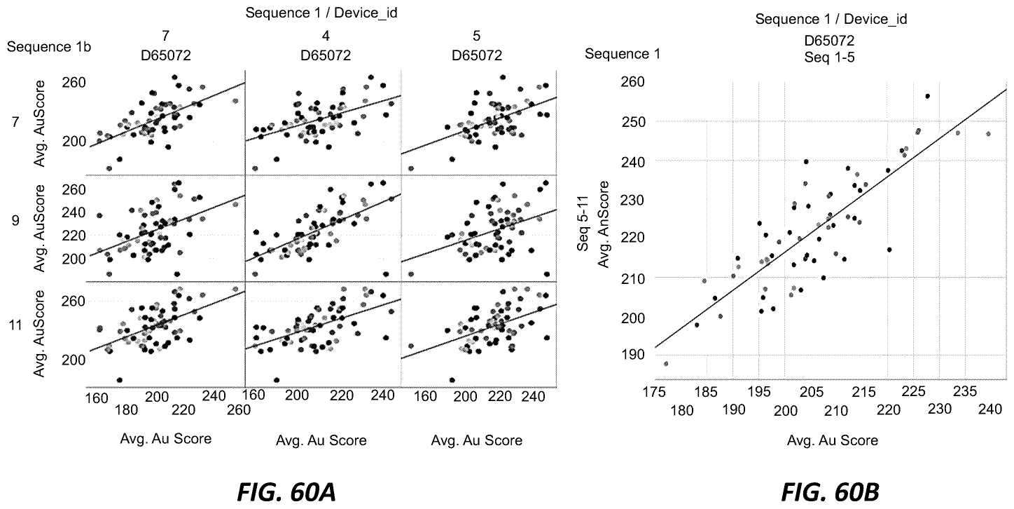

Image analysis. Image averaging to reduce noise and improved scoring metric. As discussed previously, acquiring multiple images can further assist in averaging out noise, thus improving signal-to-noise ratio. Averaging N images reduces the noise by the square root of N. For example, averaging four images together would reduce the noise by a factor of two while averaging nine images would reduce the noise by a factor of three. For example, to demonstrate the improvement in the consistency of the rank order of clones and therefore the accuracy of the measurement, a set of 11 sequential images were taken 10 minutes apart after the system was fully equilibrated. Referring to FIG. 60A and FIG. 60B, analysis was performed of the correlation of a subset of top secreting clones (63 of the top 65 clones, excluding 2 high outliers). FIG. 60A illustrates the correlation between single images while FIG. 60B illustrates the comparison of the average of the score from the first 5 images with the average of the last 6 images. Currently the score is calculated using the regions 9-13 in the scoring area. Extending the region to 5-15 to calculate the score (i.e. slope of the gradient) further reduced the noise. The average correlation between single images improved to R2=0.75+/−0.1 and comparing the 5 and 6 image average improved correlation to R2=0.90. Further, comparing a selection of single images the average correlation (R2) was 0.33+/−0.25 (095% CI), with a minimum of 0.15 and a maximum of 0.62. Comparison of the average score from the first 5 images with the average of the last 6 images, the R2 improved to 0.71.

Selection. A biological micro-object producing a protein of interest having more than one type of binding regions can be selected based on the method of the present disclosure. In some embodiments, the microfluidic device comprises a first chamber and a second chamber. In those embodiments, introducing a biological micro-object comprises: introducing a first biological micro-object into the first chamber and introducing a second biological micro-object into the second chamber. Preferably, the first biological micro-object and the second biological micro-object are expanded into a clonal population respectively and allowed to secret analytes as described above.

Furthermore, in those embodiments, detecting a first signal associated with the first detectable label and detecting a second signal associated with the second detectable label comprises detecting the first signal and the second signal in the first chamber and detecting the first signal and the second signal in the second chamber; and analyzing a ratio of the first absolute quantitation value and the second absolute quantitation comprises analyzing the ratio in the first chamber and analyzing the ratio in the second chamber. In other words, a ratio of the first absolute quantitation value and the second absolute quantitation can be obtained respectively for the first chamber and the second chamber. Accordingly, the first biological micro-object and/or the second biological micro-object can be selected or dis-selected by comparing the ratio thereof with a pre-selected ratio. In some embodiments, the method can further comprises exporting the selected biological micro-object from the chamber and, optionally, from the microfluidic device. The exporting can be conducted by OEP as described herein.

Kits. Kits for identifying a biological micro-object producing a protein of interest having more than one type of binding regions are provided. The kits may comprise: a first reporter molecule configured to emit a detectable signal (e.g., intrinsically or via a first detectable label) and having a first binding component configured to bind an analyte of interest (e.g., a first portion of a multiconfiguration multidomain biomolecule); and a second reporter molecule configured to emit a detectable signal (e.g., intrinsically or via a second detectable label) and include a second binding component configured to bind the analyte of interest (e.g., a second portion of a multiconfiguration multidomain biomolecule). In certain embodiments, the first reporter molecule comprises a first detectable label, which may be covalently bound to the first binding component. In certain embodiments, the second reporter molecule comprises a second detectable label, which may be covalently bound to the second binding component. The reporter molecules and the detectable labels can be as described elsewhere herein. In some embodiments, the kit further comprises a microfluidic device comprising an enclosure having a flow region and a chamber fluidically connected and opening to the flow region. In some embodiments, the microfluidic device is as described herein.

Machine-readable storage device. In certain embodiments, the disclosure further provides machine-readable storage devices for storing non-transitory machine-readable instructions for carrying out the disclosed methods of assaying for production of an analyte of interest such as a multiconfiguration multidomain biomolecule. In order to perform these methods automatically, a non-transitory computer-readable medium is provided, including a program for causing a computer to perform an image processing method for determining an intrinsic diffusion gradient as described above. The method can include receiving imaging data of a microfluidic device that includes a flow region and a plurality of chambers that are fluidically connected to the flow region; wherein the imaging data comprises an diffusion gradient image of the reporter molecules and optionally, one or both of a background noise image and a signal reference image; defining an area of interest for each chamber; and determining scores that are indicative of the secretion of the protein of interest in each chamber. In some embodiments, the non-transitory computer readable medium may include any method as described herein.

### III. ASSESSING THE QUALITY OF SECRETED BIOMOLECULES

Despite a growing need for earlier information on quality and manufacturability, initial clone screening in mammalian cell line development continues to focus on selection for growth and titer. Yet the fastest-growing and highest-producing clones may not secrete product that meets target quality profiles, potentially leading to costly processing inefficiencies downstream. As a consequence, large numbers of clones must be expanded and characterized through repeated rounds of selection in order to maximize the probability of finding cell line that makes high titers of manufacturable product.

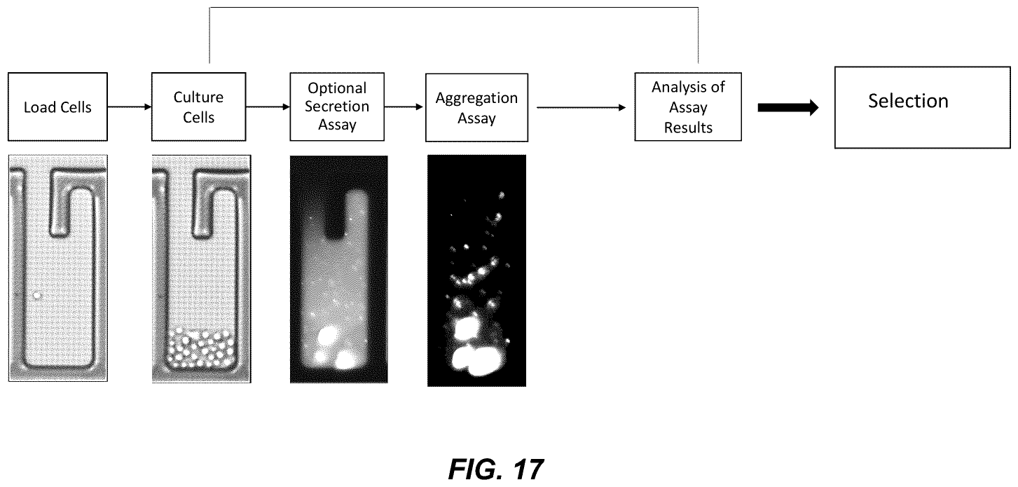

The present disclosure teaches a workflow for identifying cell lines that secrete a biomolecule of interest. As shown in an embodiment of FIG. 17 the workflow includes, loading a biological micro-object into a microfluidic device; culturing cells inside the microfluidic device and assaying the biomolecule of interest secreted by the biological micro-object. In some embodiments, the assaying the biomolecule of interest includes assaying the amount and/or the quality of the secretion. In some embodiments, the amount of the secretion is evaluated by the diffusion gradient assay discussed above or as disclosed in WO2017181135, filed on Apr. 14, 2017, WO2019075476, filed on Oct. 15, 2018, and WO2021183458, filed on Mar. 8, 2021, each of which disclosures are herein incorporated by reference in its entirety.

In some embodiments, assaying the quality of the secretion includes assaying the aggregation of the secreted biomolecule of interest (e.g. the aggregation assay recited in the present disclosure). In some embodiments, the biological micro-object to be loaded includes a population of cells, and the loading includes identifying the expressors among the population of cells before loading. In some embodiments, the aggregation assay is conducted following the diffusion assay.

Additionally, improved methods of selective penning are described that integrates high-throughput, multi-parameter analysis and sorting of individual cells with desired attributes from a heterogeneous population. Improvements providing selective penning can ameliorate long development times seen in traditional cell line development, which are due, in part, to low cell viability observed post-transfection. There is often a long recovery period where the stable pools are allowed to recover until there is a high percentage of viable cells to avoid screening dead clones This can take several weeks before the clones are ready for screening. During this recovery time, there is a potential loss in the genetic diversity of the screened pool since faster-growing clones can out-compete the slower-growing counterparts. Pre-enrichment, particularly in the microfluidic systems described herein, increases screening capacity up to about 100,000 cells in a single workflow, for example by performing a four chip experiment. More capacity means more access to relevant diversity in transfection pools than traditional cloning methods. Pre-enrichment directly from bulk stable pools, bypasses the need for mini-pool selection, reducing cell line development timelines by several weeks while additionally permitting identifying the best production cell lines.

Automated imaging and in-process controls possible within the microfluidic system described herein affords the ability to visually track each clone throughout the workflow, correlate quality measurements including those described below, and enable recovery of top producing clones having high levels, e.g., >99%, monoclonality.

Aggregation assay: Biologics are becoming increasingly complex to manufacture and regulatory requirements are becoming more stringent. Quality issues, such as aggregation, in highly engineered proteins have implications for drug manufacturability, shelf life, and patient safety. Identifying manufacturing cell lines that secrete quality product, free of aggregates and unwanted by-products, is emerging as a critical challenge in cell line development. Therefore, there is a need to develop additional methods to assess such quality issues as aggregation, when attempting to identify clonal populations that produce a biomolecule of interest.

Applicant has discovered that early, multi-parameter assessment of clones using the methods described herein enables early elimination of clones that are susceptible to quality issues like aggregation. Only top producers with best quality profiles are selected for initial scale-up, minimizing process risk and saving valuable development time. These methods enable detection of product aggregates within days of single cell cloning, e.g., within a week. Early elimination of clones susceptible to aggregation not only increases likelihood of identifying more optimal production cell lines, but also helps speed development by reducing the number of clones that must be selected and processed.

After synthesis, a protein folds into a three-dimensional structure primarily driven by the hydrophobic effect. The structure is stabilized by non-covalent interactions (e.g., hydrogen bonds in alpha helixes and beat sheets) and disulfide bonds. Protein aggregation occurs when the three-dimensional structure and the non-covalent interactions are disrupted, resulting in the protein unfolding or misfolding. In this state the protein can aggregate into three types of aggregates: amorphous aggregates, oligomers, or amyloid fibrils. When producing clonal or subclonal populations of cells within a microfluidic device, it would be desirable to be able to assess how much aggregation is occurring within the microfluidic environment along with assessing the secretion rates of the clonal/sub-clonal population. This is an important problem to solve, in order to predict which clones or subclones may more efficiently produce proteins with less propensity to producing aggregation products of those proteins.

There are several different mechanisms by which aggregates can form. In practice, aggregates do not always grow to macroscopic length scales (>10 to 10² microns), and instead remain as soluble oligomers and multimers. Aggregation can result in larger oligomers which can result in a slowed diffusion out of the chamber in which the protein has been produced. Fluorescent punctate, which are not part of a cell, can be observed when performing various types of diffusion-based assays, such as those described herein. These punctate could be a result of fluorescent labelling reagent binding to large protein aggregates in the chamber.

Possible explanations for the variation in the sub-clones could be due to phenotypic variation in either the quality or amount of the protein. As aggregation is concentration dependent, high secreting sub-clones may be more prone to form punctate. In chambers with sub-clones that do not exhibit the punctate, there may be the presence of smaller, soluble aggregates or oligomers that are not visually detectable.

Current general methods to quantitate or detect aggregation fall in to two general categories, a size measurement or a fluorescent measurement. Size measurements include ultracentrifugation, size-exclusion chromatography, gel electrophoresis, dynamic light scattering, or turbidity measurements. However, these techniques typically are not compatible with in-situ microfluidic culturing techniques. Applicant has surprisingly discovered that fluorescence measurements may be applied to the problem of assessing aggregation within microfluidic culturing and assay systems. It was known that using specifically developed fluorescent stains, such as Thioflavin-T, that amyloid fibrils may be preferentially stained. However, Applicant has advantageously devised methods of size-based measurement that is agnostic to the type of aggregate. Fluorescent stains, such as described for in-pen assays as described herein, may be employed to assess protein aggregation as well as secretion rate. These methods can rapidly identify and assess quality of clonal cell lines with the potential to select for the best downstream product and/or prevent product failure in downstream bioprocess development.

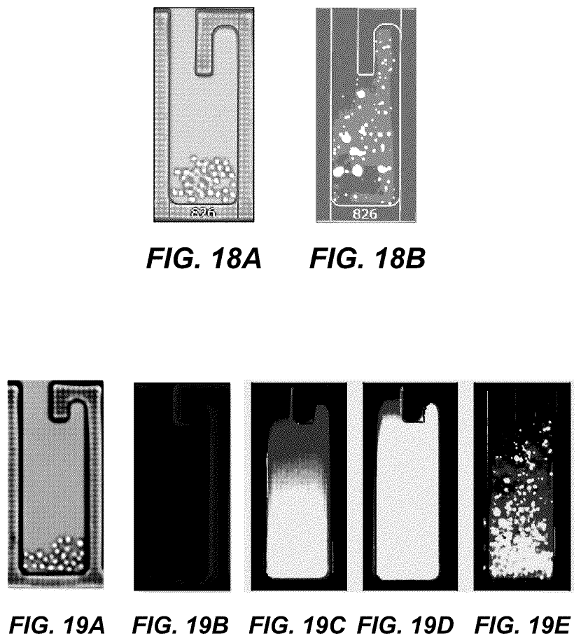

In FIG. 18A, a brightfield image is shown of a selected chamber of the microfluidic device, showing cells which are secreting a protein of interest. Various assays as described here and also described in U.S. application Ser. No. 16/160,816, published as US2019/0240665, and filed on Oct. 15, 2018; U.S. application Ser. No. 16/849,811, published as US20200408751, and filed on Apr. 15, 2020; and International Application Serial No. PCT/US2021/021417, filed on Mar. 8, 2021, each of which disclosures is herein incorporated by reference in its entirety, introduce a fluorescent labelling agent which binds to the secreted protein of interest. In FIG. 18B, a fluorescence image taken of the same chamber, shows fluorescent punctate regions, which are believed to be aggregated secreted protein suspended in the pen and include regions clearly not inhabited by cells. This may not occur in every chamber of the microfluidic devices, and the amounts of punctate fluorescence may vary considerably.

Applicant has discovered methods assessing the amounts of punctate regions in order to rank clonal or sub-clonal populations of cells that can most efficiently produce soluble proteins of interest. This method may further be used to predict which clonal or sub-clonal populations may be the highest value clones or subclones for large scale production of the protein of interest. Clonal or sub-clonal populations having lower amounts of insoluble aggregated secreted bioproducts may generally provide cell lines that are more efficiently scaled for commercialization. These methods may significantly reduce risk, cost and time of development of cell lines that can produce proteins of commercial interest. As described in further detail in Example 3 below, Applicant has shown that the methods of detecting insoluble aggregates of secreted bioproducts do in fact detect these aggregates specifically: fluorescently labelled punctate regions are not an artifact of the detection molecules; are specific for the type of secreted bioproduct; do not contain cell debris; and may be reproduced experimentally to demonstrate that the insoluble aggregates are the result of aggregated secreted bioproducts of the cells.

Accordingly, a method is provided for selecting populations biological micro-objects, e.g., cells, producing soluble biomolecules, including: introducing a plurality of biological micro-objects into a plurality of chambers of a microfluidic device, where the microfluidic device has an enclosure having a flow region. Each chamber of the plurality of chambers may be fluidically connected to the flow region, and the plurality of chambers may contain a first fluidic medium. The plurality of biological micro-objects, or the populations of biological micro-objects generated therefrom may be allowed to secrete an analyte, e.g., a molecule of interest, into the first fluidic medium within the plurality of chambers. Typically, the biological micro-objects may be cultured during the period of secretion, while media is flowing either continuously or intermittently. An intrinsic steady state diffusion gradient is established when the analyte, e.g., soluble biomolecule, is secreted by the biological micro-object(s) in the chamber and then diffuses into the channel. In some embodiments, the analyte secreted by the biological micro-object may be a protein, a saccharide, a nucleic acid, an organic molecule other than a protein, saccharide, or nucleic acid, a vesicle, or a virus. In some further embodiments, the analyte secreted by the biological micro-object is an antibody or, optionally, a glycosylated antibody.

A second fluidic medium may be introduced into the flow region, where the second fluidic medium comprises a plurality of soluble reporter molecules. The reporter molecule can be as described above. Each reporter molecule includes a detectable label and a binding component configured to bind the secreted analyte. Flow of the second fluidic medium in the channel is selected, as described herein, to be at a flow rate, where fluidic exchange between the channel and the chamber is primarily driven by diffusion, and greater than about 90%, 93%, 95%, 97%, or greater than about 99% of the fluidic exchange is driven by diffusion.

A portion of the plurality of soluble reporter molecules is allowed to diffuse into the plurality of chambers and bind to the analyte, e.g., protein of interest, secreted within the chamber by the cells, and produces reporter molecule:secreted analyte (RMSA) complexes. The RMSA complex is described above. Punctate regions of detectable label located within an area of interest within each chamber of the plurality may be detected at a selected period of time after the portion of soluble reporter molecules has been permitted to diffuse into the plurality of chambers. In some embodiments, after equilibrium has been established between binding of the soluble reporter molecule to the analyte and diffusion out of the chamber of unbound soluble reporter molecule and RMSA into the channel, media containing no reporter molecules may be flowed in the channel, creating a sink for molecules diffusing out of the chamber (soluble unbound reporter molecules, and soluble RMSA molecules. The period of time of media flushing/perfusion can be determined for the reporter molecule and specific RMSA, and images (brightfield and fluorescent) may be obtained to optimize identification and quantitation of the amount of labeled aggregated, e.g., insoluble, analyte retained in the chamber, but is not required to identify and quantify punctate regions accurately. The punctate regions of detectable label may be quantified within each chamber of the plurality. In some embodiments, the plurality of chambers may be ranked according to those containing the fewest punctate regions of detectable label. The quantification of punctate regions of detectable label may be used to select populations of biological micro-objects having desired levels of punctate regions, e.g., amounts of aggregated analyte produced therefrom.

As shown in FIG. 19A to 19E, a comprehensive array of images shows exemplar images that can be obtained. In FIG. 19A, a brightfield image shows a selected chamber, e.g., pen, having a population of cells growing within. When contacted with a reporter molecule configured to bind to the secreted analyte produced by the cells, and imaged under conditions where the label of the reporter molecule, a variety of possible images may be obtained. As shown in FIG. 8B, a negative control population will display no fluorescence at all. In FIG. 19C, cell populations having a low level of expression of the analyte shows a low level of fluorescence in the image. In contrast, FIG. 19D shows cells having a high level of expression and greatly increased fluorescent image. In FIG. 19E, however, it can be seen that in some chambers having fluorescence, the fluorescence is distributed in a punctate manner, and indicates that unlike the chamber in FIG. 19D, the complex of the reporter molecule and analyte is not a soluble complex, but has aggregated. This characteristic of the analyte being produced by cells in such an exemplary chamber, is less desirable than the highly expressed analyte of the chamber of FIG. 19D. Determining relative quantities of aggregative secretion demonstrated by punctate fluorescent regions is needed.

In some embodiments, these methods may be performed automatically, e.g., by a computer controlled process. In some embodiments, these methods may further be performed in combination with, prior to, or subsequently to performing methods of assaying for a level of secretion of the analyte, e.g., biomolecule of interest, some nonlimiting examples of which are described herein. In some embodiments, the aggregation assay is conducted after a diffusion assay has been performed. The diffusion assay may be any suitable diffusion assay, including diffusion assays described herein, and may be performed to identify cells secreting a desired amount of the protein of interest. The aggregation assay may then be performed to identify cells secreting the protein of interest having a desired quality or characteristic, such as, degree of aggregation below a selected level. In some embodiment, the aggregation assay is performed after the cells have been introduced into chambers, e.g., sequestration pens, using methods of enhancing penning of cells that secrete the analyte, to thereby increase the proportion of cells having selected levels of secretion finally disposed within the chamber. In some embodiments, the enhanced introduction of secretor cells, the diffusion assay, and the aggregation assay may be performed sequentially. However, the methods are not so limited, and in some embodiments, an aggregation assay may not be performed in combination with either enhanced introduction of secretor cells and/or any kind of assay of the analyte secreted by the secretor cells.

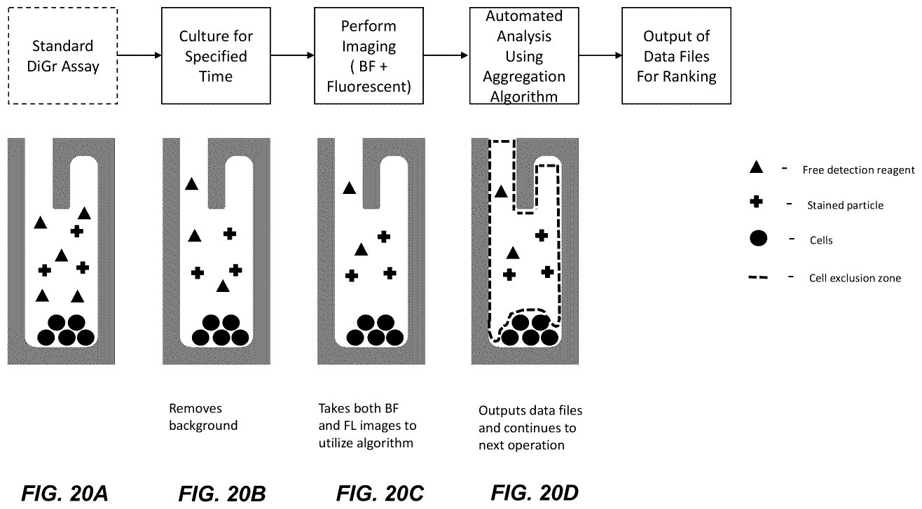

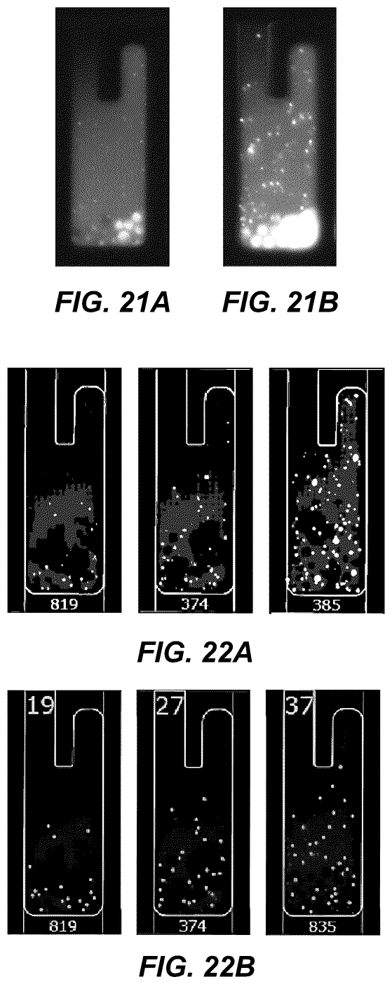

In some embodiments, an aggregation assay may be conducted immediately after a diffusion assay for determining the amount of an analyte is performed. As shown in FIGS. 20A to 20D, after completion of a diffusion assay (FIG. 20A), the reporter molecule:secreted analyte (RMSA) complexes (stained particles) are already formed in the chamber and it is not necessary to perfuse another flow of soluble reporter molecules or a detection reagent (e.g. a fluorescent labeling agent). Specifically, the following components within the chamber may be sufficiently retained: free secreted protein, free reporter molecules, and/or the RMSA. In addition, if aggregation is formed within some of the pens, there may also exist soluble aggregates of varying sizes, both bound and unbound to reporter molecules and/or insoluble aggregates both bound and unbound to reporter molecules. The signal from both the free reporter molecules and soluble bound reporter molecules create a background signal. Minimizing this signal maximizes detection capability for the detection reagent bound to insoluble particles or aggregates. Therefore, in some optional embodiments, a further culture period may be employed where standard media, e.g, containing no reporter molecules, may be flowed through the channel of the microfluidic device, and the free and soluble bound detection reagent slowly diffuses out of the pens (FIG. 20B). The detection reagent bound to the insoluble particles remains in the pen (there may be a decrease in signal due to Koff rate of detection reagent). FIG. 21A shows an image taken of a selected chamber having punctate regions, after a diffusion assay is performed and no additional media flow is performed before analyzing for punctate regions. FIG. 21B shows an image taken of the same chamber, after flowing media containing no reporter molecules, increasing the proportion of the soluble free reporter molecule and soluble RMSA diffusion out of the pen. Additional image filtering may be employed in either scenario to enhance the contrast between punctate regions and surrounding regions of the chamber forming part of the AOI. Resultingly the punctate regions within the pen become more obvious.

After a specified time, an imaging sequences then takes a brightfield and fluorescent image in quick succession (FIG. 20C). This minimizes potential drift of cells, and permits the preparation of an imaging mask that excludes regions having cells from punctate region detection as described in more detail below. A higher exposure time can also be used due to the lower background. More accurate detection of stained insoluble particles in the non-cell areas of the pens results from any of these features, providing a lower limit of detection and high signal to noise ratio. While these features can improve detection but are not required for successful identification and scoring of pens of interest. The standard images taken as part of the diffusion assay (in both BF and fluorescent channels) are adequate to utilize the aggregation detection algorithm and generate measurements. Then, area of interest (e.g., cell exclusion zone) is determined and punctate regions within the pen are identified and counted by algorithm for scoring each pen (FIG. 20D). The determination of the area of interest and the scoring are described in more details in the following paragraphs.

A fluorescent cell detection algorithm (traditional thresholding or use of a Convolutional Neural Network (CNN) detecting fluorescent regions) having highly sensitive settings (e.g., MinCellDiameterMicrons: 0) may be used to detect a range of sizes of punctate regions. In some embodiments, the CNN or a threshold limitation setting detects fluorescence within a region having an area corresponding to a minimum diameter greater than a sub-micron diameter, greater than about 1 micron diameter, greater than about 2 micron diameter, greater than about 3 micron diameter, greater than about 5 micron diameter, greater than about 10 micron diameter or greater than about 15 micron diameter. An example where the CNN identifies punctate regions, based on a threshold is shown in FIGS. 22A and 22B, where FIG. 22A shows punctate regions for a group of individual chambers labelled 819, 374, and 385 respectively. In FIG. 22B, the CNN detects punctate regions having an intensity greater than a set level, and shows that for chamber 819, 19 punctate regions were identified; for chamber 374, 27 punctate regions were identified; and for chamber 835, 37 punctate regions were identified. In some embodiments, the scoring of aggregation assay conducted for each pen involves the number of punctate regions identified. In some embodiments, the scoring is solely based on the number of the punctate regions; specifically, the higher the number of the punctate regions, the higher the scoring is. In other words, pens of high scores are unfavorable. Alternatively, the scoring involves considering not only the number of the punctate regions but also other data, such as cell count, e.g. to normalize data across a plurality of chambers of the microfluidic device; total area of all punctate regions identified (TotalAreaPixels); average area of all punctate regions (AvgAreaPixels); or combinations thereof. The scoring may be determined by an algorithm that includes these additional characteristics.

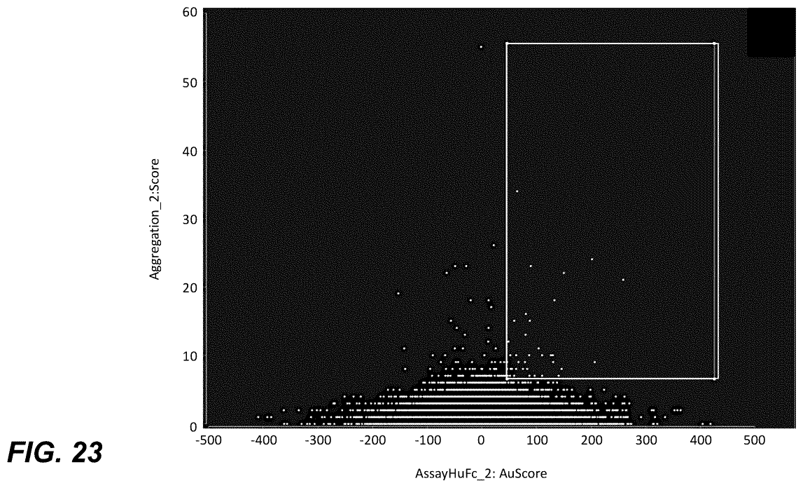

In the embodiments where the diffusion assay and the aggregation assay described herein are both conducted, the scores of the two assays are determined respectively and considered together for selecting the pens from which the cells cultured within are exported. Generally, pens selected possess high diffusion assay score and low aggregation assay score, suggesting that cells within those pens secret relative high amount and quality of the protein of interest. In FIG. 23, the results of both diffusion assay (amount of secreted molecule, plotted along the x axis) and the aggregation assay (amount of aggregate, e.g. punctate regions, plotted along the y axis) are shown, having a data point for each of the sequestration pens in the chip. The boxed region at the right hand side of the graph identifies pens having cells which both secrete greater quantities of product molecule while producing relatively less aggregation product. Cells from these pens may prove to be more successfully scaled and commercialized. Clones can be filtered and sorted by the algorithm based on their aggregation score along with ranking by productivity, cell growth, and other user-defined parameters to identify and select the best clones for export from the microfluidic device and further evaluation, scale up or other processing.

In some embodiments, the area of interest within each chamber of the plurality may exclude a region containing the plurality of biological micro-objects or the populations of biological micro-objects generated therefrom. In some embodiments, the area of interest may lie along an axis of diffusion between the plurality of chambers and the flow region (e.g., along an axis of diffusion between the isolation region of the plurality of chambers and the microfluidic channel, such as along an axis of diffusion defined by the connection region). In some embodiments, the area of interest may not lie along an axis of diffusion between the plurality of chambers and the flow region (e.g., not along an axis of diffusion between the isolation region of the plurality of chambers and the microfluidic channel, or not along an axis of diffusion defined by the connection region). The area of interest may include an image arca corresponding to an area within a chamber of the plurality of chambers that is most sensitive for measuring analyte concentration fluctuations, and least sensitive to the position of biological micro-objects in the chamber when analyte fluctuations are measured.

In some embodiments, the area of interest may be a functional punctate detection region. A mask, which may be in some embodiments, a pixel mask, can be used in order to create a functional punctate detection region. This mask can be either defined statically (e.g., top half of the pen), or dynamically using cells detected in a brightfield imaging sequence. Although using a dynamic approach increases time required for computation, it may provide better masks to ensure exclusion of the secreting cells, since some chambers may have a higher population of cells than others.

One nonlimiting example of a dynamically defined area of interest may be obtained. A first brightfield image may be obtained of each chamber of the plurality prior to introducing the second fluidic medium, thereby identifying each of the plurality of biological micro-objects or the populations of biological micro-objects generated therefrom and assigning each a respective cell-containing area. A second brightfield image may then be obtained of each chamber of the plurality of chambers at the selected period of time after reporter molecules have diffused into the plurality of chambers, thereby confirming each of the cell-containing areas. A dynamically defined area of interest may be created by creating a pixel mask including each of the cell-containing areas in each chamber; and defining the area of interest within each of the chambers to exclude each pixel mask in each chamber. That is for determining the number of punctate regions, none of the area within the chamber occupied by the biological micro-objects is included within the dynamically defined area of interest, e.g., the functional punctate detection region.

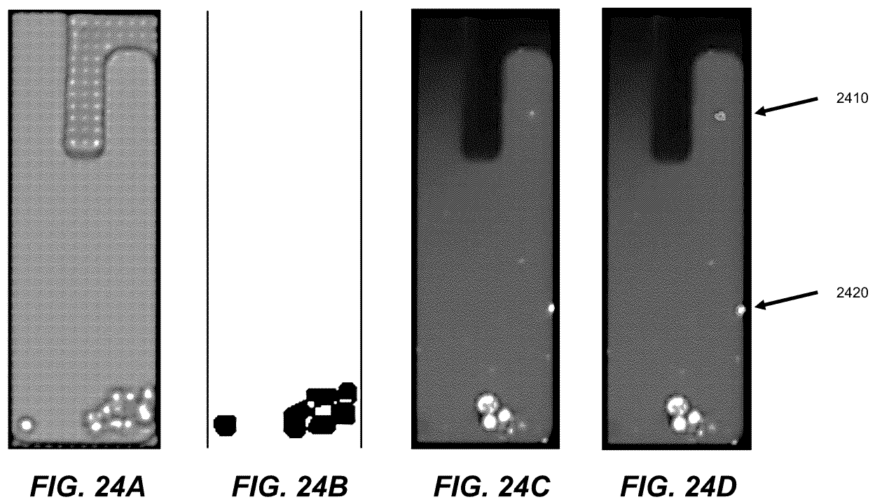

An example is shown in FIG. FIG. 24A, where a brightfield image of the biological micro-objects, e.g., cells secreting the analyte, a protein of interest, is shown. In FIG. 24B, the dynamically defined area of interest is shown, where the uncolored region permits quantification of punctate regions, while the blackened regions, here seen at the bottom of the chamber, masks out the area occupied by the cells themselves, i.e., the functional punctate region explicitly excludes the areas occupied by cells. Any label that attaches to the cells will not be included in the punctate detection. FIG. 24C shows the base fluorescent image showing both regions containing cells and regions potentially containing aggregated protein. FIG. 24D shows the finalized image, where the two encircled small punctate regions 2410, 2420 are properly identified as punctate region. While there are highly fluorescent areas near the base of the chamber, these are not included in the image FIG. 24D, used to quantify the punctate regions. Since these are recognized as cells from the brightfield image, the functional punctate detection region is defined to be outside of these areas, and those regions of fluorescence are not included in the punctate quantification.

In some embodiments, the selected period of time after the soluble reporter molecules diffuses into the plurality of chambers may be as described above or from about 20 min to about 60 min, or any value therebetween. In some embodiments, the selected period of time may be about 45 min.

In some embodiments, the method may further include detecting detectable label located within the area of interest within the microfluidic device after the diffusion of reporter molecules into the plurality of chambers and the binding of reporter molecules to the analyte secreted therein is at or near steady state conditions; and ranking each chamber of the plurality of chambers based on the level of detectable label within the area of interest and based on the number of punctate regions within the area of interest.

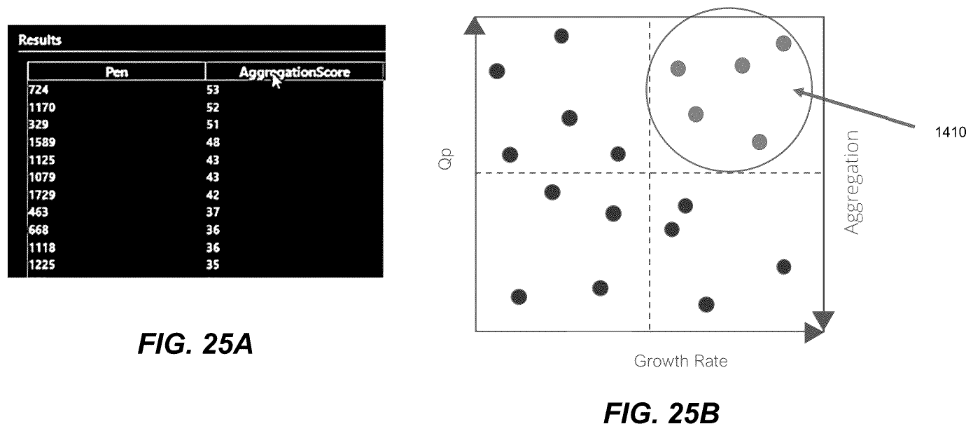

In some other embodiments, the method may further include: introducing a third fluidic medium into the flow region, where the third fluidic medium does not comprise reporter molecules. At least a portion of unbound soluble reporter molecules may be allowed to diffuse out of the plurality of chambers. Detecting the detectable label within the area of interest may be performed after the portion of unbound soluble reporter molecules have diffused out of the chamber, and ranking each chamber of the plurality of chambers based on the level of detectable label within the area of interest and based on the number of punctate regions within the area of interest may then be performed. The ranking may be relative or may be a score which includes the number of punctate regions detected, as shown in FIG. 25A.

In some embodiments, ranking may be based on a combination of the highest level of detectable label and on the lowest number of punctate regions, e.g., the highest amount of secretion by the biological micro-objects and the lowest amount of aggregation, quantified as the fewest number of punctate regions within a chamber. In particular, it is desirable to arrive at selection of cells that have both high levels of secretion (rQp, relative productivity), good growth rates (x axis) as well as relatively low levels of aggregation (right hand downward axis). This relationship is dually graphed for 9 populations in FIG. 25B, where on the left-hand side of the graph x increases as growth increases and y increases as productivity increases. In the right-hand panel, the same 9 populations are shown, again as x increases, growth rate increases, but y is an inverse mapping function with aggregation increasing downward towards the x axis. Use of these methods permits early selection of cell lines, in region 1410 having favorable manufacturability profiles-high growth, high productivity and high-quality product.

Machine-readable storage device. In order to perform these methods automatically, a non-transitory computer-readable medium is provided, including a program for causing a computer to perform an image processing method for determining a quantity of aggregation products produced by a biological micro-object, where the method includes receiving imaging data of a microfluidic device that includes a flow region and a plurality of chambers that are fluidically connected to the flow region; wherein the imaging data comprises an aggregation assay image and optionally, one or both of a background noise image and a signal reference image; defining an area of interest for each chamber; and determining scores that are indicative of the quantity of aggregation products in each chamber. In some embodiments, the non-transitory computer readable medium may include any method as described herein, e.g., any variation of the method of determining amounts of aggregated analyte, e.g., punctate regions. In some embodiments of the non-transitory computer-readable method, the method further comprises ranking the plurality of chambers corresponding to chambers comprising the fewest punctate regions of detectable label. In some embodiments, the method of the non-transitory computer readable medium may define the area of interest dynamically.

### IV. EXPRESSOR ENHANCED PENNING

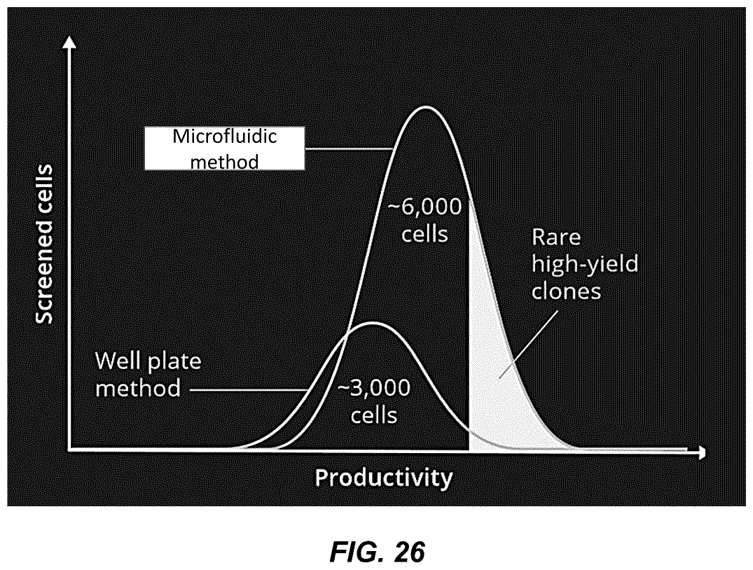

Microfluidic culture of cells that secrete a protein of interest can be particularly useful to identify clonal populations with high levels of secretion, thus decreasing cost, time and risk in campaigns to identify new or improved cells lines for protein (or other molecule) production. The problem is illustrated in FIG. 26, where typical commercial well plate methods of screening pools of cells, may present perhaps 3000 cells that can be screened. However, there is high cost in terms of time, reagents and lack of phenotypic information. In contrast, performing such clonal identification programs in a microfluidic device can increase the screening pools to about 6000 individual cells that can be expanded and assayed for detailed phenotypic information. Nevertheless, what is most desirable is to find the rare high yield clones, while decreasing the cost and time wasted on testing dead or non-expressing cells. There is need to further improve the process of identifying productive clonal populations to limit the number of dead or non-expressing cells that are penned at the start of the experiment. To this end, Applicant has discovered methods that enable in-channel identification, selection, and targeted penning of cells based on viability and/or productivity. More viable and productive clones may be available to screen for productivity, when pre-enrichment methods as described herein are utilized.

In some embodiments, cells that have detectable product expression are selected for penning, while in some other embodiments, cells may be prioritized for penning based on relative level of expression, e.g., the cells showing the highest level of expression may be penned first. These methods can be combined with staining, such as with Annexin V, propidium iodide, or the like, or combination thereof, to enable dual-channel selection of viable, high expressors.

Accordingly, a method is provided for enhanced loading of biological micro-objects secreting a molecule of interest into a plurality of chambers of a microfluidic device. The microfluidic device may be any suitable microfluidic device, including any of the microfluidic device embodiments disclosed herein. The microfluidic device may have an enclosure having a flow region and each chamber of the plurality of chambers is fluidically connected to the flow region. In some embodiments, the microfluidic device may be any microfluidic device as described herein. In some embodiments, the biological micro-objects are labelled with a first label configured to positively or negatively label a live cell, thus providing a first subset of the biological micro-objects. Alternatively, or in addition, the biological micro-objects may be analyzed with regard to one or more physical characteristics (e.g., size and/or shape) associated with cell viability. The first subset of the biological micro-objects includes biological micro-objects that are healthy and less likely to die during when cloned and assayed. The first subset of the biological micro-objects excludes biological micro-objects that are not viable, e.g., dead. The biological micro-objects are then labelled with a second label configured to label either the molecule of interest at a cell surface of a secreting biological micro-object or the molecule of interest in a region in proximity to the secreting biological micro-object, thus providing a second subset of the biological micro-objects. The second subset therefore includes biological micro-objects that express the molecule of interest. The second sub-set of biological micro-objects excludes biological micro-objects that do not express the molecule of interest.

The biological micro-objects, including the first and second sub-sets of biological micro-objects that have been labeled, are introduced into the flow region of the microfluidic device. The flow region and chambers are filled with a first fluidic medium into which the biological micro-objects are introduced. Selective penning is then performed, where a biological micro-object is selected for penning only if it is a member of both the first and the second sub-sets. That is, only biological micro-objects that are both viable and expressors of the molecule of interest are selected and penned. Manual or automatic penning can select only viable cells and can select only cells that express or secrete the molecule of interest.

In another aspect, a method is provided for enhancing loading biological micro-objects secreting a molecule of interest into a plurality of chambers of a microfluidic device, which may be any suitable microfluidic device. Biological micro-objects are introduced into a flow region of the microfluidic device, where the microfluidic device has an enclosure having the flow region and each chamber of the plurality of chambers is fluidically connected to the flow region. In some embodiments, the microfluidic device may be any microfluidic device as described herein. The plurality of chambers and the flow region contains a first fluidic medium. The biological micro-objects, e.g., cells, are labelled with a first label configured to positively or negatively label a live cell, thus creating a first sub-set of the biological micro-objects labelled such that selection for live cells can be made. The biological micro-objects are then labelled with a second label configured to label either the molecule of interest at a cell surface of a secreting biological micro-object or the molecule of interest in a region in proximity to the secreting biological micro-object, to provide a second sub-set of the biological micro-objects. Manual or automatic penning can select only viable cells and can select only cells that express or secrete the molecule of interest. Thus, penning can be selective, penning only biological micro-objects that are member of both the first sub-set and the second sub-set.

In yet another aspect, the biological micro-objects are labelled with at least two of: labelling a first sub-set of the biological micro-objects with a first label configured to positively or negatively label a live cell; labelling a second sub-set of the biological micro-objects with a second label configured to label either the molecule of interest at a cell surface of a secreting biological micro-object or the molecule of interest in a region in proximity to the secreting biological micro-object; and labeling a third sub-set of the biological micro-objects based on size thereof. The first label and the second label are as defined in the preceding paragraphs. Then, the biological micro-objects that are members of at least two of the first sub-set, the second sub-set, and the third sub-set the biological micro-objects are selectively penned.

In some embodiments, the third label can be conducted using brightfield image of the cells and gating the biological micro-objects of a pre-determined threshold of cell diameter. The pre-determined threshold can vary depending on the needs and cell types. In some embodiments, the threshold can be 3 to 4 um or any value therebetween and only cells of size larger than or equal to the threshold are selected (labeled). In an embodiment, the threshold is about 3.65 um. In some embodiment, the labeling of the third label can be performed by an algorithm. It is noted that the term “labeling” used herein does not necessarily mean the biological micro-objects are bound with any visible dyes or fluorescent molecules; instead, it can simply mean that the biological micro-objects are identified from other biological micro-objects through algorithm.

In some embodiments, the biological micro-objects are labelled with at least two of the first label, the second label, and the third label before being imported into the microfluidic device. In some embodiments, the biological micro-objects are labelled with at least two of the first label, the second label, and the third label after being imported into the microfluidic device. In yet some embodiments, one or some of the labelling are performed before the biological micro-objects are imported into the microfluidic device, and the rest labelling are performed after the biological micro-objects are imported into the microfluidic device. For instance, the biological micro-objects are labelled with the first label before being imported into the microfluidic device and then are labeled with the second label or the third label after being imported into the microfluidic device.

The method may further include not penning the remainder of the biological micro-objects (e.g., not members of both the first and the second sub-sets of the biological micro-objects). The method may further include exporting the remainder of the biological micro-objects from the microfluidic device.

In the method, the first label may be a label that may include a label that indicates viability or lack thereof. The label may indicate apoptotic or necrotic cells or may indicate a healthy and viable cell. The label may be, but is not limited to, Annexin V, propidium iodide, or the like, or combination thereof. In some embodiments, the first label may be a mitochondrial potential reagent and a member of the first sub-set may include the first label indicating energized and intact mitochondria. In some embodiments, the first sub-set may be negatively labelled, e.g., not labeled) by the first label.

In some embodiments, labelling the first sub-set of the plurality of biological micro-objects may be performed for about 30 min, 60 min, 90 min, 120 min, 150 min, 180 min, 240 min, 300 min, or more. In some embodiments, labelling the first sub-set of a plurality of the biological micro-objects may be performed at 4° C. to 37° C., 4° C. to 25° C., 25° C. to 37° C., or any value therebetween.

In the method, the second label may be an IgG expression reagent. In some embodiments, the IgG expression reagent may be a Protein A reagent having a fluorescent label, anti-Fc reagent having a fluorescent label, or the like. In some embodiments, the second label comprises an antigen-specific cell surface marker, a glucose uptake reagent, or the like. In some embodiments, labelling the second sub-set of the plurality of biological micro-objects may be performed for about 30 min, 60 min, 90 min 120 min, 150 min, 180 min, 240 min, 300 min, or more. In some embodiments, labelling the second sub-set of a plurality of the biological micro-objects may be performed at 4° C. to 37° C., 4° C. to 25° C., 25° C. to 37° C., or any value therebetween.

In some embodiments, the first label and/or the second label are formulated as a staining solution, and the staining solution further include a staining enhancer, such as Polyvinylpyrrolidone (PVP) or Ficoll. In some embodiments, a PVP solution used for formulating the staining solution is of a concentration of 0.5%, 1%, 1.5%, 2%, or 2.5% (w/v). In some embodiments, the volume of the first label or the second label to the PVP solution is 1:1 to 1:20, 1:4 to 1:20, or 1:8 to 1:20. In some embodiments, a working concentration of the PVP solution in the staining solution is 0.01 to 0.02, 0.012 to 0.02, or 0.016 to 0.019% (w/v). In some embodiments, a working concentration of the PVP solution in the staining solution is 0.016, 0.017, or 0.019% (w/v).

In some embodiments, selectively penning biological micro-objects that are members of both the first and the second sub-sets of the biological micro-objects may further include penning a single biological micro-object into each of the plurality of chambers.

In some embodiments, the method may further include culturing the selectively penned biological micro-objects, thereby expanding one or more clonal populations of biological micro-objects configured to actively produce the molecule of interest.

In another aspect, a method is provided for prioritizing a loading of biological micro-objects secreting a molecule of interest into a plurality of chambers of a microfluidic device. The biological micro-objects are labelled with the first label and the second label to identify the biological micro-objects belong to the member of both the first sub-set and the second sub-set as described above. Then, those biological micro-objects are differentiated into multiple tiers by the secreting level of the molecule of interest based on the second label. In some embodiments, the second label has a fluorescent label, and the multiple tiers are differentiated by the intensity of the fluorescent label. In some embodiments, an order of penning is decided as to which the penning is prioritized to pen the first tiers (i.e., a sub-set of biological micro-objects that are viable and secreting the relatively highest level of molecule of interest) into a plurality of chambers of a microfluidic device and so on. In some embodiments, three tiers are determined including high secretors, low secretors, and non-secretors; wherein the high secretors are penned firstly, the low secretors are penned secondly, and the non-secretors are not penned. In this aspect, the method ensures the high secretors are penned, and if all chambers of the microfluidic device are occupied or the chambers left (i.e., the chambers that have not been occupied) are not enough for penning all the low secretors, all or some of the low secretors can be discarded.

Machine-readable storage device. In another aspect, a non-transitory computer-readable medium is provided including a program for causing a computer to perform an image processing method for enhancing loading a plurality of biological micro-objects secreting a molecule of interest into a plurality of chambers of a microfluidic device, where the method includes receiving imaging data of a microfluidic device that includes a flow region and a plurality of chambers that are fluidically connected to the flow region; where the imaging data includes a loading image including an image of the plurality of biological micro-objects disposed within the flow region, and one or more fluorescent images of a same image area; defining from the loading image and the one or more fluorescent images a selected portion of the plurality of biological micro-objects comprising one or more selected characteristics; selecting each biological micro-object of the selected portion; determining a trajectory to deliver each biological micro-object of the selected portion to a corresponding chamber of the plurality of chambers; and disposing each biological micro-object of the selected portion of the plurality of biological micro-objects within the corresponding chamber of the plurality. In some embodiments, a non-transitory computer-readable medium comprising a program for causing a computer to perform an image processing method for enhancing loading a plurality of biological micro-objects secreting a molecule of interest into a plurality of chambers of a microfluidic device is provided, wherein the method may further include any method of enhancing loading of biological micro-objects as described herein.

### V. POPULATION DYNAMICS

Phenotypic variation in subclones within clonal cell line (e.g., cells derived from a single cell) is important for understanding the stability of cell lines used in the production of proteins and small molecules. Such variation and insights derived therefrom can be critical for supporting the effective production and scaling of products produced from such cell lines, which can comprise a broad and growing class of molecules used in the production of everything from drugs to cosmetics.

Phenotypic variations can, for example, result from genetic changes (e.g., mutations) or non-genetic variation in the expression (e.g., intrinsic stochastic noise) of the phenotype. The intrinsic stochastic phenotypic variation can be a function of the numerous processes involved in expression of a molecule (e.g., a small molecule synthesized by the clonal cell line, a macro-molecule produced by the clonal cell line). For cell lines designed to secrete proteins (e.g., antibody secreting cells, cells engineered to produce enzymes, etc.). such processes involved in variations in the phenotype of a subclone can be for example: transcription, translation, post-translational modifications, etc. A phenotypic state for the subclone can be temporal, such that over a given time period a particular subclone may sample a range of phenotypic variants. Depending on the processes involved, the lifetime of a given phenotype variant can vary greatly and can persist anywhere from hours to spanning multiple generations. In some embodiments, the methods systems and devices disclosed herein can be applied to assess phenotypic variation in subclones of a particular cell line (e.g., clonal or non-clonal cell lines). Examples provide below are for clonal cell lines, however non-clonal heterogenous cell lines could also be sampled using similar or equivalent method. Comparison between primary subclones or secondary subclone populations derived from non-clonal cell lines could be useful for measuring or monitoring distribution of phenotypes. Distribution of different phenotypes across non-clonal or heterogenous cell lines could be useful for the purpose of screening rare outliers.

In some specific embodiments, a clonal cell line is selected in an initial screen and is scaled up to a higher density, typically taking 4-6 weeks. At this point the clonal cell line is banked creating a master cell line bank. This clonal master cell line is further characterized over an additional scale up period to ensure that the secretion phenotype, specifically titer, is stable over time. A clone is determined to be stable if the titer does not drop by more than 20% to 25% over the lifetime of the culture through fermentation, typically 50-80 generations. This helps ensure a predictable yield and quality of the desired product (e.g. protein therapeutic) in the fermentation manufacturing process and further is required by regulatory bodies.

In such an example, a single cell or small set of cells derived from a single cell (e.g., a clonal cell line) are characterized for the phenotypes of interest, such as secretion and growth rate (e.g., rQp). These measurements reflect the sub-set of the phenotypic states available to subsequent sub-clones over the time period of the measurement. This clonal cell line is then scaled up and allowed to expand to a larger number of primary subclone cells, in some instances reaching densities of millions of cells after 4 to 6 weeks (˜30-60 generations assuming an average doubling time of approximately 24 hours). During this time, even if there are no mutations that effect the phenotype of interest, there will be a reversion to the mean phenotype as the sub-clones explore the distribution of phenotypic states. To measure the population average and the distribution, a set of primary sub-clones are isolated and measured for one or more cell lines, and using the methods disclosed herein, by performing a screen of sub-clones from a cell line (e.g., a clonal cell line selected from a previous workflow), after the primary clonal cells of that cell line have been allowed to divide and expand into a large number of cells (e.g., a secondary sub-clone population). Following expansion of the number of cells, an assessment of the population distribution of the expanded sub-clone population can be performed by measuring one or more phenotypic features of the population or subpopulation of subclones, such features can include but are not to be limited to: secretion and growth rate.

In some embodiments, assessment of cell line stability can comprise one or more methods performed on a microfluidic apparatus. In other embodiments, assessment of cell line stability can comprise one or more steps for analyzing data obtained from methods performed on a microfluidic apparatus. In further instances, embodiments for assessing cell line stability can comprise a system configured to perform steps of the method on a microfluidic apparatus followed by or in concert with automated analysis of data obtained from methods performed on the microfluidic apparatus.

Methods. Methods for determining relative stability for a plurality of clonal cell lines can comprise: receiving imaging data of a microfluidic device that includes a flow region and a first plurality of chambers, which may be sequestration pens. The first plurality of chambers is fluidically connected to the flow region, wherein the imaging data includes a first analyte assay image taken of a plurality of first subclones of a first clonal cell line, wherein each subclone of the first clonal cell line is disposed in an individual chamber of the first plurality of chambers, defining an area of interest for each chamber, wherein the area of interest includes an image area within the chamber that is most sensitive for measuring analyte concentration fluctuations, is least sensitive to the position of biological micro-objects in the chamber when analyte fluctuations are measured, and extends along an axis of diffusion between the chamber and the flow region, and generating a prediction of clonal cell line stability based on a signal obtained from the axis of diffusion between the chamber and the flow region, wherein the signal is an indicator of clonal cell line stability.

In some instances, imaging data comprises a plurality of subclones of a second clonal cell line, wherein each subclone of the second clonal cell line are disposed in individual chambers of a second plurality of chambers. In further embodiments, the first analyte assay image comprises a plurality of subclones of a second clonal cell line, wherein each subclone of the second clonal cell line are disposed in an individual chamber of the second plurality of chambers. Alternatively, the imaging data can comprise a second analyte assay image comprising the plurality of subclones of a second clonal cell line, wherein each subclone of the second clonal cell line are disposed in an individual chamber of the second plurality of chambers.

In some instances, each of the individual cell lines may have been selected from a previous workflow, where the cell lines were selected based on an original rQp score that was higher than other cell lines for that workflow, and that original score (e.g., high rQp score), can be factored into analysis.

In some embodiments, the signal obtained from the axis of diffusion represents the level of secretion of an analyte of interest and/or the number of viable cells disposed in the chambers. In further embodiments, a rQP value correlated with secretion of a target molecule, is determined from imaging data of the signal obtained from the axis of diffusion.

In some embodiments an analyte assay image is obtained. Analyte assay images can be taken at various time points. For example, an analyte assay image can be taken prior to or after loading a primary subclone cell into a single chamber. In some embodiments, the analyte assay image is taken after the primary subclone cell has undergone at least one division event. Alternatively, or additionally, the analyte assay image can be taken of the microfluidic device between 1 and 10 days of loading the plurality of subclones of the clonal cell line into the plurality of chambers.

In some non-limiting embodiments, a calculation, algorithm, or ranking can be generated for assessing productivity, stability, or other features related to cell line production. For example, in some embodiments a machine learning algorithm may be applied to an analyte assay image to obtain a cell count for the plurality of subclones of the clonal cell lines disposed in individual chambers of the plurality of chambers. Alternatively, or additionally, a calculation, algorithm, or ranking can be used to generate a prediction of clonal cell line stability comprises calculating a doubling time for each subclone. Alternatively, or additionally, a counting algorithm can be applied to the analyte assay image to generate a count of the number of cells in the chamber at the time that the analyte assay image is taken.

Measurement of the phenotypes of the subclone population can comprise defining a subset of chambers dedicated to a particular clonal cell line, this can be performed for a single clonal cell line, or for two or more clonal cell lines (e.g., 2, 3, 4, 5, 6, 7, 8, 9, 10, 11, 12, 14, 16, 18, 20, different cell lines or cell types). The total number of chambers from which the subset is derived, can comprise a portion of chambers in a given microfluidic chip, the total number of chambers in a given microfluidic chip, or the total number of chambers across two or more microfluidic chips. In instances where two or more clonal cell lines are being assessed, each cell line can have a defined number of chambers allocated to the cell line (e.g., greater than or equal to 10, 25, 50, 100, 200, 250, 300, 400, 500, 600, 700, 800, 1000, 2500, 5000). In some instances, the number of chambers allocated to each cell line are equivalent, in other cases each cell line may have a different number of allocated chambers, in further instances where multiple cell lines are being monitored, some cell lines may have the same number of chambers allocated and other cell lines of the group being monitored may a different number of chambers allocated. For example, a comparison can comprise 12 different cell types used for comparison, with at least 500 chambers loaded with single primary subclone cells for each cell type—with the requirements that and at least 50% of the loaded primary subclone cells grow into viable colonies by last assay day (e.g., sample size of 250 growing chambers per cell type).

In some embodiments, the number of chambers allocated to the cell line may be configured based on the cell line characteristics. For example, a cell line that produces a higher percentage of viable secondary clonal cell populations (i.e., viable colonies) by the last day of a particular assay, can be allocated a smaller number of chambers, than a cell line suspected to produce a lower percentage of viable secondary clonal cell populations (i.e., viable colonies) by the last day of a particular assay. The definition of viable colonies or viable secondary clonal cell populations can be defined by one or more exclusionary criteria, inclusionary criteria, or thresholds where the thresholds or criterion can be any metric, value, measurement, characteristic, or combination thereof that can be reasonably determined or derived from imaging of signal produced from a chamber of the microfluidic device disclosed herein.

Following allocation of a subset of chambers for a particular clonal cell line, single cells of the expanded clonal cell line (i.e., subclones of said clonal cell line), are loaded into the chambers allocated to the clonal cell line. This operation is performed until a satisfactory percentage of (e.g., greater than: 50%, 60%, 70%, 80%, 90%), or the entirety of the allocated chambers are filled with single sub-clonal cells of the selected cell line are loaded. This step may be repeated until all the clonal cell lines being assessed have single cells loaded into all or a portion of the allocated subset of chambers.

After loading the subclones onto the microfluidic chip, the clonal cell lines may be incubated under conditions that support expansion (e.g., division to increase cell number) of the single subclones. For example, oxygen rich and/or carbon source rich medium may be perfused or aspirated through the channel of the microfluidic chip. Additionally, or alternatively, the microfluidic chip may be kept at a fixed temperature or temperature range (e.g., 18° to 37° Celsius) for a period of time (e.g., 1 to 10, 1 to 9, 1 to 8, 1 to 7, 1 to 6, or 1 to 5 days), and images may be taken of the clonal cell lines at particular increments (e.g., a set frequency for example, x images/sec or x images per day, or at discrete time points in the assay or workflow). Examples of discrete time points in the assay or workflow include but are not limited to one or more of: after loading the primary subclones on the microfluidic chip, after illumination of the clonal cell lines after illumination of the clonal cell lines for example broad spectrum illumination or illumination at a particular wavelength or band of wavelengths (e.g., after illumination with FITC, Texas Red, DAPI, etc. filter cubes), and/or at any time during a time period when one or more primary subclones are undergoing expansion. A cell count may be performed using one or more images. For example, cell counts can be performed at one or more time points including but not limited to: at the time of measuring a secretion phenotype but can be measured multiple times per day. Additionally or alternatively a cell count can be deductively determined using one or more other features of the microfluidic chip, for example a generalized cell count can be determined by obtaining an optical density measurement or chip OD (e.g., absorption measured at a particular wavelength (e.g., in a range of 600 nm) and/or at for example a particular region of the chip (e.g., chamber, such as a sequestration pen or defined region within a chamber, such as an isolation region of a sequestration pen). One or more other features of each sub-clonal cell disposed within each chamber, or a population of sub-clonal cells disposed across the allocated chambers can be assessed according to a defined threshold. The threshold can be applied as exclusionary or inclusionary. In instances where the threshold is exclusionary, subclones that meet the threshold are excluded. In instances where the threshold is inclusionary, subclones that meet the threshold are included. In some embodiments, combinations of thresholds can be applied to determine a target population, wherein the target population is utilized for the assessment of cell line stability.

Exemplary exclusionary criterion can comprise for example a threshold that assesses the number of cells in the chamber after a given period of time or the rate of division of a particular subclone cell. Exemplary exclusionary criterion may be, for example, if a subclone cell does not grow above a given threshold of cells after a period of time (e.g., if a chamber loaded with a single subclone has 5 or less cells after 4 days, or if a chamber loaded with a single subclone presents with less than three doubling events after 96 hours), then any subclones that meet that criterion are excluded from further analysis. These non-viable clones are excluded from analysis as they are either dead or extremely slow growing and so do not significantly contribute to the secretion of the cell line.

Cells that meet the threshold criterion are considered viable subclone cells for the given cell line, representing collectively a representative property of the cell line (e.g., a signature, or “fingerprint”) that is intrinsic to and/or characteristic of the cell line itself and/or the cell line relative to other cell lines assessed under similar or equivalent conditions. This representative property of the cell line functions as an indirect representation of the processes involved in the phenotype being monitored across time, in a way that includes the stochastic aspects of the cell line.

In instances where secretion of a target molecule and growth rates of the subclone cells are characteristics of interest amongst the pool or population of viable subclone cells for the given cell line, there is a range of secretion and growth rates that represent a distribution of secretion and grow rates across the sub-clone cell line, and this distribution is a representative property of the cell line. Further assessment can be performed on the subclones for the purpose of assessing stability of the cell line, and/or the relative stability of cell lines across a set of two or more cell lines.

Additional analysis can comprise, determining a percentage of remaining sub-clones that have detectable secretion levels. To this end, a limit of detection can be determined by using the signal obtained from an empty chamber (e.g., an empty neighboring chamber). In these instances, a subset of the total number of chambers can be left empty after the microfluidic chip is loaded with the subclone cells from the one or more clonal cell lines. For example, in some instances hundreds of chambers (e.g., greater than 99 chambers) can be left empty (intentionally or unintentionally) after allocating chambers to subclones for the particular cell line(s) and/or loading the chip with the subclone cells. Each empty chamber or a subset of the empty chambers, can be used to derive a blank measurement during the secretion assay, and the signal threshold that sets the limit of detection can be defined as proportional to or equivalent to the average empty chamber score plus a number of standard deviations. In further embodiments, the number of standard deviations used to set the threshold can be dependent on the acceptable false positive rate, where the false positive rate may be selected based on the number of subclone cells being assessed or a value proportional to the number of subclone cells being assessed (e.g., the number of chambers loaded with a single subclone cell, the number of chambers located with any number of subclone cells, the number of chambers allocated to a particular cell line being assessed, the number of chambers loaded with subclone cells from a particular cell line, etc.). Using a determined limit of detection threshold, a percentage of secreting sub-clones (i.e., hit rate) can be determined for each cell line. In some embodiments a correlation between hit rate and stability can be used as a basis for determining cell line stability. In such instances, for some instable cell lines, as the titer or target production drops, a percent of subclones secretion rate will drop below the limit of detection, thereby reducing the average secretion rate as well as the hit rate.

For example, assuming a normal distribution based on the central limit theorem, a threshold of the average blank score plus 3.3 standard deviations would give a false positive rate of 1 in 2000. This threshold may be chosen as it results in a false positive rate based on the total chamber population or some subset of chambers.

Analysis. Analysis of cell lines can be performed with the aim of determining a rank order of performance of the cell line, with the rank order indicating a relative likelihood of performance (e.g., performance in a fermentation system, performance under certain environmental conditions, etc.). For example, the output of an analysis as described under these contexts can comprise a rank order, wherein the higher a cell link ranks in the rank order, the better the given cell line is at producing high quantities of the desirable product (e.g., titer) over an extend period of time. In some embodiments, the rank order may be determined in accordance with a set of environmental conditions and the rank order may provide an indicator of performance of the cell line relative to other cell lines, under the particular environmental conditions being tested. Additionally, or alternatively, a rank order of performance of the cell line can be used to determine an intrinsic property of a cell line, for example stability of the cell line across multiple generations or under certain environmental conditions. In some instances, each of the individual cell lines may have been selected from a previous workflow, where the cell lines were selected based on an original rQp score that was higher than other cell lines for that workflow, and that original score (e.g., high rQp score), can be factored into analysis. For example, the original score can be compared with the rQp score generated from the expanded secondary population derived from the primary subclone that was obtained from the cell line from which the original rQp score was derived.

Stability. Additionally, measurements taken from a single primary subclonal cell may be used in further comparative analyses. The primary subclonal cell is a daughter cell derived from the original cell (e.g., an originating cell which founds the clonal cell line), where the original cell has undergone clonal cell expansion to produce the population of primary clonal cells that are loaded onto the microfluidic chip (e.g., as single cells) in the methods described herein. Once introduced into an individual pen (e.g., no pen has more than one primary subclonal cell) within the microfluidic chip, this primary subclonal cell is clonally expanded into a population of secondary subclonal cells. This population of secondary subclonal cells can be used to determine a rank of the stability of the clonal cell line. When this secondary subclonal cell population is unstable, the average titer for the subclonal cell may typically decrease over time, and the decrease may occur regardless of the mechanism (genetic or phenotypic). Population measurements can be made using cell counts of the secondary subclonal cells over time. With this measurement taken during clonal cell expansion resulting in the secondary subclonal cell population, a ranking for each of the individual chambers can be generated. This ranking provides an indication of stability of the cell line; a stable cell line will on average rank higher than an unstable cell line. In some instances, subsampling can be performed on clones from a clonal cell line, for the purpose of measuring the heterogeneity present in the clonal cell line. In such instances, for example, the average of the distribution defines the population average, within in the error of subsampling. Cell counts of the secondary subclone population can be used in conjunction with the subsampled data to measure growth rate. Additionally or alternatively, the subsampled data can also be used to look at any phenotype (e.g., size, secretion, etc.), which can provide information on stability of the particular cell line, and/or other aspects of a given cell line relative to other cell lines.

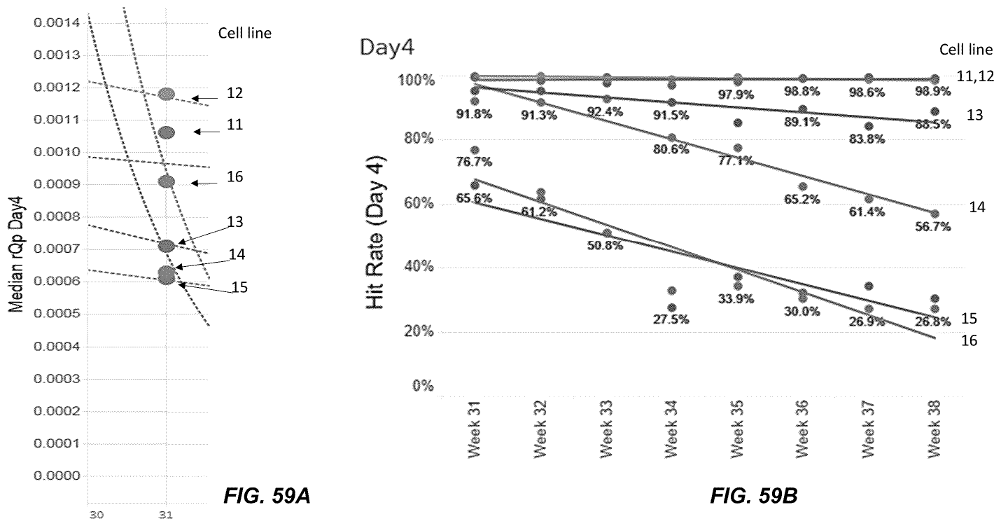

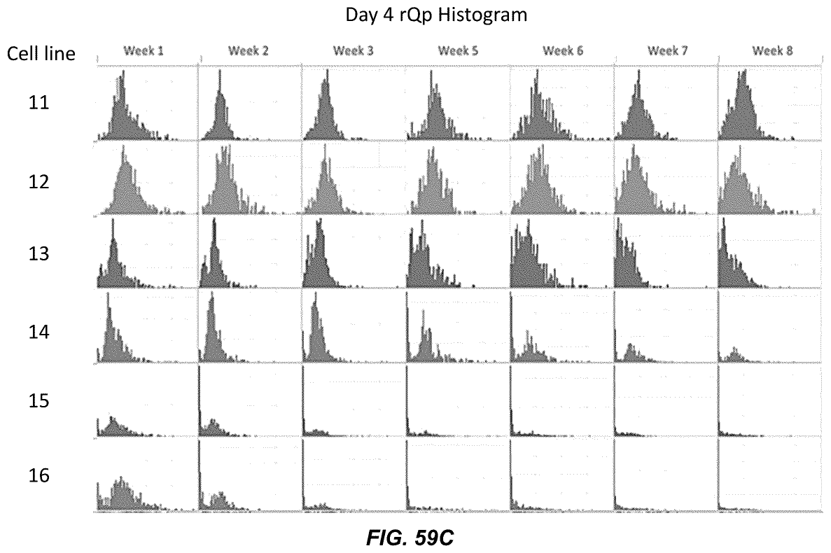

Data collected using a set of clones of varying stabilities is shown in Example 10, and FIGS. 59A to 59C, which illustrates the rank order of the median rQP correlated with the rank order of the stability of each cell line. This ranking effectively provides a measure of key characteristics which can be important in identifying cell lines that can be used to effectively produce a product (e.g., small molecule, drug, etc. . . . ). Specifically this method of analysis highly ranks cell lines that are both high secreting and stable, while eliminating unstable clones whose secretion has dropped significantly since the initial screen and eliminating stable low secretion clones, where the secretion has regressed to the mean secretion after the initial screen.

Measuring the distribution of the sub-clonal rQp values provides additional information beyond just the average secretion and growth rates and provides other ways to rank the stability of the clones.

Using the secretion distribution, the percent of secreting sub-clones above the limit of detection (i.e. hit rate) also correlated with stability with more stable cell lines having a higher percent of secreting sub clones (FIG. 59C). The difference in secretion rate and therefore the resolution between cell lines increases with time as unstable clone secretion may continue to decrease. In addition, other features of the distribution, such as a bimodal distribution, correlate with instability. This may indicate that there may be distinct populations of sub-clones within the cell line increasing the likelihood of instability. Using a combination of the these metrics and features may increase the confidence in identifying unstable clones.

Additionally, or alternatively, a model or algorithm can be generated using the distributions of sub-clone secretion (i.e., rQp) and growth rates. This model can assume that for a given sub-clone the secretion and growth rate are constant over the time period being measured. The model then can be configured to make estimations or projections based on the frequency of the sub-clones, how the culture will evolve as fast-growing sub-clones become a larger percentage of the culture as they put compete the slow growing sub-clones and therefore determine the future average secretion rate.

In some embodiments, a software program can be used to generate and implement an automated algorithm that uses measured rQp and growth rate or doubling time of each chamber in the workflow and simulates future behavior performance over time. The algorithm can work using two primary assumptions: (a) rQp cells in a respective chamber continue to have same rQp over time; and (b) growth rate of cells in a respective chamber continues to have same growth rate over time. So, from a clonal cell line made up of a mixture of slow to fast growing chamber, the fast-growing cells (secondary subclonal population produced from the primary subclone that was loaded in the respective chamber) will become the dominant population over time as it grows exponentially faster than slower sub-clones in respective chambers. In instance where this fast-growing sub-clone has high measured rQp, then the future dominant population will also have high rQp resulting in a simulated stable secreting clone. Alternatively, if the fast-growing sub-clone has low measured rQp, then the future dominant population will have low rQp resulting in a simulated unstable secreting clone.

For better simulated comparison of clones, the software module can be configured to provide scatter and line plots for various presentations of the data including but not limited to: Simulated average rQp at a given week “n” and Normalized average rQp at given week “n”. Using these two values, the user can optionally downselect clones based on simulated behaviors. For example, definitions for Simulated Average rQp and Normalized Average rQp can be made using the following calculations:

$${Score}_{[ Pen M ; Week N ]} = {rQp}_{[ Pen M ]} \times Number of {cells}_{[ Week N ]}$$

$$Simulated Average {rQp}_{[ Week N ]} = \frac{\sum {Score}_{[ Pen M ; Week N ]}}{\sum Number of {cells}_{[ Week N ]}}$$

$$Normalized Average {rQp}_{[ Week N ]} = \frac{Simulated Average {rQp}_{[ Week N ]}}{Simulated Average {rQp}_{[ Week 1 ]}} \times 100 \%$$

More specifically, the software can be configured to analyze the imaging data derived from the secondary subclone population disposed in each chamber, where the secondary subclone population is derived from a single primary subclone cell. The software can perform cell counts at one or more time points during the clonal cell expansion of the single primary subclone cell. The software can, additionally or alternatively, at one or more time points concurrent with, prior to, or after to clonal cell expansion of the single primary subclone cell, perform one or more the following: use one or more imaging methods to measure an electromagnetic signal (e.g., fluorescence signal), apply a threshold base on one or more detected features of the images obtained from the region of interest within the chamber, and perform an assessment of what portion of those subclones are viable and high secreting. The assessment can comprise one or more of performing a calculation using any of the features (e.g., growth rate, rQp for single chamber, rQp across a range of chambers, etc.). The software can further calculate the fraction of viable/high secreting subclone from one cell line and compares it to the fraction of viable/high secreting subclones derived from another cell line.

The data resulting from an analysis of the subclone population data can operate as a “fingerprint” representing the stability of the population for comparison across cell lines—this can be done, for example, by computing a frequency histogram and looking for bimodal distribution or shifts between the median/mode/average between different generations of subclone populations (e.g., subclones at week 1 verses week 8, etc.). There are many ways of analyzing and representing these fractions of viable/high secreting subclones and the shifts in the secretion/viability of the subclone populations through subsequent generations, similarly there are many ways of comparing them-any methods commonly known in the field for presenting data across an array of samples can be applied to methods, systems and devices disclosed herein, and to the results thereof.

### VI. MICROFLUIDIC DEVICES & SYSTEMS (CROSS-APPLICABILITY)

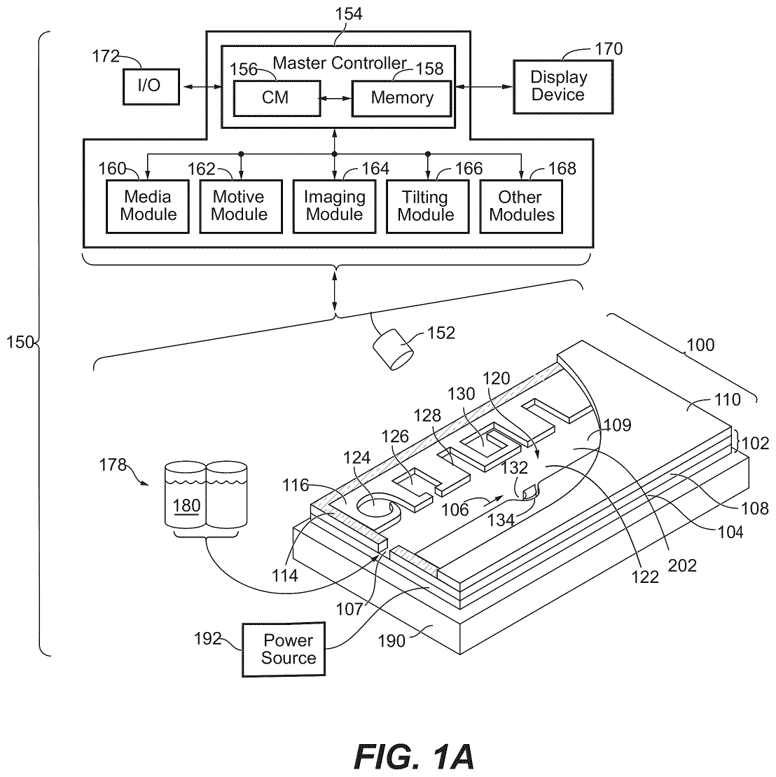

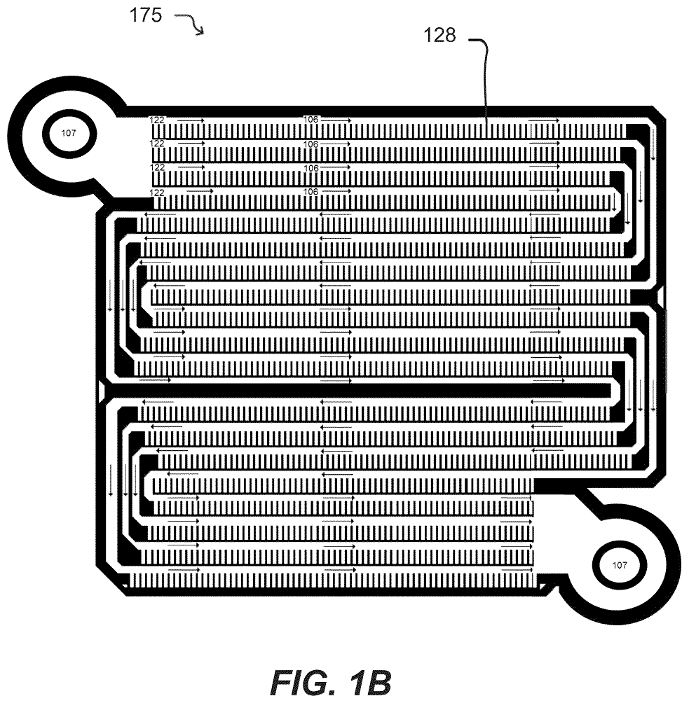

It should be appreciated that various features of microfluidic devices, systems, and motive technologies described herein may be combinable or interchangeable. For example, features described herein with reference to the microfluidic device 100, 175, 200, 300, 320, 400, 450, 520 and system attributes as described in FIGS. 1A-5B may be combinable or interchangeable.

Microfluidic devices. FIG. 1A illustrates an example of a microfluidic device 100. A perspective view of the microfluidic device 100 is shown having a partial cut-away of its cover 110 to provide a partial view into the microfluidic device 100. The microfluidic device 100 generally comprises a microfluidic circuit 120 comprising a flow path 106 through which a fluidic medium 180 can flow, optionally carrying one or more micro-objects (not shown) into and/or through the microfluidic circuit 120.

As generally illustrated in FIG. 1A, the microfluidic circuit 120 is defined by an enclosure 102. Although the enclosure 102 can be physically structured in different configurations, in the example shown in FIG. 1A the enclosure 102 is depicted as comprising a support structure 104 (e.g., a base), a microfluidic circuit structure 108, and a cover 110. The support structure 104, microfluidic circuit structure 108, and cover 110 can be attached to each other. For example, the microfluidic circuit structure 108 can be disposed on an inner surface 109 of the support structure 104, and the cover 110 can be disposed over the microfluidic circuit structure 108. Together with the support structure 104 and cover 110, the microfluidic circuit structure 108 can define the elements of the microfluidic circuit 120, forming a three-layer structure.

The support structure 104 can be at the bottom and the cover 110 at the top of the microfluidic circuit 120 as illustrated in FIG. 1A. Alternatively, the support structure 104 and the cover 110 can be configured in other orientations. For example, the support structure 104 can be at the top and the cover 110 at the bottom of the microfluidic circuit 120. Regardless, there can be one or more ports 107 each comprising a passage into or out of the enclosure 102. Examples of a passage include a valve, a gate, a pass-through hole, or the like. As illustrated, port 107 is a pass-through hole created by a gap in the microfluidic circuit structure 108. However, the port 107 can be situated in other components of the enclosure 102, such as the cover 110. Only one port 107 is illustrated in FIG. 1A but the microfluidic circuit 120 can have two or more ports 107. For example, there can be a first port 107 that functions as an inlet for fluid entering the microfluidic circuit 120, and there can be a second port 107 that functions as an outlet for fluid exiting the microfluidic circuit 120. Whether a port 107 function as an inlet or an outlet can depend upon the direction that fluid flows through flow path 106.

The support structure 104 can comprise one or more electrodes (not shown) and a substrate or a plurality of interconnected substrates. For example, the support structure 104 can comprise one or more semiconductor substrates, each of which is electrically connected to an electrode (e.g., all or a subset of the semiconductor substrates can be electrically connected to a single electrode). The support structure 104 can further comprise a printed circuit board assembly (“PCBA”). For example, the semiconductor substrate(s) can be mounted on a PCBA.

The microfluidic circuit structure 108 can define circuit elements of the microfluidic circuit 120. Such circuit elements can comprise spaces or regions that can be fluidly interconnected when microfluidic circuit 120 is filled with fluid, such as flow regions (which may include or be one or more flow channels), chambers (which class of circuit elements may also include sub-classes including sequestration pens), traps, and the like. Circuit elements can also include barriers, and the like. In the microfluidic circuit 120 illustrated in FIG. 1A, the microfluidic circuit structure 108 comprises a frame 114 and a microfluidic circuit material 116. The frame 114 can partially or completely enclose the microfluidic circuit material 116. The frame 114 can be, for example, a relatively rigid structure substantially surrounding the microfluidic circuit material 116. For example, the frame 114 can comprise a metal material. However, the microfluidic circuit structure need not include a frame 114. For example, the microfluidic circuit structure can consist of (or consist essentially of) the microfluidic circuit material 116.

The microfluidic circuit material 116 can be patterned with cavities or the like to define the circuit elements and interconnections of the microfluidic circuit 120, such as chambers, pens and microfluidic channels. The microfluidic circuit material 116 can comprise a flexible material, such as a flexible polymer (e.g., rubber, plastic, elastomer, silicone, polydimethylsiloxane (“PDMS”), or the like), which can be gas permeable. Other examples of materials that can form the microfluidic circuit material 116 include molded glass, an etchable material such as silicone (e.g., photo-patternable silicone or “PPS”), photo-resist (e.g., SU8), or the like. In some embodiments, such materials—and thus the microfluidic circuit material 116—can be rigid and/or substantially impermeable to gas. Regardless, microfluidic circuit material 116 can be disposed on the support structure 104 and inside the frame 114.

The microfluidic circuit 120 can include a flow region in which one or more chambers can be disposed and/or fluidically connected thereto. A chamber can have one or more openings fluidically connecting the chamber with one or more flow regions. In some embodiments, a flow region comprises or corresponds to a microfluidic channel 122. Although a single microfluidic circuit 120 is illustrated in FIG. 1A, suitable microfluidic devices can include a plurality (e.g., 2 or 3) of such microfluidic circuits. In some embodiments, the microfluidic device 100 can be configured to be a nanofluidic device. As illustrated in FIG. 1A, the microfluidic circuit 120 may include a plurality of microfluidic sequestration pens 124, 126, 128, and 130, where each sequestration pens may have one or more openings. In some embodiments of sequestration pens, a sequestration pen may have only a single opening in fluidic communication with the flow path 106. In some other embodiments, a sequestration pen may have more than one opening in fluidic communication with the flow path 106, e.g., n number of openings, but with n−1 openings that are valved, such that all but one opening is closable. When all the valved openings are closed, the sequestration pen limits exchange of materials from the flow region into the sequestration pen to occur only by diffusion. In some embodiments, the sequestration pens comprise various features and structures (e.g., isolation regions) that have been optimized for retaining micro-objects within the sequestration pen (and therefore within a microfluidic device such as microfluidic device 100) even when a medium 180 is flowing through the flow path 106.

The cover 110 can be an integral part of the frame 114 and/or the microfluidic circuit material 116. Alternatively, the cover 110 can be a structurally distinct element, as illustrated in FIG. 1A. The cover 110 can comprise the same or different materials than the frame 114 and/or the microfluidic circuit material 116. In some embodiments, the cover 110 can be an integral part of the microfluidic circuit material 116. Similarly, the support structure 104 can be a separate structure from the frame 114 or microfluidic circuit material 116 as illustrated, or an integral part of the frame 114 or microfluidic circuit material 116. Likewise, the frame 114 and microfluidic circuit material 116 can be separate structures as shown in FIG. 1A or integral portions of the same structure. Regardless of the various possible integrations, the microfluidic device can retain a three-layer structure that includes a base layer and a cover layer that sandwich a middle layer in which the microfluidic circuit 120 is located.

In some embodiments, the cover 110 can comprise a rigid material. The rigid material may be glass or a material with similar properties. In some embodiments, the cover 110 can comprise a deformable material. The deformable material can be a polymer, such as PDMS. In some embodiments, the cover 110 can comprise both rigid and deformable materials. For example, one or more portions of cover 110 (e.g., one or more portions positioned over sequestration pens 124, 126, 128, 130) can comprise a deformable material that interfaces with rigid materials of the cover 110. Microfluidic devices having covers that include both rigid and deformable materials have been described, for example, in U.S. Pat. No. 10,058,865 (Breinlinger et al.), the contents of which are incorporated herein by reference. In some embodiments, the cover 110 can further include one or more electrodes. The one or more electrodes can comprise a conductive oxide, such as indium-tin-oxide (ITO), which may be coated on glass or a similarly insulating material. Alternatively, the one or more electrodes can be flexible electrodes, such as single-walled nanotubes, multi-walled nanotubes, nanowires, clusters of electrically conductive nanoparticles, or combinations thereof, embedded in a deformable material, such as a polymer (e.g., PDMS). Flexible electrodes that can be used in microfluidic devices have been described, for example, in U.S. Pat. No. 9,227,200 (Chiou et al.), the contents of which are incorporated herein by reference. In some embodiments, the cover 110 and/or the support structure 104 can be transparent to light. The cover 110 may also include at least one material that is gas permeable (e.g., PDMS or PPS).

In the example shown in FIG. 1A, the microfluidic circuit 120 is illustrated as comprising a microfluidic channel 122 and sequestration pens 124, 126, 128, 130. Each pen comprises an opening to channel 122, but otherwise is enclosed such that the pens can substantially isolate micro-objects inside the pen from fluidic medium 180 and/or micro-objects in the flow path 106 of channel 122 or in other pens. The walls of the sequestration pen extend from the inner surface 109 of the base to the inside surface of the cover 110 to provide enclosure. The opening of the sequestration pen to the microfluidic channel 122 is oriented at an angle to the flow 106 of fluidic medium 180 such that flow 106 is not directed into the pens. The vector of bulk fluid flow in channel 122 may be tangential or parallel to the plane of the opening of the sequestration pen, and is not directed into the opening of the pen. In some instances, pens 124, 126, 128, 130 are configured to physically isolate one or more micro-objects within the microfluidic circuit 120. Sequestration pens in accordance with the present disclosure can comprise various shapes, surfaces and features that are optimized for use with DEP, OET, OEW, fluid flow, magnetic forces, centripetal, and/or gravitational forces, as will be discussed and shown in detail below.

The microfluidic circuit 120 may comprise any number of microfluidic sequestration pens. Although five sequestration pens are shown, microfluidic circuit 120 may have fewer or more sequestration pens. As shown, microfluidic sequestration pens 124, 126, 128, and 130 of microfluidic circuit 120 each comprise differing features and shapes which may provide one or more benefits useful for maintaining, isolating, assaying or culturing biological micro-objects. In some embodiments, the microfluidic circuit 120 comprises a plurality of identical microfluidic sequestration pens.

In the embodiment illustrated in FIG. 1A, a single flow path 106 containing a single channel 122 is shown. However, other embodiments may contain multiple channels 122 within a single flow path 106, as shown in FIG. 1B. The microfluidic circuit 120 further comprises an inlet valve or port 107 in fluid communication with the flow path 106, whereby fluidic medium 180 can access the flow path 106 (and channel 122). In some instances, the flow path 106 comprises a substantially straight path. In other instances, the flow path 106 is arranged in a non-linear or winding manner, such as a zigzag pattern, whereby the flow path 106 travels across the microfluidic device 100 two or more times, e.g., in alternating directions. The flow in the flow path 106 may proceed from inlet to outlet or may be reversed and proceed from outlet to inlet.

One example of a multi-channel device, microfluidic device 175, is shown in FIG. 1B, which may be like microfluidic device 100 in other respects. Microfluidic device 175 and its constituent circuit elements (e.g., channels 122 and sequestration pens 128) may have any of the dimensions discussed herein. The microfluidic circuit illustrated in FIG. 1B has two inlet/outlet ports 107 and a flow path 106 containing four distinct channels 122. The number of channels into which the microfluidic circuit is sub-divided may be chosen to reduce fluidic resistance. For example, the microfluidic circuit may include 1, 2, 3, 4, 5, 6, 7, 8, 9, 10, or more channels to provide a selected range of fluidic resistance. Microfluidic device 175 further comprises a plurality of sequestration pens opening off of each channel 122, where each of the sequestration pens is similar to sequestration pen 128 of FIG. 1A, and may have any of the dimensions or functions of any sequestration pen as described herein. However, the sequestration pens of microfluidic device 175 can have different shapes, such as any of the shapes of sequestration pens 124, 126, or 130 of FIG. 1A or as described anywhere else herein. Moreover, microfluidic device 175 can include sequestration pens having a mixture of different shapes. In some instances, a plurality of sequestration pens is configured (e.g., relative to a channel 122) such that the sequestration pens can be loaded with target micro-objects in parallel.

Returning to FIG. 1A, microfluidic circuit 120 further may include one or more optional micro-object traps 132. The optional traps 132 may be formed in a wall forming the boundary of a channel 122, and may be positioned opposite an opening of one or more of the microfluidic sequestration pens 124, 126, 128, 130. The optional traps 132 may be configured to receive or capture a single micro-object from the flow path 106, or may be configured to receive or capture a plurality of micro-objects from the flow path 106. In some instances, the optional traps 132 comprise a volume approximately equal to the volume of a single target micro-object. In some instances, the trap 132 comprises a side passage 134 that is smaller than the target micro-object in order to facilitate flow through the trap 132.

Sequestration pens. The microfluidic devices described herein may include one or more sequestration pens, where each sequestration pen is suitable for holding one or more micro-objects (e.g., biological cells, or groups of cells that are associated together). The sequestration pens may be disposed within and open to a flow region, which in some embodiments is a microfluidic channel. Each of the sequestration pens can have one or more openings for fluidic communication to one or more microfluidic channels. In some embodiments, a sequestration pen may have only one opening to a microfluidic channel.

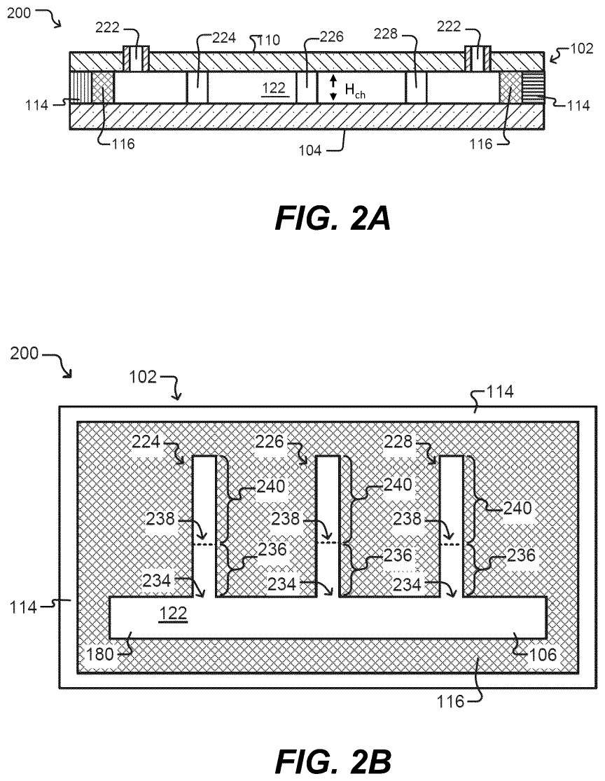

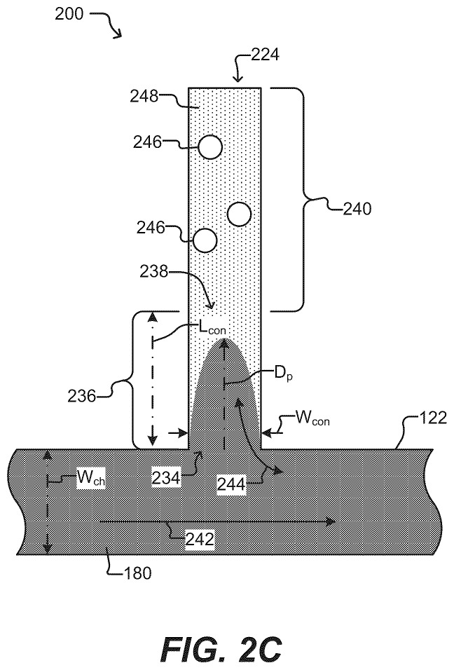

FIGS. 2A-2C show sequestration pens 224, 226, and 228 of a microfluidic device 200, which may be like sequestration pen 128 of FIG. 1A. Each sequestration pen 224, 226, and 228 can comprise an isolation region 240 and a connection region 236 fluidically connecting the isolation region 240 to a flow region, which may, in some embodiments include a microfluidic channel, such as channel 122. The connection region 236 can comprise a proximal opening 234 to the flow region (e.g., microfluidic channel 122) and a distal opening 238 to the isolation region 240. The connection region 236 can be configured so that the maximum penetration depth of a flow of a fluidic medium (not shown) flowing in the microfluidic channel 122 past the sequestration pen 224, 226, and 228 does not extend into the isolation region 240, as discussed below for FIG. 2C. In some embodiments, streamlines from the flow in the microfluidic channel do not enter the isolation region. Thus, due to the connection region 236, a micro-object (not shown) or other material (not shown) disposed in the isolation region 240 of a sequestration pen 224, 226, and 228 can be isolated from, and not substantially affected by, a flow of fluidic medium 180 in the microfluidic channel 122.

The sequestration pens 224, 226, and 228 of FIGS. 2A-2C each have a single opening which opens directly to the microfluidic channel 122. The opening of the sequestration pen may open laterally from the microfluidic channel 122, as shown in FIG. 2A, which depicts a vertical cross-section of microfluidic device 200. FIG. 2B shows a horizontal cross-section of microfluidic device 200. An electrode activation substrate 206 can underlie both the microfluidic channel 122 and the sequestration pens 224, 226, and 228. The upper surface of the electrode activation substrate 206 within an enclosure of a sequestration pen, forming the floor of the sequestration pen, can be disposed at the same level or substantially the same level of the upper surface the of electrode activation substrate 206 within the microfluidic channel 122 (or flow region if a channel is not present), forming the floor of the flow channel (or flow region, respectively) of the microfluidic device. The electrode activation substrate 206 may be featureless or may have an irregular or patterned surface that varies from its highest elevation to its lowest depression by less than about 3 micrometers (microns), 2.5 microns, 2 microns, 1.5 microns, 1 micron, 0.9 microns, 0.5 microns, 0.4 microns, 0.2 microns, 0.1 microns or less. The variation of elevation in the upper surface of the substrate across both the microfluidic channel 122 (or flow region) and sequestration pens may be equal to or less than about 10%, 7%, 5%, 3%, 2%, 1%, 0.9%, 0.8%, 0.5%, 0.3% or 0.1% of the height of the walls of the sequestration pen. Alternatively, the variation of elevation in the upper surface of the substrate across both the microfluidic channel 122 (or flow region) and sequestration pens may be equal to or less than about 2%, 1%. 0.9%, 0.8%, 0.5%, 0.3%, 0.2%, or 0.1% of the height of the substrate. While described in detail for the microfluidic device 200, this may also apply to any of the microfluidic devices described herein.

The microfluidic channel 122 and connection region 236 can be examples of swept regions, and the isolation regions 240 of the sequestration pens 224, 226, and 228 can be examples of unswept regions. Sequestration pens like 224, 226, 228 have isolation regions wherein each isolation region has only one opening, which opens to the connection region of the sequestration pen. Fluidic media exchange in and out of the isolation region so configured can be limited to occurring substantially only by diffusion. As noted, the microfluidic channel 122 and sequestration pens 224, 226, and 228 can be configured to contain one or more fluidic media 180. In the example shown in FIGS. 2A-2B, ports 222 are connected to the microfluidic channel 122 and allow the fluidic medium 180 to be introduced into or removed from the microfluidic device 200. Prior to introduction of the fluidic medium 180, the microfluidic device may be primed with a gas such as carbon dioxide gas. Once the microfluidic device 200 contains the fluidic medium 180, the flow 242 (see FIG. 2C) of fluidic medium 180 in the microfluidic channel 122 can be selectively generated and stopped. For example, as shown, the ports 222 can be disposed at different locations (e.g., opposite ends) of the flow region (microfluidic channel 122), and a flow 242 of the fluidic medium can be created from one port 222 functioning as an inlet to another port 222 functioning as an outlet.

FIG. 2C illustrates a detailed view of an example of a sequestration pen 224, which may contain one or more micro-objects 246, according to some embodiments. The flow 242 of fluidic medium 180 in the microfluidic channel 122 past the proximal opening 234 of the connection region 236 of sequestration pen 224 can cause a secondary flow 244 of the fluidic medium 180 into and out of the sequestration pen 224. To sequester the micro-objects 246 in the isolation region 240 of the sequestration pen 224 from the secondary flow 244, the length L_{con} of the connection region 236 of the sequestration pen 224 (i.e., from the proximal opening 234 to the distal opening 238) should be greater than the penetration depth Dₚ of the secondary flow 244 into the connection region 236. The penetration depth Dₚ depends upon a number of factors, including the shape of the microfluidic channel 122, which may be defined by a width W_{con} of the connection region 236 at the proximal opening 234; a width W_{ch} of the microfluidic channel 122 at the proximal opening 234; a height H_{ch} of the channel 122 at the proximal opening 234; and the width of the distal opening 238 of the connection region 236. Of these factors, the width W_{con} of the connection region 236 at the proximal opening 234 and the height H_{ch} of the channel 122 at the proximal opening 234 tend to be the most significant. In addition, the penetration depth Dₚ can be influenced by the velocity of the fluidic medium 180 in the channel 122 and the viscosity of fluidic medium 180. However, these factors (i.e., velocity and viscosity) can vary widely without dramatic changes in penetration depth Dₚ. For example, for a microfluidic chip 200 having a width W_{con} of the connection region 236 at the proximal opening 234 of about 50 microns, a height H_{ch} of the channel 122 at the proximal opening 122 of about 40 microns, and a width W_{ch} of the microfluidic channel 122 at the proximal opening 122 of about 100 microns to about 150 microns, the penetration depth Dₚ of the secondary flow 244 ranges from less than 1.0 times W_{con} (i.e., less than 50 microns) at a flow rate of 0.1 microliters/sec to about 2.0 times W_{con} (i.e., about 100 microns) at a flow rate of 20 microliters/sec, which represents an increase in Dₚ of only about 2.5-fold over a 200-fold increase in the velocity of the fluidic medium 180.

In some embodiments, the walls of the microfluidic channel 122 and sequestration pen 224, 226, or 228 can be oriented as follows with respect to the vector of the flow 242 of fluidic medium 180 in the microfluidic channel 122: the microfluidic channel width W_{ch} (or cross-sectional area of the microfluidic channel 122) can be substantially perpendicular to the flow 242 of medium 180; the width W_{con} (or cross-sectional area) of the connection region 236 at opening 234 can be substantially parallel to the flow 242 of medium 180 in the microfluidic channel 122; and/or the length L_{con} of the connection region can be substantially perpendicular to the flow 242 of medium 180 in the microfluidic channel 122. The foregoing are examples only, and the relative position of the microfluidic channel 122 and sequestration pens 224, 226 and 228 can be in other orientations with respect to each other.

In some embodiments, for a given microfluidic device, the configurations of the microfluidic channel 122 and the opening 234 may be fixed, whereas the rate of flow 242 of fluidic medium 180 in the microfluidic channel 122 may be variable. Accordingly, for each sequestration pen 224, a maximal velocity Vₘₐₓ for the flow 242 of fluidic medium 180 in channel 122 may be identified that ensures that the penetration depth Dₚ of the secondary flow 244 does not exceed the length L_{con} of the connection region 236. When Vₘₐₓ is not exceeded, the resulting secondary flow 244 can be wholly contained within the connection region 236 and does not enter the isolation region 240. Thus, the flow 242 of fluidic medium 180 in the microfluidic channel 122 (swept region) is prevented from drawing micro-objects 246 out of the isolation region 240, which is an unswept region of the microfluidic circuit, resulting in the micro-objects 246 being retained within the isolation region 240. Accordingly, selection of microfluidic circuit element dimensions and further selection of the operating parameters (e.g., velocity of fluidic medium 180) can prevent contamination of the isolation region 240 of sequestration pen 224 by materials from the microfluidic channel 122 or another sequestration pen 226 or 228. It should be noted, however, that for many microfluidic chip configurations, there is no need to worry about Vₘₐₓ per se, because the chip will break from the pressure associated with flowing fluidic medium 180 at high velocity through the chip before Vₘₐₓ can be achieved.

Components (not shown) in the first fluidic medium 180 in the microfluidic channel 122 can mix with the second fluidic medium 248 in the isolation region 240 substantially only by diffusion of components of the first medium 180 from the microfluidic channel 122 through the connection region 236 and into the second fluidic medium 248 in the isolation region 240. Similarly, components (not shown) of the second medium 248 in the isolation region 240 can mix with the first medium 180 in the microfluidic channel 122 substantially only by diffusion of components of the second medium 248 from the isolation region 240 through the connection region 236 and into the first medium 180 in the microfluidic channel 122. In some embodiments, the extent of fluidic medium exchange between the isolation region of a sequestration pen and the flow region by diffusion is greater than about 90%, 91%, 92%, 93%, 94%95%, 96%, 97%, 98%, or greater than about 99% of fluidic exchange.

In some embodiments, the first medium 180 can be the same medium or a different medium than the second medium 248. In some embodiments, the first medium 180 and the second medium 248 can start out being the same, then become different (e.g., through conditioning of the second medium 248 by one or more cells in the isolation region 240, or by changing the medium 180 flowing through the microfluidic channel 122).

As illustrated in FIG. 2C, the width W_{con} of the connection region 236 can be uniform from the proximal opening 234 to the distal opening 238. The width W_{con} of the connection region 236 at the distal opening 238 can be any of the values identified herein for the width W_{con} of the connection region 236 at the proximal opening 234. In some embodiments, the width of the isolation region 240 at the distal opening 238 can be substantially the same as the width W_{con} of the connection region 236 at the proximal opening 234. Alternatively, the width W_{con} of the connection region 236 at the distal opening 238 can be different (e.g., larger or smaller) than the width W_{con} of the connection region 236 at the proximal opening 234. In some embodiments, the width W_{con} of the connection region 236 may be narrowed or widened between the proximal opening 234 and distal opening 238. For example, the connection region 236 may be narrowed or widened between the proximal opening and the distal opening, using a variety of different geometries (e.g., chamfering the connection region, beveling the connection region). Further, any part or subpart of the connection region 236 may be narrowed or widened (e.g., a portion of the connection region adjacent to the proximal opening 234).

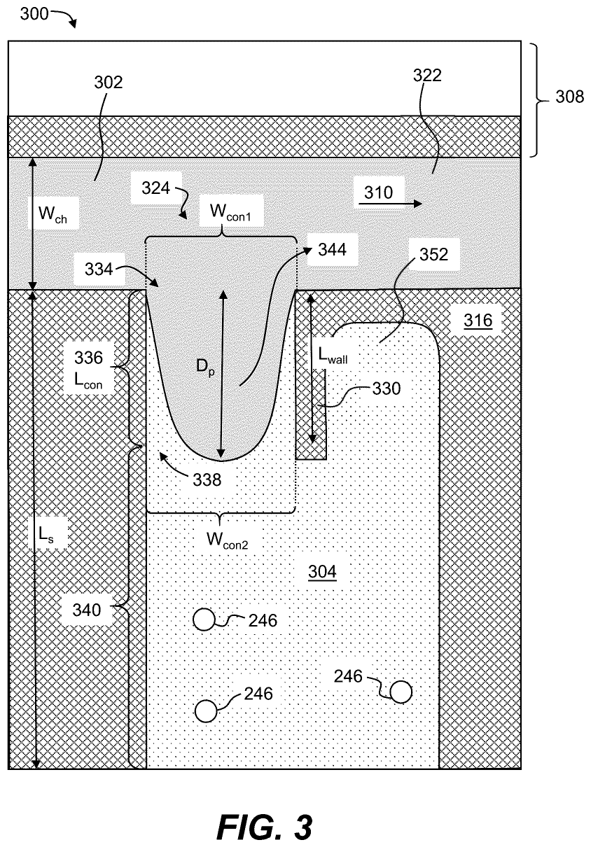

FIG. 3 depicts another exemplary embodiment of a microfluidic device 300 containing microfluidic circuit structure 308, which includes a channel 322 and sequestration pen 324, which has features and properties like any of the sequestration pens described herein for microfluidic devices 100, 175, 200, 400, 520 and any other microfluidic devices described herein.

The exemplary microfluidic devices of FIG. 3 include a microfluidic channel 322, having a width W_{ch}, as described herein, and containing a flow 310 of first fluidic medium 302 and one or more sequestration pens 324 (only one illustrated in FIG. 3). The sequestration pens 324 each have a length Ls, a connection region 336, and an isolation region 340, where the isolation region 340 contains a second fluidic medium 304. The connection region 336 has a proximal opening 334, having a width W_{con1}, which opens to the microfluidic channel 322, and a distal opening 338, having a width W_{con2}, which opens to the isolation region 340. The width W_{con1} may or may not be the same as W_{con2}, as described herein. The walls of each sequestration pen 324 may be formed of microfluidic circuit material 316, which may further form the connection region walls 330. A connection region wall 330 can correspond to a structure that is laterally positioned with respect to the proximal opening 334 and at least partially extends into the enclosed portion of the sequestration pen 324. In some embodiments, the length L_{con} of the connection region 336 is at least partially defined by length L_{wall} of the connection region wall 330. The connection region wall 330 may have a length L_{wall}, selected to be more than the penetration depth Dₚ of the secondary flow 344. Thus, the secondary flow 344 can be wholly contained within the connection region without extending into the isolation region 340.

The connection region wall 330 may define a hook region 352, which is a sub-region of the isolation region 340 of the sequestration pen 324. Since the connection region wall 330 extends into the inner cavity of the sequestration pen, the connection region wall 330 can act as a physical barrier to shield hook region 352 from secondary flow 344, with selection of the length of L_{wall}, contributing to the extent of the hook region. In some embodiments, the longer the length L_{wall} of the connection region wall 330, the more sheltered the hook region 352.

In sequestration pens configured like those of FIGS. 2A-2C and 3, the isolation region may have a shape and size of any type, and may be selected to regulate diffusion of nutrients, reagents, and/or media into the sequestration pen to reach to a far wall of the sequestration pen, e.g., opposite the proximal opening of the connection region to the flow region (or microfluidic channel). The size and shape of the isolation region may further be selected to regulate diffusion of waste products and/or secreted products of a biological micro-object out from the isolation region to the flow region via the proximal opening of the connection region of the sequestration pen. In general, the shape of the isolation region is not critical to the ability of the sequestration pen to isolate micro-objects from direct flow in the flow region.

In some other embodiments of sequestration pens, the isolation region may have more than one opening fluidically connecting the isolation region with the flow region of the microfluidic device. However, for an isolation region having a number of n openings fluidically connecting the isolation region to the flow region (or two or more flow regions), n−1 openings can be valved. When the n−1 valved openings are closed, the isolation region has only one effective opening, and exchange of materials into/out of the isolation region occurs only by diffusion.

Examples of microfluidic devices having pens in which biological micro-objects can be placed, cultured, and/or monitored have been described, for example, in U.S. Pat. No. 9,857,333 (Chapman, et al.), U.S. Pat. No. 10,010,882 (White, et al.), and U.S. Pat. No. 9,889,445 (Chapman, et al.), each of which is incorporated herein by reference in its entirety.

Microfluidic circuit element dimensions. Various dimensions and/or features of the sequestration pens and the microfluidic channels to which the sequestration pens open, as described herein, may be selected to limit introduction of contaminants or unwanted micro-objects into the isolation region of a sequestration pen from the flow region/microfluidic channel; limit the exchange of components in the fluidic medium from the channel or from the isolation region to substantially only diffusive exchange; facilitate the transfer of micro-objects into and/or out of the sequestration pens; and/or facilitate growth or expansion of the biological cells. Microfluidic channels and sequestration pens, for any of the embodiments described herein, may have any suitable combination of dimensions, may be selected by one of skill from the teachings of this disclosure.

For any of the microfluidic devices described herein, a microfluidic channel may have a uniform cross sectional height along its length that is a substantially uniform cross sectional height, and may be any cross sectional height as described herein. At any point along the microfluidic channel, the substantially uniform cross sectional height of the channel, the upper surface of which is defined by the inner surface of the cover and the lower surface of which is defined by the inner surface of the base, may be substantially the same as the cross sectional height at any other point along the channel, e.g., having a cross sectional height that is no more than about 10%, about 9%, about 8%, about 7%, about 6%, about 5%, about 4%, about 3%, about 2% or about 1% or less, different from the cross-sectional height of any other location within the channel.

Additionally, the chamber(s), e.g., sequestration pen(s), of the microfluidic devices described herein, may be disposed substantially in a coplanar orientation relative to the microfluidic channel into which the chamber(s) open. That is, the enclosed volume of the chamber(s) is formed by an upper surface that is defined by the inner surface of the cover, a lower surface defined by the inner surface of the base, and walls defined by the microfluidic circuit material. Therefore, the lower surface of the chamber(s) may be coplanar to the lower surface of the microfluidic channel, e.g., substantially coplanar. The upper surface of the chamber may be coplanar to the upper surface of the microfluidic channel, e.g., substantially coplanar. Accordingly, the chamber(s) may have a cross-sectional height, which may have any values as described herein, that is the same as the channel, e.g., substantially the same, and the chamber(s) and microfluidic channel(s) within the microfluidic device may have a substantially uniform cross sectional height throughout the flow region of the microfluidic device, and may be substantially coplanar throughout the microfluidic device.

Coplanarity of the lower surfaces of the chamber(s) and the microfluidic channel(s) can offer distinct advantage with repositioning micro-objects within the microfluidic device using DEP or magnetic force. Penning and unpenning of micro-objects, and in particular selective penning/selective unpenning, can be greatly facilitated when the lower surfaces of the chamber(s) and the microfluidic channel to which the chamber(s) open have a coplanar orientation.

The proximal opening of the connection region of a sequestration pen may have a width (e.g., W_{con} or W_{con1}) that is at least as large as the largest dimension of a micro-object (e.g., a biological cell, which may be a plant cell, such as a plant protoplast) for which the sequestration pen is intended. In some embodiments, the proximal opening has a width (e.g., W_{con} or W_{con1}) of about 20 microns, about 40 microns, about 50 microns, about 60 microns, about 75 microns, about 100 microns, about 150 microns, about 200 microns, or about 300 microns. The foregoing are examples only, and the width (e.g., W_{con} or W_{con1}) of a proximal opening can be selected to be a value between any of the values listed above (e.g., about 20-200 microns, about 20-150 microns, about 20-100 microns, about 20-75 microns, about 20-60 microns, about 50-300 microns, about 50-200 microns, about 50-150 microns, about 50-100 microns, about 50-75 microns, about 75-150 microns, about 75-100 microns, about 100-300 microns, about 100-200 microns, or about 200-300 microns).

In some embodiments, the connection region of the sequestration pen may have a length (e.g., Leon) from the proximal opening to the distal opening to the isolation region of the sequestration pen that is at least 0.5 times, at least 0.6 times, at least 0.7 times, at least 0.8 times, at least 0.9 times, at least 1.0 times, at least 1.1 times, at least 1.2 times, at least 1.3 times, at least 1.4 times, at least 1.5 times, at least 1.75 times, at least 2.0 times, at least 2.25 times, at least 2.5 times, at least 2.75 times, at least 3.0 times, at least 3.5 times, at least 4.0 times, at least 4.5 times, at least 5.0 times, at least 6.0 times, at least 7.0 times, at least 8.0 times, at least 9.0 times, or at least 10.0 times the width (e.g., W_{con} or W_{con1}) of the proximal opening. Thus, for example, the proximal opening of the connection region of a sequestration pen may have a width (e.g., W_{con} or W_{con1}) from about 20 microns to about 200 microns (e.g., about 50 microns to about 150 microns), and the connection region may have a length L_{con} that is at least 1.0 times (e.g., at least 1.5 times, or at least 2.0 times) the width of the proximal opening. As another example, the proximal opening of the connection region of a sequestration pen may have a width (e.g., W_{con} or W_{con1}) from about 20 microns to about 100 microns (e.g., about 20 microns to about 60 microns), and the connection region may have a length L_{con} that is at least 1.0 times (e.g., at least 1.5 times, or at least 2.0 times) the width of the proximal opening.

The microfluidic channel of a microfluidic device to which a sequestration pen opens may have specified size (e.g., width or height). In some embodiments, the height (e.g., H_{ch}) of the microfluidic channel at a proximal opening to the connection region of a sequestration pen can be within any of the following ranges: 20-100 microns, 20-90 microns, 20-80 microns, 20-70 microns, 20-60 microns, 20-50 microns, 30-100 microns, 30-90 microns, 30-80 microns, 30-70 microns, 30-60 microns, 30-50 microns, 40-100 microns, 40-90 microns, 40-80 microns, 40-70 microns, 40-60 microns, or 40-50 microns. The foregoing are examples only, and the height (e.g., H_{ch}) of the microfluidic channel (e.g., 122) can be selected to be between any of the values listed above. Moreover, the height (e.g., H_{ch}) of the microfluidic channel 122 can be selected to be any of these heights in regions of the microfluidic channel other than at a proximal opening of a sequestration pen.

The width (e.g., W_{ch}) of the microfluidic channel at the proximal opening to the connection region of a sequestration pen can be within any of the following ranges: about 20-500 microns, 20-400 microns, 20-300 microns, 20-200 microns, 20-150 microns, 20-100 microns, 20-80 microns, 20-60 microns, 30-400 microns, 30-300 microns, 30-200 microns, 30-150 microns, 30-100 microns, 30-80 microns, 30-60 microns, 40-300 microns, 40-200 microns, 40-150 microns, 40-100 microns, 40-80 microns, 40-60 microns, 50-1000 microns, 50-500 microns, 50-400 microns, 50-300 microns, 50-250 microns, 50-200 microns, 50-150 microns, 50-100 microns, 50-80 microns, 60-300 microns, 60-200 microns, 60-150 microns, 60-100 microns, 60-80 microns, 70-500 microns, 70-400 microns, 70-300 microns, 70-250 microns, 70-200 microns, 70-150 microns, 70-100 microns, 80-100 microns, 90-400 microns, 90-300 microns, 90-250 microns, 90-200 microns, 90-150 microns, 100-300 microns, 100-250 microns, 100-200 microns, 100-150 microns, 100-120 microns, 200-800 microns, 200-700 microns, or 200-600 microns. The foregoing are examples only, and the width (e.g., W_{ch}) of the microfluidic channel can be a value selected to be between any of the values listed above. Moreover, the width (e.g., W_{ch}) of the microfluidic channel can be selected to be in any of these widths in regions of the microfluidic channel other than at a proximal opening of a sequestration pen. In some embodiments, the width W_{ch} of the microfluidic channel at the proximal opening to the connection region of the sequestration pen (e.g., taken transverse to the direction of bulk flow of fluid through the channel) can be substantially perpendicular to a width (e.g., W_{con} or W_{con1}) of the proximal opening.

A cross-sectional area of the microfluidic channel at a proximal opening to the connection region of a sequestration pen can be about 500-50,000 square microns, 500-40,000 square microns, 500-30,000 square microns, 500-25,000 square microns, 500-20,000 square microns, 500-15,000 square microns, 500-10,000 square microns, 500-7,500 square microns, 500-5,000 square microns, 1,000-25,000 square microns, 1,000-20,000 square microns, 1,000-15,000 square microns, 1,000-10,000 square microns, 1,000-7,500 square microns, 1,000-5,000 square microns, 2,000-20,000 square microns, 2,000-15,000 square microns, 2,000-10,000 square microns, 2,000-7,500 square microns, 2,000-6,000 square microns, 3,000-20,000 square microns, 3,000-15,000 square microns, 3,000-10,000 square microns, 3,000-7,500 square microns, or 3,000 to 6,000 square microns. The foregoing are examples only, and the cross-sectional area of the microfluidic channel at the proximal opening can be selected to be between any of the values listed above. In various embodiments, and the cross-sectional area of the microfluidic channel at regions of the microfluidic channel other than at the proximal opening can also be selected to be between any of the values listed above. In some embodiments, the cross-sectional area is selected to be a substantially uniform value for the entire length of the microfluidic channel.

In some embodiments, the microfluidic chip is configured such that the proximal opening (e.g., 234 or 334) of the connection region of a sequestration pen may have a width (e.g., W_{con} or W_{con1}) from about 20 microns to about 200 microns (e.g., about 50 microns to about 150 microns), the connection region may have a length L_{con} (e.g., 236 or 336) that is at least 1.0 times (e.g., at least 1.5 times, or at least 2.0 times) the width of the proximal opening, and the microfluidic channel may have a height (e.g., H_{ch}) at the proximal opening of about 30 microns to about 60 microns. As another example, the proximal opening (e.g., 234 or 334) of the connection region of a sequestration pen may have a width (e.g., W_{con} or W_{con1}) from about 20 microns to about 100 microns (e.g., about 20 microns to about 60 microns), the connection region may have a length L_{con} (e.g., 236 or 336) that is at least 1.0 times (e.g., at least 1.5 times, or at least 2.0 times) the width of the proximal opening, and the microfluidic channel may have a height (e.g., H_{ch}) at the proximal opening of about 30 microns to about 60 microns. The foregoing are examples only, and the width (e.g., W_{con} or W_{con1}) of the proximal opening (e.g., 234 or 274), the length (e.g., L_{con}) of the connection region, and/or the width (e.g., W_{ch}) of the microfluidic channel (e.g., 122 or 322), can be a value selected to be between any of the values listed above. Generally, however, the width (W_{con} or W_{con1}) of the proximal opening of the connection region of a sequestration pen is less than the width (W_{ch}) of the microfluidic channel. In some embodiments, the width (W_{con} or W_{con1}) of the proximal opening is about 8%, 9%, 10%, 11%, 12%, 13%, 14%, 15%, 16%, 17%, 18%, 19%, 20%, 22%, 24%, 25%, or 30% of the width (W_{ch}) of the microfluidic channel. That is, the width (W_{ch}) of the microfluidic channel may be at least 2.5 times, 3.0 times, 3.5 times, 4.0 times, 4.5 times, 5.0 times, 6.0 times, 7.0 times, 8.0 times, 9.0 times or at least 10.0 times the width (W_{con} or W_{con1}) of the proximal opening of the connection region of the sequestration pen.

In some embodiments, the size W_{C} (e.g., cross-sectional width W_{ch}, diameter, area, or the like) of the channel 122, 322, 618, 718 can be about one and a quarter (1.25), about one and a half (1.5), about two, about two and a half (2.5), about three (3), or more times the size Wo (e.g., cross-sectional width W_{con}, diameter, area, or the like) of a chamber opening, e.g., sequestration pen opening 234, 334, and the like. This can reduce the extent of secondary flow and the rate of diffusion (or diffusion flux) through the opening 234, 334 for materials diffusing from a selected chamber (e.g., like sequestration pens 224, 226 of FIG. 2B) into channel 122, 322, 618, 718 and subsequently re-entering a downstream or adjacent chamber (e.g., like sequestration pen 228). The rate of diffusion of a molecule (e.g., an analyte of interest, such as an antibody) is dependent on a number of factors, including (without limitation) temperature, viscosity of the medium, and the coefficient of diffusion Do of the molecule. For example, the Do for an IgG antibody in aqueous solution at about 20° C. is about 4.4×10⁻⁷ cm²/sec, while the kinematic viscosity of cell culture medium is about 9×10⁻⁴ m²/sec. Thus, an antibody in cell culture medium at about 20° C. can have a rate of diffusion of about 0.5 microns/sec. Accordingly, in some embodiments, a time period for diffusion from a biological micro-object located within a sequestration pen such as 224, 226, 228, 324 into the channel 122, 322, 618, 718 can be about 10 minutes or less (e.g., about 9, 8, 7, 6, 5 minutes, or less). The time period for diffusion can be manipulated by changing parameters that influence the rate of diffusion. For example, the temperature of the media can be increased (e.g., to a physiological temperature such as about 37° C.) or decreased (e.g., to about 15° C., 10° C., or 4° C.) thereby increasing or decreasing the rate of diffusion, respectively. Alternatively, or in addition, the concentrations of solutes in the medium can be increased or decreased as discussed herein to isolate a selected pen from solutes from other upstream pens.

Accordingly, in some variations, the width (e.g., W_{ch}) of the microfluidic channel at the proximal opening to the connection region of a sequestration pen may be about 50 to 500 microns, about 50 to 300 microns, about 50 to 200 microns, about 70 to 500 microns, about to 70-300 microns, about 70 to 250 microns, about 70 to 200 microns, about 70 to 150 microns, about 70 to 100 microns, about 80 to 500 microns, about 80 to 300 microns, about 80 to 250 microns, about 80 to 200 microns, about 80 to 150 microns, about 90 to 500 microns, about 90 to 300 microns, about 90 to 250 microns, about 90 to 200 microns, about 90 to 150 microns, about 100 to 500 microns, about 100 to 300 microns, about 100 to 250 microns, about 100 to 200 microns, or about 100 to 150 microns. In some embodiments, the width W_{ch} of the microfluidic channel at the proximal opening to the connection region of a sequestration pen may be about 70 to 250 microns, about 80 to 200 microns, or about 90 to 150 microns. The width W_{con} of the opening of the chamber (e.g., sequestration pen) may be about 20 to 100 microns; about 30 to 90 microns; or about 20 to 60 microns. In some embodiments, W_{ch} is about 70-250 microns and W_{con} is about 20 to 100 microns; W_{ch} is about 80 to 200 microns and W_{con} is about 30 to 90 microns; W_{ch} is about 90 to 150 microns, and W_{con} is about 20 to 60 microns; or any combination of the widths of W_{ch} and W_{con} thereof.

In some embodiments, the proximal opening (e.g., 234 or 334) of the connection region of a sequestration pen has a width (e.g., W_{con} or W_{con1}) that is 2.0 times or less (e.g., 2.0, 1.9, 1.8, 1.5, 1.3, 1.0, 0.8, 0.5, or 0.1 times) the height (e.g., H_{ch}) of the flow region/microfluidic channel at the proximal opening, or has a value that lies within a range defined by any two of the foregoing values.

In some embodiments, the width W_{con1} of a proximal opening (e.g., 234 or 334) of a connection region of a sequestration pen may be the same as a width W_{con2} of the distal opening (e.g., 238 or 338) to the isolation region thereof. In some embodiments, the width W_{con1} of the proximal opening may be different than a width W_{con2} of the distal opening, and W_{con1} and/or W_{con2} may be selected from any of the values described for W_{con} or W_{con1}. In some embodiments, the walls (including a connection region wall) that define the proximal opening and distal opening may be substantially parallel with respect to each other. In some embodiments, the walls that define the proximal opening and distal opening may be selected to not be parallel with respect to each other.

The length (e.g., Leon) of the connection region can be about 1-600 microns, 5-550 microns, 10-500 microns, 15-400 microns, 20-300 microns, 20-500 microns, 40-400 microns, 60-300 microns, 80-200 microns, about 100-150 microns, about 20-300 microns, about 20-250 microns, about 20-200 microns, about 20-150 microns, about 20-100 microns, about 30-250 microns, about 30-200 microns, about 30-150 microns, about 30-100 microns, about 30-80 microns, about 30-50 microns, about 45-250 microns, about 45-200 microns, about 45-100 microns, about 45-80 microns, about 45-60 microns, about 60-200 microns, about 60-150 microns, about 60-100 microns or about 60-80 microns. The foregoing are examples only, and length (e.g., L_{con}) of a connection region can be selected to be a value that is between any of the values listed above.

The connection region wall of a sequestration pen may have a length (e.g., L_{wall}) that is at least 0.5 times, at least 0.6 times, at least 0.7 times, at least 0.8 times, at least 0.9 times, at least 1.0 times, at least 1.1 times, at least 1.2 times, at least 1.3 times, at least 1.4 times, at least 1.5 times, at least 1.75 times, at least 2.0 times, at least 2.25 times, at least 2.5 times, at least 2.75 times, at least 3.0 times, or at least 3.5 times the width (e.g., W_{con} or W_{con1}) of the proximal opening of the connection region of the sequestration pen. In some embodiments, the connection region wall may have a length L_{wall} of about 20-200 microns, about 20-150 microns, about 20-100 microns, about 20-80 microns, or about 20-50 microns. The foregoing are examples only, and a connection region wall may have a length L_{wall} selected to be between any of the values listed above.

A sequestration pen may have a length Ls of about 40-600 microns, about 40-500 microns, about 40-400 microns, about 40-300 microns, about 40-200 microns, about 40-100 microns or about 40-80 microns. The foregoing are examples only, and a sequestration pen may have a length Ls selected to be between any of the values listed above.

According to some embodiments, a sequestration pen may have a specified height (e.g., Hₛ). In some embodiments, a sequestration pen has a height Hₛ of about 20 microns to about 200 microns (e.g., about 20 microns to about 150 microns, about 20 microns to about 100 microns, about 20 microns to about 60 microns, about 30 microns to about 150 microns, about 30 microns to about 100 microns, about 30 microns to about 60 microns, about 40 microns to about 150 microns, about 40 microns to about 100 microns, or about 40 microns to about 60 microns). The foregoing are examples only, and a sequestration pen can have a height Hₛ selected to be between any of the values listed above.

The height H_{con} of a connection region at a proximal opening of a sequestration pen can be a height within any of the following heights: 20-100 microns, 20-90 microns, 20-80 microns, 20-70 microns, 20-60 microns, 20-50 microns, 30-100 microns, 30-90 microns, 30-80 microns, 30-70 microns, 30-60 microns, 30-50 microns, 40-100 microns, 40-90 microns, 40-80 microns, 40-70 microns, 40-60 microns, or 40-50 microns. The foregoing are examples only, and the height H_{con} of the connection region can be selected to be between any of the values listed above. Typically, the height H_{con} of the connection region is selected to be the same as the height H_{ch} of the microfluidic channel at the proximal opening of the connection region. Additionally, the height Hₛ of the sequestration pen is typically selected to be the same as the height H_{con} of a connection region and/or the height H_{ch} of the microfluidic channel. In some embodiments, Hₛ, H_{con}, and H_{ch} may be selected to be the same value of any of the values listed above for a selected microfluidic device.

The isolation region can be configured to contain only one, two, three, four, five, or a similar relatively small number of micro-objects. In other embodiments, the isolation region may contain more than 10, more than 50 or more than 100 micro-objects. Accordingly, the volume of an isolation region can be, for example, at least 1×10⁴, 1×10⁵, 5×10⁵, 8×10⁵, 1×10⁶, 2×10⁶, 4×10⁶, 6×10⁶, 1×10⁷, 3×10⁷, 5×10⁷ 1×10⁸, 5×10⁸, or 8×10⁸ cubic microns, or more. The foregoing are examples only, and the isolation region can be configured to contain numbers of micro-objects and volumes selected to be between any of the values listed above (e.g., a volume between 1×10⁵ cubic microns and 5×10⁵ cubic microns, between 5×10⁵ cubic microns and 1×10⁶ cubic microns, between 1×10⁶ cubic microns and 2×10⁶ cubic microns, or between 2×10⁶ cubic microns and 1×10⁷ cubic microns).

According to some embodiments, a sequestration pen of a microfluidic device may have a specified volume. The specified volume of the sequestration pen (or the isolation region of the sequestration pen) may be selected such that a single cell or a small number of cells (e.g., 2-10 or 2-5) can rapidly condition the medium and thereby attain favorable (or optimal) growth conditions. In some embodiments, the sequestration pen has a volume of about 5×10⁵, 6×10⁵, 8×10⁵, 1×10⁶, 2×10⁶, 4×10⁶, 8×10⁶, 1×10⁷, 3×10⁷, 5×10⁷, or about 8×10⁷ cubic microns, or more. In some embodiments, the sequestration pen has a volume of about 1 nanoliter to about 50 nanoliters, 2 nanoliters to about 25 nanoliters, 2 nanoliters to about 20 nanoliters, about 2 nanoliters to about 15 nanoliters, or about 2 nanoliters to about 10 nanoliters. The foregoing are examples only, and a sequestration pen can have a volume selected to be any value that is between any of the values listed above.

According to some embodiments, the flow of fluidic medium within the microfluidic channel (e.g., 122 or 322) may have a specified maximum velocity (e.g., Vₘₐₓ). In some embodiments, the maximum velocity (e.g., Vₘₐₓ) may be set at around 0.2, 0.5, 0.7, 1.0, 1.3, 1.5, 2.0, 2.5, 3.0, 3.5, 4.0, 4.5, 5.0, 5.5, 6.0, 6.7, 7.0, 7.5, 8.0, 8.5, 9.0, 10, 11, 12, 13, 14, 15, 16, 17, 18, 19, 20, 22, 24, or 25 microliters/sec. The foregoing are examples only, and the flow of fluidic medium within the microfluidic channel can have a maximum velocity (e.g., Vₘₐₓ) selected to be a value between any of the values listed above. The flow of fluidic medium within the microfluidic channel typically may be flowed at a rate less than the Vₘₐₓ. While the Vₘₐₓ may vary depending on the specific size and numbers of channel and sequestration pens opening thereto, a fluidic medium may be flowed at about 0.1 microliters/sec to about 20 microliters/sec; about 0.1 microliters/sec to about 15 microliters/sec; about 0.1 microliters/sec to about 12 microliters/sec, about 0.1 microliters/sec to about 10 microliters/sec; about 0.1 microliter/sec to about 7 microliters/sec without exceeding the Vₘₐₓ. In some portions of a typical workflow, a flow rate of a fluidic medium may be about 0.1 microliters/sec; about 0.5 microliters/sec; about 1.0 microliters/sec; about 2.0 microliters/sec; about 3.0 microliters/sec; about 4.0 microliters/sec; about 5.0 microliters/sec; about 6.0 microliters/sec; about 7.0 microliters/sec; about 8.0 microliters/sec; about 9.0 microliters/sec; about 10.0 microliters/sec; about 11.0 microliters/sec; or any range defined by two of the foregoing values, e.g., 1-5 microliters/sec or 5-10 microliters/sec. The flow rate of a fluidic medium in the microfluidic channel may be equal to or less than about 12 microliters/sec; about 10 microliters/sec; about 8 microliters/sec, or about 6 microliters/sec.

In various embodiment, the microfluidic device has sequestration pens configured as in any of the embodiments discussed herein where the microfluidic device has about 5 to about 10 sequestration pens, about 10 to about 50 sequestration pens, about 25 to about 200 sequestration pens, about 100 to about 500 sequestration pens, about 200 to about 1000 sequestration pens, about 500 to about 1500 sequestration pens, about 1000 to about 2500 sequestration pens, about 2000 to about 5000 sequestration pens, about 3500 to about 7000 sequestration pens, about 5000 to about 10,000 sequestration pens, about 7,500 to about 15,000 sequestration pens, about 12,500 to about 20,000 sequestration pens, about 15,000 to about 25,000 sequestration pens, about 20,000 to about 30,000 sequestration pens, about 25,000 to about 35,000 sequestration pens, about 30,000 to about 40,000 sequestration pens, about 35,000 to about 45,000 sequestration pens, or about 40,000 to about 50,000 sequestration pens. The sequestration pens need not all be the same size and may include a variety of configurations (e.g., different widths, different features within the sequestration pen).

Coating solutions and coating agents. In some embodiments, at least one inner surface of the microfluidic device includes a coating material that provides a layer of organic and/or hydrophilic molecules suitable for maintenance, expansion and/or movement of biological micro-object(s) (i.e., the biological micro-object exhibits increased viability, greater expansion and/or greater portability within the microfluidic device). The conditioned surface may reduce surface fouling, participate in providing a layer of hydration, and/or otherwise shield the biological micro-objects from contact with the non-organic materials of the microfluidic device interior.

In some embodiments, substantially all the inner surfaces of the microfluidic device include the coating material. The coated inner surface(s) may include the surface of a flow region (e.g., channel), chamber, or sequestration pen, or a combination thereof. In some embodiments, each of a plurality of sequestration pens has at least one inner surface coated with coating materials. In other embodiments, each of a plurality of flow regions or channels has at least one inner surface coated with coating materials. In some embodiments, at least one inner surface of each of a plurality of sequestration pens and each of a plurality of channels is coated with coating materials. The coating may be applied before or after introduction of biological micro-object(s), or may be introduced concurrently with the biological micro-object(s). In some embodiments, the biological micro-object(s) may be imported into the microfluidic device in a fluidic medium that includes one or more coating agents. In other embodiments, the inner surface(s) of the microfluidic device (e.g., a microfluidic device having an electrode activation substrate such as, but not limited to, a device including dielectrophoresis (DEP) electrodes) may be treated or “primed” with a coating solution comprising a coating agent prior to introduction of the biological micro-object(s) into the microfluidic device. Any convenient coating agent/coating solution can be used, including but not limited to: serum or serum factors, bovine serum albumin (BSA), polymers, detergents, enzymes, and any combination thereof.

Synthetic polymer-based coating materials. The at least one inner surface may include a coating material that comprises a polymer. The polymer may be non-covalently bound (e.g., it may be non-specifically adhered) to the at least one surface. The polymer may have a variety of structural motifs, such as found in block polymers (and copolymers), star polymers (star copolymers), and graft or comb polymers (graft copolymers), all of which may be suitable for the methods disclosed herein. A wide variety of alkylene ether containing polymers may be suitable for use in the microfluidic devices described herein, including but not limited to Pluronic® polymers such as Pluronic® L44, L64, P85, and F127 (including F127NF). Other examples of suitable coating materials are described in US2016/0312165, the contents of which are herein incorporated by reference in their entirety.

Covalently linked coating materials. In some embodiments, the at least one inner surface includes covalently linked molecules that provide a layer of organic and/or hydrophilic molecules suitable for maintenance/expansion of biological micro-object(s) within the microfluidic device, providing a conditioned surface for such cells. The covalently linked molecules include a linking group, wherein the linking group is covalently linked to one or more surfaces of the microfluidic device, as described below. The linking group is also covalently linked to a surface modifying moiety configured to provide a layer of organic and/or hydrophilic molecules suitable for maintenance/expansion/movement of biological micro-object(s).

In some embodiments, the covalently linked moiety configured to provide a layer of organic and/or hydrophilic molecules suitable for maintenance/expansion of biological micro-object(s) may include alkyl or fluoroalkyl (which includes perfluoroalkyl) moieties; mono- or polysaccharides (which may include but is not limited to dextran); alcohols (including but not limited to propargyl alcohol); polyalcohols, including but not limited to polyvinyl alcohol; alkylene ethers, including but not limited to polyethylene glycol; polyelectrolytes (including but not limited to polyacrylic acid or polyvinyl phosphonic acid); amino groups (including derivatives thereof, such as, but not limited to alkylated amines, hydroxyalkylated amino group, guanidinium, and heterocyclic groups containing an unaromatized nitrogen ring atom, such as, but not limited to morpholinyl or piperazinyl); carboxylic acids including but not limited to propiolic acid (which may provide a carboxylate anionic surface); phosphonic acids, including but not limited to ethynyl phosphonic acid (which may provide a phosphonate anionic surface); sulfonate anions; carboxybetaines; sulfobetaines; sulfamic acids; or amino acids.

In various embodiments, the covalently linked moiety configured to provide a layer of organic and/or hydrophilic molecules suitable for maintenance/expansion of biological micro-object(s) in the microfluidic device may include non-polymeric moieties such as an alkyl moiety, amino acid moiety, alcohol moiety, amino moiety, carboxylic acid moiety, phosphonic acid moiety, sulfonic acid moiety, sulfamic acid moiety, or saccharide moiety. Alternatively, the covalently linked moiety may include polymeric moieties, which may include any of these moieties.

In some embodiments, a microfluidic device may have a hydrophobic layer upon the inner surface of the base which includes a covalently linked alkyl moiety. The covalently linked alkyl moiety may comprise carbon atoms forming a linear chain (e.g., a linear chain of at least 10 carbons, or at least 14, 16, 18, 20, 22, or more carbons) and may be an unbranched alkyl moiety. In some embodiments, the alkyl group may include a substituted alkyl group (e.g., some of the carbons in the alkyl group can be fluorinated or perfluorinated). In some embodiments, the alkyl group may include a first segment, which may include a perfluoroalkyl group, joined to a second segment, which may include a non-substituted alkyl group, where the first and second segments may be joined directly or indirectly (e.g., by means of an ether linkage). The first segment of the alkyl group may be located distal to the linking group, and the second segment of the alkyl group may be located proximal to the linking group.

In other embodiments, the covalently linked moiety may include at least one amino acid, which may include more than one type of amino acid. Thus, the covalently linked moiety may include a peptide or a protein. In some embodiments, the covalently linked moiety may include an amino acid which may provide a zwitterionic surface to support cell growth, viability, portability, or any combination thereof.

In other embodiments, the covalently linked moiety may further include a streptavidin or biotin moiety. In some embodiments, a modified biological moiety such as, for example, a biotinylated protein or peptide may be introduced to the inner surface of a microfluidic device bearing covalently linked streptavidin, and couple via the covalently linked streptavidin to the surface, thereby providing a modified surface presenting the protein or peptide.

In other embodiments, the covalently linked moiety may include at least one alkylene oxide moiety and may include any alkylene oxide polymer as described above. One useful class of alkylene ether containing polymers is polyethylene glycol (PEG Mw<100,000 Da) or alternatively polyethylene oxide (PEO, M_{w}>100,000). In some embodiments, a PEG may have an M_{w} of about 1000 Da, 5000 Da, 10,000 Da or 20,000 Da. In some embodiments, the PEG polymer may further be substituted with a hydrophilic or charged moiety, such as but not limited to an alcohol functionality or a carboxylic acid moiety.

The covalently linked moiety may include one or more saccharides. The covalently linked saccharides may be mono-, di-, or polysaccharides. The covalently linked saccharides may be modified to introduce a reactive pairing moiety which permits coupling or elaboration for attachment to the surface. One exemplary covalently linked moiety may include a dextran polysaccharide, which may be coupled indirectly to a surface via an unbranched linker.

The coating material providing a conditioned surface may comprise only one kind of covalently linked moiety or may include more than one different kind of covalently linked moiety. For example, a polyethylene glycol conditioned surface may have covalently linked alkylene oxide moieties having a specified number of alkylene oxide units which are all the same, e.g., having the same linking group and covalent attachment to the surface, the same overall length, and the same number of alkylene oxide units. Alternatively, the coating material may have more than one kind of covalently linked moiety attached to the surface. For example, the coating material may include the molecules having covalently linked alkylene oxide moieties having a first specified number of alkylene oxide units and may further include a further set of molecules having bulky moieties such as a protein or peptide connected to a covalently attached alkylene oxide linking moiety having a greater number of alkylene oxide units. The different types of molecules may be varied in any suitable ratio to obtain the surface characteristics desired. For example, the conditioned surface having a mixture of first molecules having a chemical structure having a first specified number of alkylene oxide units and second molecules including peptide or protein moieties, which may be coupled via a biotin/streptavidin binding pair to the covalently attached alkylene linking moiety, may have a ratio of first molecules:second molecules of about 99:1; about 90:10; about 75:25; about 50:50; about 30:70; about 20:80; about 10:90; or any ratio selected to be between these values. In this instance, the first set of molecules having different, less sterically demanding termini and fewer backbone atoms can help to functionalize the entire substrate surface and thereby prevent undesired adhesion or contact with the silicon/silicon oxide, hafnium oxide or alumina making up the substrate itself. The selection of the ratio of mixture of first molecules to second molecules may also modulate the surface modification introduced by the second molecules bearing peptide or protein moieties.

Conditioned surface properties. Various factors can alter the physical thickness of the conditioned surface, such as the manner in which the conditioned surface is formed on the substrate (e.g., vapor deposition, liquid phase deposition, spin coating, flooding, and electrostatic coating). In some embodiments, the conditioned surface may have a thickness of about 1 nm to about 10 nm. In some embodiments, the covalently linked moieties of the conditioned surface may form a monolayer when covalently linked to the surface of the microfluidic device (which may include an electrode activation substrate having dielectrophoresis (DEP) or electrowetting (EW) electrodes) and may have a thickness of less than 10 nm (e.g., less than 5 nm, or about 1.5 to 3.0 nm). These values are in contrast to that of a surface prepared by spin coating, for example, which may typically have a thickness of about 30 nm. In some embodiments, the conditioned surface does not require a perfectly formed monolayer to be suitably functional for operation within a DEP-configured microfluidic device. In other embodiments, the conditioned surface formed by the covalently linked moieties may have a thickness of about 10 nm to about 50 nm.

Unitary or Multi-part conditioned surface. The covalently linked coating material may be formed by reaction of a molecule which already contains the moiety configured to provide a layer of organic and/or hydrophilic molecules suitable for maintenance/expansion of biological micro-object(s) in the microfluidic device, and may have a structure of Formula I, as shown below. Alternatively, the covalently linked coating material may be formed in a two-part sequence, having a structure of Formula II, by coupling the moiety configured to provide a layer of organic and/or hydrophilic molecules suitable for maintenance and/or expansion of biological micro-object(s) to a surface modifying ligand that itself has been covalently linked to the surface. In some embodiments, the surface may be formed in a two-part or three-part sequence, including a streptavidin/biotin binding pair, to introduce a protein, peptide, or mixed modified surface.

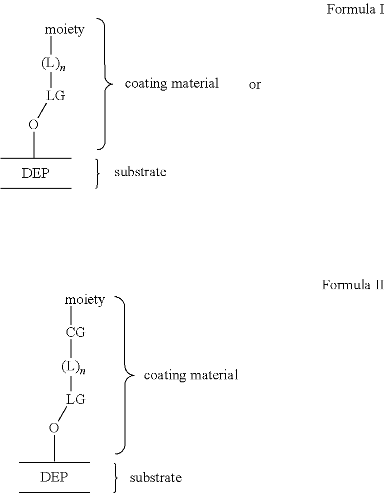

The coating material may be linked covalently to oxides of the surface of a DEP-configured or EW-configured substrate. The coating material may be attached to the oxides via a linking group (“LG”), which may be a siloxy or phosphonate ester group formed from the reaction of a siloxane or phosphonic acid group with the oxides. The moiety configured to provide a layer of organic and/or hydrophilic molecules suitable for maintenance/expansion of biological micro-object(s) in the microfluidic device can be any of the moieties described herein. The linking group LG may be directly or indirectly connected to the moiety configured to provide a layer of organic and/or hydrophilic molecules suitable for maintenance/expansion of biological micro-object(s) in the microfluidic device. When the linking group LG is directly connected to the moiety, optional linker (“L”) is not present and n is 0. When the linking group LG is indirectly connected to the moiety, linker L is present and n is 1. The linker L may have a linear portion where a backbone of the linear portion may include 1 to 200 non-hydrogen atoms selected from any combination of silicon, carbon, nitrogen, oxygen, sulfur and/or phosphorus atoms, subject to chemical bonding limitations as is known in the art. It may be interrupted with any combination of one or more moieties, which may be chosen from ether, amino, carbonyl, amido, and/or phosphonate groups, arylene, heteroarylene, or heterocyclic groups. In some embodiments, the coupling group CG represents the resultant group from reaction of a reactive moiety Rₓ and a reactive pairing moiety Rₚₓ (i.e., a moiety configured to react with the reactive moiety Rₓ). CG may be a carboxamidyl group, a triazolylene group, substituted triazolylene group, a carboxamidyl, thioamidyl, an oxime, a mercaptyl, a disulfide, an ether, or alkenyl group, or any other suitable group that may be formed upon reaction of a reactive moiety with its respective reactive pairing moiety. In some embodiments, CG may further represent a streptavidin/biotin binding pair.

Further details of suitable coating treatments and modifications, as well as methods of preparation, may be found at U.S. Patent Application Publication No. US2016/0312165 (Lowe, Jr., et al.), U.S. Patent Application Publication No US2017/0173580 (Lowe, Jr., et al), International Patent Application Publication WO2017/205830 (Lowe, Jr., et al.), and International Patent Application Publication WO2019/01880 (Beemiller et al.), each of which disclosures is herein incorporated by reference in its entirety.

Microfluidic device motive technologies. The microfluidic devices described herein can be used with any type of motive technology. As described herein, the control and monitoring equipment of the system can comprise a motive module for selecting and moving objects, such as micro-objects or droplets, in the microfluidic circuit of a microfluidic device. The motive technology(ies) may include, for example, dielectrophoresis (DEP), electrowetting (EW), and/or other motive technologies. The microfluidic device can have a variety of motive configurations, depending upon the type of object being moved and other considerations. Returning to FIG. 1A, for example, the support structure 104 and/or cover 110 of the microfluidic device 100 can comprise DEP electrode activation substrates for selectively inducing motive forces on micro-objects in the fluidic medium 180 in the microfluidic circuit 120 and thereby select, capture, and/or move individual micro-objects or groups of micro-objects.

In some embodiments, motive forces are applied across the fluidic medium 180 (e.g., in the flow path and/or in the sequestration pens) via one or more electrodes (not shown) to manipulate, transport, separate and sort micro-objects located therein. For example, in some embodiments, motive forces are applied to one or more portions of microfluidic circuit 120 in order to transfer a single micro-object from the flow path 106 into a desired microfluidic sequestration pen. In some embodiments, motive forces are used to prevent a micro-object within a sequestration pen from being displaced therefrom. Further, in some embodiments, motive forces are used to selectively remove a micro-object from a sequestration pen that was previously collected in accordance with the embodiments of the current disclosure.

In some embodiments, the microfluidic device is configured as an optically-actuated electrokinetic device, such as in optoelectronic tweezer (OET) and/or optoelectrowetting (OEW) configured device. Examples of suitable OET configured devices (e.g., containing optically actuated dielectrophoresis electrode activation substrates) can include those illustrated in U.S. Pat. No. RE 44,711 (Wu, et al.) (originally issued as U.S. Pat. No. 7,612,355), U.S. Pat. No. 7,956,339 (Ohta, et al.), U.S. Pat. No. 9,908,115 (Hobbs et al.), and U.S. Pat. No. 9,403,172 (Short et al), each of which is incorporated herein by reference in its entirety. Examples of suitable OEW configured devices can include those illustrated in U.S. Pat. No. 6,958,132 (Chiou, et al.), and U.S. Pat. No. 9,533,306 (Chiou, et al.), each of which is incorporated herein by reference in its entirety. Examples of suitable optically-actuated electrokinetic devices that include combined OET/OEW configured devices can include those illustrated in U.S. Patent Application Publication No. 2015/0306598 (Khandros, et al.), U.S. Patent Application Publication No 2015/0306599 (Khandros, et al.), and U.S. Patent Application Publication No. 2017/0173580 (Lowe, et al.), each of which is incorporated herein by reference in its entirety.

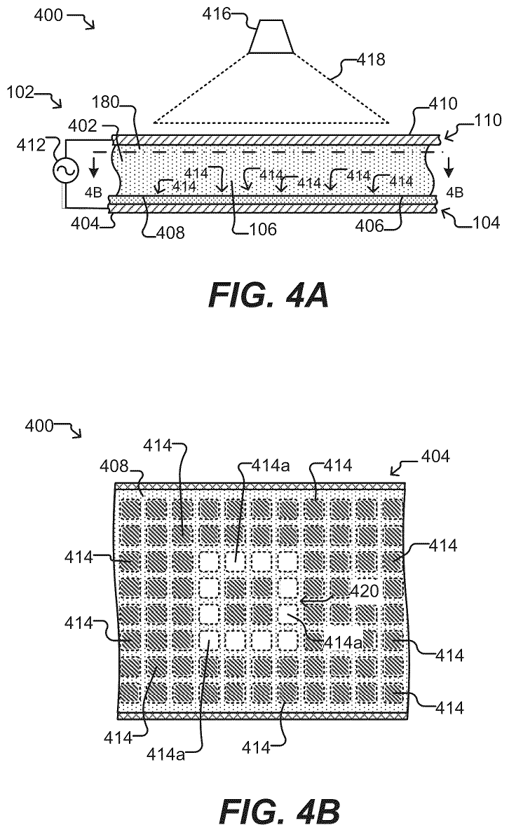

It should be understood that, for purposes of simplicity, the various examples of FIGS. 1-5B may illustrate portions of microfluidic devices while not depicting other portions. Further, FIGS. 1-5B may be part of, and implemented as, one or more microfluidic systems. In one non-limiting example, FIGS. 4A and 4B show a side cross-sectional view and a top cross-sectional view, respectively, of a portion of an enclosure 102 of the microfluidic device 400 having a region/chamber 402, which may be part of a fluidic circuit element having a more detailed structure, such as a growth chamber, a sequestration pen (which may be like any sequestration pen described herein), a flow region, or a flow channel. For instance, microfluidic device 400 may be similar to microfluidic devices 100, 175, 200, 300, 520 or any other microfluidic device as described herein. Furthermore, the microfluidic device 400 may include other fluidic circuit elements and may be part of a system including control and monitoring equipment 152, described above, having one or more of the media module 160, motive module 162, imaging module 164, optional tilting module 166, and other modules 168. Microfluidic devices 175, 200, 300, 520 and any other microfluidic devices described herein may similarly have any of the features described in detail for FIGS. 1A-1B and 4A-4B.

As shown in the example of FIG. 4A, the microfluidic device 400 includes a support structure 104 having a bottom electrode 404 and an electrode activation substrate 406 overlying the bottom electrode 404, and a cover 110 having a top electrode 410, with the top electrode 410 spaced apart from the bottom electrode 404. The top electrode 410 and the electrode activation substrate 406 define opposing surfaces of the region/chamber 402. A fluidic medium 180 contained in the region/chamber 402 thus provides a resistive connection between the top electrode 410 and the electrode activation substrate 406. A power source 412 configured to be connected to the bottom electrode 404 and the top electrode 410 and create a biasing voltage between the electrodes, as required for the generation of DEP forces in the region/chamber 402, is also shown. The power source 412 can be, for example, an alternating current (AC) power source.

In certain embodiments, the microfluidic device 400 illustrated in FIGS. 4A and 4B can have an optically-actuated DEP electrode activation substrate. Accordingly, changing patterns of light 418 from the light source 416, which may be controlled by the motive module 162, can selectively activate and deactivate changing patterns of DEP electrodes at regions 414 of the inner surface 408 of the electrode activation substrate 406. (Hereinafter the regions 414 of a microfluidic device having a DEP electrode activation substrate are referred to as “DEP electrode regions.”) As illustrated in FIG. 4B, a light pattern 418 directed onto the inner surface 408 of the electrode activation substrate 406 can illuminate select DEP electrode regions 414a (shown in white) in a pattern, such as a square. The non-illuminated DEP electrode regions 414 (cross-hatched) are hereinafter referred to as “dark” DEP electrode regions 414. The relative electrical impedance through the DEP electrode activation substrate 406 (i.e., from the bottom electrode 404 up to the inner surface 408 of the electrode activation substrate 406 which interfaces with the fluidic medium 180 in the flow region 106) is greater than the relative electrical impedance through the fluidic medium 180 in the region/chamber 402 (i.e., from the inner surface 408 of the electrode activation substrate 406 to the top electrode 410 of the cover 110) at each dark DEP electrode region 414. An illuminated DEP electrode region 414a, however, exhibits a reduced relative impedance through the electrode activation substrate 406 that is less than the relative impedance through the fluidic medium 180 in the region/chamber 402 at each illuminated DEP electrode region 414a.

With the power source 412 activated, the foregoing DEP configuration creates an electric field gradient in the fluidic medium 180 between illuminated DEP electrode regions 414a and adjacent dark DEP electrode regions 414, which in turn creates local DEP forces that attract or repel nearby micro-objects (not shown) in the fluidic medium 180. DEP electrodes that attract or repel micro-objects in the fluidic medium 180 can thus be selectively activated and deactivated at many different such DEP electrode regions 414 at the inner surface 408 of the region/chamber 402 by changing light patterns 418 projected from a light source 416 into the microfluidic device 400. Whether the DEP forces attract or repel nearby micro-objects can depend on such parameters as the frequency of the power source 412 and the dielectric properties of the fluidic medium 180 and/or micro-objects (not shown). Depending on the frequency of the power applied to the DEP configuration and selection of fluidic media (e.g., a highly conductive media such as PBS or other media appropriate for maintaining biological cells), negative DEP forces may be produced. Negative DEP forces may repel the micro-objects away from the location of the induced non-uniform electrical field. In some embodiments, a microfluidic device incorporating DEP technology may generate negative DEP forces.

The square pattern 420 of illuminated DEP electrode regions 414a illustrated in FIG. 4B is an example only. Any pattern of the DEP electrode regions 414 can be illuminated (and thereby activated) by the pattern of light 418 projected into the microfluidic device 400, and the pattern of illuminated/activated DEP electrode regions 414 can be repeatedly changed by changing or moving the light pattern 418.

In some embodiments, the electrode activation substrate 406 can comprise or consist of a photoconductive material. In such embodiments, the inner surface 408 of the electrode activation substrate 406 can be featureless. For example, the electrode activation substrate 406 can comprise or consist of a layer of hydrogenated amorphous silicon (a-Si:H). The a-Si:H can comprise, for example, about 8% to 40% hydrogen (calculated as 100*the number of hydrogen atoms/the total number of hydrogen and silicon atoms). The layer of a-Si:H can have a thickness of about 500 nm to about 2.0 μm. In such embodiments, the DEP electrode regions 414 can be created anywhere and in any pattern on the inner surface 408 of the electrode activation substrate 406, in accordance with the light pattern 418. The number and pattern of the DEP electrode regions 414 thus need not be fixed, but can correspond to the light pattern 418. Examples of microfluidic devices having a DEP configuration comprising a photoconductive layer such as discussed above have been described, for example, in U.S. Pat. No. RE 44,711 (Wu, et al.) (originally issued as U.S. Pat. No. 7,612,355), each of which is incorporated herein by reference in its entirety.

In other embodiments, the electrode activation substrate 406 can comprise a substrate comprising a plurality of doped layers, electrically insulating layers (or regions), and electrically conductive layers that form semiconductor integrated circuits, such as is known in semiconductor fields. For example, the electrode activation substrate 406 can comprise a plurality of phototransistors, including, for example, lateral bipolar phototransistors, with each phototransistor corresponding to a DEP electrode region 414. Alternatively, the electrode activation substrate 406 can comprise electrodes (e.g., conductive metal electrodes) controlled by phototransistor switches, with each such electrode corresponding to a DEP electrode region 414. The electrode activation substrate 406 can include a pattern of such phototransistors or phototransistor-controlled electrodes. The pattern, for example, can be an array of substantially square phototransistors or phototransistor-controlled electrodes arranged in rows and columns. Alternatively, the pattern can be an array of substantially hexagonal phototransistors or phototransistor-controlled electrodes that form a hexagonal lattice. Regardless of the pattern, electric circuit elements can form electrical connections between the DEP electrode regions 414 at the inner surface 408 of the electrode activation substrate 406 and the bottom electrode 404, and those electrical connections (i.e., phototransistors or electrodes) can be selectively activated and deactivated by the light pattern 418, as described above.

Examples of microfluidic devices having electrode activation substrates that comprise phototransistors have been described, for example, in U.S. Pat. No. 7,956,339 (Ohta et al.) and U.S. Pat. No. 9,908,115 (Hobbs et al.), the entire contents of each of which are incorporated herein by reference. Examples of microfluidic devices having electrode activation substrates that comprise electrodes controlled by phototransistor switches have been described, for example, in U.S. Pat. No. 9,403,172 (Short et al.), which is incorporated herein by reference in its entirety.

In some embodiments of a DEP configured microfluidic device, the top electrode 410 is part of a first wall (or cover 110) of the enclosure 402, and the electrode activation substrate 406 and bottom electrode 404 are part of a second wall (or support structure 104) of the enclosure 102. The region/chamber 402 can be between the first wall and the second wall. In other embodiments, the electrode 410 is part of the second wall (or support structure 104) and one or both of the electrode activation substrate 406 and/or the electrode 410 are part of the first wall (or cover 110). Moreover, the light source 416 can alternatively be used to illuminate the enclosure 102 from below.

With the microfluidic device 400 of FIGS. 4A-4B having a DEP electrode activation substrate, the motive module 162 of control and monitoring equipment 152, as described for FIG. 1A herein, can select a micro-object (not shown) in the fluidic medium 180 in the region/chamber 402 by projecting a light pattern 418 into the microfluidic device 400 to activate a first set of one or more DEP electrodes at DEP electrode regions 414a of the inner surface 408 of the electrode activation substrate 406 in a pattern (e.g., square pattern 420) that surrounds and captures the micro-object. The motive module 162 can then move the in situ-generated captured micro-object by moving the light pattern 418 relative to the microfluidic device 400 to activate a second set of one or more DEP electrodes at DEP electrode regions 414. Alternatively, the microfluidic device 400 can be moved relative to the light pattern 418.

In other embodiments, the microfluidic device 400 may be a DEP configured device that does not rely upon light activation of DEP electrodes at the inner surface 408 of the electrode activation substrate 406. For example, the electrode activation substrate 406 can comprise selectively addressable and energizable electrodes positioned opposite to a surface including at least one electrode (e.g., cover 110). Switches (e.g., transistor switches in a semiconductor substrate) may be selectively opened and closed to activate or inactivate DEP electrodes at DEP electrode regions 414, thereby creating a net DEP force on a micro-object (not shown) in region/chamber 402 in the vicinity of the activated DEP electrodes. Depending on such characteristics as the frequency of the power source 412 and the dielectric properties of the medium (not shown) and/or micro-objects in the region/chamber 402, the DEP force can attract or repel a nearby micro-object. By selectively activating and deactivating a set of DEP electrodes (e.g., at a set of DEP electrodes regions 414 that forms a square pattern 420), one or more micro-objects in region/chamber 402 can be selected and moved within the region/chamber 402. The motive module 162 in FIG. 1A can control such switches and thus activate and deactivate individual ones of the DEP electrodes to select, and move particular micro-objects (not shown) around the region/chamber 402. Microfluidic devices having a DEP electrode activation substrate that includes selectively addressable and energizable electrodes are known in the art and have been described, for example, in U.S. Pat. No. 6,294,063 (Becker, et al.) and U.S. Pat. No. 6,942,776 (Medoro), each of which is incorporated herein by reference in its entirety.

Regardless of whether the microfluidic device 400 has a dielectrophoretic electrode activation substrate, an electrowetting electrode activation substrate or a combination of both a dielectrophoretic and an electrowetting activation substrate, a power source 412 can be used to provide a potential (e.g., an AC voltage potential) that powers the electrical circuits of the microfluidic device 400. The power source 412 can be the same as, or a component of, the power source 192 referenced in FIG. 1A. Power source 412 can be configured to provide an AC voltage and/or current to the top electrode 410 and the bottom electrode 404. For an AC voltage, the power source 412 can provide a frequency range and an average or peak power (e.g., voltage or current) range sufficient to generate net DEP forces (or electrowetting forces) strong enough to select and move individual micro-objects (not shown) in the region/chamber 402, as discussed above, and/or to change the wetting properties of the inner surface 408 of the support structure 104 in the region/chamber 202, as also discussed above. Such frequency ranges and average or peak power ranges are known in the art. See, e.g., U.S. Pat. No. 6,958,132 (Chiou, et al.), U.S. Pat. No. RE44,711 (Wu, et al.) (originally issued as U.S. Pat. No. 7,612,355), and U.S. Patent Application Publication Nos. 2014/0124370 (Short, et al.), 2015/0306598 (Khandros, et al.), 2015/0306599 (Khandros, et al.), and 2017/0173580 (Lowe, Jr. et al.), each of which disclosures are herein incorporated by reference in its entirety.

Other forces may be utilized within the microfluidic devices, alone or in combination, to move selected micro-objects. Bulk fluidic flow within the microfluidic channel may move micro-objects within the flow region. Localized fluidic flow, which may be operated within the microfluidic channel, within a sequestration pen, or within another kind of chamber (e.g., a reservoir) can also be used to move selected micro-objects. Localized fluidic flow can be used to move selected micro-objects out of the flow region into a non-flow region such as a sequestration pen or the reverse, from a non-flow region into a flow region. The localized flow can be actuated by deforming a deformable wall of the microfluidic device, as described in U.S. Pat. No. 10,058,865 (Breinlinger, et al.), which is incorporated herein by reference in its entirety.

Gravity may be used to move micro-objects within the microfluidic channel, into a sequestration pen, and/or out of a sequestration pen or other chamber, as described in U.S. Pat. No. 9,744,533 (Breinlinger, et al.), which is incorporated herein by reference in its entirety. Use of gravity (e.g., by tilting the microfluidic device and/or the support to which the microfluidic device is attached) may be useful for bulk movement of cells into or out of the sequestration pens from/to the flow region. Magnetic forces may be employed to move micro-objects including paramagnetic materials, which can include magnetic micro-objects attached to or associated with a biological micro-object. Alternatively, or in additional, centripetal forces may be used to move micro-objects within the microfluidic channel, as well as into or out of sequestration pens or other chambers in the microfluidic device.

In another alternative mode of moving micro-objects, laser-generated dislodging forces may be used to export micro-objects or assist in exporting micro-objects from a sequestration pen or any other chamber in the microfluidic device, as described in International Patent Publication No. WO2017/117408 (Kurz, et al.), which is incorporated herein by reference in its entirety.

In some embodiments, DEP forces are combined with other forces, such as fluidic flow (e.g., bulk fluidic flow in a channel or localized fluidic flow actuated by deformation of a deformable surface of the microfluidic device, laser generated dislodging forces, and/or gravitational force), so as to manipulate, transport, separate and sort micro-objects and/or droplets within the microfluidic circuit 120. In some embodiments, the DEP forces can be applied prior to the other forces. In other embodiments, the DEP forces can be applied after the other forces. In still other instances, the DEP forces can be applied in an alternating manner with the other forces. For the microfluidic devices described herein, repositioning of micro-objects may not generally rely upon gravity or hydrodynamic forces to position or trap micro-objects at a selected position. Gravity may be chosen as one form of repositioning force, but the ability to reposition of micro-objects within the microfluidic device does not rely solely upon the use of gravity. While fluid flow in the microfluidic channels may be used to introduce micro-objects into the microfluidic channels (e.g., flow region), such regional flow is not relied upon to pen or unpen micro-objects, while localized flow (e.g., force derived from actuating a deformable surface) may, in some embodiments, be selected from amongst the other types of repositioning forces described herein to pen or unpen micro-objects or to export them from the microfluidic device.

When DEP is used to reposition micro-objects, bulk fluidic flow in a channel is generally stopped prior to applying DEP to micro-objects to reposition the micro-objects within the microfluidic circuit of the device, whether the micro-objects are being repositioned from the channel into a sequestration pen or from a sequestration pen into the channel. Bulk fluidic flow may be resumed thereafter.

System. Returning to FIG. 1A, a system 150 for operating and controlling microfluidic devices is shown, such as for controlling the microfluidic device 100. The electrical power source 192 can provide electric power to the microfluidic device 100, providing biasing voltages or currents as needed. The electrical power source 192 can, for example, comprise one or more alternating current (AC) and/or direct current (DC) voltage or current sources.

System 150 can further include a media source 178. The media source 178 (e.g., a container, reservoir, or the like) can comprise multiple sections or containers, each for holding a different fluidic medium 180. Thus, the media source 178 can be a device that is outside of and separate from the microfluidic device 100, as illustrated in FIG. 1A. Alternatively, the media source 178 can be located in whole or in part inside the enclosure 102 of the microfluidic device 100. For example, the media source 178 can comprise reservoirs that are part of the microfluidic device 100.

FIG. 1A also illustrates simplified block diagram depictions of examples of control and monitoring equipment 152 that constitute part of system 150 and can be utilized in conjunction with a microfluidic device 100. As shown, examples of such control and monitoring equipment 152 can include a master controller 154 comprising a media module 160 for controlling the media source 178, a motive module 162 for controlling movement and/or selection of micro-objects (not shown) and/or medium (e.g., droplets of medium) in the microfluidic circuit 120, an imaging module 164 for controlling an imaging device (e.g., a camera, microscope, light source or any combination thereof) for capturing images (e.g., digital images), and an optional tilting module 166 for controlling the tilting of the microfluidic device 100. The control equipment 152 can also include other modules 168 for controlling, monitoring, or performing other functions with respect to the microfluidic device 100. As shown, the monitoring equipment 152 can further include a display device 170 and an input/output device 172.

The master controller 154 can comprise a control module 156 and a digital memory 158. The control module 156 can comprise, for example, a digital processor configured to operate in accordance with machine executable instructions (e.g., software, firmware, source code, or the like) stored as non-transitory data or signals in the memory 158. Alternatively, or in addition, the control module 156 can comprise hardwired digital circuitry and/or analog circuitry. The media module 160, motive module 162, imaging module 164, optional tilting module 166, and/or other modules 168 can be similarly configured. Thus, functions, processes acts, actions, or steps of a process discussed herein as being performed with respect to the microfluidic device 100 or any other microfluidic apparatus can be performed by any one or more of the master controller 154, media module 160, motive module 162, imaging module 164, optional tilting module 166, and/or other modules 168 configured as discussed above. Similarly, the master controller 154, media module 160, motive module 162, imaging module 164, optional tilting module 166, and/or other modules 168 may be communicatively coupled to transmit and receive data used in any function, process, act, action or step discussed herein.

The media module 160 controls the media source 178. For example, the media module 160 can control the media source 178 to input a selected fluidic medium 180 into the enclosure 102 (e.g., through an inlet port 107). The media module 160 can also control removal of media from the enclosure 102 (e.g., through an outlet port (not shown)). One or more media can thus be selectively input into and removed from the microfluidic circuit 120. The media module 160 can also control the flow of fluidic medium 180 in the flow path 106 inside the microfluidic circuit 120. The media module 160 may also provide conditioning gaseous conditions to the media source 178, for example, providing an environment containing 5% CO₂ (or higher). The media module 160 may also control the temperature of an enclosure of the media source, for example, to provide feeder cells in the media source with proper temperature control.

Motive module. The motive module 162 can be configured to control selection and movement of micro-objects (not shown) in the microfluidic circuit 120. The enclosure 102 of the microfluidic device 100 can comprise one or more electrokinetic mechanisms including a dielectrophoresis (DEP) electrode activation substrate, optoelectronic tweezers (OET) electrode activation substrate, electrowetting (EW) electrode activation substrate, and/or an opto-electrowetting (OEW) electrode activation substrate, where the motive module 162 can control the activation of electrodes and/or transistors (e.g., phototransistors) to select and move micro-objects and/or droplets in the flow path 106 and/or within sequestration pens 124, 126, 128, and 130. The electrokinetic mechanism(s) may be any suitable single or combined mechanism as described within the paragraphs describing motive technologies for use within the microfluidic device. A DEP configured device may include one or more electrodes that apply a non-uniform electric field in the microfluidic circuit 120 sufficient to exert a dielectrophoretic force on micro-objects in the microfluidic circuit 120. An OET configured device may include photo-activatable electrodes to provide selective control of movement of micro-objects in the microfluidic circuit 120 via light-induced dielectrophoresis.

The imaging module 164 can control the imaging device. For example, the imaging module 164 can receive and process image data from the imaging device. Image data from the imaging device can comprise any type of information captured by the imaging device (e.g., the presence or absence of micro-objects, droplets of medium, accumulation of label, such as fluorescent label, etc.). Using the information captured by the imaging device, the imaging module 164 can further calculate the position of objects (e.g., micro-objects, droplets of medium) and/or the rate of motion of such objects within the microfluidic device 100.

The imaging device (part of imaging module 164, discussed below) can comprise a device, such as a digital camera, for capturing images inside microfluidic circuit 120. In some instances, the imaging device further comprises a detector having a fast frame rate and/or high sensitivity (e.g., for low light applications). The imaging device can also include a mechanism for directing stimulating radiation and/or light beams into the microfluidic circuit 120 and collecting radiation and/or light beams reflected or emitted from the microfluidic circuit 120 (or micro-objects contained therein). The emitted light beams may be in the visible spectrum and may, e.g., include fluorescent emissions. The reflected light beams may include reflected emissions originating from an LED or a wide spectrum lamp, such as a mercury lamp (e.g., a high-pressure mercury lamp) or a Xenon arc lamp. The imaging device may further include a microscope (or an optical train), which may or may not include an eyepiece.

Support Structure. System 150 may further comprise a support structure 190 configured to support and/or hold the enclosure 102 comprising the microfluidic circuit 120. In some embodiments, the optional tilting module 166 can be configured to activate the support structure 190 to rotate the microfluidic device 100 about one or more axes of rotation. The optional tilting module 166 can be configured to support and/or hold the microfluidic device 100 in a level orientation (i.e., at 0° relative to x- and y-axes), a vertical orientation (i.e., at 90° relative to the x-axis and/or the y-axis), or any orientation therebetween. The orientation of the microfluidic device 100 (and the microfluidic circuit 120) relative to an axis is referred to herein as the “tilt” of the microfluidic device 100 (and the microfluidic circuit 120). For example, support structure 190 can optionally be used to tilt the microfluidic device 100 (e.g., as controlled by optional tilting module 166) to 0.1°, 0.2°, 0.3°, 0.4°, 0.5°, 0.6°, 0.7°, 0.8°, 0.9°, 1°, 2°, 3°, 4°, 5°, 10°, 15°, 20°, 25°, 30°, 35°, 40°. 45°, 50°, 55°, 60°, 65°, 70°, 75°, 80°, 90° relative to the x-axis or any degree therebetween. When the microfluidic device is tilted at angles greater than about 15, tilting may be performed to create bulk movement of micro-objects into/out of sequestration pens from/into the flow region (e.g., microfluidic channel). In some embodiments, the support structure 190 can hold the microfluidic device 100 at a fixed angle of 0.1°. 0.2°. 0.3°, 0.4°, 0.5°, 0.6°, 0.7°, 0.8°, 0.9°, 1°, 2°, 3°, 4°, 5°, or 10° relative to the x-axis (horizontal), so long as DEP is an effective force to move micro-objects out of the sequestration pens into the microfluidic channel. Since the surface of the electrode activation substrate is substantially flat, DEP forces may be used even when the far end of the sequestration pen, opposite its opening to the microfluidic channel, is disposed at a position lower in a vertical direction than the microfluidic channel.

In some embodiments where the microfluidic device is tilted or held at a fixed angle relative to horizontal, the microfluidic device 100 may be disposed in an orientation such that the inner surface of the base of the flow path 106 is positioned at an angle above or below the inner surface of the base of the one or more sequestration pens opening laterally to the flow path. The term “above” as used herein denotes that the flow path 106 is positioned higher than the one or more sequestration pens on a vertical axis defined by the force of gravity (i.e., an object in a sequestration pen above a flow path 106 would have a higher gravitational potential energy than an object in the flow path), and inversely, for positioning of the flow path 106 below one or more sequestration pens. In some embodiments, the support structure 190 may be held at a fixed angle of less than about 5°, about 4°, about 3° or less than about 2° relative to the x-axis (horizontal), thereby placing the sequestration pens at a lower potential energy relative to the flow path. In some other embodiments, when long term culturing (e.g., for more than about 2, 3, 4, 5, 6, 7 or more days) is performed within the microfluidic device, the device may be supported on a culturing support and may be tilted at a greater angle of about 10°, 15°, 20°, 25°, 30°, or any angle therebetween to retain biological micro-objects within the sequestration pens during the long-term culturing period. At the end of the culturing period, the microfluidic device containing the cultured biological micro-objects may be returned to the support 190 within system 150, where the angle of tilting is decreased to values as described above, affording the use of DEP to move the biological micro-objects out of the sequestration pens. Further examples of the use of gravitational forces induced by tilting are described in U.S. Pat. No. 9,744,533 (Breinlinger et al.), the contents of which are herein incorporated by reference in its entirety.

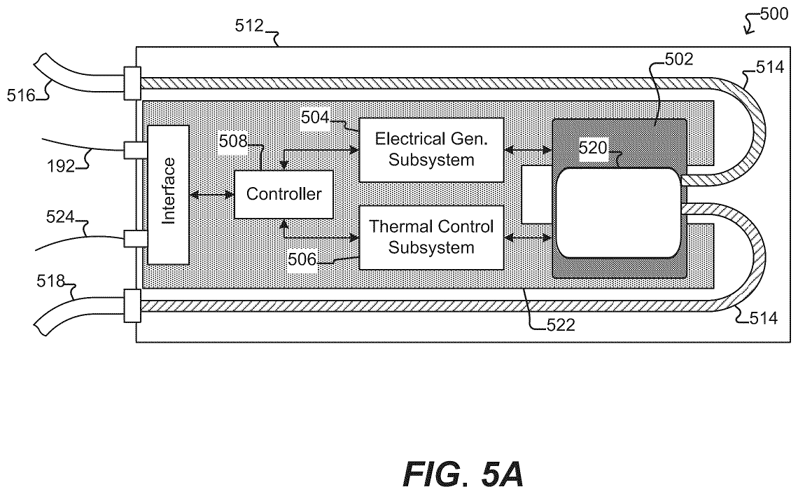

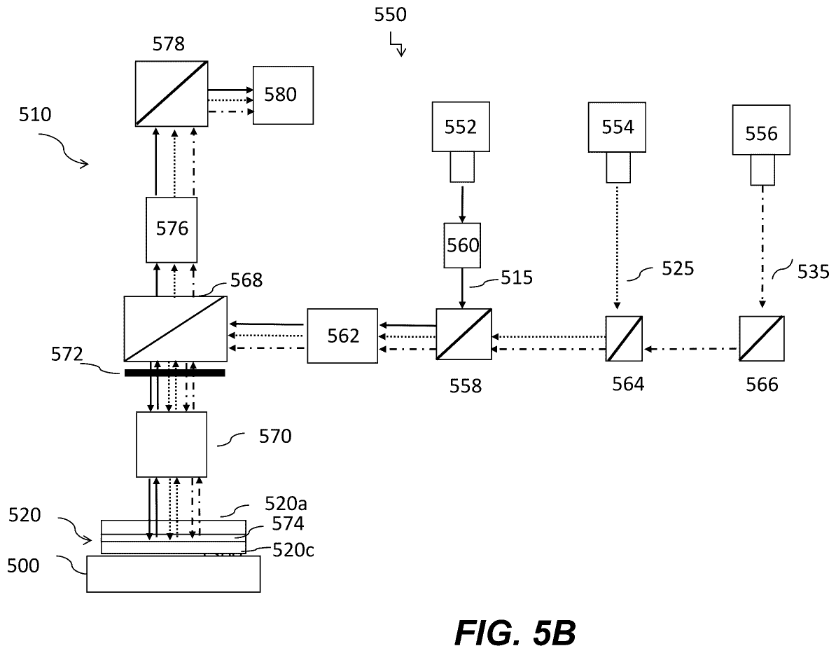

Nest. Turning now to FIG. 5A, the system 150 can include a structure (also referred to as a “nest”) 500 configured to hold a microfluidic device 520, which may be like microfluidic device 100, 200, or any other microfluidic device described herein. The nest 500 can include a socket 502 capable of interfacing with the microfluidic device 520 (e.g., an optically actuated electrokinetic device 100, 200, etc.) and providing electrical connections from power source 192 to microfluidic device 520. The nest 500 can further include an integrated electrical signal generation subsystem 504. The electrical signal generation subsystem 504 can be configured to supply a biasing voltage to socket 502 such that the biasing voltage is applied across a pair of electrodes in the microfluidic device 520 when it is being held by socket 502. Thus, the electrical signal generation subsystem 504 can be part of power source 192. The ability to apply a biasing voltage to microfluidic device 520 does not mean that a biasing voltage will be applied at all times when the microfluidic device 520 is held by the socket 502. Rather, in most cases, the biasing voltage will be applied intermittently, e.g., only as needed to facilitate the generation of electrokinetic forces, such as dielectrophoresis or electro-wetting, in the microfluidic device 520.

As illustrated in FIG. 5A, the nest 500 can include a printed circuit board assembly (PCBA) 522. The electrical signal generation subsystem 504 can be mounted on and electrically integrated into the PCBA 522. The exemplary support includes socket 502 mounted on PCBA 522, as well.

In some embodiments, the nest 500 can comprise an electrical signal generation subsystem 504 configured to measure the amplified voltage at the microfluidic device 520 and then adjust its own output voltage as needed such that the measured voltage at the microfluidic device 520 is the desired value. In some embodiments, the waveform amplification circuit can have a +6.5V to −6.5V power supply generated by a pair of DC-DC converters mounted on the PCBA 522, resulting in a signal of up to 13 Vpp at the microfluidic device 520.

In certain embodiments, the nest 500 further comprises a controller 508, such as a microprocessor used to sense and/or control the electrical signal generation subsystem 504. Examples of suitable microprocessors include the Arduino™ microprocessors, such as the Arduino Nano™. The controller 508 may be used to perform functions and analysis or may communicate with an external master controller 154 (shown in FIG. 1A) to perform functions and analysis. In the embodiment illustrated in FIG. 5A the controller 508 communicates with the master controller 154 (of FIG. 1A) through an interface (e.g., a plug or connector).

As illustrated in FIG. 5A, the support structure 500 (e.g., nest) can further include a thermal control subsystem 506. The thermal control subsystem 506 can be configured to regulate the temperature of microfluidic device 520 held by the support structure 500. For example, the thermal control subsystem 506 can include a Peltier thermoelectric device (not shown) and a cooling unit (not shown). In the embodiment illustrated in FIG. 5A, the support structure 500 comprises an inlet 516 and an outlet 518 to receive cooled fluid from an external reservoir (not shown) of the cooling unit, introduce the cooled fluid into the fluidic path 514 and through the cooling block, and then return the cooled fluid to the external reservoir. In some embodiments, the Peltier thermoelectric device, the cooling unit, and/or the fluidic path 514 can be mounted on a casing 512 of the support structure 500. In some embodiments, the thermal control subsystem 506 is configured to regulate the temperature of the Peltier thermoelectric device so as to achieve a target temperature for the microfluidic device 520. Temperature regulation of the Peltier thermoelectric device can be achieved, for example, by a thermoelectric power supply, such as a Pololu™ thermoelectric power supply (Pololu Robotics and Electronics Corp.). The thermal control subsystem 506 can include a feedback circuit, such as a temperature value provided by an analog circuit. Alternatively, the feedback circuit can be provided by a digital circuit.

The nest 500 can include a serial port 524 which allows the microprocessor of the controller 508 to communicate with an external master controller 154 via the interface. In addition, the microprocessor of the controller 508 can communicate (e.g., via a Plink tool (not shown)) with the electrical signal generation subsystem 504 and thermal control subsystem 506. Thus, via the combination of the controller 508, the interface, and the serial port 524, the electrical signal generation subsystem 504 and the thermal control subsystem 506 can communicate with the external master controller 154. In this manner, the master controller 154 can, among other things, assist the electrical signal generation subsystem 504 by performing scaling calculations for output voltage adjustments. A Graphical User Interface (GUI) (not shown) provided via a display device 170 coupled to the external master controller 154, can be configured to plot temperature and waveform data obtained from the thermal control subsystem 506 and the electrical signal generation subsystem 504, respectively. Alternatively, or in addition, the GUI can allow for updates to the controller 508, the thermal control subsystem 506, and the electrical signal generation subsystem 504.

Optical sub-system. FIG. 5B is a schematic of an optical sub-system 550 having an optical apparatus 510 for imaging and manipulating micro-objects in a microfluidic device 520, which can be any microfluidic device described herein. The optical apparatus 510 can be configured to perform imaging, analysis and manipulation of one or more micro-objects within the enclosure of the microfluidic device 520.

The optical apparatus 510 may have a first light source 552, a second light source 554, and a third light source 556. The first light source 552 can transmit light to a structured light modulator 560, which can include a digital mirror device (DMD) or a microshutter array system (MSA), either of which can be configured to receive light from the first light source 552 and selectively transmit a subset of the received light into the optical apparatus 510. Alternatively, the structured light modulator 560 can include a device that produces its own light (and thus dispenses with the need for a light source 552), such as an organic light emitting diode display (OLED), a liquid crystal on silicon (LCOS) device, a ferroelectric liquid crystal on silicon device (FLCOS), or a transmissive liquid crystal display (LCD). The structured light modulator 560 can be, for example, a projector. Thus, the structured light modulator 560 can be capable of emitting both structured and unstructured light. In certain embodiments, an imaging module and/or motive module of the system can control the structured light modulator 560.

In embodiments when the structured light modulator 560 includes a mirror, the modulator can have a plurality of mirrors. Each mirror of the plurality of mirrors can have a size of about 5 microns×5 microns to about 10 microns×10 microns, or any values therebetween. The structured light modulator 560 can include an array of mirrors (or pixels) that is 2000×1000, 2580×1600, 3000×2000, or any values therebetween. In some embodiments, only a portion of an illumination area of the structured light modulator 560 is used. The structured light modulator 560 can transmit the selected subset of light to a first dichroic beam splitter 558, which can reflect this light to a first tube lens 562.

The first tube lens 562 can have a large clear aperture, for example, a diameter larger than about 40 mm to about 50 mm, or more, providing a large field of view. Thus, the first tube lens 562 can have an aperture that is large enough to capture all (or substantially all) of the light beams emanating from the structured light modulator 560.

The structured light 515 having a wavelength of about 400 nm to about 710 nm, may alternatively or in addition, provide fluorescent excitation illumination to the microfluidic device.

The second light source 554 may provide unstructured brightfield illumination. The brightfield illumination light 525 may have any suitable wavelength, and in some embodiments, may have a wavelength of about 400 nm to about 760 nm. The second light source 554 can transmit light to a second dichroic beam splitter 564 (which also may receive illumination light 535 from the third light source 556), and the second light, brightfield illumination light 525, may be transmitted therefrom to the first dichroic beam splitter 558. The second light, brightfield illumination light 525, may then be transmitted from the first dichroic beam splitter 558 to the first tube lens 562.

The third light source 556 can transmit light through a matched pair relay lens (not shown) to a mirror 566. The third illumination light 535 may therefrom be reflected to the second dichroic beam splitter 564 and be transmitted therefrom to the first beam splitter 558, and onward to the first tube lens 562. The third illumination light 535 may be a laser and may have any suitable wavelength. In some embodiments, the laser illumination 535 may have a wavelength of about 350 nm to about 900 nm. The laser illumination 535 may be configured to heat portions of one or more sequestration pens within the microfluidic device. The laser illumination 535 may be configured to heat fluidic medium, a micro-object, a wall or a portion of a wall of a sequestration pen, a metal target disposed within a microfluidic channel or sequestration pen of the microfluidic channel, or a photoreversible physical barrier within the microfluidic device, and described in more detail in U. S. Application Publication Nos. 2017/0165667 (Beaumont, et al.) and 2018/0298318 (Kurz, et al.), each of which disclosure is herein incorporated by reference in its entirety. In other embodiments, the laser illumination 535 may be configured to initiate photocleavage of surface modifying moieties of a modified surface of the microfluidic device or photocleavage of moieties providing adherent functionalities for micro-objects within a sequestration pen within the microfluidic device. Further details of photocleavage using a laser may be found in International Application Publication No. WO2017/205830 (Lowe, Jr. et al.), which disclosure is herein incorporated by reference in its entirety.

The light from the first, second, and third light sources (552, 554, 556) passes through the first tube lens 562 and is transmitted to a third dichroic beam splitter 568 and filter changer 572. The third dichroic beam splitter 568 can reflect a portion of the light and transmit the light through one or more filters in the filter changer 572 and to the objective 570, which may be an objective changer with a plurality of different objectives that can be switched on demand. Some of the light (515, 525, and/or 535) may pass through the third dichroic beam splitter 568 and be terminated or absorbed by a beam block (not shown). The light reflected from the third dichroic beam splitter 568 passes through the objective 570 to illuminate the sample plane 574, which can be a portion of a microfluidic device 520 such as the sequestration pens described herein.

The nest 500, as described in FIG. 5A, can be integrated with the optical apparatus 510 and be a part of the apparatus 510. The nest 500 can provide electrical connection to the enclosure and be further configured to provide fluidic connections to the enclosure. Users may load the microfluidic apparatus 520 into the nest 500. In some other embodiments, the nest 500 can be a separate component independent of the optical apparatus 510.

Light can be reflected off and/or emitted from the sample plane 574 to pass back through the objective 570, through the filter changer 572, and through the third dichroic beam splitter 568 to a second tube lens 576. The light can pass through the second tube lens 576 (or imaging tube lens 576) and be reflected from a mirror 578 to an imaging sensor 580. Stray light baffles (not shown) can be placed between the first tube lens 562 and the third dichroic beam splitter 568, between the third dichroic beam splitter 568 and the second tube lens 576, and between the second tube lens 576 and the imaging sensor 580.

Objective. The optical apparatus can comprise the objective lens 570 that is specifically designed and configured for viewing and manipulating of micro-objects in the microfluidic device 520. For example, conventional microscope objective lenses are designed to view micro-objects on a slide or through 5 mm of aqueous fluid, while micro-objects in the microfluidic device 520 are inside the plurality of sequestration pens within the viewing plane 574 which have a depth of 20, 30, 40, 50, 60 70, 80 microns or any values therebetween. In some embodiments, a transparent cover 520a, for example, glass or ITO cover with a thickness of about 750 microns, can be placed on top of the plurality of sequestration pens, which are disposed above a microfluidic substrate 520c. Thus, the images of the micro-objects obtained by using the conventional microscope objective lenses may have large aberrations such as spherical and chromatic aberrations, which can degrade the quality of the images. The objective lens 570 of the optical apparatus 510 can be configured to correct the spherical and chromatic aberrations in the optical apparatus 510. The objective lens 570 can have one or more magnification levels available such as, 4X, 10X, 20X.

Modes of illumination. In some embodiments, the structured light modulator 560 can be configured to modulate light beams received from the first light source 552 and transmits a plurality of illumination light beams 515, which are structured light beams, into the enclosure of the microfluidic device, e.g., the region containing the sequestration pens. The structured light beams can comprise the plurality of illumination light beams. The plurality of illumination light beams can be selectively activated to generate a plurality of illuminations patterns. In some embodiments, the structured light modulator 560 can be configured to generate an illumination pattern, similarly as described for FIGS. 4A-4B, which can be moved and adjusted. The optical apparatus 510 can further comprise a control unit (not shown) which is configured to adjust the illumination pattern to selectively activate the one or more of the plurality of DEP electrodes of a substrate 520c and generate DEP forces to move the one or more micro-objects inside the plurality of sequestration pens within the microfluidic device 520. For example, the plurality of illuminations patterns can be adjusted over time in a controlled manner to manipulate the micro-objects in the microfluidic device 520. Each of the plurality of illumination patterns can be shifted to shift the location of the DEP force generated and to move the structured light for one position to another in order to move the micro-objects within the enclosure of the microfluidic apparatus 520.

In some embodiments, the optical apparatus 510 may be configured such that each of the plurality of sequestration pens in the sample plane 574 within the field of view is simultaneously in focus at the image sensor 580 and at the structured light modulator 560. In some embodiments, the structured light modulator 560 can be disposed at a conjugate plane of the image sensor 580. In various embodiments, the optical apparatus 510 can have a confocal configuration or confocal property. The optical apparatus 510 can be further configured such that only each interior area of the flow region and/or each of the plurality of sequestration pens in the sample plane 574 within the field of view is imaged onto the image sensor 580 in order to reduce overall noise to thereby increase the contrast and resolution of the image.

In some embodiments, the first tube lens 562 can be configured to generate collimated light beams and transmit the collimated light beams to the objective lens 570. The objective 570 can receive the collimated light beams from the first tube lens 562 and focus the collimated light beams into each interior area of the flow region and each of the plurality of sequestration pens in the sample plane 574 within the field of view of the image sensor 580 or the optical apparatus 510. In some embodiments, the first tube lens 562 can be configured to generate a plurality of collimated light beams and transmit the plurality of collimated light beams to the objective lens 570. The objective 570 can receive the plurality of collimated light beams from the first tube lens 562 and converge the plurality of collimated light beams into each of the plurality of sequestration pens in the sample plane 574 within the field of view of the image sensor 580 or the optical apparatus 510.

In some embodiments, the optical apparatus 510 can be configured to illuminate the at least a portion of sequestration pens with a plurality of illumination spots. The objective 570 can receive the plurality of collimated light beams from the first tube lens 562 and project the plurality of illumination spots, which may form an illumination pattern, into each of the plurality of sequestration pens in the sample plane 574 within the field of view. For example, each of the plurality of illumination spots can have a size of about 5 microns×5 microns; 10 microns×10 microns; 10 microns×30 microns, 30 microns×60 microns, 40 microns×40 microns, 40 microns×60 microns, 60 microns×120 microns, 80 microns×100 microns, 100 microns×140 microns and any values there between. The illumination spots may individually have a shape that is circular, square, or rectangular. Alternatively, the illumination spots may be grouped within a plurality of illumination spots (e.g., an illumination pattern) to form a larger polygonal shape such as a rectangle, square, or wedge shape. The illumination pattern may enclose (e.g., surround) an unilluminated space that may be square, rectangular or polygonal. For example, each of the plurality of illumination spots can have an area of about 150 to about 3000, about 4000 to about 10000, or 5000 to about 15000 square microns. An illumination pattern may have an area of about 1000 to about 8000, about 4000 to about 10000, 7000 to about 20000, 8000 to about 22000, 10000 to about 25000 square microns and any values there between.

The optical system 510 may be used to determine how to reposition micro-objects and into and out of the sequestration pens of the microfluidic device, as well as to count the number of micro-objects present within the microfluidic circuit of the device. Further details of repositioning and counting micro-objects are found in U. S. Application Publication No. 2016/0160259 (Du); U.S. Pat. No. 9,996,920 (Du et al.); and International Application Publication No. WO2017/102748 (Kim, et al.). The optical system 510 may also be employed in assay methods to determine concentrations of reagents/assay products, and further details are found in U.S. Pat. No. 8,921,055 (Chapman), U.S. Pat. No. 10,010,882 (White et al.), and U.S. Pat. No. 9,889,445 (Chapman et al.); International Application Publication No. WO2017/181135 (Lionberger, et al.); and International Application Serial No. PCT/US2018/055918 (Lionberger, et al.). Further details of the features of optical apparatuses suitable for use within a system for observing and manipulating micro-objects within a microfluidic device, as described herein, may be found in WO2018/102747 (Lundquist, et al), the disclosure of which is herein incorporated by reference in its entirety.

Additional system components for maintenance of viability of cells within the sequestration pens of the microfluidic device. In order to promote growth and/or expansion of cell populations, environmental conditions conducive to maintaining functional cells may be provided by additional components of the system. For example, such additional components can provide nutrients, cell growth signaling species, pH modulation, gas exchange, temperature control, and removal of waste products from cells.

### VII. EXAMPLES

System and device: An OptoSelect™ device, a nanofluidic device manufactured by Berkeley Lights, Inc. and controlled by an optical instrument which was also manufactured by Berkeley Lights, Inc. were employed. The instrument includes: a mounting stage for the chip coupled to a temperature controller; a pump and fluid medium conditioning component; and an optical train including a camera and a structured light source suitable for activating phototransistors within the chip. The OptoSelect device includes a substrate configured with OptoElectroPositioning (OEP™) technology, which provides a phototransistor-activated OET force. The chip also included a plurality of microfluidic channels, each having a plurality of NanoPen™ chambers (or chambers) fluidically connected thereto. The volume of each chamber is around 1×106 cubic microns.

Biological cells. CHO cells engineered to express a human antibody were used. Cell numbers and viability were counted and cell density was adjusted to 5×105/ml for loading the cells onto the OptoSelect device.

Device priming. 250 microliters of 100% carbon dioxide is flowed in to the OptoSelect device at a rate of 12 microliters/sec, followed by 250 microliters of PBS containing 0.1% Pluronic® F27 (Life Technologies® Cat #P6866) flowed in at 12 microliters/sec, and finally 250 microliters of PBS flowed in at 12 microliters/sec. Introduction of the culture medium follows.

Media: CD CHO medium (ThermoFisher Scientific Cat. #10743029), a commercially available protein-free and serum-free medium, chemically defined medium was used.

Media perfusion during culture. Medium is perfused through the OptoSelect device according to either of the following two methods: (1) Perfuse at 0.01 microliters/sec for 2 h; perfuse at 2 microliters/sec for 64 sec; and repeat. (2) Perfuse at 0.02 microliters/sec for 100 sec; stop flow 500 sec; perfuse at 2 microliters/sec for 64 sec; and repeat.

### Example 1: Assessing the Relative Production of an Antibody Using a Peptide Reporter Molecule

Reporter molecule. An IgG binding peptide having a molecular weight of 2.4 Kd, N-terminally labeled with HiLyte Fluor™ 555 NHS ester (AnaSpec Inc., Cat. #AS-81251, 869da (MW of free acid), Ex/Em 550/566 nm (Cy3 filter)).

Dark Reference image collection: Prior to introduction of cells, the OptoSelect device was imaged first with no medium nor reporter molecule present, obtaining the Dark Reference image used in a process as described herein which removes background and normalizes the image of each NanoPen chamber.

Signal Reference image collection: Culture medium containing the N-terminally labeled HiLyte Fluor™ 555 IgG binding peptide (reporter molecule) at a concentration of 1 microgram/ml was flowed into the OptoSelect device for 45 min at 0.005 microliters/sec until the fluorescent compound diffused and achieved an equilibrated distribution between the NanoPen chambers and the microfluidic channel. The Signal Reference image was acquired at that time. The OptoSelect device was then flushed with culture medium at 0.03 microliters/sec having no reporter molecule for 25 min. This period of flushing ensured that the reporter molecules had substantially completely diffused out of the NanoPen chamber, leaving none or insignificant amounts of reporter molecules remaining within the NanoPen chambers. The Signal Reference image may alternatively be obtained by flowing the fluorescent dye itself at the same molar concentration, and does not require that fluorescently labeled reporter molecule be employed.

Introducing secreting cells into the microfluidic device. CHO cells were introduced into the OptoSelect device and selectively placed into the Nanopen chambers using dielectrophoretic forces of the OEP technology of the device. The cells were disposed one cell per NanoPen chamber. Culture medium was perfused as above, for a period of 6 days. Brightfield images were taken daily to record cell expansion within each NanoPen chamber. The selection of a 6-day culture period prior to a first assay may be varied depending on the particular requirements of the biological cells and secreted analyte. It may be desired to assay (which may include a brightfield image) each day of an extended culture period, or one or more assays may be performed on selected days during the culturing period.

Assay Signal Collection. As an initial step of the assay, a brightfield image was obtained to correlate cell number and position within each NanoPen chamber. After collection of the brightfield image, the fluorescent reporter molecule at a concentration of 1 microgram/ml was flowed into the microfluidic channel 0.05 microliters/sec for a period of 45 min, affording sufficient time for reporter molecule to diffuse fully into each NanoPen chamber. After introduction of the reporter molecule to the NanoPen chamber, flow of culture medium containing no fluorescent reporter molecule was resumed at 0.03 microliters/sec for a period of 25 minutes, based on the diffusion rate as determined above. A fluorescence image was obtained. The assay may be repeated if desired, over additional periods of culturing/expansion as determined to be suitable for the particular cells and/or secreted analyte therefrom.

Determination of relative production of analyte. An area of interest (AOI) along the axis of diffusion from within the NanoPen chamber was identified within each Nanopen chamber which encompasses an area of about 20 pixels wide and 200 pixels in length, where the lower (first) end of the AOI was chosen to be within the isolation region at a selected distance from the base of the NanoPen chamber distal to the opening into the microfluidic channel, where no cells were disposed. The second (upper end) of the length of the AOI was selected to be within the microfluidic channel itself, which ensured that the pixels residing within the AOI and within the channel substantially have no signal. The width of the AOI is centered along a trajectory of anticipated diffusion from the isolation region of the NanoPen chamber out to the channel of the OptoSelect Device. As described herein, the AOI was sub-divided into 20 sub-regions (bins), each having a width of 20 pixels and a length along the anticipated diffusion trajectory of 10 pixels.

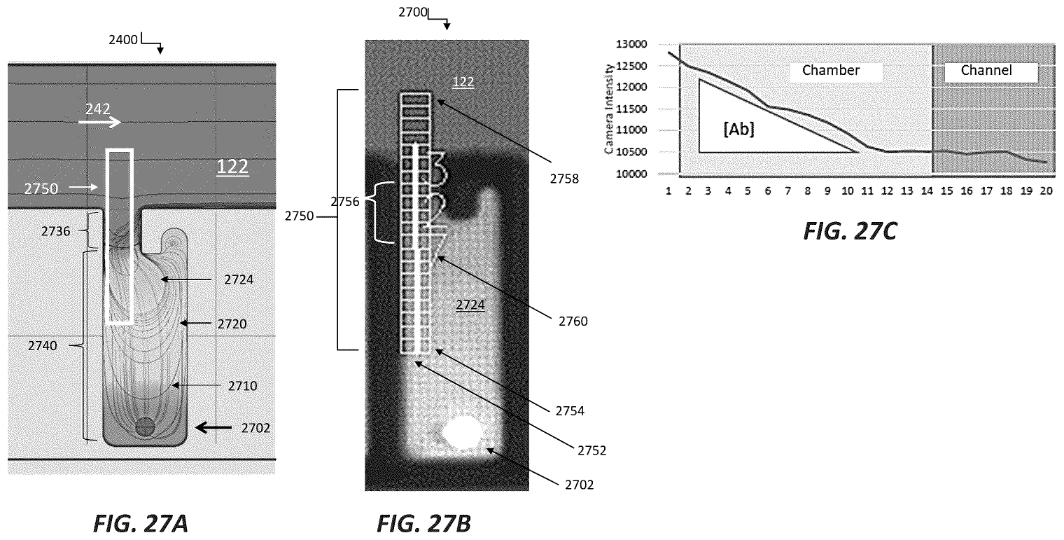

FIG. 27A shows a schematic representation of an AOI from which data is extracted for the determination of the relative or absolute amount of a secreted analyte from a biological micro-object. The AOI 2750 is selected to encompass: a region in the isolation region 2740, region in the connection region 2736 (of chamber 2724 in microfluidic device 2700); and a portion of the channel 122, all of which is aligned along the axis of diffusion from the chamber 2724 to the channel 122. In this embodiment, flow 242 is present in the microfluidic channel 122, reducing any detectable signal within the portion of the channel incorporated within the AOI. The selection of the point at which the AOI ends within the chamber is made to prevent overlap with the biological object 2702 which secretes the analyte, and from which detectable signal emanates. As shown in FIG. 27A, lines of diffusion 2710 are directed towards the connection region 2736 and become aligned with the axis of diffusion as the connection region 2736 is entered. Concentration gradient lines 2720 are shown as well.

FIG. 27B is a photograph showing the Assay Image for a chamber 2724, having an identification number 2760 of “327”, which indicates its location within the microfluidic device 2700. The identification number assists in correlating brightfield and fluorescence image locations, and also assists users to select, manipulate and export cells from a selected chamber. Chamber 2724 of FIG. 27B has one biological micro-object 2702 present within the isolation region (not labelled). The Assay Image clearly shows extensive amount of fluorescence signal within the chamber, emanating from biological micro-object 2702. The AOI 2750 is shown photographically imposed, and is aligned along the axis of diffusion and centered along the line of diffusion trajectory 2752. The AOI is 20 pixels wide, which is chosen depending on the width of the connection region 126 (not labelled in FIG. 27B) and is divided into 20 sub-regions. An AOI may have other pixel sizes to each sub-region and the number of sub-regions may vary from about 1 to about 50. The sub-region 2754 of the AOI is the sub-region located furthest away from the channel 122 of all the sub-regions of the AOI, but is selected to not overlap with the biological micro-object 2702. The sub-region at the second end of the AOI is sub-region 2758, which is located within the channel 122. Importantly, the group of sub-regions 2756 is the Cell position insensitive region, from which the detected fluorescence is used to assess the relative or absolute amount of a secreted analyte of a biological micro-object within a chamber.

FIG. 27C shows a graphical representation of the fluorescence detected in the AOI, where the values on the horizontal axis represent sub-regions 1 (corresponding to sub-region 2754 of FIG. 27B), the sub-region at the most proximal end of the AOI to the biological micro-object 2702, and sub-region 20 corresponds to the sub-region 2758 of FIG. 27B, at the most proximal end of the AOI in the channel 122. The amount of detected fluorescence in the AOI is proportional to the amount of secreted analyte.

The fluorescent Assay image was normalized/calibrated as described herein using the Dark Reference and the Signal Reference images to reduce system errors, and roll off of signal image due to imperfect illumination of the field of view. Each pixel in the AOI is processed as:

$$Normalized Assay value = \frac{Assay intensity value - Dark Reference}{Signal Reference value - Dark Reference}$$

The median intensity for each of the 20 sub-regions was determined by adding the signal intensities for each pixel in the sub-region. A representation of the curve resulting from plotting the normalized median intensity values for each sub-region, is shown in FIG. 27B, where the x axis lists sub-regions 1 to 20. Sub-region 1 is the sub-region of the AOI most distal from the channel, and sub-region 20 is the sub-region of the AOI that is furthest into the channel. A linear regression was performed upon the section of the curve plotting the normalized median intensity values for sub-regions 9-13 (region 2756, in FIG. 27B). As described above, these sub-regions were determined to be within the region where the signal intensity observed was insensitive to the position of the biological cells within the lower (most distal from the channel) portion of the isolation region of the NanoPen chamber. The value of the slope obtained from this operation was used as a score, in arbitrary units (A.U.). Larger slopes (score) indicated greater secretion of analyte by the cells within that NanoPen chamber.

An identification number and a score may be included in images for each of the NanoPen chambers and may be correlated in memory as well as the image. Either raw scores or cell-count-corrected scores may be used to more easily rank NanoPen chambers to assist in deciding on which NanoPen chambers to be further examined in the course of the effort to develop highly productive cell lines. Other methods of calculating a rate of concentration change from within the NanoPen chamber to the channel may also be employed such as area under the curve or other methods described herein to quantify the level of production of secreted analyte within each NanoPen chamber.

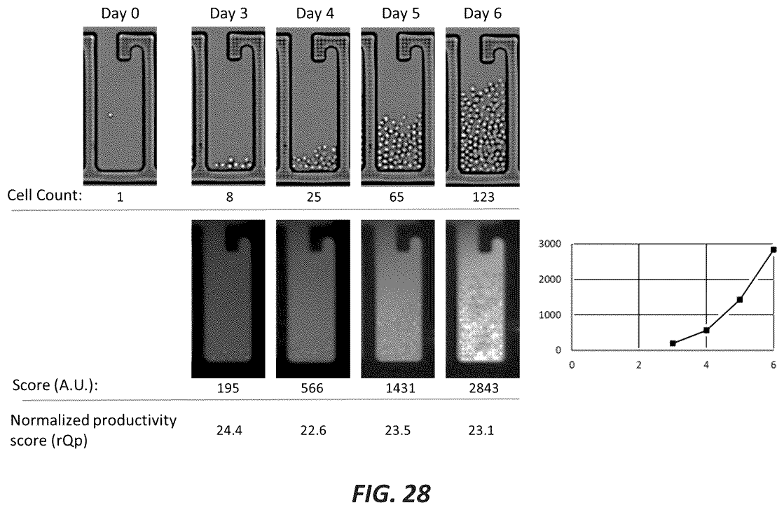

Measure of relative productivity. Scores may be corrected for the number of cells per NanoPen chamber, as shown in FIG. 28. In this experiment, the cell type, media, reporter molecule, pre-culturing image acquisition, culturing conditions, and assay conditions were the same as above. FIG. 28 shows images of a single NanoPen chamber for which a brightfield image was acquired at day 0, and on days 3, 4, 5, 6, as shown in the images in the top row of FIG. 28. Additionally, an assay as described above was performed on each of days 3, 4, 5, 6 and the fluorescent images for each of days 3, 4, 5, and 6 for the same single NanoPen chamber are shown aligned under the corresponding brightfield image for that day. The brightfield image was used to count the number of cells present, which may be performed in an automated process, showing for the selected NanoPen chamber: day 0 (1 cell); day 3 (8 cells); day 4 (25 cells); day 5 (65 cells); day 6 (123 cells), as the clonal population expanded. The assay scores, obtained as described above (representing the negative slope) steadily increased, as well, day 3(195 A.U.); day 4 (566 A.U.); day 5 (1431 A.U.); day 6 (2842). Accordingly, on day 3, eight cells in the NanoPen chamber resulted in a score of 195 (A.U.). On day 4, the same NanoPen chamber now had 25 cells, resulting in a score of 566 (A.U.). On day 5, the same NanoPen chamber had 65 cells, resulting in a score of 1431 (A.U.). Finally, on day 6, the same NanoPen chamber had 123 cells, resulting in a score of 2843 (A.U.). The graph of FIG. 28 shows the assay scores (y-axis) plotted against the assay timepoint in days (x-axis) since the start of cell culture within the NanoPen chamber. The absolute scores were divided by the number of cells present at that timepoint to provide a score normalized to the number of cells in the chamber. This yielded a normalized measure of productivity (rQp) of cells in the selected NanoPen chamber, which remained in a range between 22.6 to 24.1 A.U. per cell.

### Example 2. Assessing the Production of a Bispecific Antibody

In this experiment, four different pre-clonal cell lines were cultured on-chip. The four cell lines were expected to produce a bispecific antibody binding to both antigen I and antigen G at a stoichiometry of 1:1. At least two reporter molecules were used in this experiment to evaluate the production of the target bispecific antibody of the four cell lines.

System, device, and media: as described above.

Reporter molecules: (1) Antigen I, 17 kDa; (2) Antigen G, 15 kDa; (3) SpotLight Kappa, a fluorescent label that binds to the conserved region of the kappa light chain of the Fab arm that is able to bind to antigen I.

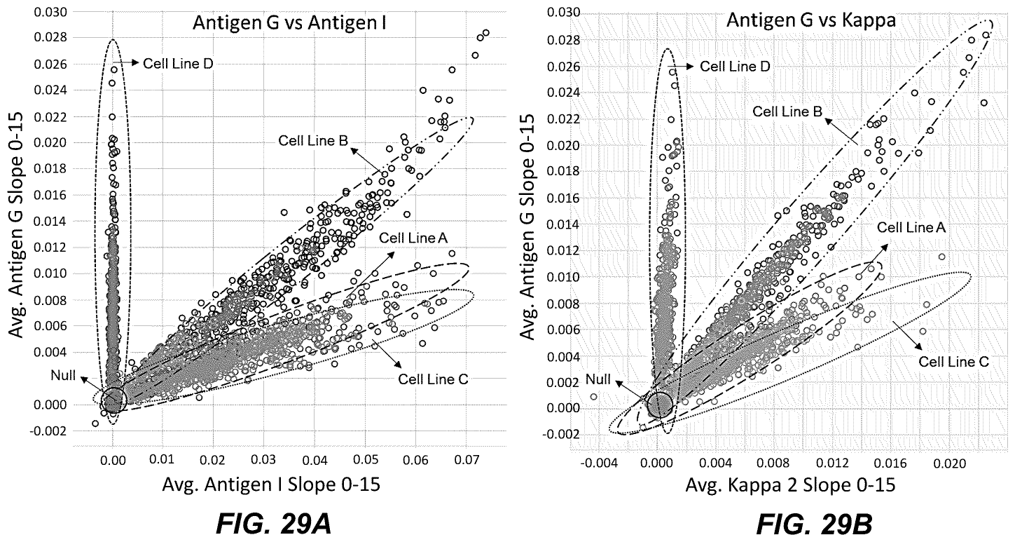

The reporter molecules were detected after a steady state equilibrium was reached. The scores of the detection were determined and the absolute quantitation values thereof were calculated as described above. FIGS. 29A-B shows the resulting comparison between the scores of antigen G vs. antigen I (FIG. 29A) and antigen G vs. SpotLight Kappa (FIG. 29B). In the antigen G vs. antigen I comparison, (FIG. 29A), both antigen G and antigen I were detected in Cell Line B. Both antigen G and antigen I were also detected in Cell Lines A and C while the intensity of antigen G was lower. Only antigen G was detected in Cell Line D, and the negative control group showed no detection of both antigen G and antigen I. The antigen G vs. SpotLight Kappa comparison (FIG. 29B) showed similar results. Together, FIGS. 29A-B suggested that three subpopulations detected have different antigen G to antigen I ratio (See the dots being circled separately into three groups in FIG. 29A).

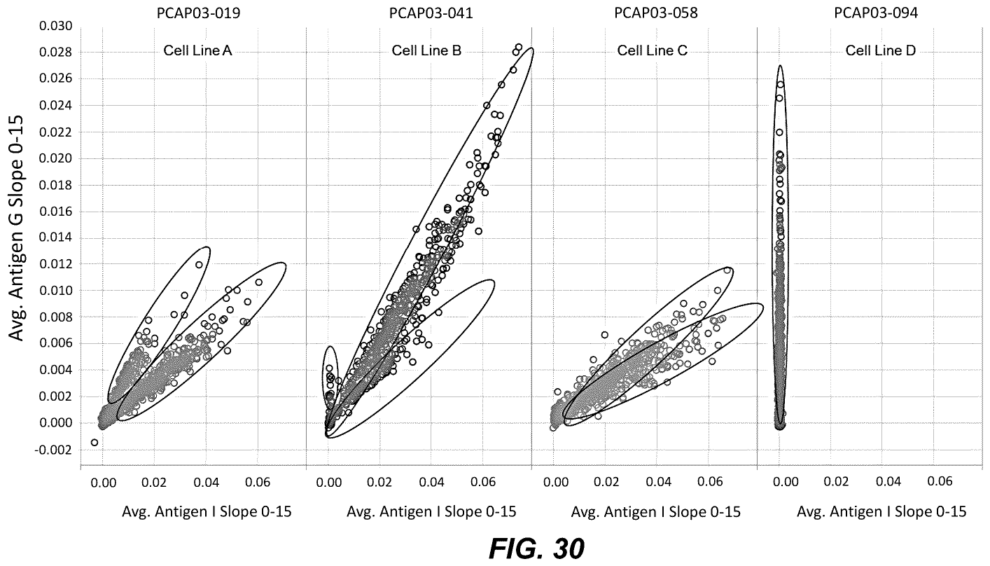

Moreover, based on the detection of antigen G and antigen I, several subpopulations can also be identified among each cell line. See FIG. 30. Two subpopulations were identified in Cell Line A; three subpopulations were identified in Cell Line B; two subpopulations were identified in Cell Line C (the distribution of these two subpopulations was closely overlapped suggesting there could be only one population); and one population was identified in Cell Line D. A follow-up off-chip experiment confirmed that Cell Line B had about 87% heterodimers, suggesting the observation in FIG. 29A was a consistent observation of correct assembly of the bispecific antibody.

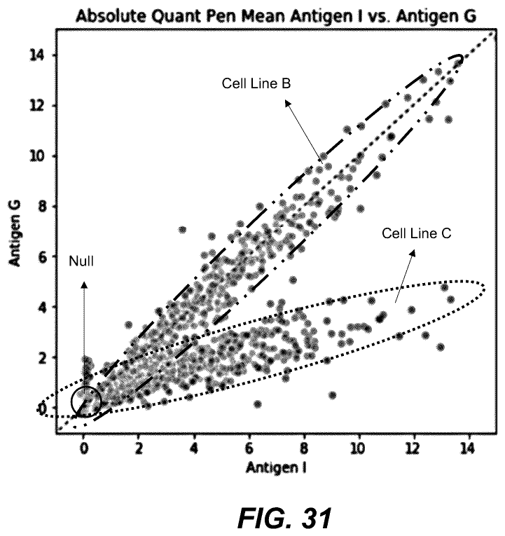

Furthermore, the scores of the detection of Cell Line B and Cell Line C were calibrated according to the formula I described above to obtain the absolute quantitation values of antigen G and antigen I detections. The comparison of the absolute quantitation values is shown in FIG. 31. Because FIG. 31 illustrates analysis of the absolute quantitation values, it is meaningful to compare the ratio of the intensity of antigen G and antigen I with the pre-selected stoichiometry of the Fab arms of the bispecific antibody. The slope of the distribution of Cell Line B was 1, which matched the pre-selected stoichiometry and verified that the antibodies produced by Cell Line B were correctly assembled. In comparison, although Cell Line C also exhibited both antigen G intensity and antigen I intensity, the ratio did not match the pre-selected stoichiometry suggesting the antibodies produced were not correctly assembled.

### Example 3. Conducting Diffusion Assay and Aggregation Assay Sequentially

System and device: as described above.

Cells: CHO cells as described above.

Media: as described above.

Reporter molecule: An IgG binding peptide having a molecular weight of 2.4 Kd, N-terminally labeled with HiLyte Fluor™ 555 NHS ester (AnaSpec Inc., Cat. #AS-81251, 869da (MW of free acid), Ex/Em 550/566 nm (Cy3 filter)).

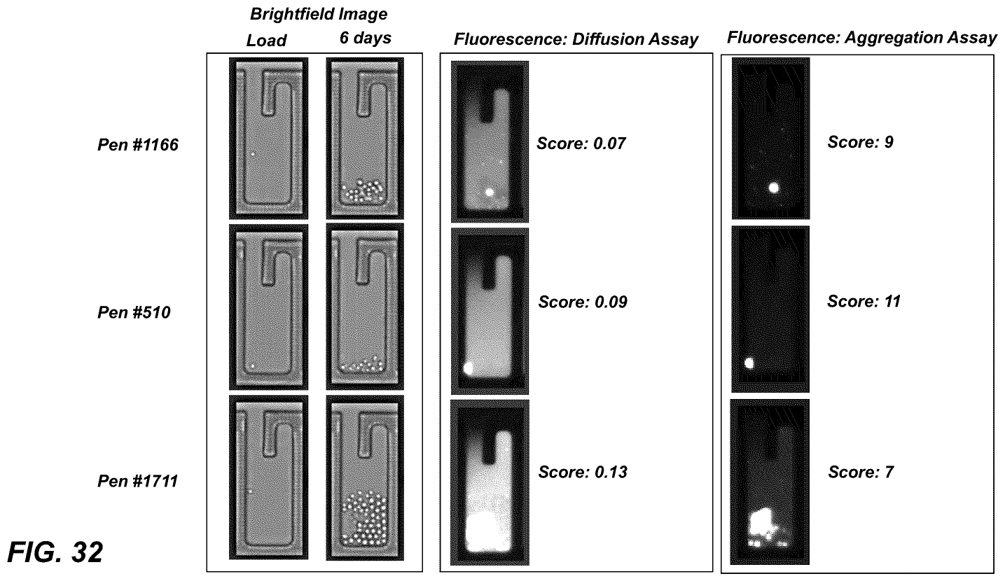

Culturing was performed for 6 days. As shown in the brightfield image in FIG. 32, cells were successfully expanded within the pens (only Pen #1166, #510, and #1711 are shown). The diffusion assay using a HiLyte Fluor™ 555 labeled IgG binding peptide having a molecular weight of 2.4 Kd was performed as in Example 1. Analysis to assign a score based on the intensities of signal observed within the AOI as defined herein was performed. Scores of the diffusion assay were assessed for each Nanopen chamber within the OptoSelect device. Pen #1166, #510, and #1711 scored 0.07, 0.09, and 0.13 respectively.

An aggregation assay was then performed. The microfluidic device was flowed with fresh media to facilitate the free and soluble bound reporter molecules to diffuse out of the pens for reducing background noise. Then, AOI was determined by excluding cell area using a pixel mask, and punctate regions were determined by CNN and the number thereof counted to generate the scoring of Pen #1166, #510, and #1711 of 9, 11, and 7, respectively. The scores were obtained using an aggregation detection algorithm based on the determination of the punctate regions. These scores represent the number of the punctate regions identified, meaning the higher the score, the more the punctate regions detected. By considering the scores of the diffusion assay and the scores of aggregation assay, the user can select or deselect any pen on the microfluidic device.

Alternatively, several images can be obtained along the culture time as well as during the diffusion assay and the aggregation assay are performing. In this way, additional information can be collected. For instance, cell expansion within each pen can be monitored; or, whether the background noise is reduced sufficiently can be observed. In other alternatives, no flow of media containing no reporter molecules is performed, and images are obtained having background levels of soluble reporter molecule and the soluble RMSA within the AOI. The images may be filtered to reduce the level of background label detection to still permit identification and scoring of punctate regions in the pens.

Further variation: It is not required to perform a diffusion assay immediately prior to performing an aggregation assay. Once the reporter molecule has been introduced, permitted to diffuse into the pens, bind to product molecules, and come to an equilibrated labeled state, the aggregation assay images may be obtained, either immediately or after a period of media flow (containing no reporter molecules).

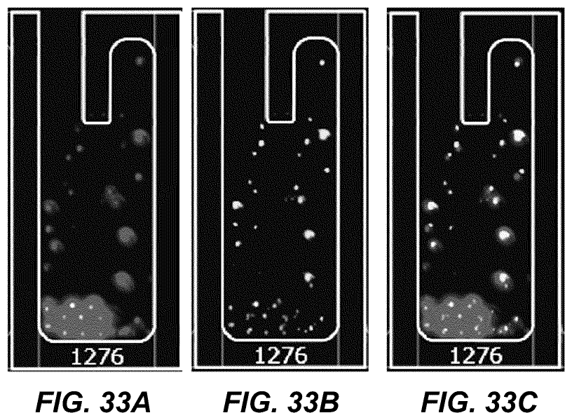

Additional experiments were conducted to verify the fluorescent punctate regions observed in the aggregation assay were representative of the quality of secreted proteins. First of all, the cultured CHO cells were stained with both SpotLight Human Fc. e.g. Spotlight Hu Fc, a fluorescent label binding to the Fc region of antibodies, and anti-human Fc Fab (Jackson ImmunoResearch #109-546-170). FIG. 33A is the image of SpotLight Human Fc staining (Texas Red), FIG. 33B is the image of anti-human Fc Fab staining (Alexa Fluor) respectively, and FIG. 33C is an overlay image thereof. The overlay of fluorescent images shows the two labels co-localize to the same localized regions, e.g., spots, verifying that the punctates observed in the aggregation assay were not formed by the reporter molecules (Spotlight Hu Fc) alone.

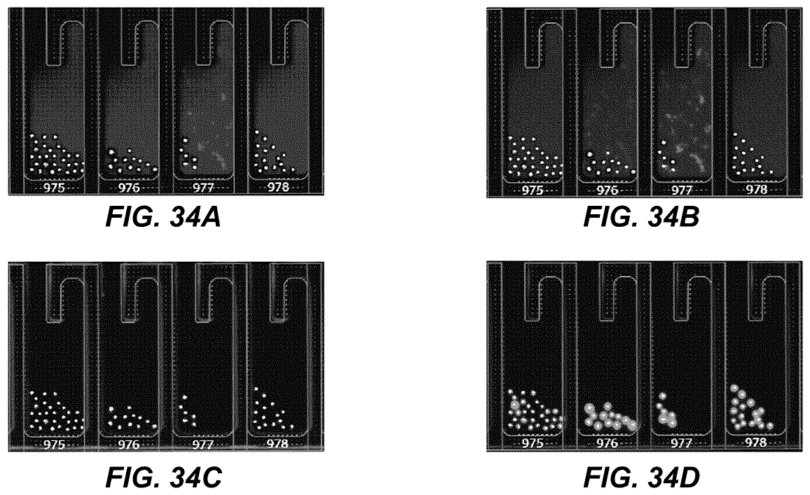

Moreover, the binding of the reporter molecules to the secreted proteins or aggregation thereof was verified to be specific. As shown in FIGS. 34A to 34D, the cultured CHO cells were stained with SpotLight Human Fc (Texas Red. FIG. 34A), anti-human Fc Fab (Jackson ImmunoResearch #109-546-170) (Alexa Fluor, FIG. 34B), a FITC labelled anti-canine Ig antibody with no cross reactivity with human IgG (FIG. 34C), and a lipophilic fluorescent label ANS (anilinonapthalene sulfonate) (FIG. 34D). FIG. 34A and FIG. 34B show positive staining and co-localization of the same localized regions, which is consistent with the observation in FIGS. 33A to 33C. FIG. 34C shows no detectable FITC signal in pens, further confirming the fluorescent aggregates contain secreted human antibody product. In addition, the image of the lipophilic fluorescent dye labelled cells, which showed cell membrane staining, stained only membranes of live cells growing at the distal end, e.g. opposite the proximal opening to the channel, of the pens, but did not stain aggregates that were visible using the first label and the second label (FIG. 34D). This demonstrated that the stained discrete regions in FIGS. 34A to 34B did not contain cell debris.

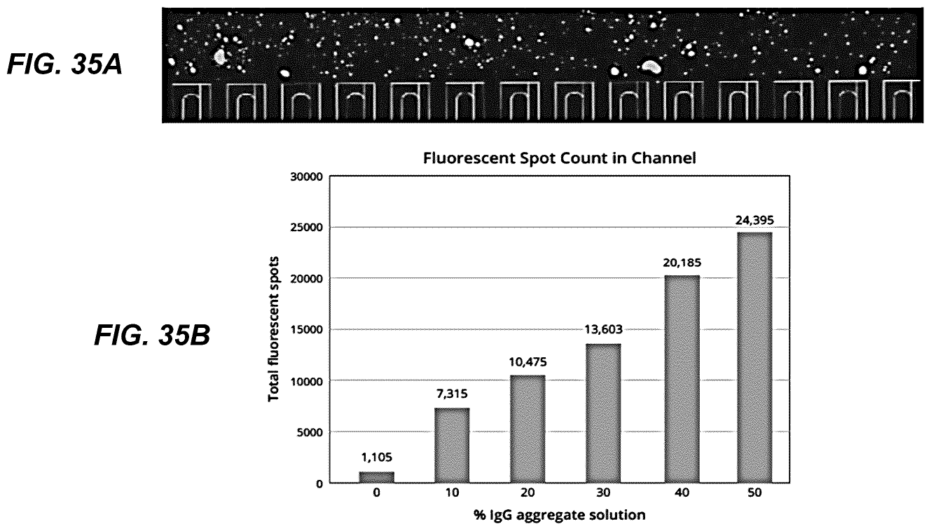

Then, an experiment was conducted to confirm the fluorescent punctate pattern observed in aggregation assays can be reproduced. Purified human IgG was subjected to heat stress at 60° C. for 48 hours to generate aggregates. Several dilutions of the heat-stressed IgG sample were loaded into the channels of OptoSelect chips along with a fluorescent label binding to the Fc region of antibodies and aggregates in the channel bound to SpotLight Human Fc (FIG. 35A). Automated counting showed that the number of punctates detected in the channel increases in a concentration dependent manner (FIG. 35B). The data demonstrates that the number of observed punctates is highly correlated with the aggregation of the secreted proteins.

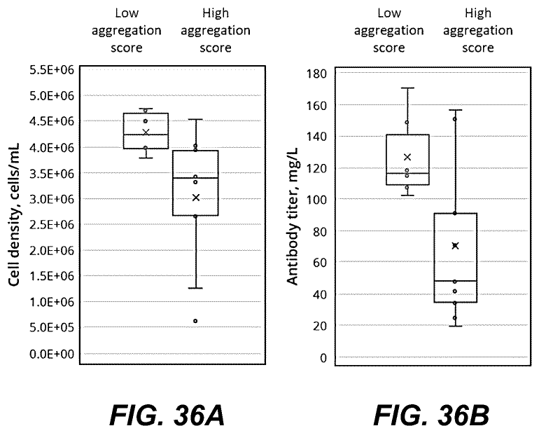

Next, in order to establish relevance of the aggregation scores with the viability and the secretion of the cells cultured in the pen, several antibody-expressing clones with a range of aggregation scores were exported from the chip into 96-well plates for scale-up and analysis. Clones were expanded to 14-day shake flask culture, at which time viable cell density and titer were assessed. Cell supernatants were harvested for subsequent analysis by size-exclusion chromatography (SE-UPLC) and quantitative laser diffraction (qLD). The titers and viable cell densities from the shake flask cultures in clones with high versus low aggregation scores were compared (FIG. 36A and FIG. 36B). FIG. 36A shows the cell densities comparison (y-axis is the concentration of cells) between the high-score clones, e.g., highly aggregated, and the low-score clones, e.g. lower amounts of aggregation. FIG. 36B shows the antibody production of the corresponding clones in the shake flask cultures. In each of the two figures, the boxplot on the left (blue) represents the low-score clones and the boxplot on the right (orange) represents the high-score clones. The low-score clones exhibit both higher viability and higher antibody titer than that of the high-score clones. These figures demonstrate that the on-chip aggregation scores correlate with growth and titer, e.g, higher aggregation scores are associated with lower growth and lower titer.

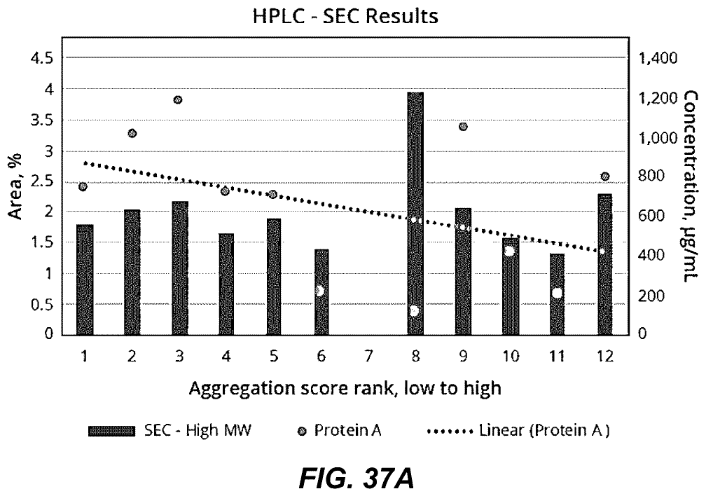

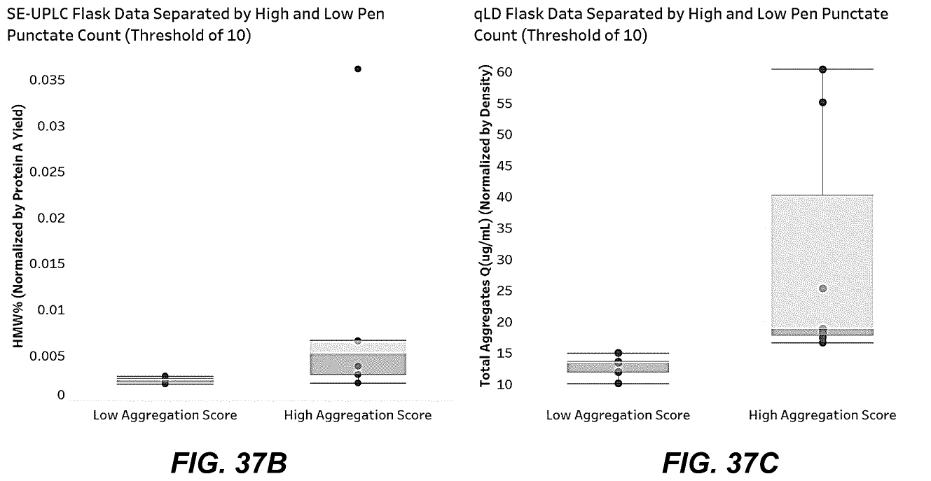

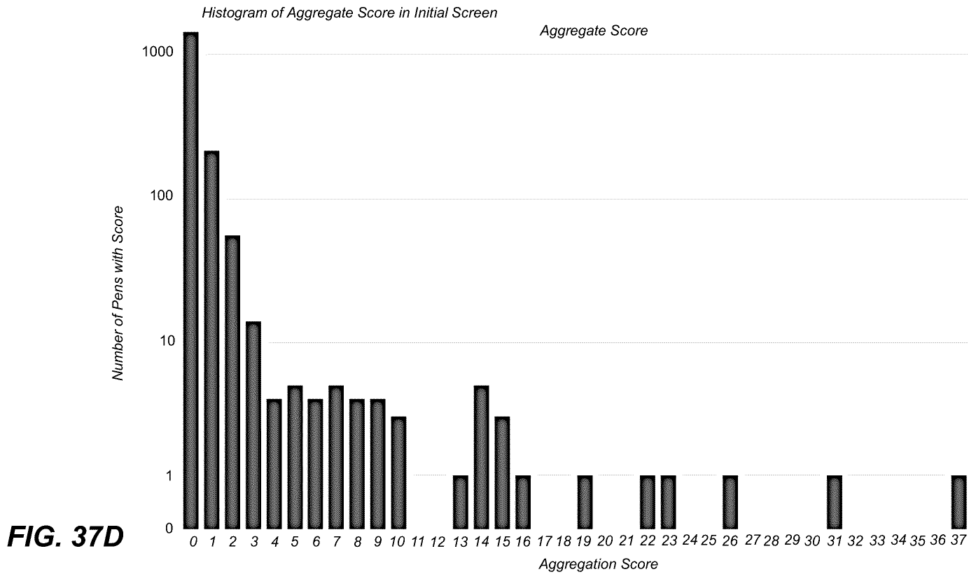

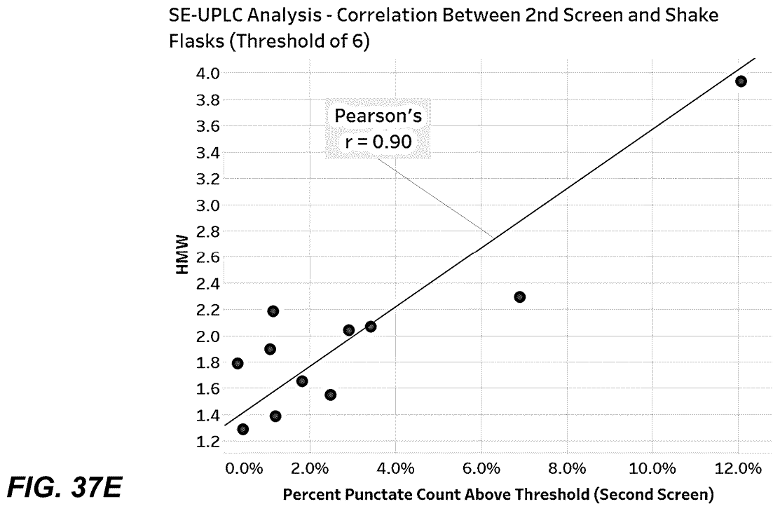

In addition, the supernatants of the shake flask cultures were collected, filtered at 0.2 um, and subjected to two rounds of Protein A purification. Then, SE-UPLC (Size-Exclusion Chromatography) was performed using a Nexera X2 HPLC/UPLC system (Shimadzu). The results were summarized in FIG. 37A. The histogram (blue) represents the levels of high molecular weight aggregates (Area %) produced by clones of various aggregation scores. The yellow circles show the yield from protein A purification, and the dotted line in yellow gives the trend of the yield along the clones. According to the results, yield from Protein A purification shows downward trend corresponding to increased aggregation score. Clones with the highest aggregation scores were more likely to have increased levels of high molecular weight aggregates and/or low product yield. FIG. 37B shows the distribution of the percent of high molecular weight species detected by the SE-UPLC for clones with on-chip aggregation score<10 (low) compared to clones with aggregation score>10 (high). As shown in the figure, the high punctate group exhibited higher percent of high molecular weight species and showed higher diversity. In comparison, the low punctate count group had lower and relatively stable percent of high molecular weight species.

The other sample was not centrifuged and was filtered with a 5 um low-protein affinity filter for use in quantitative laser diffraction (qLD) analysis. FIG. 37C shows the total aggregates for clones with on-chip aggregation score<10 (low) compared to clones with aggregation score>10 (high). The samples for qLD were analyzed, and the total measurement of particles were integrated and normalized by the cell density in the flask at time of harvest. These measurements represent total particulates and aggregates, including any non-antibody protein aggregates. High aggregation scores successfully predicted the highest levels of total aggregation.

The threshold chosen in FIG. 37B and FIG. 37C can vary depending on the needs of the experiments. FIG. 37D shows the distribution of the scores of pens. As noted, most of the pens scored no higher than 10 and the majority scored under 5. Thus, in this experiment, score 10 was used as the threshold to separate the high aggregation score group and the low aggregation score group.

FIG. 37E shows the correlation between the percent of aggregated subclones from a secondary screen and the percent of high molecular weight species values from the SE-UPLC. Each of the clones selected for this study were scaled up, and a cell sample (about 300 cells per clone) from those clones loaded onto a new chip for secondary screen assessing the level of aggregation Subclones with an aggregation score above the designated threshold of 6 for this experiment, were counted as aggregated. The results shows that the r value of the percent of aggregated subclones from secondary screen and the percent of high molecular weight species values from the SE-UPLC was 0.90.

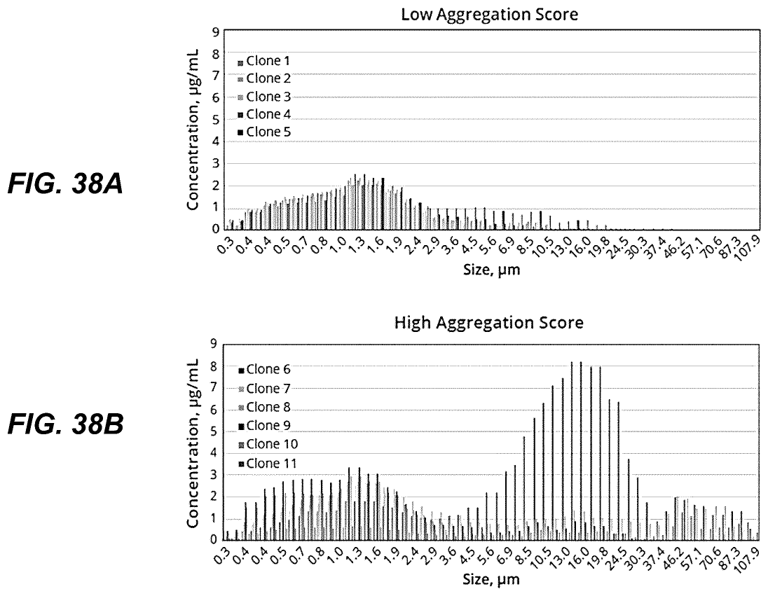

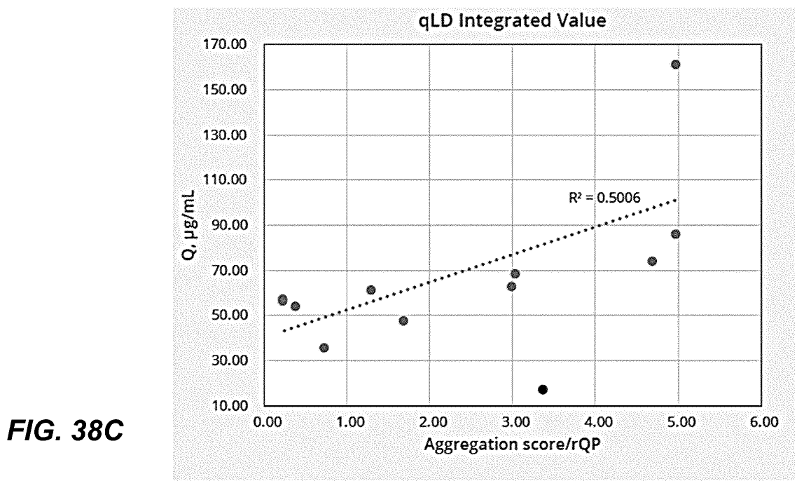

Lastly, a quantitative laser diffraction (qLD) assay was performed, which quantifies insoluble aggregate particles by size in the subvisible range. The supernatants of the shake flask cultures were collected, filtered at 0.5 um, and loaded on Shimadzu SALD-7500 nano particle size analyzer. FIG. 38A and FIG. 38B shows combined qLD histogram plots for SVP aggregates binned by size, based on high versus low Opto Assure aggregation scores. High scoring clones tend to exhibit high amounts of larger-size aggregates in qLD analysis. These larger aggregates do not appear in lower scoring clones. In addition, overall aggregate concentration (integral value) in the supernatant samples moderately correlates with the aggregation score normalized to specific productivity (FIG. 38C).

### Example 4. Enhancement by Expressor Enhanced Penning

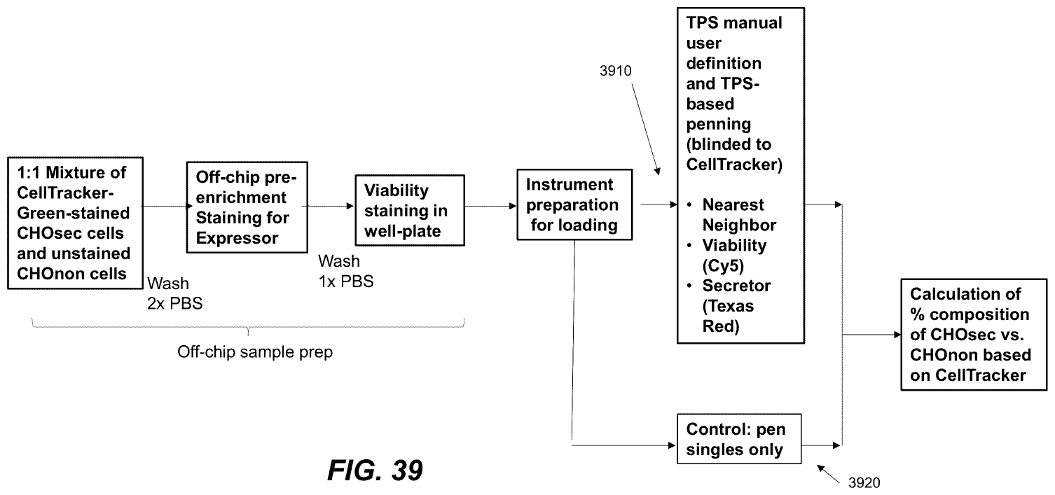

The purpose of this experiment is to enable in-channel identification, selection and targeted penning of cells based on a surface stain that is an indicator of specific productivity as well as indicator of viability. The overall experimental design is shown in FIG. 39. Penning was performed blinded to a third verification dye, which stains only the secreting cell population.

System and device: as above.

Cells: CHO-S cells (CHOnon, ThermoFisher, non-secretors), and modified CHO cells (CHOsec, which secrete a proteinaceous product).

Media: as described above.

First label (viability): Annexin V; Second label (Reporter molecule): SpotLight Kappa, a fluorescent label that binds to the conserved region of kappa light chain of antibodies, detected in the TxRed channel of the system.

Pre-Loading preparation: For conducting expressor enhanced penning, cells to be loaded were subject to pre-loading preparation wherein the cells were labelled (stained) with both a fluorescent label for observing the secretion and a fluorescent label for evaluating the viability.

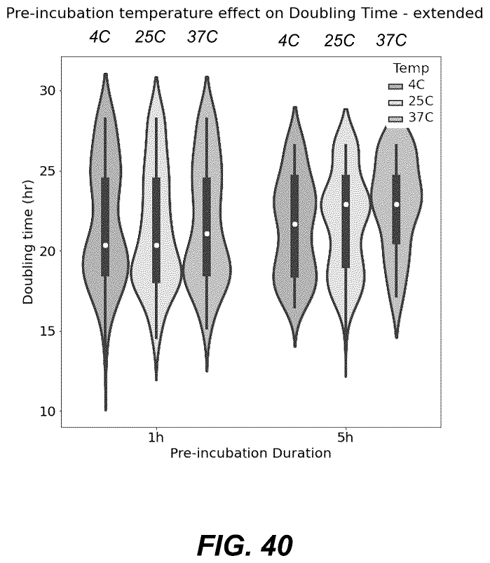

In order to determine the effect of the temperature at which the pre-loading preparation will be conducted on the viability of the cells, the cells were pre-incubated at 4° C., 25° C. (room temperature), and 37° C. respectively for 1 hour or 5 hours in Eppendorf tubes and then loaded on chip for expansion. The doubling time of the cells were recorded. FIG. 40 shows that there was no apparent temperature effect on the doubling time. The doubling time increased when the pre-loading preparation duration increased to five hours, but overall, conducting the pre-loading preparation at 4° C. 25° C., and 37° C. for 1 hour or 5 hours does not affect the viability of the cells. Based on the data, the staining process of the pre-loading preparation in the following experiments were conducted at 4° C. or 25° C. for a one hour period.

For this verification of the dual segregation strategy of the method, the CHOsec cells were stained with CellTracker Green™ (ThermoFisher), a fluorescein dye, detectable in the FITC channel of the system. CellTracker Green was retained intracellularly for multiple generations and thus was used to track cellular movement within the microfluidic environment. The CHOsec were then washed with PBS to remove unbound stain. A substantially 1:1 mixture of the pre-stained CHOsec cells and CHOnon cells was then treated with the reporter molecule, with the expectation that only the pre-stained CHOsec cells would be stained with the reporter molecule, thus having a dual label. The CHOnon cells were expected to have no staining. PBS washing followed. Then the mixture of cells was further stained with Annexin V, which provides fluorescent labelling of non-viable cells, detectable in the Cy5 channel of the system. Viable cells of each type were not labelled.

The mixed cell population was imported into the channel of the microfluidic device, and flow stopped.

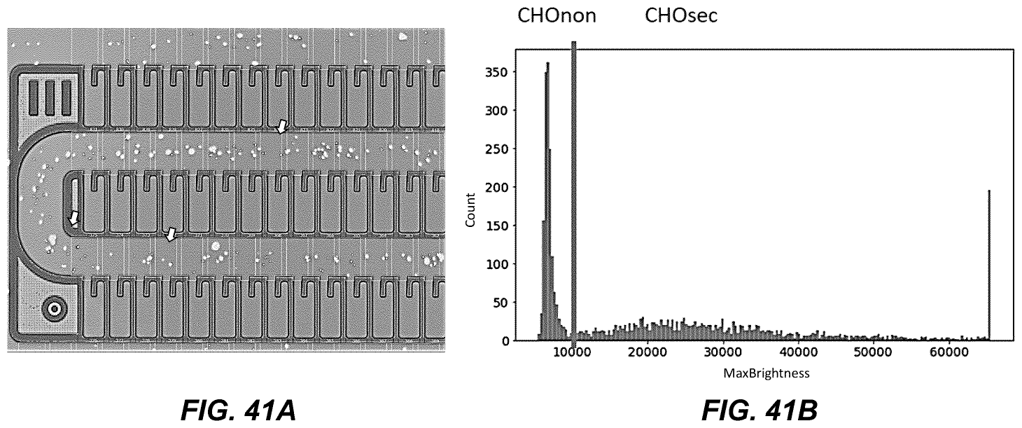

Images were taken of cells within the microfluidic channel in the FITC channel and in the TxRed channel in order to identify and verify the ratio of CHOsec:CHOnon introduced into the microfluidic device. FIG. 41A shows the image detected in FITC.

In looking at the data received from the FITC analysis, a clear delineation of these two populations was determined as shown in FIG. 41B, where the count of CNHOPnon clustered strongly vs x axis of brightness of CellTracker Green FITC signal. The threshold was set at 10,000 (maxbrightness), and was used to classify cells as CHOnon or CHOsec in both pre-penning overall population and in post-penning verification analysis.

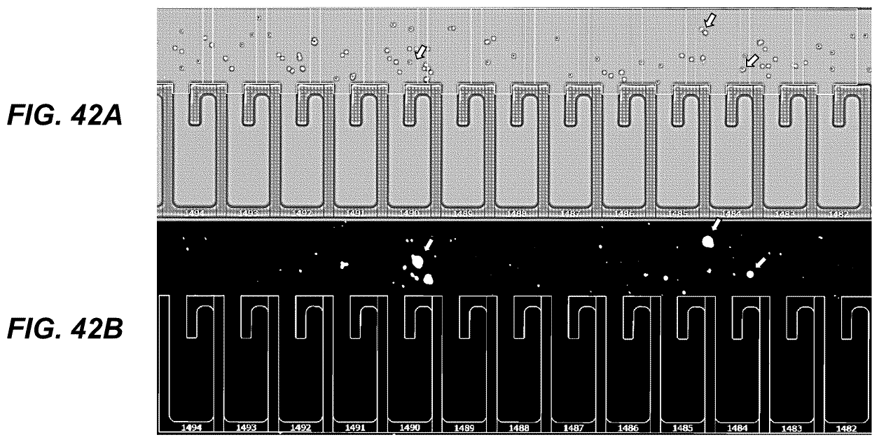

In FIG. 42A, a representative portion of a row of pens and the channel adjacent thereto is shown, showing the distribution of cells within the channel before penning. The same row of pens is shown in FIG. 42B, in a CY5 image obtained which shows those cells labeled by Annexin V (see arrows), and therefore excluded by the penning algorithm from being penned. FIG. 43A is the image of the same row of pens as in FIG. 42A, but here FIG. 43B shows cell surface staining by the secretion specific label that is imaged in the TxRed channel of the system. These are the cells of especial interest to be penned (see arrows), as long as they are also not labeled by Annexin V (e.g, are viable cells).

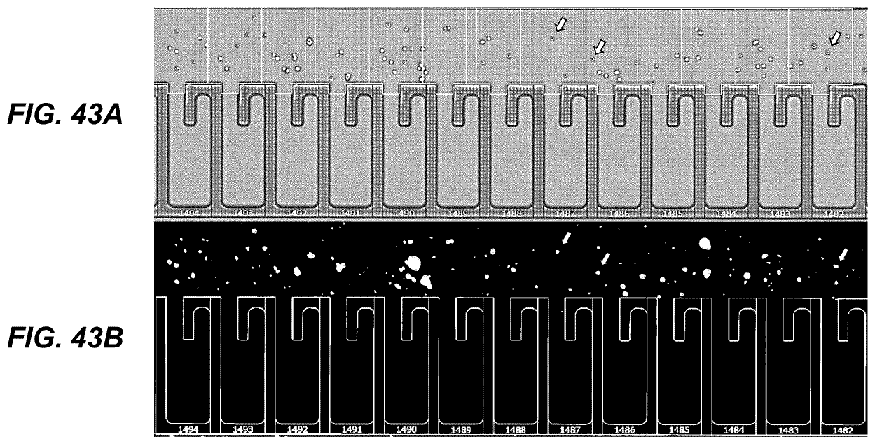

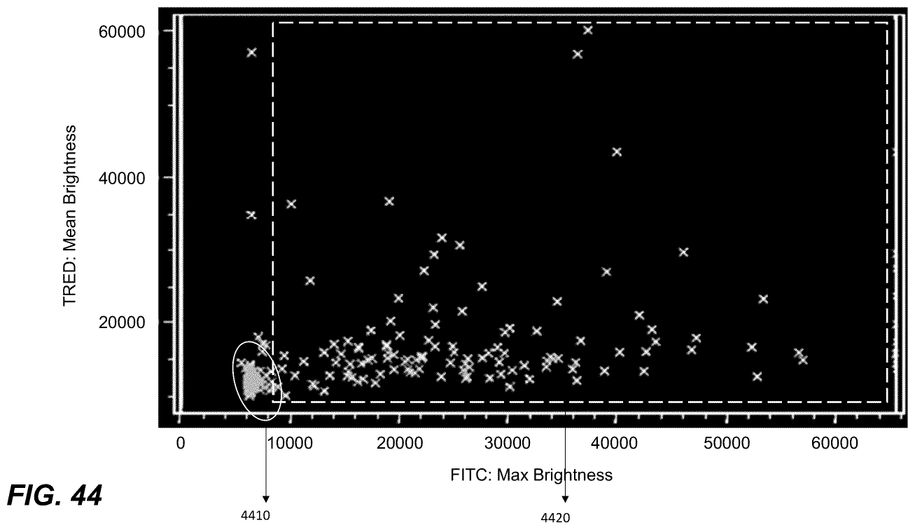

FIG. 44 shows the distribution of cells based on both FITC and TxRed images, and shows that the CellTracker Green-negative CHOnon cells show low FITC signal, and low cell surface stain (TRED), and is shown in the region 4410 near the origin of the graph. In contrast, the CellTracker Green-positive CHOsec cells show higher FITC signal, and a range of cell surface stain (TRED) distributed throughout the second region 4420 of the graph. From this analysis, it was determined that the actual ratio of CHOsec:CHOnon was about 58%; 42%.

For the penning portion of the experiment, all penning was performed blinded to the CellTracker™ Green staining, e.g., without consulting the known (stained) separation between CHOsec: CHOnon.

For a first set of chambers within the microfluidic device in the enhanced penning arm 3910 of FIG. 39, the cells were imaged in brightfield, CY5 and TxRed channels. Pre-pen selection was made based on detection of the selected combination of detectable signals: that is only cells having no detectable CY5 labelling and only cells having detectable TxRed labelling are selected for penning as shown in detail FIG. 45B. FIG. 45A shows the overall population characteristics where the graph shows TxRed stain (Y axis) and Cy5 stain (X axis). As shown, 712 cells were selected for penning and 1149 were excluded from penning, with the cells represented within region 4510 being selected (TxRed greater than threshold, and CY5 less than threshold).

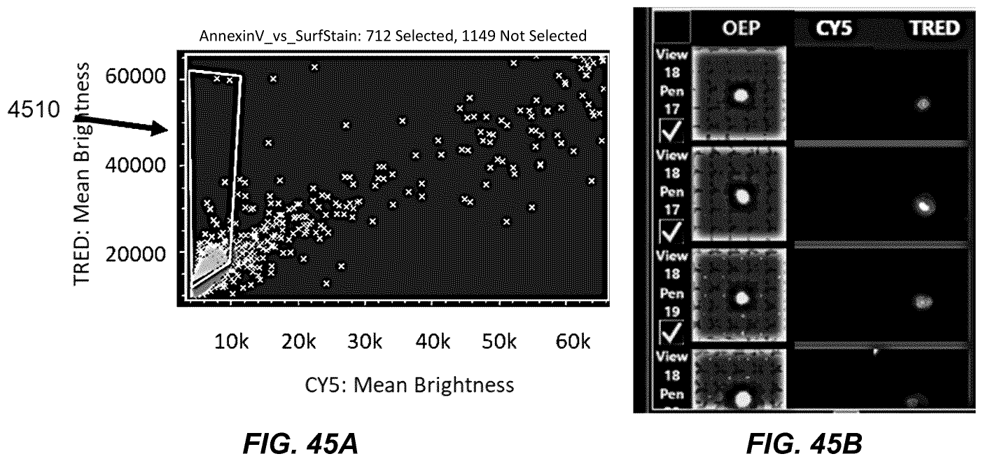

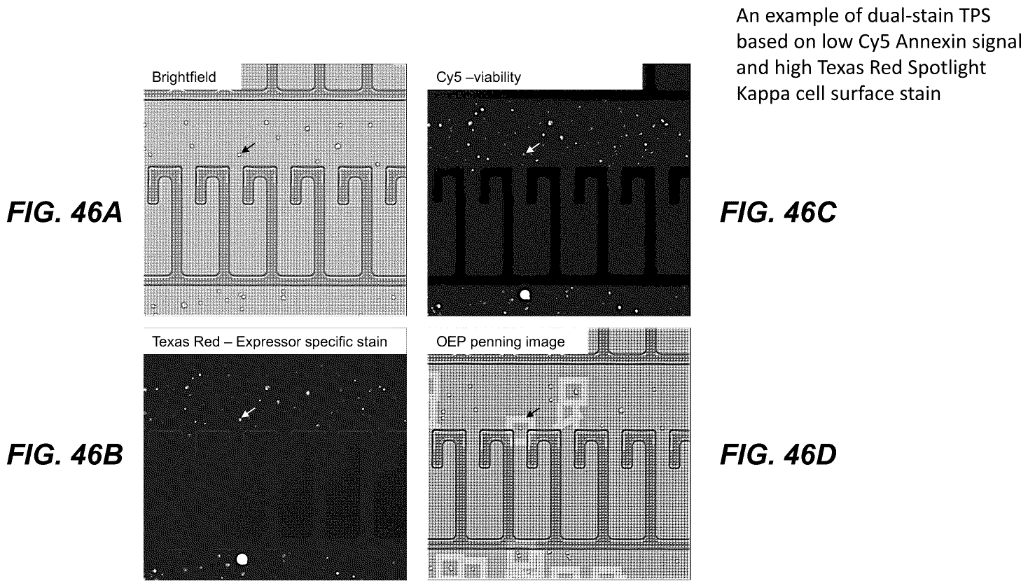

FIGS. 46A to 46C shows the respective images (brightfield, Expressor staining, Viability staining) for a single cell within the channel, which has staining attributes placing it within the selection region 4510 of FIG. 45A. FIG. 46D shows the image taken as the Optoelectronic Positioning (OEP) operation surrounds the selected cell with a light cage for penning in the nearby pen.

In the second arm 3920 of FIG. 39 of the experiment, within a second set of pens within the microfluidic device, cells are selected without any fluorescent staining criteria applied, and single cells were penned.

Post Penning Analysis. Post-penning images were taken in four channels: (OEP, TRED, CY5, FITC): to verify both the TPS-criteria were met and to verify cell identity based on positive CellTracker Green stained cells (CHOsec).

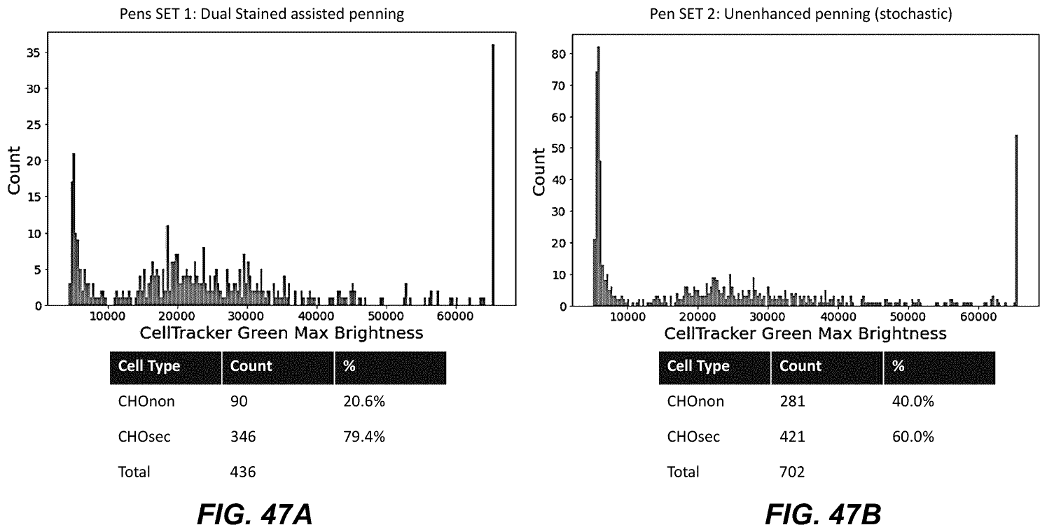

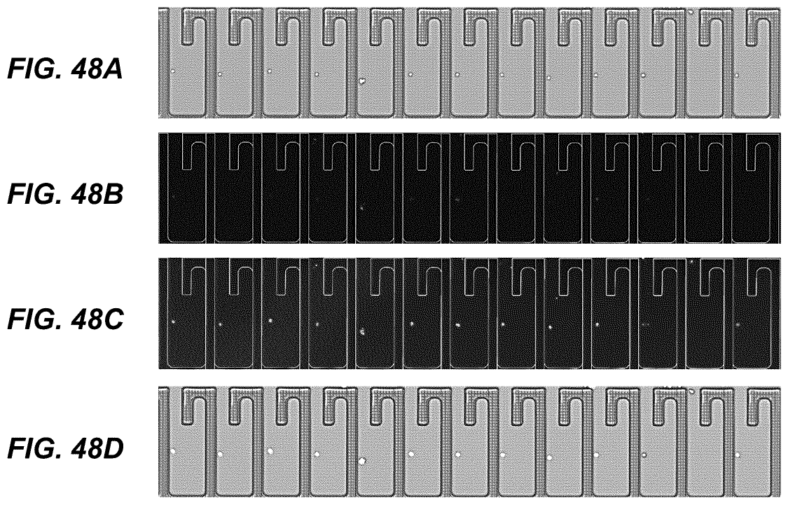

FIG. 47A and FIG. 47B shows the analysis of penning accuracy for both the staining enhanced penning conditions (arm 3910) vs. simple single cell loading (arm 3920) of the mixed population introduced into the microfluidic channel. As is seen, for the unenhanced penning, the ratio of CHOsec:CHOnon is equivalent to the overall cell distribution, where 40% of the penned cells are CHOnon and 60% are CHOsec, matching the composition detected in the channel. In contrast, in pens SET 1 (arm 3910) the number of secreting cells is improved to 79.4% relative to CHOnon of 20.6%. Further improvements were also rolled in by not penning any nonviable cells (e.g., Annexin V positive cells), The enrichment for explicitly penning secreting cells is about 1.36×standard single cell penning. FIGS. 48A to 48D show various characteristics of a set of pens (part of SET 2, arm 3920) post penning. FIG. 48A shows the brightfield image of the row of 13 pens, where a single cell may be seen within each of the pens (second from right, cell is disposed at opening of the pen, and has not been moved into the isolation region of the pen). FIG. 48B shows the image taken in the CY5 channel, which demonstrates the lack of labelling by Annexin V, indicating that every cell in this row of pens is viable, and that the pre-penning sort by the automatic penning routine has successfully only penned viable cells. FIG. 48C shows the image obtained of the same row of pens in the TxRed channel, which detects cells labeled for expression of the secreted molecule. Again, every pen has an appropriately labeled cell within it. FIG. 48D shows the verification image of the FITC labeled channel, showing that only cells stained with CellTracker Green, and hence identified as the CHOsec cells, have been penned.

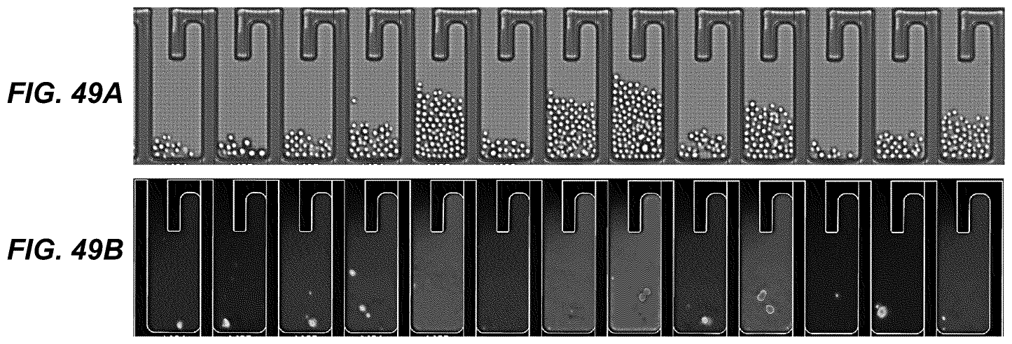

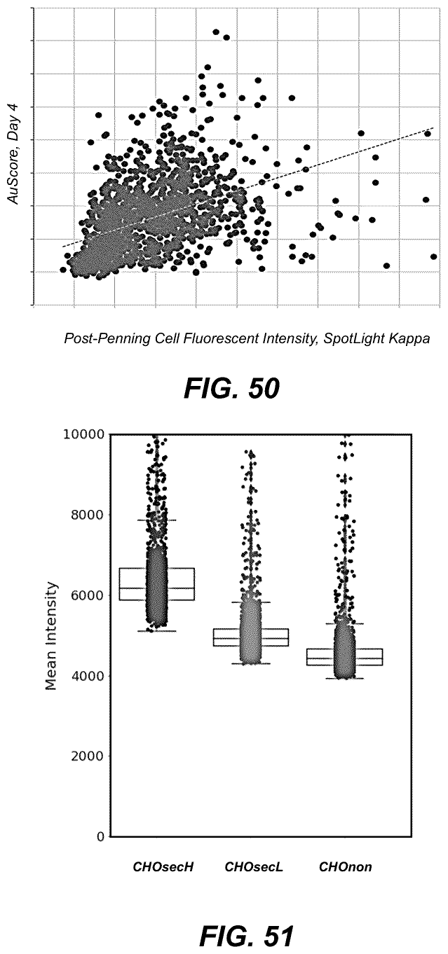

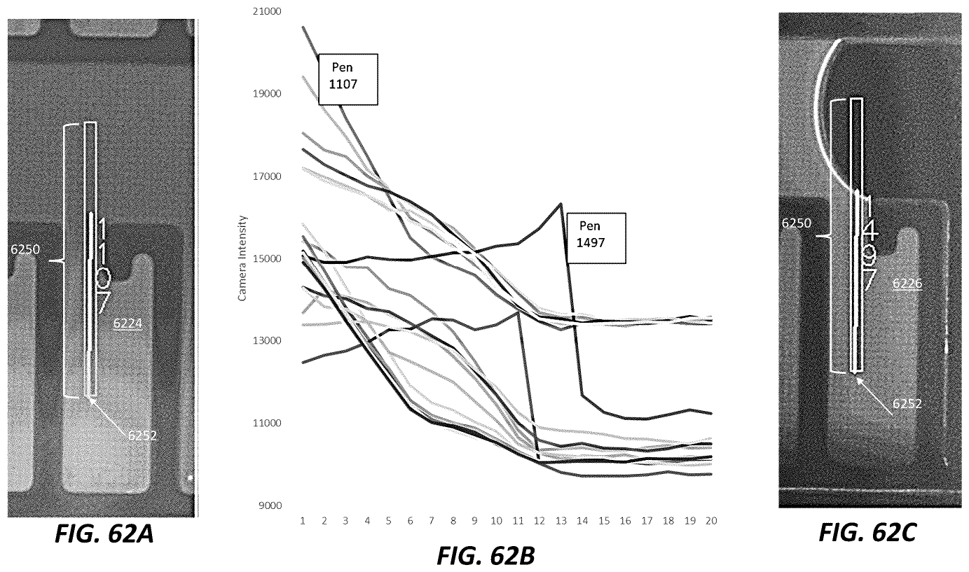

After loading the cells were cultured for 3 days. Brightfield images were obtained as shown in FIG. 49A. Diffusion assays as described herein were performed, using the same reporter molecule, to assess relative cell secretion productivity (rQp). The fluorescent image taken during the assay in the TxRed channel, shown in FIG. 49B shows the varying secretion levels across the row of pens, with the majority of the pens showing secretion. The enhanced penning method provided enhanced loading only of cells having greater likelihood of successfully demonstrating secretion and thus of higher interest for successful downstream cell line scaleup. Furthermore, as shown in FIG. 50, plotting the AuScore obtained from a diffusion assay using SpotLight Human Fc and the SpotLight Kappa intensity of post-penning cells, FIG. 50 shows that the SpotLight Kappa intensity was moderately correlated with the secretion ability of the penned cells. This data demonstrates that the SpotLight Kappa intensity is a reliable prediction for the secretion of the cells after penning. The AuScore is an arbitrary unit of diffusion assay score, which in this experiment was the slope of the curve of concentration values in FIGS. 62A to 62C (Example 11).

### Example 5. Enhancement by Expressor Enhanced Penning: Three-Tiered Differentiation

The purpose of this experiment is to test whether the expressor enhanced penning method of the present disclosure can distinguish secretors from non-secretors and differentiate high secretors from low secretors.

System and device: as described above.

Cells: CHO-S cells (CHOnon, ThermoFisher, non-secretors), and modified CHO cells (CHOsecH, which secrete a proteinaceous product and were pre-determined as high secretors, and CHOsecL, which secrete a proteinaceous product and were pre-determined as low secretors).

Media: as described above.

First label (viability): Annexin V; Second label (Reporter molecule): SpotLight Kappa, a fluorescent label that binds to the conserved region of kappa light chain of antibodies, detected in the TxRed channel of the system.

Pre-Loading preparation: as described in Example 4.

The result in FIG. 51 shows that the CHOsecH cells exhibited the highest mean intensity, which was significantly different from the mean intensity of the CHOnon. This result showed consistence with the previous experiments that the expressor enhanced penning method is selective in distinguishing secretors from non-secretors. On top of that, CHOsecL cells exhibited a middle level of intensity, which was distinguishable lower than that of CHOsecH cells and distinguishable higher than that of CHOnon cells. The result suggests that the expressor enhanced penning method was sufficiently sensitive to distinguish the high secretors, the low secretors, and the non-expressors from each other. This data suggests that the expressor enhanced penning method can not only identify cells that are viable and secreting, but also can distinguish the better cells within those secretors. That is to say, the expressor enhanced penning method can be used when prioritizing penning is needed.

### Example 6. Enhancement by Expressor Enhanced Penning: Chemical Enhancers

The purpose of this experiment is to evaluate the concentration of the staining reagent and the chemical enhancers to find better separation between non-expressor and expressor. The staining reagent was diluted with the enhancers at a ratio of 1:4, 1:8, and 1:20 respectively. Degree of separation was calculated using Cohen's D algorithm for determining effect size.

System and device: as described above.

Cells: CHO-S cells (CHOnon, ThermoFisher, non-secretors), and a known CHO secretor cell line (CHOknown).

Media: as described above.

First label (viability): Annexin V; Second label (Reporter molecule): SpotLight Kappa, detected in the TxRed channel of the system.

Pre-Loading preparation: as in Example 4, with the exception that the cells were loaded in media that included one of the chemical enhancers at the specified concentrations.

Enhancers: PVP (2% (w/v) in PBS), Bovine serum albumin (1% (w/v) in PBS), PBS (control).

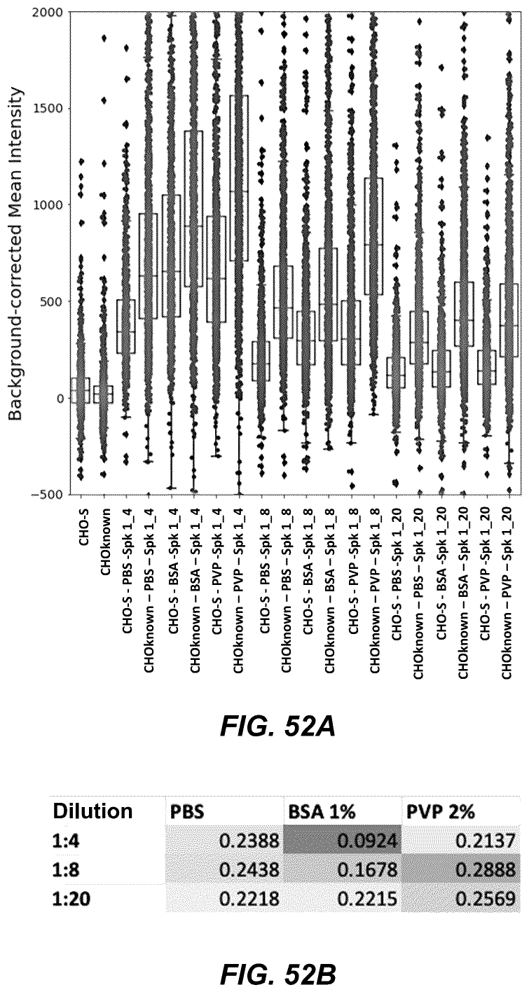

The result is shown in FIG. 52A and FIG. 52B. In FIG. 52A, the Y-axis shows the mean intensity of the fluorescence read from the cells cultured, and the X-axis indicates the cell type together with the enhancer used as well as the dilution ratio. The boxplots are to evaluate which kind of the enhancers and at which dilution ratio can the intensity of secretor and non-secretor be separated clearly. The results shows that PVP provided the best separation in this experiment at all tested dilution ratio and especially at the ratio of 1:8 and 1:20. BSA also improved the separation especially at the ration of 1:20.

The heatmap table (FIG. 52B) shows the Cohen's d value of this experiment. Cohen's d is a statistical methodology used to determine how different two means are. It is defined by calculating the difference of the two means and dividing it by a standard deviation. The higher the value is, the more distinct the two means are. The table informs that 1:8 dilution and 2% PVP was ideal in this experiment (green color). Bovine serum albumin, which is commonly used as a non-specific binding blocker in FACS cell preparation, did not provide comparable effect as PVP.

### Example 7. Enhancement by Expressor Enhanced Penning: Enrichment Evaluation

In this experiment, cells were penned respectively by the expressor enhanced penning method of the present disclosure and a basic penning method which did not differentiate between cells to be penned, and is directed to loading single cells. A diffusion assay as described in Example 1 was then performed, using SpotLight Human Fc as the reporter molecule to determine the secretion ability of the penned cells, and therefore the extend of enrichment can be evaluated.

System and device: as described above.

Cells: CHO-S cells (CHOnon, ThermoFisher, non-secretors), and modified CHO cells (CHOsec, which secrete a proteinaceous product).

Media: as described above.

First label (viability): Annexin V; Second label (Reporter molecule): SpotLight Kappa, detected in the TxRed channel of the system.

Pre-Loading preparation: as in Example 3.

Loading arrangement: 3 chips were used in this experiment. Chip #D85361 was imported with only CHOsec cells (positive control), and the cells were penned by the basic loading method. Chip #D85410 and Chip #D85411 were imported with a mixture of CHOsec cells and CHOnon cells, and the cells on each of the chips were penned by two rounds of the expressor penning method of the present disclosure.

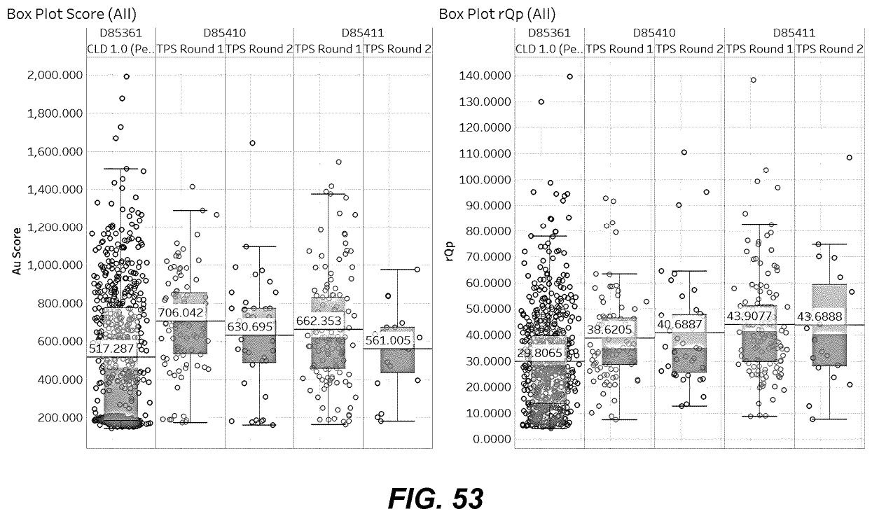

The results are shown in FIG. 53. The left figure is the AuScore (the AuScore is an arbitrary unit of diffusion assay score, which in this experiment was the slope of the curve of concentration values as taught in FIGS. 62A to 62C) and the right figure shows the relative cell secretion productivity (rQp) value. Both were determined by the diffusion assay using SpotLight Human Fc as shown in Example 1, and each spot on the figure represents the value of a pen. The data shows that the expressor enhanced penning method of the present disclosure was selective in penning cells that were viable and expressors. The expressors were enriched significantly and the overall secretion was higher than that resulting from loading only CHOsec cells using the basic, non-enriching loading method.

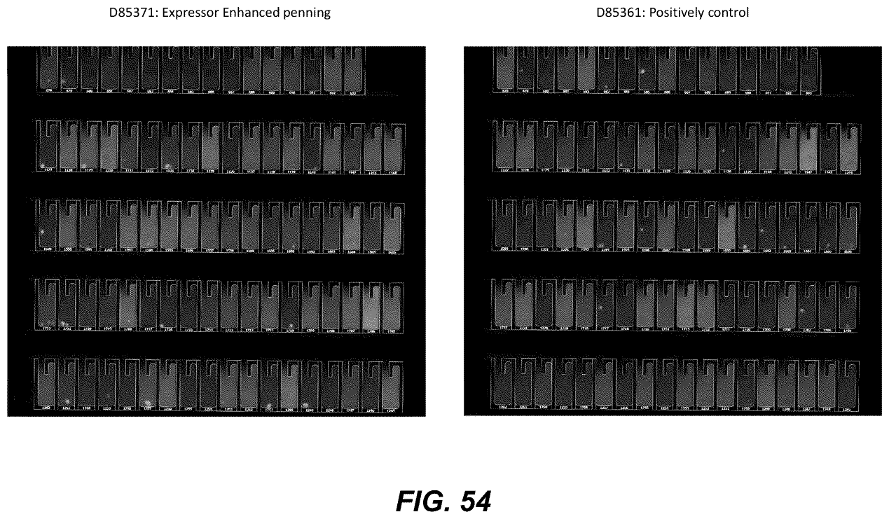

FIG. 54 shows the on-chip images taken after the diffusion assay was performed in another experiment. The figure showed that Chip #D85371 (expressor enhanced penning) had more pens exhibiting the fluorescent and the fluorescent was generally brighter than the pens of Chip #D85361 (where cells were loaded using the basic, non-enriching method).

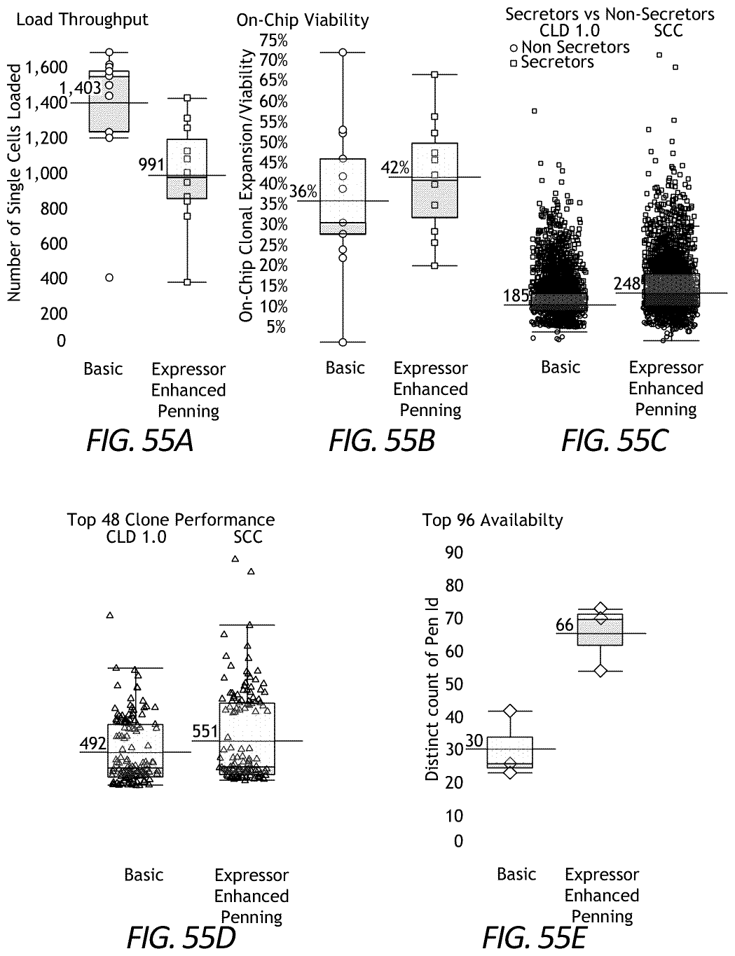

FIG. 55 presents the enrichment that the expressor enhanced penning method of the present disclosure can achieve. In this figure, data aggregated from a series of experiments, were collected for comparison, in which cells were either penned by using the basic loading method or by the expressor enhanced penning method as described in the previous experiments above. Diffusion assays as described herein were performed to obtain the AuScore of each pen.

According to FIG. 55(A) to (E), the basic loading method loaded more cells than the expressor enhanced penning method because the latter method excluded more non-viable cells (FIG. 55(A)). However, the cells penned by the expressor penning method had better overall on-chip viability (FIG. 55(B)). FIG. 55(C) shows that even though the basic loading method penned more cells, the expressor enhanced penning method captured more secretors (248 vs. 185) and overall higher AuScore (y axis) were exhibited for the secreting cells penned by the expressor enhanced penning method, which was consistent with the data shown in previous experiments.

The top 48 clones in each group were identified and the AuScores (y axis) were shown. FIG. 55(D) shows that expressor enhanced penning method not only captured more secretors, the secretors penned also performed better. Then, in FIG. 55(E), AuScores of all pens (combining the data from pens of both basic loading method and expressor enhanced penning method) were collected and the pens having the top 96 AuScore were identified. As shown, 66 out of the 96 pens were penned by the expressor enhanced penning method, proving the expressor enhanced penning method was highly selective in penning the better cells.

Table 1 contains data collected from a different set of four experiments. The data of these four experiments are consistent with the previous experiments. As shown in the table, in each of the four experiments, cells penned by using the expressor enhanced penning method exhibited higher percentages of sec of yield secretor from single loaded and yield secretor from viable clones. This trend is particularly important as it verified the expressor enhanced penning method of the present disclosure consistently selectively penned better cells in the chambers. The value of mean corrected AuScore further verified that the selective penning did translate into higher overall on-chip productivity.

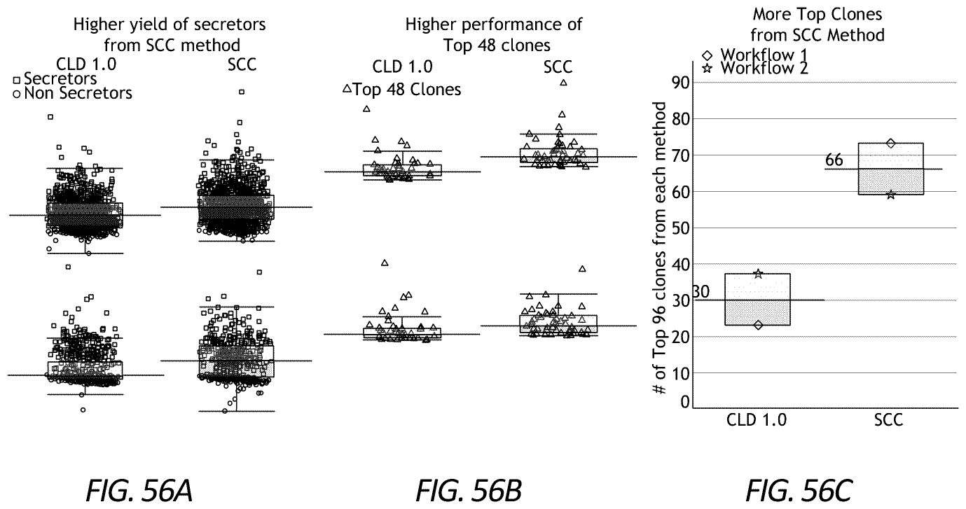

The data collected from Experiment #A and Experiment #B are analyzed in different methods in FIGS. 56A to 56C. FIG. 56A shows the distribution of secretors (green) and non-secretors (red) based on their AuScores (y axis) at 2 timepoints, Experiment #A (upper plot) and Experiment #B (lower plot), where the cells penned based only on penning single cells are shown in the left column of FIG. 56A and the cells penned based on the enhanced expressor penning methods are shown in the right column of FIG. 56A. More secretors were penned by the expressor enhanced penning method, and within those populations of cells can be found cells having higher secretion scores (Au) in both Experiment #A and Experiment #B. FIG. 56B presents only the top 48 clones on chips of basic method (left column) and expressor enhanced penning method (right column), for Experiment #A (upper plot) and Experiment #B (lower plot) respectively. This figure shows that the top 48 clones of the expressor enhanced penning chip had better performance (based on AuScore, y axis), which was consistent with the overall observation in FIG. 56A. Lastly, FIG. 56C shows that, by combining the data from these two experiments, 66 clones out of the top 96 clones were from the expressor enhanced penning chips (right column of FIG. 56C, whereas only 30 of 96 top expressing clones were found from the chips where singles only penning was used (left column of FIG. 56C). Therefore, despite penning more cells, when using the basic singles only penning methods are used, more top performing cells are found when using the enhanced loading methods. The data shown in FIGS. 56A to 56C (were consistent with that in FIG. 55(C) to (E)).

TABLE 1

| | | | % Yield | % Yield | | |
|---|---|---|---|---|---|---|
| | | | secretor | secretor | Mean | |
| | Chip | | from single | from viable | corrected | Top 96 |
| Exp # | ID# | Penning Method | loaded | clones | AuScore | contribution |
| Exp | D84639 | Expressor Enhanced | 16.2 | 64.7 | 27.63 | 59 |
| #A | | Penning | | | | |
| | D85366 | Basic method | 9.1 | 37.9 | 13.22 | 59 |
| | D85368 | Expressor Enhanced | 12.9 | 66.3 | 46.63 | 59 |
| | | Penning | | | | |
| | D85414 | Basic method | 6.7 | 31.0 | 39.70 | 59 |
| Exp | D84681 | Expressor Enhanced | 42.7 | 93.1 | 230.31 | 71 |
| #B | | Penning | | | | |
| | D84685 | Basic method | 24.9 | 80.8 | 157.35 | 71 |
| | D85372 | Expressor Enhanced | 40.8 | 96.3 | 200.77 | 71 |
| | | Penning | | | | |
| | D85440 | Basic method | 23.3 | 82.5 | 139.80 | 71 |
| Exp | D85404 | Expressor Enhanced | 15.1 | 43.1 | 34.61 | 44 |
| #C | | Penning | | | | |
| | D85413 | Basic method | 11.4 | 29.6 | 16.19 | 44 |
| | D85432 | Expressor Enhanced | 12.3 | 43.9 | 16.46 | 44 |
| | | Penning | | | | |
| | D85441 | Basic method | 8.3 | 30.3 | 14.55 | 44 |
| Exp | D85373 | Expressor Enhanced | 18.9 | 40.2 | 49.78 | 18 |
| #D | | Penning | | | | |
| | D85380 | Basic method | 12.5 | 30.2 | 12.15 | 18 |
| | D85381 | Expressor Enhanced | 19.6 | 49.9 | 34.88 | 18 |
| | | Penning | | | | |
| | D85409 | Basic method | 6.9 | 25.1 | 1.48 | 18 |

*Mean corrected AuScore was calculated by the formula: Mean AuScore of population − Au baseline

### Example 8. Enhancement of Expressor Penning: Enrichment Evaluation

In this experiment, cell mixtures having initial percent of non-secretors and secretors were used. The enrichment power of the expressor enhanced penning method of the present disclosure was further evaluated by observing the increase of the percent of the secretors after penning.

System and device: as described above.

Cells: CHO-S cells (CHOnon, ThermoFisher, non-secretors), and CHOknown cells (which secrete a proteinaceous product).

Cell mixture: Cell mixture of 50/50 (50% of secretors and 50% of non-secretors) and cell mixture of 20/80 (20% of secretors and 80% of non-secretors) were used.

Media: as described above.

First label (viability): Annexin V; Second label (Reporter molecule): SpotLight Kappa, detected in the TxRed channel of the system.

Pre-Loading preparation: as described in Example 4.

The data shows that the basic method can moderately increase the percent of secretors, but the enrichment that the expressor enhanced penning method achieved was dramatically. Nearly 40% of increase was observed in both the 50/50 groups and 20/80 groups. The data of 20/80 groups is particularly notable because it suggests that even if a cell mixture containing relatively low percentage of secretors, the expressor enhanced penning method can still effectively select and pen the better cells.

TABLE 2

| Initial | | | Num- | Num- | % of secretors |
|---|---|---|---|---|---|
| Percent | Chip ID | Penning Method | Secretor | Total | after penning |
| 50/50 | D84681 | Expressor enhanced | 542 | 605 | 89.59% |
| | | penning | | | |
| | D84685 | Basic Loading | 359 | 479 | 74.74% |
| | D85372 | Expressor Penning | 481 | 534 | 90.07% |
| | D85440 | Basic Loading | 349 | 452 | 77.21% |
| 20/80 | D84639 | Expressor enhanced | 171 | 283 | 60.42% |
| | | penning | | | |
| | D85366 | Basic Loading | 119 | 380 | 31.32% |
| | D85368 | Expressor enhanced | 123 | 196 | 62.76% |
| | | penning | | | |
| | D85414 | Basic Loading | 99 | 348 | 28.45% |

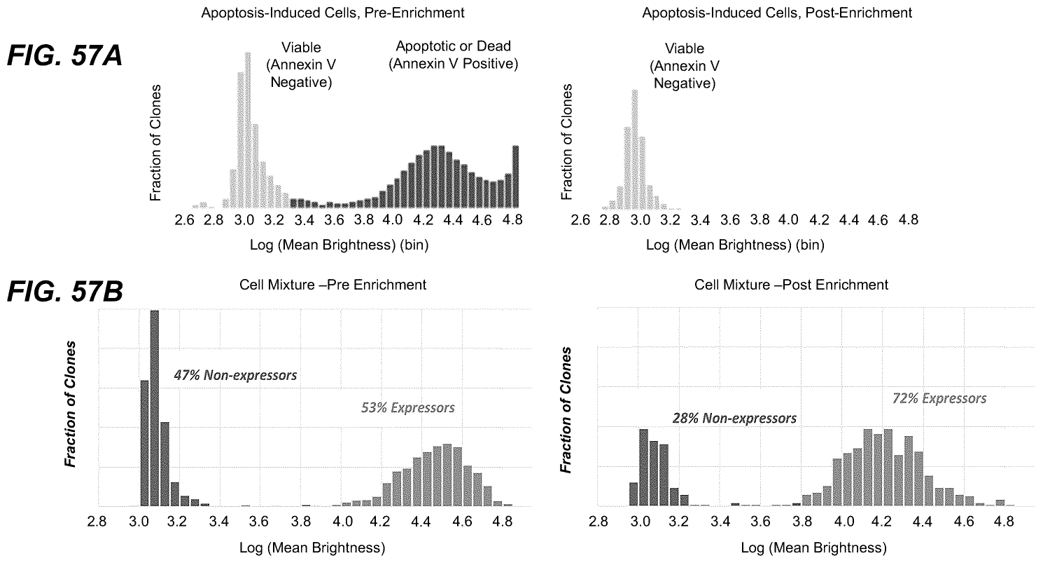

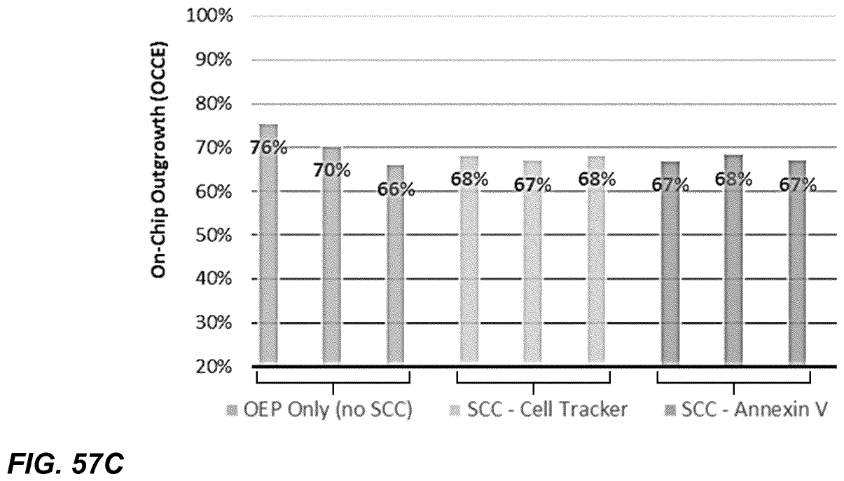

A high viability cell suspension was spun down pelleted and resuspended to a final density of 3-4×10\hat{ }6 cells/mL EX-CELL Advanced culture media. The cells were treated with a final concentration of 10 uM Staurosporine, a non-specific general kinase inhibitor, to induce apoptosis and then incubated at 37° C. for 16 hours. Excess Staurosporine was removed by two rounds of media exchange and then resuspended in 1 mL load media. To stain Staurosporine treated cells with Annexin V-Cy5, cells were resuspended in 1 mL of load media at a density of 6×10\hat{ }6 cells/mL in load with a final concentration of 0.5 μg/mL Cy5 Annexin V (Biotium, PN 29008R) and 2.5 mM CaCl₂). The cells were incubated for a minimum of 15 minutes at 37° C. right before they were analyzed on the Beacon system. The apoptotic and non-apoptotic cells were then combined to create a sample with 38.5% viable cells as a model for a post-transfection sample pool. A selection threshold of 3.3 was defined based on the results generated when the two cell populations were run separately. Each Annexin-V negative cell was transferred to a single NanoPen chamber for clone expansion. The result is shown in FIG. 57A. The x-axis of FIG. 57A is the log value of the brightness (intensity) of Annexin V, and the y-axis is the fraction of clones. FIG. 57A erifies that, with an incoming Annexin-V negative population of 38.5% (left), the post-penning analysis of the single cells loaded showed 97% Annexin V negative (right), which translates to 2.5× enrichment. When the on-chip viability of the SCC-enriched samples was compared to the unenriched control after 4 days of culture, higher viability was measured for the enriched sample (77%) compared to the unenriched sample (67%) indicating that SCC enrichment improved the on-chip viability of the sample, increasing the genetic diversity of the cells available for screening (figure not shown).

Another set of data of experiment testing the 50/50 ratio (47% v. 53%) is shown in FIGS. 57B to 57C. FIG. 57B analyzes the log value of the brightness and the fraction of clones but detects the brightness of SpotLight Kappa. This figure shows that the percent of secretors was increased from 53% to 72% after penning. FIG. 57C confirms the selective penning methods in the present disclosure are gentle on cells and do not damage then like FACS sorting, which typically has low outgrowth (<30%) . . . . The x axis lists twelve experiments using four different penning methods (each of the four penning methods was performed in triplicate), and the y axis shows the outgrowth percent of the cells penned. Comparing with the OEP only group (which the cells were penned randomly without staining or labelling described herein), no matter cells were stained with the CellTracker or Annexin V, all sorted cells consistently show robust colony formation at a similar rate as penning without selection regardless of sorting criteria or stain used. There were no significant adverse effects observed.

### Example 9. Correlation of Average rQP with Rank Order Performance in a Fermentation

A population of subclonal cells derived from an individual clonal cell line were introduced into a set of allocated pens. This importation was repeated with respective populations of subclonal cells from four other cell lines, where each respective population was introduced to a respective set of allocated pens. The five cell lines were well characterized cell lines, with known titer performance and stability in macroscale fermentation. The five sets of populations were cultured for five days, during which period the population expanded and secreted bioproduct protein.

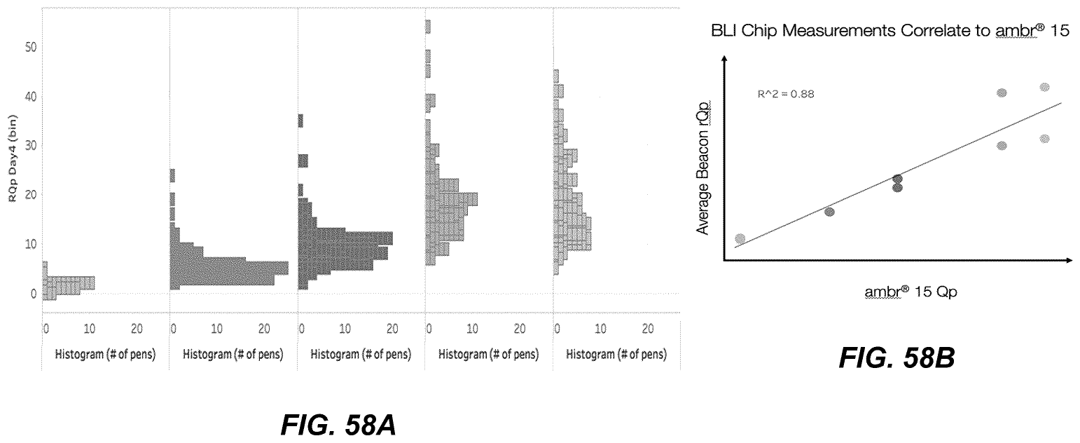

The values of rQp (normalized) were calculated for the resultant populations of subclonal cells derived from each clonal cell line, as described in Example 1, based on the diffusion, e.g., secretion, assay described here and in WO2017/181135, filed on Apr. 14, 2017 and WO2019/075476, filed on Oct. 15, 2018, each of which disclosures are herein incorporated by reference in its entirety. FIG. 58A shows a histogram of the distribution of rQp values for each of the five subject cell lines, with the x axis representing the number of pens having a particular rQp and the y axis representing the values of rQp calculated across the set of allocated pens housing the populations of subclonal cells. The population average rQp for each of the 5 subject cell lines was calculated across the individual pens of each of mapped to a fermentation titer for the respective cell line (e.g., obtained upon expansion in an Ambx® bioreactor, and titer obtained as described in Example 1), and is shown in FIG. 58B. The average rQp mapped to the respective fermentation titer (N=2) with a coefficient of determination (R squared) value of around 0.9. As shown, cell line 1, having a low Qp titer (on the x-axis) maps to the relatively low average rQp found for cells from that cell line cultured within the microfluidic environment. Cell line 2, having an increased Qp, is found further along the x axis and correlates with a correspondingly increased rQp. The same relationship is found with cell line 3, a moderately expressing cell line. The relationship between Qp and rQp has a larger error as shown for cell lines 4 and 5, which is partially due to the low number of replicates and the increased level of production in cell lines 4 and 5. However, the correlation is supported across the five different host cell lines and secreted proteins, confirming that microfluidic rQp is a useful measurement of relative productivity and relates to productivity seen at macroscale.

### Example 10. Stability Ranking

A set of clonal cell lines of known stability (Highly stable (11, 12, Unstable (16 and 13), and highly unstable (14, 15) were characterized within the microfluidic system as described in the Examples above to obtain rQp, and relevant relationships are shown in FIGS. 59A to 59C. The average rQp for each cell line is shown in FIG. 59A; the Hit rate (percent of secreting cells) as a function of time is shown in FIG. 59B and histograms of the sub-clone rQp distributions for each cell line 11-16 over 8 weeks, from Week 31 to Week 38, is shown in FIG. 59C, showing bimodal distributions in the unstable clones 13-16. The histogram displays Day 4 rQp measurements for each of the eight weeks.

Measuring the distribution of the sub-clonal rQp values provides additional information beyond just the average secretion and growth rates and provides other ways to rank the stability of the clones.

Using the secretion distribution, the percent of secreting sub-clones above the limit of detection (i.e. hit rate) also correlated with stability with more stable cell lines having a higher percent of secreting sub clones (FIG. 59B). The difference in secretion rate and therefore the resolution between cell lines increases with time as the unstable clones secretion continued to decrease. In addition, other features of the distribution, such as a bimodal distribution, correlated with instability. This may indicate that there were two distinct populations of sub-clones within the cell line increasing the likelihood of instability. For this data set all four unstable clones had a detectable bimodal distribution in the first measurement (Week 31).

While the present teachings are described in conjunction with various embodiments, it is not intended that the present teachings be limited to such embodiments. On the contrary, the present teachings encompass various alternatives, modifications, and equivalents, as will be appreciated by those of skill in the art.

Further, in describing various embodiments, the specification may have presented a method and/or process as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process should not be limited to the performance of their steps in the order written, and one skilled in the art can readily appreciate that the sequences may be varied and still remain within the spirit and scope of the various embodiments.

The embodiments described herein, can be practiced with other computer system configurations including hand-held devices, microprocessor systems, microprocessor-based or programmable consumer electronics, minicomputers, mainframe computers and the like. The embodiments can also be practiced in distributing computing environments where tasks are performed by remote processing devices that are linked through a network.

It should also be understood that the embodiments described herein can employ various computer-implemented operations involving data stored in computer systems. These operations are those requiring physical manipulation of physical quantities. Usually, though not necessarily, these quantities take the form of electrical or magnetic signals capable of being stored, transferred, combined, compared, and otherwise manipulated. Further, the manipulations performed are often referred to in terms, such as producing, identifying, determining, or comparing.

Any of the operations that form part of the embodiments described herein are useful machine operations. The embodiments, described herein, also relate to a device or an apparatus for performing these operations. The systems and methods described herein can be specially constructed for the required purposes or it may be a general-purpose computer selectively activated or configured by a computer program stored in the computer. In particular, various general-purpose machines may be used with computer programs written in accordance with the teachings herein, or it may be more convenient to construct a more specialized apparatus to perform the required operations.

Certain embodiments can also be embodied as computer readable code on a computer readable medium. The computer readable medium is any data storage device that can store data, which can thereafter be read by a computer system. Examples of the computer readable medium include hard drives, network attached storage (NAS), read-only memory, random-access memory, CD-ROMs, CD-Rs, CD-RWs, magnetic tapes, and other optical, FLASH memory and non-optical data storage devices. The computer readable medium can also be distributed over a network coupled computer systems so that the computer readable code is stored and executed in a distributed fashion.

### Example 11. Assays Conducted Under Flow and Non-Flow Conditions

Variation A: assays under flow conditions.

In one embodiment, the raw Assay Image may be normalized by subtracting both a Dark Reference image and a Signal Reference image correction from each pixel in the raw Assay Image as in the following equation:

Signal Reference value−Dark Reference

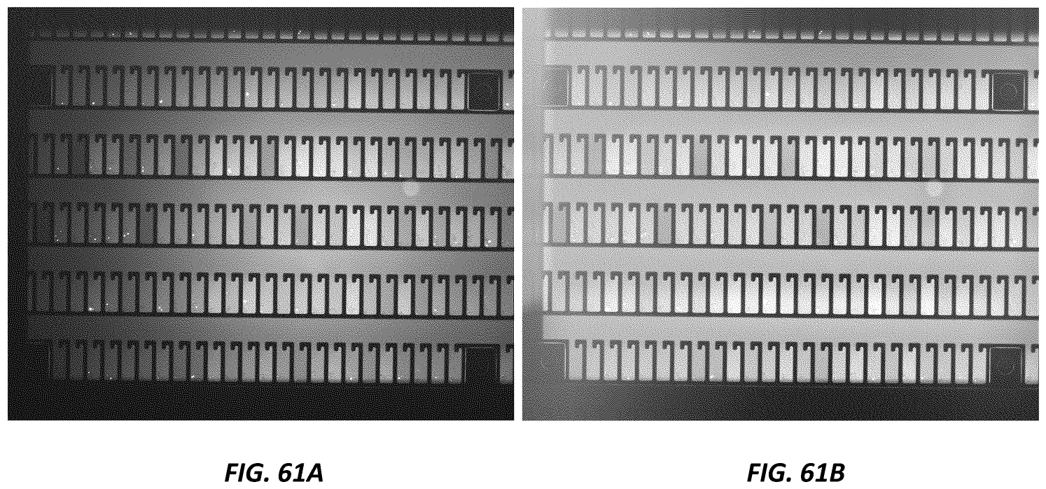

The Dark Reference image may be obtained by imaging the microfluidic device before flowing any medium into the device. Autofluorescence errors and other system errors can be corrected by subtracting the Dark Reference value at each pixel. The Signal Reference Image may correct for roll off, photobleaching errors or camera errors, and is obtained by flowing reporter molecule, or just the reporter molecule throughout the microfluidic device to reach an equilibrated concentration of the reporter molecule or fluorescent label. Each pixel in the raw Assay Image may be corrected in this manner, before extracting the fluorescence data for quantitation purposes. The normalized Assay Image is shown in FIG. 61B.

Variation B: assays under non-flow conditions.

As a first step in normalization, the Dark Reference image, as described above, was subtracted from the image of the microfluidic device with the bound and unbound reporter molecules present to produce an “dark reference subtracted image.”

As a second step, portions of the raw Assay image of FIG. 61A where the bound and unbound reporter molecules are not present (i.e. walls defining the chambers and channels in the microfluidic device) were removed or “masked” from the auto-fluorescence subtracted image to produce a “masked dark reference subtracted image.” As understood by those skilled in the art, this step also could be performed before the subtraction of auto-fluorescence.

As a third step in generating the normalized image of FIG. 61A, the intensity value for each pixel in the masked, auto-fluorescence subtracted image was divided by the global average intensity calculated based on all pixels in the masked, auto-fluorescence subtracted image. By dividing the intensity value for each pixel by the global average intensity, an image or similar data structure (e.g. a matrix) comprising a gain correction factor for each pixel is generated (“gain correction image”) is for each pixel of the image is produced. Other methods of producing a gain correction image are well known to those skilled in the art.

As a fourth step in generating the normalized image depicted in FIG. 61A, the gain-correction image was subject to a smoothing algorithm to reduce random noise. This step may not be employed in some embodiments of the method. Specifically, the gain-correction image was subject to a box-filter smoothing algorithm that used a 9-pixel by 9-pixel box-filter that accounts for the masked portions of the image in generating a local average for each pixel. As can be appreciated by those skilled in the art, other smoothing algorithms such as mean filtering, Gaussian filtering, gradient weighting filtering, sequence statistical filtering, robust smoothing filtering, Crimmins noise removal filtering, edge preserved filtering and self-adaptive median filtering may be used.

As a fifth step in generating the normalized photograph depicted in FIG. 61B, the smoothed gain-correction image may be multiplied by the auto-fluorescence subtracted image to produce a normalized image.

These methods may combine any of the foregoing steps and methods in the same or different sequence.

Variation C: assays under non-flow conditions.

Another method of normalizing the image may be used, depending on the substantially uniform concentration unbound reporter molecule within the channel due to its greater rate of diffusion over that of the bound RMSA complex. The brightness of the channels may be used to normalize the image to correct for the errors described above.

Therefore, in an alternate embodiment, the normalized image of FIG. 61B can be obtained using the brightness in the channels proximal to the chambers to correct for any variance in the amount of brightness across the view of regions of the microfluidic device. This method of normalization relies on the fact that the channels are not expected to have any analyte (or any RMSA complex) present and therefore can be performed using any area of the microfluidic device that does not have the analyte present.

In order to normalize based on the channel intensity, as a first step, a region of the channel R that is not expected to have any analyte present in it is identified for each chamber. In some embodiments, this region R can be a pre-defined region R corresponding to an area of the channel above the chamber. In other embodiments, the region R for each chamber can be identified based on other information or calculated based on the image.

For each region of the channel R, a brightness value B_{R} is computed based on the pixels within the region. Prior to computing the brightness values, the image used to calculate the brightness value may be subtracted, masked or otherwise processed as discussed above. In some embodiments, B_{R} is the average brightness value of the pixels within the region R.

After the average brightness value B_{R} for each region R is computed, the image of the chambers and channels may be partitioned into a series of areas A, where each area A encompasses a respective region R. This area may be computed so that a region R is in the center of an area A. In a specific embodiment, the areas A may be computed by generating a Voronoi diagram or a Delauney triangulation of the centroids of each region R. In other embodiments, each region R need not be centered in its respective area A and can be computed based on pre-defined areas segmenting the microfluidic device. For each area A, a gain-correction factor is calculated based on the maximum brightness value calculated for the brightest region B_{Rmax} divided by the brightness value B_{R} for the region R associated with the area A. The gain-correction factor may be used to generate a gain-correction image which can be multiplied against another image (e.g. the auto-fluorescence subtracted image) to produce a normalized image. The gain-correction factor image may also be smoothed as described above prior to use in normalization.

Quantification of the assay signal. In some embodiments, the diffusion profile of the RMSA may be used to quantify the amount of the RMSA complex present in the chamber. The diffusion profile provides a series of values (“concentration values”) that represent the concentration of the RMSA complex as it diffuses from its source to the channel.

After identification of the AOI, other transformations may be applied. For example, the pixels in each line may be processing by discarding outlier and/or aberrant pixels, other forms of global/local normalization, space conversion, and transforming the space of the pixel (e.g. from a multi-dimensional space to a two-dimensional space or vice-versa).

Depending on the embodiment, the intensity values may be used in different ways to calculate the concentration values. In some embodiments, the AOI may be sampled at fixed points to generate a set of concentration values corresponding to the intensity values at the fixed points. In some embodiments, the AOI may be segmented in a series of segments and the median or mean intensity of each segment may be calculated. Based on the embodiment and the degree of resolution required, the number of concentration values calculated can be as low as 1 and as high as the number of pixels in the line representing the diffusion trajectory.

Depending on the embodiment, the concentration values may be combined in different ways in order to quantify the amount of signal from the bound reporter molecule (and therefore the amount of secreted analyte) present. In some embodiments, the concentration values may be plotted to assess whether concentration values exhibit characteristics consistent with a diffusion profile. Depending on the embodiment, a number of algorithms may be used to fit a line to the concentration values and calculate characteristics of the line such as the slope and error associated with the line. Suitable line-fitting algorithms include: least-squares, polynomial fit, curve-fitting, and erfc fitting. Other algorithms are known to those skilled in the art. Methods of transforming fluorescence intensity values to obtain concentration values is described more fully below.

FIG. 62A is an Assay Image (photograph) of one chamber 6224, having an identification number “1107”, and wherein a line of anticipated diffusion trajectory 6252 is shown. An AOI 6250 is projected onto the Assay Image, and in this example, has a width of about 12 pixels, and it was segmented into 20 equal segments along the axis defined by the line (segments not shown). The median intensity for each of the 20 equal segments was calculated and then plotted as the concentration value in the graph of FIG. 62B. On the horizontal axis of the graphs, the segment numbers 1-20 are numbered according to their distance from the source (i.e. the cells secreting the secreted analyte), with the segment numbers having a low number representing the segment of the AOI closest to the cells in the region of the chamber most distal from the channel.

FIG. 62B depicts a series of curves representing concentration values for a set of chambers, which were generated according to the method discussed in the previous paragraph and other sections following. To generate the series of curves shown in FIG. 62B, the concentration values generated for each chamber were not normalized based on the number of cells in the chamber. However, in alternate embodiments, the concentration values and resultant curves may be normalized based on the number of cells in each chamber. As shown in FIG. 62B, the slope of the curve (of concentration values) for each chamber may be used to assess the relative amount of the secreted analyte present in each chamber. In other words, the slope may be used as a score such that chambers can be ranked and ordered relative to each other, and “slope” and “score”, in some embodiments herein, may be used interchangeably. In some instances, the score may be referred to as a secretion score. More specifically, in instances where the secreted analyte is produced by a biological micro-object (e.g. cell) present in the chambers, the slopes may be used to assess the relative ability of the cells in each chamber to produce the secreted analyte (e.g. the relative ability of cells to secrete an antibody). As discussed below, a relative or absolute amount of the secreted analyte may be calculated using different methods, including summing all the points in the sub-region of the AOI which is insensitive to the positions of the cells in the chamber and is most sensitive to variance in fluorescence intensity observed.

In addition, the shape of the curve may be evaluated to assess whether the concentration values for each chamber conform to expected parameters or indicate systemic error. For example, the shape of the curve labelled “Pen 1497” in FIG. 62B does not correspond to the shape of the curves observed for the other chambers whereas the shape of the curve labelled “Pen 1107” does corresponds to the expected diffusion profile. As shown in FIG. 62A, Pen 1107 had a visible gradient of reporter molecule from its chamber to the channel which resulted in its curve corresponding to an expected diffusion profile. As shown in FIG. 62C, a chamber 6226, having identification no. Pen 1497, has a line of anticipated diffusion trajectory 6252 and AOI 6250. However, chamber 6226 is proximal to a channel containing a bubble, where the meniscus 6201 of the bubble appears in the image as a white ellipse. The presence of the bubble results in the aberrant curve for Pen 1497 depicted in FIG. 62B. In various embodiments, the region of the segmented AOI that linear regression may be applied may be selected to be segments (sub-regions) 9-13, which as discussed above encompass portions of the connection region and have been identified to be most sensitive to fluorescence intensity variance and most insensitive to the location of biological micro-objects within the chamber.

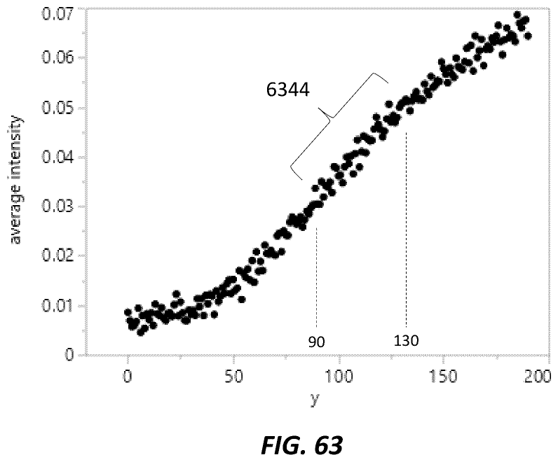

FIG. 63 shows an overlay of a plurality of curves representing intensity values (and thereby concentration values) derived via any of the methods described herein, obtained from a plurality of chambers within a microfluidic device. The intensity values of each point in each curve, plotted against the vertical axis of the graph, have been normalized for case of overlay. The values along the horizontal axis start with a value of “y” equal to zero, representing the first pixel in the y dimension of each AOI (and is physically located within the channel of the microfluidic device and outside of the chamber, similarly to the AOIs shown in FIG. 62A and FIG. 62C. The points along the horizontal axis marked “200” correspond to the last pixel in each AOI of the plurality of chambers, which is the boundary of the AOI closest to the cells secreting analyte, and hence the source from which the detectable signal from RMSA complex emanates. The concentration values obtained from the portion 6344 of the AOI that is least sensitive to the position of cells within the chamber and most sensitive to the variance in fluorescence intensities is shown in the portion of the curve associated with y values between about 90 and about 130, as shown. It can be seen that a mathematical operation imposing a linear shape in this region, and extracting the slope thereof, closely represents the state of the data.

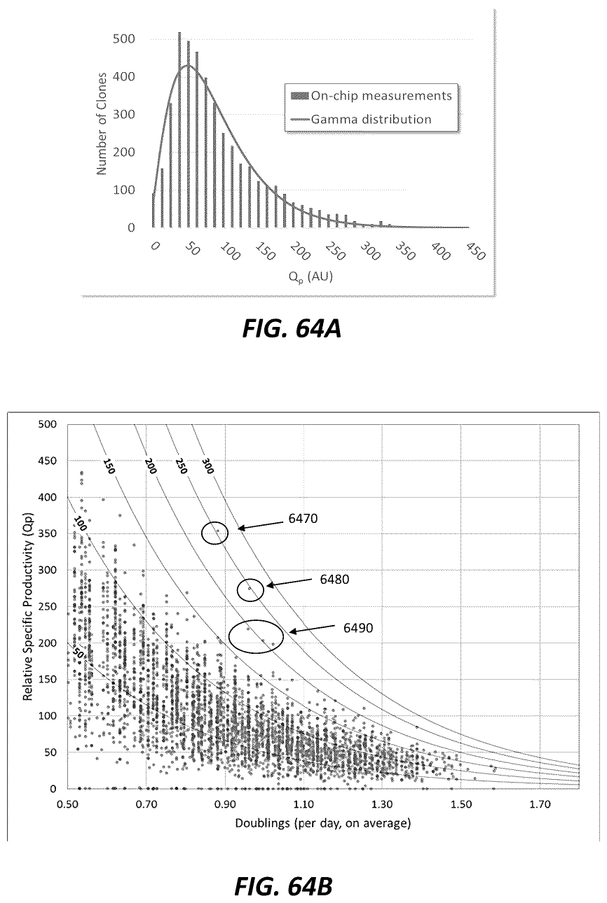

Performing the assay across the nanofluidic device containing thousands of clonal populations, each derived from a single cell placed into a separate chamber, can provide quantification of each of the clonal populations. As shown in FIG. 64A and FIG. 64B, the ability to find rare high producing clones is enhanced. If it is assumed that distribution of titers from a randomly secreting pool of cells is well described by Poisson statistics, then the titer distribution should fit to a gamma distribution. In FIG. 64A, the curve superimposed over the bar graph distribution of titers (which are obtained from the scores and normalized for number of cells present in each chamber of the plurality, and expressed in Arbitrary Units (A.U.) shows good agreement. There is a great majority of clonal populations expressing analyte from less than 50 to less than 100 A.U, and very few individual titers out in the high range of 250 A. U. and over. The same data is now shown plotting the relative specific productivity against rate of growth (along the horizontal axis). The curves superimposed on the graph show lines of constant titer, which again show that the majority of clones whether they are fast or slow growing clones, express the analyte at less than 100 A.U. and are not the desirable highly producing clones sought for cell line development. Only a few clones identified within the regions 6470, 6480, and 6490 are the rare high producers. However, these clones are not the fastest producing clones arising out of the originally seeded single cells. If these cells were mixed in with other cells as part of a larger growth environment, such as a well plate or a shaker flask, these rare, highly producing clones would most likely be overgrown by the faster growing, less productive clones. Trying to identify these clones if one attempted selection of random single cell sets for expansion, would require a massive sampling effort with massive input of resources to grow up the number of cells that would be required to have the probability of seeing them. In the system provided here, the titer (or score), may be obtained for all of the clonal populations, and the physical location of the productive clones is known. Further, only the selected clones may be selected and physically moved for further expansion/subcloning; selection and movement may be performed individually to prevent contamination by other cell populations. The opportunity to screen all of the clones arising from the originally seeded cells provides a greatly improved process for screening and selecting cells that secrete a desired analyte.

In some embodiments, fluorescence recovery after photobleaching (FRAP) can be another technique for measuring on-chip concentration measurements (i.e., secretion rate) of secreted molecules. The concentration and/or binding affinity of an unlabeled molecule secreted from a cell may be detectable by monitoring fluorescence recovery after photobleaching. Further details of FRAP techniques are found in WO2019/075476, filed on Oct. 15, 2018, the disclosure of which is herein incorporated by reference in its entirety.

### VIII. RECITATION OF SELECTED EMBODIMENTS

Embodiment 1. A method for characterizing a biological micro-object producing an analyte of interest, wherein the analyte of interest includes at least a first portion and a second portion different from the first portion, the method including: introducing the biological micro-object into a chamber of a microfluidic device, wherein the microfluidic device includes an enclosure having a flow region, and wherein the chamber is fluidically connected to the flow region; allowing the biological micro-object (or a clonal population of biological micro-objects generated therefrom) to secret the analyte of interest within the chamber; introducing a plurality of first reporter molecules into the flow region and allowing a portion of the plurality of first reporter molecules to diffuse into the chamber, wherein each of the plurality of first reporter molecules is configured to emit a detectable label (e.g., via a first detectable label) and includes a first binding component configured to bind the first portion of the secreted analyte of interest and thereby form a first reporter molecule: secreted analyte complex (first RMSA complex); introducing a plurality of second reporter molecules into the flow region and allowing a portion of the plurality of second reporter molecules to diffuse into the chamber, wherein each of the plurality of second reporter molecules is configured to emit a detectable signal (e.g., via a second detectable label) and includes a second binding component configured to bind the second portion of the secreted analyte of interest and thereby form a second reporter molecule: secreted analyte complex (second RMSA complex); detecting a first signal associated with the first detectable label within a first area of interest within the microfluidic device; detecting a second signal associated with the second detectable label within a second area of interest within the microfluidic device; and determining whether a ratio of the detected first signal to the detected second signal falls within a pre-selected range.

Embodiment 2. The method of embodiment 1 wherein the first area of interest and the second area of interest are substantially the same.

Embodiment 3. The method of embodiment 1 or 2, wherein: detecting the first signal includes determining a first absolute quantitation value of the detected first signal; detecting the second signal includes determining a second absolute quantitation value of the detected second signal; and determining whether the ratio of the detected first signal to the detected second signal falls within a pre-selected range includes determining whether a ratio of the first absolute quantitation value to the second absolute quantitation value falls within the pre-selected range.

Embodiment 4. The method of any one of embodiments 1 to 3, wherein allowing the portion of the plurality of first reporter molecules to diffuse into the chamber includes allowing the plurality of first reporter molecules to attain a steady state equilibrium between the flow region and the chamber.

Embodiment 5. The method of embodiment 4, wherein the steady state equilibrium of the plurality of first reporter molecules is attained within 3 hours (e.g., within 2.5 hours, within 2 hours, between about 2 hours and about 3 hours, or between about 2 hours and about 2.5 hours) of introducing the plurality of first reporter molecules into the flow region.

Embodiment 6. The method of embodiment 4 or 5, wherein detecting the first signal is performed after the steady state equilibrium of the plurality of first reporter molecules is reached.

Embodiment 7. The method of any one of embodiments 1 to 6, wherein allowing the portion of the plurality of second reporter molecules to diffuse into the chamber includes allowing the plurality of second reporter molecules to attain a steady state equilibrium between the flow region and the chamber.

Embodiment 8. The method of embodiment 7, wherein the steady state equilibrium of the plurality of second reporter molecules is attained within 3 hours (e.g., within 2.5 hours, within 2 hours, between about 2 hours and about 3 hours, or between about 2 hours and about 2.5 hours) or introducing the plurality of second reporter molecules into the flow region.

Embodiment 9. The method of embodiment 8, wherein detecting the second signal is performed after the steady state equilibrium of the plurality of second reporter molecules is reached.

Embodiment 10. The method of any one of embodiments 1 to 9, wherein: introducing a plurality of first reporter molecules includes introducing a first fluidic medium including the plurality of first reporter molecules into the flow region; and introducing a plurality of second reporter molecules includes introducing a second fluidic medium including the plurality of second reporter molecules into the flow region.

Embodiment 11. The method of embodiment 10, wherein: a concentration of the plurality of first reporter molecules in the first fluidic medium is about 1 to 10 times (e.g., about 1 to 5 times, or about 1 to 3 times) a dissociation constant (K_{D}) between the first binding component of the first reporter molecules and the first portion of the secreted analyte of interest; and/or a concentration of the plurality of second reporter molecules in the second fluidic medium is about 1 to 10 times (e.g., about 1 to 5 times, or about 1 to 3 times) a dissociation constant (K_{D}) between the second binding component of the second reporter molecules and the second portion of the secreted analyte of interest.

Embodiment 12. The method of embodiment 10 or 11, wherein the first fluidic medium and the second fluidic medium are introduced at the same time (e.g., the first fluidic medium and the second fluidic medium are the same).

Embodiment 13. The method of embodiment 10 or 11, wherein the first fluidic medium and the second fluidic medium are introduced at different times (e.g., sequentially).

Embodiment 14. The method of any one of embodiments 10 to 13, further including: introducing a third fluidic medium that is different than the first fluidic medium and the second fluidic medium.

Embodiment 15. The method of embodiment 14, wherein the third fluidic medium does not include first reporter molecules.

Embodiment 16. The method of embodiment 15, wherein the third fluidic medium is introduced after introducing the plurality of first reporter molecules (e.g., after introducing the first fluidic medium and, optionally, after steady state equilibrium of the plurality of first reporter molecules is reached).

Embodiment 17. The method of embodiment 16, wherein the third fluidic medium is introduced before introducing the plurality of second reporter molecules (e.g., before introducing the second fluidic medium).

Embodiment 18. The method of embodiment 16 or 17 further including: allowing at least a portion of unbound first reporter molecules to diffuse out of the chamber.

Embodiment 19. The method of any one of embodiments 14 to 18 further including: detecting a third signal associated with the first detectable label within a third area of interest within the microfluidic device.

Embodiment 20. The method of embodiment 19, wherein the first area of interest and the third area of interest are substantially the same.

Embodiment 21. The method of embodiment 19, wherein the first area of interest and the third area of interest are different.

Embodiment 22. The method of any one of embodiments 14 to 21, wherein the third fluidic medium does not include second reporter molecules.

Embodiment 23. The method of embodiment 22, wherein the third fluidic medium is introduced after introducing the plurality of second reporter molecules (e.g., after introducing the second fluidic medium and, optionally, after steady state equilibrium of the plurality of second reporter molecules is reached).

Embodiment 24. The method of embodiment 23, wherein the third fluidic medium is introduced before introducing the plurality of second reporter molecules (e.g., before and after introducing the second fluidic medium).

Embodiment 25. The method of embodiment 24 further including: allowing at least a portion of unbound second reporter molecules to diffuse out of the chamber.

Embodiment 26. The method of any one of embodiments 14 to 25 further including: detecting a fourth signal associated with the second detectable label within a fourth area of interest within the microfluidic device.

Embodiment 27. The method of embodiment 26, wherein the second area of interest and the fourth area of interest are substantially the same.

Embodiment 28. The method of embodiment 26, wherein the second area of interest and the fourth area of interest are different.

Embodiment 29. The method of any one of embodiments 1 to 28, wherein the flow region includes a microfluidic channel and wherein the chamber opens to the microfluidic channel.

Embodiment 30. The method of any one of embodiments 1 to 29, wherein the chamber includes an isolation region and a connection region fluidically connecting the isolation region to the flow region (or microfluidic channel), and further wherein the isolation region and the connection region are configured such that components of a fluidic medium in the isolation region are exchanged with components of a fluidic medium in the flow region (or microfluidic channel) substantially only by diffusion.

Embodiment 31. The method of embodiment 30, wherein the chamber includes an opening to the flow region (or microfluidic channel), and wherein the opening is oriented substantially parallel to a direction of flow of a fluidic medium in the microfluidic channel (e.g., when the fluidic medium is flowing in the microfluidic channel).

Embodiment 32. The method of embodiment 30 or 31, wherein the first area of interest includes: a portion of the isolation region of the chamber; a portion of the connection region; a portion of the flow region (or microfluidic channel); or any combination thereof.

Embodiment 33. The method of embodiment 32, wherein the first area of interest is along an axis of diffusion between the chamber and the flow region (e.g., along an axis of diffusion between the isolation region of the chamber and the microfluidic channel, such as along an axis of diffusion defined by the connection region).

Embodiment 34. The method of embodiment 32, wherein the first area of interest includes a portion of the isolation region of the chamber which, optionally, is not along an axis of diffusion between the chamber and the flow region (e.g., a portion of a hook region of the chamber).

Embodiment 35. The method of any one of embodiments 30 to 34, wherein the second area of interest includes: a portion of the isolation region of the chamber; a portion of the connection region; a portion of the flow region (or microfluidic channel); or any combination thereof.

Embodiment 36. The method of embodiment 35, wherein the second area of interest is along an axis of diffusion between the chamber and the flow region (e.g., along an axis of diffusion between the isolation region of the chamber and the microfluidic channel, such as along an axis of diffusion defined by the connection region).

Embodiment 37. The method of embodiment 35, wherein the second area of interest includes a portion of the isolation region of the chamber which, optionally, is not along an axis of diffusion between the chamber and the flow region (e.g., a portion of a hook region of the chamber).

Embodiment 38. The method of any one of embodiments 1 to 37, wherein allowing the biological micro-object (or clonal population of biological micro-objects generated therefrom) to secrete the analyte of interest including allowing the biological micro-object to secrete an analyte mixture including a plurality of analytes each having a molecule weight of about 1 kDa to about 600 kDa.

Embodiment 39. The method of any one of embodiments 1 to 38, wherein: a molecular weight of the first reporter molecule is equal to or less than about 150 kDa (e.g., about 2 kDa to about 150 kDa, about 2 kDa to about 100 kDa, about 2 kDa to about 75 kDa, about 2 kDa to about 50 kDa, about 2 kDa to about 25 kDa, or about 2 kDa to about 10 kDa); and/or a molecular weight of the second reporter molecule is equal to or less than about 150 kDa (e.g., about 2 kDa to about 150 kDa, about 2 kDa to about 100 kDa, about 2 kDa to about 75 kDa, about 2 kDa to about 50 kDa, about 2 kDa to about 25 kDa, or about 2 kDa to about 10 kDa).

Embodiment 40. The method of any one of embodiments 1 to 39, wherein the analyte of interest is a multi-specific antibody (e.g., a bispecific antibody, a trispecific antibody, etc.).

Embodiment 41. The method of any one of embodiments 1 to 40, wherein the first region of the analyte of interest is configured to recognize a first motif of a first target biomolecule, and wherein the first motif includes an amino acid, a nucleic acid, and/or a glycan.

Embodiment 42. The method of embodiment 41, wherein the first region of the analyte of interest is configured to bind to a region of glycosylation in the target biomolecule.

Embodiment 43. The method of embodiment 41 or 42, wherein the second region of the analyte of interest is configured to recognize a second motif of a second target biomolecule, and wherein the second motif includes an amino acid, a nucleic acid, and/or a glycan.

Embodiment 44. The method of embodiment 43, wherein the first target biomolecule and the second target biomolecule are different biomolecules.

Embodiment 45. The method of any one of embodiments 1 to 44, wherein the first binding component of the first reporter molecule includes an amino acid, a polypeptide, a nucleotide, a nucleic acid, or a combination thereof; and/or wherein the second binding component of the second reporter molecule includes an amino acid, a polypeptide, a nucleotide, a nucleic acid, or a combination thereof.

Embodiment 46. The method of embodiment 45, wherein the first binding component of the first reporter molecule includes a protein; and/or wherein second binding component of the second reporter molecule includes a protein.

Embodiment 47. The method of any one of embodiments 1 to 46, wherein the first detectable label of the first reporter molecule includes a visible, luminescent, phosphorescent, or fluorescent detectable label; and/or wherein the second detectable label of the second reporter molecule includes a visible, luminescent, phosphorescent, or fluorescent detectable label.

Embodiment 48. The method of any one of embodiments 1 to 47, wherein the chamber is a first chamber of the microfluidic device, and wherein the microfluidic device further includes at least a second chamber.

Embodiment 49. The method of embodiment 48, wherein: introducing a biological micro-object into the chamber includes: introducing a first biological micro-object into the first chamber; and introducing a second biological micro-object into the second chamber; detecting a first signal associated with the first detectable label within a first area of interest within the microfluidic device includes: detecting a first signal associated with the first detectable label within a first area of interest within and/or proximal to the first chamber; and detecting a fifth signal associated with the first detectable label within a fifth area of interest within and/or proximal to the second chamber; detecting a second signal associated with the second detectable label within a second area of interest within the microfluidic device includes: detecting a second signal associated with the second detectable label within a second area of interest within and/or proximal to the first chamber; and detecting a sixth signal associated with the second detectable label within a sixth area of interest within and/or proximal to the second chamber; and determining whether a ratio of the detected first signal to the detected second signal falls within a pre-selected range includes: determining whether a first ratio of the detected first signal to the detected second signal in the first chamber falls within the pre-selected range; and determining whether a second ratio of the detected fifth signal to the detected sixth signal in the second chamber falls within the pre-selected range.

Embodiment 50. The method of embodiment 49, further including: selecting the first biological micro-object, the second biological micro-object, both, or neither based on comparing each of the first ratio and the second ratio with the pre-selected range.

Embodiment 51. The method of any one of embodiments 1 to 50, further including exporting the biological micro-object (e.g., the first biological micro-object, the second biological micro-object, or both), or one or more biological micro-objects of a clonal population generated therefrom, from the chamber (e.g., the first chamber, the second chamber, or both) and, optionally, from the microfluidic device.

Embodiment 52. A method of assessing a secretion level of a biological micro-object secreting an analyte of interest, the method including: introducing a biological micro-object into a chamber of a microfluidic device, wherein the microfluidic device includes an enclosure having a flow region and the chamber, and wherein the chamber is fluidically connected to the flow region; allowing the biological micro-object (or a population of biological micro-objects generated therefrom) to secret an analyte mixture within the chamber, wherein the analyte mixture includes a plurality of analytes each having a molecule weight from about 1 kDa to about 600 kDa; introducing a first fluidic medium including a plurality of reporter molecules into the flow region, wherein each reporter molecule of the plurality of reporter molecules includes a detectable label and a binding component configured to bind the analyte of interest, and further wherein a concentration of the plurality of reporter molecules in the first fluidic medium is about 1 to 10 times (e.g., about 1 to about 5 times, or about 1 to about 3 times) a dissociation constant (K_{D}) between the reporter molecule and the analyte of interest, and a molecular weight of the reporter molecule equal to or less than about 150 kDa (e.g., about 2 kDa to about 150 kDa, about 2 kDa to about 100 kDa, about 2 kDa to about 75 kDa, about 2 kDa to about 50 kDa, about 2 kDa to about 25 kDa, or about 2 kDa to about 10 kDa); allowing the plurality of reporter molecules to diffuse into the chamber for a selected period of time, wherein the selected period of time is sufficient for establishing a steady state equilibrium of the reporter molecules between the flow region and the chamber and is within 3 hours (e.g., within 2.5 hours, within 2 hours, from about 2 hours to about 3 hours, or from about 2 hours to about 2.5 hours); and detecting a signal associated with the detectable label of the reporter molecule located within an area of interest within the microfluidic device.

Embodiment 53. The method of embodiment 52, further including: after the first fluidic medium is introduced into the flow region, introducing a second fluidic medium; wherein the second fluidic medium does not include the reporter molecule.

Embodiment 54. The method of embodiment 52 or 53 further including: allowing at least a portion of unbound reporter molecules to diffuse out of the chamber.

Embodiment 55. The method of any one of embodiments 52 to 54, wherein the flow region includes a microfluidic channel and wherein the chamber opens to the microfluidic channel.

Embodiment 56. The method of any one of embodiments 52 to 55, wherein the chamber includes an isolation region and a connection region fluidically connecting the isolation region to the flow region (or microfluidic channel), and further wherein the isolation region and the connection region are configured such that components of a fluidic medium in the isolation region are exchanged with components of a fluidic medium in the flow region (or microfluidic channel) substantially only by diffusion.

Embodiment 57. The method of embodiment 56, wherein the chamber includes an opening to the flow region (or microfluidic channel), and wherein the opening is oriented substantially parallel to a direction of flow of a fluidic medium in the microfluidic channel (e.g., when the fluidic medium is flowing in the microfluidic channel).

Embodiment 58. The method of embodiment 56 or 57, wherein the area of interest includes: a portion of the isolation region of the chamber; a portion of the connection region; a portion of the flow region (or microfluidic channel); or any combination thereof.

Embodiment 59. The method of embodiment 58, wherein the area of interest is along an axis of diffusion between the chamber and the flow region (e.g., along an axis of diffusion between the isolation region of the chamber and the microfluidic channel, such as along an axis of diffusion defined by the connection region).

Embodiment 60. The method of embodiment 58, wherein the area of interest includes a portion of the isolation region of the chamber which, optionally, is not along an axis of diffusion between the chamber and the flow region (e.g., a portion of a hook region of the chamber).

Embodiment 61. The method of any one of embodiments 52 to 60 further including: exporting the biological micro-object from the chamber and, optionally, from the microfluidic device.

Embodiment 62. The method of any one of embodiments 52 to 60, wherein the binding component of the reporter molecule includes an amino acid, a polypeptide, a nucleotide, a nucleic acid, or a combination thereof.

Embodiment 63. The method of embodiment 62, wherein the binding component of the reporter molecule includes a protein.

Embodiment 64. The method of any one of embodiments 52 to 63, wherein the detectable label of the reporter molecule includes a visible, luminescent, phosphorescent, or fluorescent detectable label.

Embodiment 65. The method of any one of embodiments 52 to 64, wherein introducing the biological micro-object into the chamber includes introducing a plurality of biological micro-object into the flow region of the microfluidic device and disposing a selected biological micro-object into the chamber.

Embodiment 66. A method for selecting a biological micro-object producing an analyte of interest, the method including: introducing a biological micro-object into a chamber of a microfluidic device, wherein the microfluidic device includes an enclosure having a flow region, and wherein the chamber is fluidically connected to the flow region; allowing the biological micro-object (or a clonal population of biological micro-objects generated therefrom) to secrete the analyte of interest within the chamber; introducing a plurality of reporter molecules into the flow region, wherein each reporter molecule of the plurality reporter molecules is configured to emit a detectable signal and includes a binding component configured to bind the analyte of interest; allowing a portion of the plurality of reporter molecules to diffuse into the chamber and bind to the secreted analyte of interest therein, thereby producing a plurality of reporter molecule:secreted analyte (RMSA) complexes; identifying one or more (e.g., plurality of) punctate regions emitting the detectable signal in an area of interest within the microfluidic device; and quantifying the one or more (e.g., plurality of) punctate regions in the area of interest.

Embodiment 67. The method of embodiment 66, wherein the one or more (e.g., plurality of) punctate regions include aggregated analytes of interest produced by the biological micro-object(s).

Embodiment 68. The method of embodiment 66 or 67, wherein the area of interest includes a region within the chamber that does not contain the biological micro-object (or the clonal population of biological micro-objects generated therefrom).

Embodiment 69. The method of embodiment 68, wherein the area of interest lies along an axis of diffusion between the chamber and the flow region (e.g., along an axis of diffusion between an isolation region of the chamber and a microfluidic channel of the flow region, such as along an axis of diffusion defined by a connection region of the chamber).

Embodiment 70. The method of embodiment 68, wherein the area of interest does not lie along an axis of diffusion between the chamber and the flow region (e.g., not along an axis of diffusion between an isolation region of the chamber and a microfluidic channel of the flow region, or not along an axis of diffusion defined by a connection region of the chamber).

Embodiment 71. The method of any one of embodiments 66 to 70, wherein the area of interest includes an image area corresponding to an area within the chamber that is most sensitive for measuring analyte concentration fluctuations, and/or least sensitive to a position of the biological micro-object(s) in the chamber when measuring analyte concentration fluctuations.

Embodiment 72. The method of any one of embodiments 66 to 71, wherein the area of interest includes a statically defined region of the chamber.

Embodiment 73. The method of any one of embodiments 66 to 71, wherein the area of interest is defined dynamically.

Embodiment 74. The method of embodiment 73, wherein defining the area of interest dynamically includes: acquiring a first brightfield image of the chamber prior to introducing the first fluidic medium, identifying a first position of the biological micro-object (or the population of biological micro-objects generated therefrom) in the chamber, and defining a respective cell-containing region within the chamber; and defining the area of interest within the chamber such that it excludes the cell-containing region within the chamber.

Embodiment 75. The method of embodiment 74, wherein defining the area of interest dynamically further includes: acquiring a second brightfield image of the chamber after allowing the portion of the plurality of reporter molecules to diffuse into the chamber, identifying a second position of the biological micro-object (or the population of biological micro-objects generated therefrom) in the chamber, and confirming that the biological micro-object (or the population of biological micro-objects is still contained within the cell-containing region within the chamber; and defining the area of interest within the chamber such that it excludes the confirmed cell-containing region within the chamber.

Embodiment 76. The method of any one of embodiments 66 to 75, wherein the binding component of the reporter molecules includes an amino acid, a polypeptide, a nucleotide, a nucleic acid, or any combination thereof.

Embodiment 77. The method of embodiment 76, wherein the binding component of the reporter molecules includes a protein.

Embodiment 78. The method of any one of embodiments 66 to 77, wherein each reporter molecule of the plurality of reporter molecules further includes a detectable label.

Embodiment 79. The method of embodiment 78, wherein the detectable label including a visible, luminescent, phosphorescent, or fluorescent detectable label.

Embodiment 80. The method of any one of embodiments 66 to 79, wherein identifying the one or more punctate regions emitting the detectable signal includes detecting a fluorescent signal.

Embodiment 81. The method of embodiment 80, wherein detecting the fluorescent signal includes detecting fluorescent signal having an intensity greater than a selected (e.g., threshold) level.

Embodiment 82. The method of any one of embodiments 66 to 81, wherein identifying the one or more punctate regions emitting the detectable signal includes using a Convolutional Neural Network (CNN).

Embodiment 83. The method of embodiment 82, wherein the CNN detects the detectable signal within a region having an area corresponding to an area having a minimum diameter of about 1 micron diameter or greater (e.g., a minimum diameter of about 2 microns, about 3 microns, about 4 microns, about 5 microns, about 10 microns, about 15 microns, or greater).

Embodiment 84. The method of any one of embodiments 66 to 83, wherein allowing the portion of the plurality of reporter molecules to diffuse into the chamber is performed for at least about 20 minutes (e.g., at least about 30 minutes, 40 minutes, 50 minutes, 60 minutes, or for about 20 minutes to about 60 minutes, or any value therebetween.

Embodiment 85. The method of embodiment 84, wherein allowing the portion of the plurality of reporter molecules to diffuse into the chamber is performed for about 45 minutes.

Embodiment 86. The method of any one of embodiments 66 to 85, wherein the plurality of reporter molecules is introduced in a first fluidic medium, and wherein a concentration of the plurality of reporter molecules in the first fluidic medium is from about 1 to about 10 times a dissociation constant (KD) of the binding component of the reporter molecules for the analyte of interest.

Embodiment 87. The method of any one of embodiments 66 to 86, wherein introducing the biological micro-object into the chamber includes introducing a plurality of biological micro-object into the flow region of the microfluidic device and disposing a selected biological micro-object into the chamber.

Embodiment 88. The method of any one of embodiments 66 to 87, wherein the microfluidic device includes a plurality of chambers and the method further includes: disposing each of a plurality of biological micro-objects into respective chambers of the plurality of chambers; allowing each disposed biological micro-object to secrete the analyte of interest within its respective chamber of the plurality of chambers; allowing a portion of the plurality of reporter molecules to diffuse into each respective chamber and bind the secreted analyte of interest therein, thereby producing a plurality of RMSA complexes in each respective chamber of the plurality of the chambers; identifying one or more (e.g., plurality of) punctate regions emitting the detectable signal in an area of interest within each respective chamber of the plurality of chambers; and quantifying the one or more (e.g., plurality of) punctate region within the area of interest within each respective chamber of the plurality of chambers.

Embodiment 89. The method of embodiment 88 further including: ranking the respective chambers of the plurality of chambers based on, at least in part, a number of the punctate regions within the area of interest of each respective chamber of the plurality of chambers.

Embodiment 90. The method of embodiment 88 or embodiment 89, wherein allowing a portion of the plurality of reporter molecules to diffuse into each respective chamber includes allowing the plurality of reporter molecules to reach a steady state equilibrium between the flow region and each respective chamber of the plurality of chambers, and wherein the method further includes: detecting, within the area of interest of each respective chamber of the plurality of chambers, a first diffuse signal emitted by each respective portion of the plurality of reporter molecules after the steady state equilibrium is reached; and ranking the respective chambers of the plurality of chambers based on, at least in part, an intensity of the respective detected first diffuse signals and a number of punctate regions within the respective areas of interest.

Embodiment 91. The method of any one of embodiments 88 to 90 further including: introducing a fluidic medium (e.g., a second fluidic medium) into the flow region of the microfluidic device, wherein the fluidic medium does not include the reporter molecules; allowing at least a portion of unbound reporter molecules to diffuse out of each respective chamber of the plurality of chambers; detecting, within the area of interest of each respective chamber of the plurality of chambers, a second diffuse signal emitted by reporter molecules retained within the respective chamber; and ranking the respective chambers of the plurality of chambers based on, at least in part, an intensity of the respective detected second diffuse signals and a number of punctate regions within the respective areas of interest.

Embodiment 92. The method of any one of embodiments 88 to 91, wherein a respective chamber of the plurality chambers is ranked highest provided the respective chamber has: a lowest number of punctate regions (e.g., a lowest amount of aggregation of analyte of interest); and a highest level of the detected first diffuse signal and/or the detected second diffuse signal (e.g., a highest level of secretion by the biological micro-object(s) disposed within the respective chamber).

Embodiment 93. The method of any one of embodiments 65 or 87 to 92, wherein before disposing the biological micro-object (or each biological micro-object) into the chamber (or its respective chamber of the plurality of the chambers), the method further includes: identifying a first subset of the plurality of biological micro-objects having a viable phenotype (e.g., by labeling the plurality of biological micro-objects with a first label); and/or identifying a second subset of the plurality of the biological micro-objects having an expressor cell phenotype (e.g., by labeling the plurality of biological micro-objects with a second label).

Embodiment 94. The method of embodiment 93, wherein identifying biological micro-objects having a viable phenotype includes labeling the plurality of biological micro-objects with a first label configured to positively or negatively label a live cell and/or determining a physical characteristic (e.g., size and/or shape) of each biological micro-object of the plurality of the biological micro-objects.

Embodiment 95. The method of embodiment 93 or 94, wherein identifying biological micro-objects having an expressor cell phenotype includes labeling the plurality of biological micro-objects with a second label configured to label a molecule of interest (e.g., the analyte of interest) at either a cell surface of a secreting biological micro-object or in a region proximal to (e.g., surrounding) the secreting biological micro-object.

Embodiment 96. The method any one of embodiments 93 to 95, wherein disposing each of the plurality of biological micro-objects into its respective chamber of the plurality of the chambers includes selectively disposing biological micro-objects that are members of both the first subset and the second subset of the plurality of biological micro-objects into respective chambers of the plurality of chambers.

Embodiment 97. The method of embodiment 96, wherein selectively disposing biological micro-objects that are members of both the first sub-set and the second sub-set of the plurality of biological micro-objects further includes: differentiating the biological micro-objects into at least two tiers based on the extent of the expressor cell phenotype (e.g., labeling of the second label); and prioritizing the disposing of biological micro-objects exhibiting a superior expressor cell phenotype (e.g., greater labeling of the second label).

Embodiment 98. The method of any one of embodiments 93 to 97, further including exporting biological micro-objects that are not members of both the first subset and the second subset from the microfluidic device without disposing any such biological micro-objects in a chamber of the plurality of chambers.

Embodiment 99. The method of any one of embodiments 93 to 98, wherein the first label includes Annexin V, propidium iodide, or a combination thereof.

Embodiment 100. The method of any one of embodiments 93 to 99, wherein the first sub-set is negatively labelled (e.g., not labeled) by the first label.

Embodiment 101. The method of any one of embodiments 93 to 98, wherein the first label includes a mitochondrial potential reagent (e.g., a reagent indicating energized and intact mitochondria).

Embodiment 102. The method of any one of embodiments 93 to 101, wherein the second label includes an IgG-binding reagent and, optionally, wherein the IgG-binding reagent includes a fluorescent label.

Embodiment 103. The method of embodiment 102, wherein the IgG-binding reagent includes a Protein A reagent or similar Fc domain-binding reagent.

Embodiment 104. The method of any one of embodiments 93 to 103, wherein the second label includes an antigen-specific cell surface marker, a glucose uptake reagent, or the like.

Embodiment 105. The method of any one of embodiments 93 to 104, wherein labelling the first subset of the plurality of the biological micro-objects is performed at 4° C. to 37° C. (e.g., 4° C. to 25° C., or 25° C. to 37° C.).

Embodiment 106. The method of any one of embodiments 93 to 105, wherein labelling the second subset of the plurality of the biological micro-objects is performed at 4° C. to 37° C. (e.g., 4° C. to 25° C., or 25° C. to 37° C.).

Embodiment 107. The method of any one of embodiments 93 to 106, wherein labelling the first subset of the plurality of biological micro-objects is performed for at least about 30 minutes (e.g., at least about 60 minutes, at least about 90 minutes, at least about 120 minutes, at least about 150 minutes, at least about 180 minutes, at least about 240 minutes, at least about 300 minutes, or more).

Embodiment 108. The method of any one of embodiments 93 to 107, wherein labelling the second subset of the plurality of biological micro-objects is performed for at least about 30 minutes (e.g., at least about 60 minutes, at least about 90 minutes, at least about 120 minutes, at least about 150 minutes, at least about 180 minutes, at least about 240 minutes, at least about 300 minutes, or more).

Embodiment 109. The method of any one of embodiments 93 to 108, wherein labelling the first subset is performed after labelling the second sub-set is performed.

Embodiment 110. The method of any one of embodiments 93 to 109, wherein the first label and/or the second label is formulated as a staining solution; and, optionally, wherein the staining solution further includes a staining enhancer.

Embodiment 111. The method of embodiment 110, wherein the staining enhancer includes Polyvinylpyrrolidone (PVP), Ficoll, Bovine serum albumin, or any combination thereof.

Embodiment 112. The method of embodiment 111, wherein the staining enhancer is Polyvinylpyrrolidone (PVP), and, optionally, wherein a concentration of the Polyvinylpyrrolidone (PVP) is about 0.01% to about 0.02% (w/v), about 0.012% to about 0.02% (w/v), or about 0.016% to about 0.019% (w/v).

Embodiment 113. The method of any one of embodiments 66 to 112, wherein introducing a biological micro-object into a chamber includes introducing a single biological micro-object into the chamber.

Embodiment 114. The method of any one of embodiments 66 to 113, further including culturing the biological micro-object introduced into the chamber (or biological micro-objects introduced into their respective chambers) thereby expanding the biological micro-object(s) into a clonal population (or clonal populations) thereof.

Embodiment 115. The method of any one of embodiments 66 to 114, wherein the flow region includes a microfluidic channel, and wherein an opening of the chamber opens to the microfluidic channel and is oriented substantially parallel to a direction of flow of a fluidic medium in the microfluidic channel (e.g., when the fluidic medium is flowing in the microfluidic channel).

Embodiment 116. The method of any one of embodiments 66 to 115, wherein the chamber includes an isolation region and a connection region fluidically connecting the isolation region to the flow region; and wherein the connection region includes the opening to the flow region.

Embodiment 117. The method of embodiment 116, wherein the isolation region is isolated from secondary flow that results from the flowing of fluidic medium in the flow region.

Embodiment 118. The method of embodiment 116 or embodiment 117, wherein the connection region is isolated from direct flow of fluidic medium flowing in the flow region.

Embodiment 119. A non-transitory computer-readable medium including a program for causing a computer to perform an image processing method for determining a quantity of aggregation products produced by a biological micro-object, the method including: receiving imaging data of a microfluidic device that includes a flow region and a plurality of chambers that are fluidically connected and open to the flow region; wherein the imaging data includes an aggregation assay image and, optionally, one or both of a background noise image and a signal reference image; defining an area of interest for each chamber; and determining scores that are indicative of the quantity of aggregation products in each chamber.

Embodiment 120. The non-transitory computer readable medium of embodiment 119, wherein the method includes the method of any one of embodiments 66 to 118.

Embodiment 121. The non-transitory computer readable medium of embodiment 120, wherein the quantity of aggregation products corresponds to a number of punctate regions in the respective chamber.

Embodiment 122. A method for enhanced loading of biological micro-objects secreting a molecule of interest (e.g., an analyte of interest) into a plurality of chambers of a microfluidic device, the method including (in any order): identifying a first subset of a plurality of biological micro-objects having a viable phenotype (e.g., by labeling the plurality of biological micro-objects with a first label); and/or identifying a second subset of the plurality of the biological micro-objects having an expressor cell phenotype (e.g., by labeling the plurality of biological micro-objects with a second label), wherein the method further includes: introducing the plurality of biological micro-objects into a flow region of the microfluidic device; and selectively disposing biological micro-objects that are members of both the first subset and the second subset of the plurality of biological micro-objects into respective chambers of the plurality of chambers.

Embodiment 123. The method of embodiment 122, wherein identifying biological micro-objects having a viable phenotype includes labeling the plurality of biological micro-objects with a first label configured to positively or negatively label a live cell and/or determining a physical characteristic (e.g., size and/or shape) of each biological micro-object of the plurality of the biological micro-objects.

Embodiment 124. The method of embodiment 122 or 123, wherein identifying biological micro-objects having an expressor cell phenotype includes labeling the plurality of biological micro-objects with a second label configured to label a molecule of interest (e.g., the analyte of interest) at either a cell surface of a secreting biological micro-object or in a region proximal to (e.g., surrounding) the secreting biological micro-object.

Embodiment 125. The method any one of embodiments 122 to 124, wherein selectively disposing biological micro-objects that are members of both the first sub-set and the second sub-set of the plurality of biological micro-objects further includes: differentiating the biological micro-objects into at least two tiers based on the extent of the expressor cell phenotype (e.g., labeling of the second label); and prioritizing the disposing of biological micro-objects exhibiting a superior expressor cell phenotype (e.g., greater labeling of the second label).

Embodiment 126. The method of any one of embodiments 122 to 125, further including exporting biological micro-objects that are not members of both the first subset and the second subset from the microfluidic device without disposing any such biological micro-objects in a chamber of the plurality of chambers.

Embodiment 127. The method of any one of embodiments 122 to 126, wherein the first label includes Annexin V, propidium iodide, or a combination thereof.

Embodiment 128. The method of any one of embodiments 122 to 127, wherein the first subset is negatively labelled (e.g., not labeled) by the first label.

Embodiment 129. The method of any one of embodiments 122 to 126, wherein the first label includes a mitochondrial potential reagent (e.g., a reagent indicating energized and intact mitochondria).

Embodiment 130. The method of any one of embodiments 122 to 129, wherein the second label includes an IgG-binding reagent and, optionally, wherein the IgG-binding reagent includes a fluorescent label.

Embodiment 131. The method of embodiment 130, wherein the IgG-binding reagent includes a Protein A reagent or a similar Fc domain-binding reagent.

Embodiment 132. The method of any one of embodiments 122 to 131, wherein the second label includes an antigen-specific cell surface marker, a glucose uptake reagent, or the like.

Embodiment 133. The method of any one of embodiments 122 to 132, wherein labelling the first subset of the plurality of the biological micro-objects is performed at 4° C. to 37° C. (e.g., 4° C. to 25° C., or 25° C. to 37° C.).

Embodiment 134. The method of any one of embodiments 122 to 133, wherein labelling the second subset of the plurality of the biological micro-objects is performed at 4° C. to 37° C. (e.g., 4° C. to 25° C., or 25° C. to 37° C.).

Embodiment 135. The method of any one of embodiments 122 to 134, wherein labelling the first subset of the plurality of biological micro-objects is performed for at least about 30 minutes (e.g., at least about 60 minutes, at least about 90 minutes, at least about 120 minutes, at least about 150 minutes, at least about 180 minutes, at least about 240 minutes, at least about 300 minutes, or more).

Embodiment 136. The method of any one of embodiments 122 to 135, wherein labelling the second subset of the plurality of biological micro-objects is performed for at least about 30 minutes (e.g., at least about 60 minutes, at least about 90 minutes, at least about 120 minutes, at least about 150 minutes, at least about 180 minutes, at least about 240 minutes, at least about 300 minutes, or more).

Embodiment 137. The method of any one of embodiments 122 to 136, wherein labelling the first subset is performed after labelling the second sub-set is performed.

Embodiment 138. The method of any one of embodiments 122 to 137, wherein the first label and/or the second label is formulated as a staining solution; and, optionally, wherein the staining solution further includes a staining enhancer.

Embodiment 139. The method of embodiment 138, wherein the staining enhancer includes Polyvinylpyrrolidone (PVP), Ficoll, Bovine serum albumin, or any combination thereof.

Embodiment 140. The method of embodiment 138, wherein the staining enhancer is Polyvinylpyrrolidone (PVP), and, optionally, wherein a concentration of the Polyvinylpyrrolidone (PVP) is about 0.01% to about 0.02% (w/v), about 0.012% to about 0.02% (w/v), or about 0.016% to about 0.019% (w/v).

Embodiment 141. The method of any one of embodiments 122 to 140, wherein selectively disposing a biological micro-object into its respective chamber includes introducing a single biological micro-object into the respective chamber.

Embodiment 142. The method of embodiment 141, further including culturing the biological micro-object selectively disposed into its respective chamber (or biological micro-objects selectively introduced into their respective chambers) thereby expanding the biological micro-object(s) into a clonal population (or clonal populations) thereof.

Embodiment 143. A non-transitory computer-readable medium including a program for causing a computer to perform an image processing method for enhanced loading of a plurality of biological micro-objects secreting a molecule of interest (e.g., an analyte of interest) into a respective plurality of chambers of a microfluidic device, wherein the method includes: receiving imaging data of a microfluidic device that includes a flow region and a plurality of chambers that are fluidically connected and open to the flow region, wherein the imaging data includes a loading image including an image of the plurality of biological micro-objects disposed within a portion of the flow region, and one or more fluorescent images of the portion of the flow region; defining from the loading image and the one or more fluorescent images a selected portion of the plurality of biological micro-objects including one or more selected characteristics; selecting individual biological micro-objects from the selected portion of the plurality of biological micro-objects; determining a trajectory to deliver each selected individual biological micro-object of the selected portion to a corresponding chamber of the plurality of chambers; and disposing each selected individual biological micro-object of the selected portion within its corresponding chamber.

Embodiment 144. The nontransitory computer-readable medium of embodiment 143, wherein the method further includes the method of any one of embodiments 122 to 142.

Embodiment 145. A method for determining relative stability for a plurality of clonal cell lines, the method including: receiving imaging data of a microfluidic device that includes a flow region and a first plurality of chambers that are fluidically connected and open to the flow region, wherein the imaging data includes a first analyte assay image taken of a plurality of subclones of a first cell line, wherein each subclone of the first cell line is disposed in an individual chamber of the first plurality of chambers; defining an area of interest for each chamber of the first plurality of chambers, wherein the area of interest includes an image area within the chamber that is suitable for assessing the level of secretion of an analyte of interest by the subclone disposed in the chamber (e.g., the area of interest may be a region that is most sensitive for measuring analyte concentration fluctuations, is least sensitive to the position of biological micro-objects in the chamber when analyte fluctuations are measured, and/or extends along an axis of diffusion between the chamber and the flow region); and generating a prediction of cell line stability based on a signal obtained from the area of interest, wherein the signal is an indicator of cell line stability.

Embodiment 146. The method of embodiment 145, wherein the microfluidic device further includes a second plurality of chambers that are fluidically connected and open to the flow region, and wherein the imaging data further includes a second analyte assay image taken of a plurality of subclones of a second cell line, wherein each subclone of the second cell line is disposed in an individual chamber of the second plurality of chambers.

Embodiment 147. The method of embodiment 146, wherein the microfluidic device further includes additional pluralities of chambers that are fluidically connected and open to the flow region (e.g., third, fourth, fifth, tenth, twelfth, etc. pluralities of chambers), and wherein the imaging data further includes additional analyte assay images taken of additional pluralities of subclones of additional cell lines (e.g., third, fourth, fifth, tenth, twelfth, etc. cell lines, respectively), and wherein each of the additional cell lines includes a set of subclones disposed in individual chambers of the corresponding plurality of chambers.

Embodiment 148. The method of embodiment 147, wherein the respective plurality of chambers allocated to a cell line (e.g., a first, second, third, fourth, fifth, tenth, twelfth, etc. cell line) is dependent on an assessment of the likelihood that the respective plurality of chambers will contain a number of viable clonal populations sufficient for determining the relative stability of the cell line.

Embodiment 149. The method of any one of embodiments 145 to 148, wherein the signal obtained from the area of interest represents a level of secretion of an analyte of interest, wherein the obtained signal is generated by assaying an intrinsic diffusion gradient (e.g., which may be an equilibration assay and/or a flush assay).

Embodiment 150. The method of any one of embodiments 145 to 149, wherein the first (and/or second, third, fourth, fifth, tenth, twelfth, etc.) analyte assay image is taken after the subclones have been cultured for sufficient time to undergo at least one cell division event.

Embodiment 151. The method of any one of embodiments 145 to 150, wherein the first (and/or second, third, fourth, fifth, tenth, twelfth, etc.) analyte assay image is taken between 1 and 10 days after loading the plurality of subclones of the first (and/or second, third, fourth, fifth, tenth, twelfth, etc. respective) clonal cell line into the first (and/or second, third, fourth, fifth, tenth, twelfth, etc. respective) plurality of chambers.

Embodiment 152. The method any one of embodiments 145 to 151, wherein generating a prediction of clonal cell line stability includes calculating a doubling time for each subclone of the first (and/or second, third, fourth, fifth, tenth, twelfth, etc.) cell line, wherein the prediction is based on a first (and/or second, third, fourth, fifth, tenth, twelfth, etc. respective) cell counting image (e.g., brightfield images) of the corresponding chamber of the subclone, and optionally, wherein the first (and/or second, third, fourth, fifth, tenth, twelfth, etc. respective) cell counting image includes a plurality of first cell counting images of the corresponding chamber obtained at a plurality of timepoints during the formation of the subclone cell population.

Embodiment 153. The method of embodiment 152, wherein a counting algorithm is applied to the first (and/or second, third, fourth, fifth, tenth, twelfth, etc.) cell counting image to generate a count of the number of cells in each chamber.

Embodiment 154. The method of embodiment 152, wherein a machine learning algorithm is used to analyze the first (and/or second, third, fourth, fifth, tenth, twelfth, etc.) cell counting image to obtain a cell count for each subclone of the first (and/or second, third, fourth, fifth, tenth, twelfth, etc.) cell line.

Embodiment 155. The method of embodiment 153 or 154, wherein the cell counting image, to which the counting algorithm is applied or the machine learning algorithm is used to analyze, is taken immediately before or immediately after the analyte assay image is taken.

Embodiment 156. The method of any one of embodiments 145 to 155, wherein generating a prediction of cell line stability includes calculating a simulated average rQp and/or a normalized average rQp.

Embodiment 157. The method of any one of embodiments 145 to 156, wherein a ranking of the relative stability of the first cell line and the second cell line (and/or third, fourth, fifth, tenth, twelfth, etc. cell line) is generated.

1. A method for characterizing a biological micro-object producing an analyte of interest, wherein the analyte of interest comprises at least a first portion and a second portion different from the first portion, the method comprising:
introducing the biological micro-object into a chamber of a microfluidic device, wherein the microfluidic device comprises an enclosure having a flow region, and wherein the chamber is fluidically connected to the flow region;
allowing the biological micro-object or a clonal population of biological micro-objects generated therefrom to secret the analyte of interest within the chamber;
introducing a plurality of first reporter molecules into the flow region and allowing a portion of the plurality of first reporter molecules to diffuse into the chamber, wherein each of the plurality of first reporter molecules comprises a first detectable label and a first binding component configured to bind the first portion of the secreted analyte of interest and thereby form a first reporter molecule: secreted analyte complex; and
introducing a plurality of second reporter molecules into the flow region and allowing a portion of the plurality of second reporter molecules to diffuse into the chamber, wherein each of the plurality of second reporter molecules comprises a second detectable label and a second binding component configured to bind the second portion of the secreted analyte of interest and thereby form a second reporter molecule: secreted analyte complex;
detecting a first signal associated with the first detectable label within a first area of interest within the microfluidic device;
detecting a second signal associated with the second detectable label within a second area of interest within the microfluidic device; and
determining whether a ratio of the detected first signal to the detected second signal falls within a pre-selected range.

2. The method of claim 1 wherein the first area of interest and the second area of interest are substantially the same.

3. The method of claim 1, wherein:
detecting the first signal comprises determining a first absolute quantitation value of the detected first signal;
detecting the second signal comprises determining a second absolute quantitation value of the detected second signal; and
determining whether the ratio of the detected first signal to the detected second signal falls within a pre-selected range comprises determining whether a ratio of the first absolute quantitation value to the second absolute quantitation value falls within the pre-selected range.

4. The method of claim 1, wherein allowing the portion of the plurality of first reporter molecules to diffuse into the chamber comprises allowing the plurality of first reporter molecules to attain a steady state equilibrium between the flow region and the chamber.

5. The method of claim 4, wherein detecting the first signal is performed after the steady state equilibrium of the plurality of first reporter molecules is reached.

6. The method of claim 5, wherein allowing the portion of the plurality of second reporter molecules to diffuse into the chamber comprises allowing the plurality of second reporter molecules to attain a steady state equilibrium between the flow region and the chamber.

7. The method of claim 1, wherein:
introducing a plurality of first reporter molecules comprises introducing a first fluidic medium comprising the plurality of first reporter molecules into the flow region; and
introducing a plurality of second reporter molecules comprises introducing a second fluidic medium comprising the plurality of second reporter molecules into the flow region.

8. The method of claim 7, wherein:
a concentration of the plurality of first reporter molecules in the first fluidic medium is about 1 to 10 times a dissociation constant (K_{D}) between the first binding component of the first reporter molecules and the first portion of the secreted analyte of interest; and/or
a concentration of the plurality of second reporter molecules in the second fluidic medium is about 1 to 10 times a dissociation constant (K_{D}) between the second binding component of the second reporter molecules and the second portion of the secreted analyte of interest.

9. The method of claim 7, further comprising: introducing a third fluidic medium that is different than the first fluidic medium and the second fluidic medium.

10. The method of claim 9, wherein the third fluidic medium does not comprise first reporter molecules.

11. The method of claim 10, further comprising: detecting a third signal associated with the first detectable label within a third area of interest within the microfluidic device.

12. The method of claim 9, wherein the third fluidic medium does not comprise second reporter molecules.

13. The method of claim 12, further comprising: detecting a fourth signal associated with the second detectable label within a fourth area of interest within the microfluidic device.

14. The method of claim 1, wherein the flow region comprises a microfluidic channel and wherein the chamber opens to the microfluidic channel.

15. The method of claim 1, wherein the chamber comprises an isolation region and a connection region fluidically connecting the isolation region to the flow region, and further wherein the isolation region and the connection region are configured such that components of a fluidic medium in the isolation region are exchanged with components of a fluidic medium in the flow region substantially only by diffusion.

16. The method of claim 15, wherein the chamber comprises an opening to the flow region, and wherein the opening is oriented substantially parallel to a direction of flow of a fluidic medium in the microfluidic channel.

17. The method of claim 15, wherein the first area of interest comprises: a portion of the isolation region of the chamber; a portion of the connection region; a portion of the flow region; or any combination thereof.

18. The method of claim 15, wherein the second area of interest comprises: a portion of the isolation region of the chamber; a portion of the connection region; a portion of the flow region (or microfluidic channel); or any combination thereof.

19. The method of claim 1, wherein the analyte of interest is a multi-specific antibody.

20. The method of claim 1, wherein the first region of the analyte of interest is configured to recognize a first motif of a first target biomolecule, and wherein the first motif comprises an amino acid, a nucleic acid, and/or a glycan.

21. The method of claim 20, wherein the first region of the analyte of interest is configured to bind to a region of glycosylation in the target biomolecule.

22. The method of claim 20, wherein the second region of the analyte of interest is configured to recognize a second motif of a second target biomolecule, and wherein the second motif comprises an amino acid, a nucleic acid, and/or a glycan.

23. The method of claim 22, wherein the first target biomolecule and the second target biomolecule are different biomolecules.

24.-56. (canceled)
